(19) United States
(12) Patent Application Publication
(10) Pub. No.: US 2026/0041378 A1
(43) Pub. Date: Feb. 12, 2026

(54) METHODS OF AND DEVICES/APPARATUS FOR DETECTING, TRIGGERING, AND USING CELL-TO-CELL COMMUNICATION

(71) Applicant: Immunolight, LLC, Detroit, MI (US)

(72) Inventors: Frederic A. BOURKE, JR., Aspen, CO (US); Zakaryae FATHI, Raleigh, NC (US); Harold WALDER, Oak Island, NC (US); Wayne F. BEYER, JR., Ivanhoe, VA (US); Joseph H. SIMMONS, Tucson, AZ (US); Ronald RUDDER, Clarksville, VA (US)

(73) Assignee: Immunolight, LLC, Detroit, MI (US)

(21) Appl. No.: 19/100,044

(22) PCT Filed: Aug. 23, 2023
(86) PCT No.: PCT/US2023/072705

§ 371 Date: Jan. 30, 2025

Related U.S. Application Data
(60) Provisional application No. 63/373,475, filed on Aug. 25, 2022.

(51) Int. Cl. A61B 5/00

(52) U.S. Cl. CPC A61B 5/7271; A61B 5/0071; A61B 5/4836

(57) ABSTRACT

Methods for detecting, stimulating, and/or monitoring communication within a cell or between cells are provided, and methods of treating a subject having a disease, disorder or condition by causing communication within a cell or between cells, along with devices for detecting communication within a cell and between cells.

### CROSS REFERENCE TO RELATED APPLICATIONS

This application claims priority to U.S. Provisional Application Ser. No. 63/373,475, filed Aug. 25, 2022, pending. This application is related to U.S. Ser. No. 16/599,732, filed Oct. 11, 2019, the entire contents of which are hereby incorporated herein by reference. This application is related to U.S. Ser. No. 62/745,057, filed Oct. 12, 2018, the entire contents of which are hereby incorporated herein by reference. This application is related to each of U.S. Ser. No. 15/874,426, filed Jan. 18, 2018, pending; U.S. Ser. No. 15/649,956, filed Jul. 14, 2017, now U.S. Pat. No. 9,993,661; U.S. Ser. No. 14/131,564, filed Jul. 11, 2014, now U.S. Pat. No. 9,907,976; PCT application PCT/US12/045930, filed Jul. 9, 2012; U.S. Provisional Ser. No. 61/505,849 filed Jul. 8, 2011, expired; U.S. Ser. No. 15/151,642, filed May 11, 2016, pending; U.S. Ser. No. 12/417,779, filed Apr. 3, 2009, abandoned; U.S. Provisional Ser. No. 61/042,561, filed Apr. 4, 2008, expired; U.S. Provisional Ser. No. 60/954,263, filed Aug. 6, 2007, expired; U.S. Provisional Ser. No. 61/030,437, filed Feb. 21, 2008, expired; U.S. Ser. No. 12/059,484, filed Mar. 31, 2008, abandoned; U.S. Ser. No. 11/935,655, filed Nov. 6, 2007, now U.S. Pat. No. 9,358,292; U.S. Provisional Ser. No. 61/042,561, filed Apr. 4, 2008, expired; U.S. Provisional Ser. No. 61/035,559, filed Mar. 11, 2008, expired; U.S. Provisional Ser. No. 61/080,140, filed Jul. 11, 2008, expired; U.S. Ser. No. 12/401,478 filed Mar. 10, 2009, now U.S. Pat. No. 8,376,013; U.S. Ser. No. 12/059,484, filed Mar. 31, 2008, abandoned; U.S. Ser. No. 12/389,946, filed Feb. 20, 2009, now U.S. Pat. No. 8,951,561; U.S. Ser. No. 12/417,779, filed Apr. 3, 2009, abandoned; U.S. Provisional Ser. No. 61/161,328, filed Mar. 18, 2009, expired; PCT application PCT/US2009/050514, filed Jul. 14, 2009, expired; U.S. Ser. No. 12/725,108, filed Mar. 16, 2010, now U.S. Pat. No. 8,389,958; U.S. Ser. No. 12/764,184, filed Apr. 21, 2010, now U.S. Pat. No. 9,302,116; U.S. Provisional Ser. No. 61/443,019, filed Feb. 15, 2011, expired; U.S. Ser. No. 13/732,882, filed Jan. 2, 2013, now U.S. Pat. No. 8,618,509; U.S. Ser. No. 14/688,687, filed Apr. 16, 2015, now U.S. Pat. No. 10,080,276; U.S. Ser. No. 15/307,766, filed Oct. 28, 2016, pending; PCT application PCT/US2017/029300, filed Apr. 25, 2017, pending; U.S. Provisional No. 62/897,677, filed Sep. 9, 2019, pending; and U.S. Provisional No. 62/327,121, filed Apr. 25, 2016, expired; the entire contents of each of which is incorporated herein by reference.

### BACKGROUND OF THE INVENTION

### Field of Invention

The invention pertains to ways to detect and use cell-to-cell communication to induce, affect, or monitor a biological change in a medium, particularly a living subject.

### Discussion of the Background

Light modulation from a deeply penetrating radiation like X-ray to a photo-catalytic or therapeutic radiation like UV or IR, opens the possibility for activating bio-therapeutic agents and processes of various kinds within living subjects, particularly mammalian bodies. Other possibilities include the activation of photo-catalysts in mediums for cross-linking reactions in polymeric chains and polymer based adhesives. These examples are but two examples of a number of possibilities that can be more generally described as the use of a conversion material to convert an initiating radiation that is deeply penetrating to another useful radiation possessing the capability of promoting photo-based chemical reactions. The photo-chemistry is driven inside media of far ranging types including organic, inorganic or composited from organic and inorganic materials.

Photo-activation with no line of site required can be done in-vivo and ex-vivo such as those carried out in cell cultures. In turn, the photo activation of a select bio-therapeutic agent, and conceivably more than one agent at a time, can lead to the onset of a desirable chemical reaction, or a cascade of reactions, that in turn lead to a beneficial therapeutic outcome. As an example, the binding of psoralen compounds to DNA through the formation of monoadducts and/or crosslinked adducts is well known to engender an immune response if done properly.

As background as to the physical and biological structures that the present invention can address, below is a summary of the various anatomical cell structures present in subjects to which the techniques of the present invention disclosed below can apply. Further details of anatomical structures can be found in U.S. Pat. No. 9,295,835 (the entire contents of which are incorporated herein by reference).

As described in the '835 patent, humans and animals are constructed of cells. Cells are the smallest fundamental unit of life. A cell is considered to be the smallest living structure capable of performing all of the processes that define life. The human body is made up of some 100 trillion cells representing perhaps some 300 cell-types. Each cell-type performs a specific function such as operating muscles, glands, and vital organs. In addition, nerves, which are made of communicating-cells called neurons, provide electrical regulating signals to operate and adjust enormous amounts of functional activities throughout the body to maintain homeostasis (life equilibrium).

FIG. 1 is a schematic illustrating various cellular components of an example cell 100. The depiction shown in FIG. 1 illustrates, for example, cellular components such as mitochondria, ribosomes, centrosome, centrioles, the nucleus, and so on.

FIG. 2 illustrates a schematic drawing of the structure of a plasma membrane 100 of the cell 100 shown in FIG. 1. Cells are known to have a complex cellular wall referred to in the art as a plasma membrane, an example of which is shown in FIG. 2. A portion 200 of the plasma membrane is shown in FIG. 2 with respect to the cell 100. The plasma membrane separates the internal structures and operating organelles from the cell's external environment. It houses and protects the contents of the cell. It is made of a bi-layer of phospholipids and various proteins, which are attached or embedded.

The plasma membrane is a semi permeable structure that allows passage of nutrients, ions, water, and other materials into the cell. It also allows an exit pathway for waste products and for functional two-way passage of many kinds of molecules to adjust cell chemistry. The principal purpose of the cell membrane is to provide a barrier that contains all of the processes and components within the living cell and to simultaneously repel unwanted substances from invading or entering the cell.

There are some 300 types of ion pores in a plasma membrane for purposes of transporting the raw materials used by the cell to live and to perform its duties.

The plasma membrane may have from a relatively small number of ion channels up to approximately 200 to 400 molecular channels or more and of different dimension through which the passage of nutriments and electrolytic ions can enter the cell. The thickness of a plasma membrane is estimated to be about 7-8 nanometers. In addition, selected ion channels can rid the individual cells of waste products in a process called autophagy to transport, excrete or see to the expulsion of waste products from the cellular interior into the extracellular space.

The molecular channels within the plasma membrane have molecular sized openings for the different molecules of extracellular ions and other nutriments or materials required by the cell. An example of the materials desired by the cell to transport through these channels include, but is not limited to, sodium, potassium, magnesium, calcium ions, and water. The openings are provided for example by specifically sized pores through which ions can travel between extracellular space and cell interior. The channels are typically specific (selective) for one ion; for example, most potassium channels are characterized by 1000:1 selectivity ratio for potassium over sodium, though potassium and sodium ions have the same charge and differ only slightly in their radius. The channel pores are typically so small that ions must pass through in a single-file order.

A channel may have several different states (corresponding to different conformations of a protein), with each state considered to be either open or closed. In general, closed states correspond either to a contraction of the pore—making it impassable to the ion—or to a separate part of the protein, stoppering the pore. For example, the voltage-dependent sodium channel undergoes inactivation, in which a portion of the protein swings into the pore, sealing it. This inactivation shuts off the sodium current.

Ion channels can also be classified by how the channels respond to their environment. For example, the ion channels involved in an action potential are voltage-sensitive channels; they open and close in response to the voltage across the membrane. Ligand-gated channels form another important class; these ion channels open and close in response to the binding of a ligand molecule, such as a neurotransmitter. Other ion channels open and close with mechanical forces. Still other ion channels—such as those of sensory neurons—open and close in response to other stimuli, such as light, temperature or pressure. Light (an electromagnetic radiation) was therefore demonstrated to enable the triggering of certain cellular functions.

The passage of ions through the cellular membrane participates, generates, and/or creates a flow of electric currents within the membrane and/or on the inner surface of the plasma membrane. At points where the cytoskeleton, intermediate-filaments or microfilaments are attached to the plasma membranes, those points allow “signals” to gain entry onto the cytoskeleton so as to be able to serve as a pathway to transport the signals around the cell. Such signals may travel to trigger or adjust chemical reaction areas and to various organelles and the nucleus to trigger reactions and pass along cellular communication instructions, at a minimum. Cells were therefore demonstrated to be equipped with an enabling infrastructure to sense and react to stimuli including electrical and electromagnetic stimuli.

Since cells are electrochemical in nature, the plasma membrane is the site for generating the cells electrical signals for metabolic and other operations and to serve as a means to communicate, relay and receive signals with other cells, especially those of similar type. The nucleus and plasma membrane communicate with electrical signals. The nucleus determines how the cell functions and also determines the architecture of the cell and its contents. The plasma membrane uses electrical signaling to open passageways and ion channels to allow the intake of chemicals as well as the outflow of cellular waste products.

The electric signaling exists by virtue of potential gradients and the establishment of currents that exist within cells and between cells within biological bodies. The displacement of charged species encompasses electrons, ions, anions, low, mid and high molecular weight biological polymers, which in turn includes, but is not limited to, proteins. It is well known that the displacement of charged species (current) is almost always accompanied by the establishment of magnetic fields during the transient states associated with motion.

The outside of the cell membrane is coated with a defensive glycocalyx, which is designed and produced by the cell to protect it and allow it to be recognized. The nucleus has input into the crafting of membrane defensive characteristics. The glycocalyx can produce a negative electric surface charge in cancer cells so as to repel the body's immune system.

The cell membrane regulates the flow of materials into and out of the cell. Also, it can detect external signals and mediate interactions between other cells. Membrane carbohydrates installed on the outer surface function as cell markers to distinguish itself from other cells.

This plasma membrane contains the sites where the electrical energy is created and the cellular communication signals are formed. These signals are transmitted over the cytoskeleton, which acts like wires, to regulate and trigger metabolic and functional processes within the cell. The cell nucleus communicates with all organelles and operating structures located within the cell. FIG. 3, for example, illustrates a junction view 300 of the attachments between tumor cells.

FIG. 4 illustrates a pictorial drawing of the internal framework 400 of a cell, such as the cell 100 shown in FIG. 1.

The cytoskeleton in a cell maintains the shape of all cells from the inside. It is like a geodesic structure that provides strength and internal areas for electro-chemical timed reactions. Noteworthy is that the cytoskeleton extends into other cells and links up with their cytoskeleton to maintain and form communication links into adjacent cells. This structure is made up of a network of hollow-microtubules, solid-microfilaments, and solid-intermediate filaments. The cytoskeleton is anchored to the plasma membrane and serves as the ‘wiring’ to transmit the cellular communication signals. The cellular environment is highly networked and the transmission of chemical and electrical information is made more efficient as a result if this interconnectivity.

The cytoskeleton is made up of actin and myosin, which are also found in muscle structures. The cytoskeleton also controls the circulation of the cytosol, which is the fluid and semi-fluid that suspends the organelles. Organelles are the functioning entities of the cell that manufacture and distribute cellular products and processes necessary for the cell to live.

The cytoplasm in a cell is a fluid, that can be rather gel-like, which surrounds the nucleus, which is considered an organelle. The nucleus contains the DNA genetic information and hence, controls both the activity of the cell and its structural nature. The nucleus is spherical and is surrounded by a double membrane, the nuclear membrane and envelope, which is perforated by a significant number of pores that allow the exchange of materials and substances with the cell's cytoplasm and the extra moist environment outside which contains the ionic minerals and chemicals that feed the cells and provides the necessary water.

The nucleus in the cell is an electrical body which contains the cell's DNA and carries programs to operate its electrical signals and the opening and closing of channels in the wall of the cell's plasma membrane. The nucleus also contains the apoptosis program for cell suicide. Depending on the duties of the cell, some use ion channels that function electrically and others are influenced by chemicals that it obtains from the extra cellular media. Ion pumps and ion channels are electrically equivalent to a set of batteries and resistors inserted in the membrane wall, and thus create voltage differences between the inner and outer sides of the membrane. Such differences in the electrical values range from −40 mV to −80 mV. Because the cell acts as a battery, it provides the power to operate molecular devices that are embedded in the plasma membrane. As described in the '835 patent, the electrical activity sends signals that communicate with adjoining cells of the tumor to regulate the cancer as an intra grail living body.

An important organelle in the cell is the mitochondrion, which serves as the power station for the cell. Mitochondria are rod or oval shaped structures functioning as respiration for the cell. A number of mitochondria are distributed within the cytoplasm and move in accordance with its flow. The product produced as a biological fuel is called adenosine tri-phosphate (ATP). The manufacture of ATP results from the processing of proteins, fats, and carbohydrates through the Krebs cycle. The ATP once produced is distributed to other organelles that require this bio-fuel to provide processing energy as needed.

The mechanism of energy production is known as oxidative phosphorylation. The membrane of the live biological cell and the membrane of the mitochondria are analogous to plate-like condensers with defined capacitance related to the surface area, the permittivity of the biological media and is inversely proportional to the distance between the surfaces. The pumping of ions into the intermembrane space leads to a voltage build up and the process is analogous to a metabolic pump with a defined voltage gradient and hence a power supply to drive an electromotive force.

The endoplasmic reticulum (ER) in a cell is a network of membranes that form channels that crisscross the cytoplasm utilizing its tubular and vesicular structures to manufacture various molecules. The network of membranes is dotted with small granular structures called ribosomes for the synthesis of proteins. Ribosomes are tiny spherical organelles distributed around the cell in large numbers to synthesize cell proteins. They also create amino acid chains for protein manufacture. Ribosomes are created within the nucleoli at the level of the nucleolus and then released into the cytoplasm.

Smooth ER makes fat compounds and deactivates certain chemicals like alcohol or detected undesirable chemicals such as pesticides. Rough ER makes and modifies proteins and stores them until notified by the cell communication system to send them to organelles that require the substances. Cells in humans, except erythrocytes (red blood cells), are equipped with endoplasmic reticulum.

The Golgi apparatus is made of Golgi bodies, which are located close to the nucleus and are made of flattened membranes stacked atop one another like a stack of plates. The Golgi apparatus sorts and modifies proteins and fats made by the ER, after which it surrounds and packs them in a membranous vesicle so they can be moved around the cell as needed. Similarly, there is a process to pack up cell waste products for expulsion from the cell via ports in the plasma membrane into the extra cellular spaces.

Lysosomes are the digestive system for the cell. They contain copious quantities of acids, enzymes, and phosphates to break down microbes and other undesirable substances that have entered the cell. They also digest and recycle worn-out organelles to make new cellular structures or parts.

As described in the '835 patent, the cytoskeleton is composed and constructed of intermediate sized filaments, which actually serve as the internal structure to maintain cellular shape. The filamentous structure serves to provide a highway for electrical signals to travel to sites of chemical process that reside on shelves constructed by the cytoskeleton assembly within the cell. The intermediate filaments are composed of compounds that are similar to the structures of muscles, which have their own electrical properties. The electrical signals traveling through or on the cytoskeleton most likely initiate and stop the chemical reactions, as required. The electrical signals may skip and travel along the surface of the filamentous network rather than within the central framework, again on some sort of scheduled or timed basis or in response to some event or instruction. Access to all systems within the cell by nucleus operations is made possible by electrical signals residing within the individual cells.

As described in the '835 patent, cells become more electro-negative in the course of cancerization. Cancer cells seem to reconstruct the cellular membrane access ports to allow the importation of more sodium and sugars than non-cancerous cells of the same size. The electrical potential between the inner and exterior layers of the plasma membrane serve as a sort of electrical generator to supply the power to operate the individual cancer cell.

The cytoskeleton intermediate filaments are considered to be hooked together with a sort of “Velcro” at its connection points throughout the cells interior to allow some flexing of the overall cellular structure. Importantly, the intermediate filaments continue protruding through the desmosome which allows a connection to an adjoining cancer cell. This piercing of the cell wall within the desmosome is considered to one way explaining how signals are sent and received from adjoining cells. There can be several desmosome connections on different aspects of the cell wall (plasma membrane) so as to connect to cells over, under, and beside for example a given cancerous cell, so as to provide a connected network for communication. In the alternative, other types of cellular attachment for signal transduction or transmission are likely.

Normal cells reproduce by going through a cell cycle that leads to reproduction of similar cells by a process of mitosis which is where a single cell divides and then splits into two daughter cells that are exact replications of the mother cell. Normal cells are limited as to how many times they can reproduce by mitosis, which is probably no more than 70 times.

Cancer occurs in normal cells in which birth-defected distorted chromosomes and abnormal genes can lead to the formation of a defective cell which exhibits a severe disorder of mitosis (cell division). The thrust of a cancerized cell is to continuously reproduce by splitting into similar daughter cells uncontrollably for its entire life. Some species of cancer cells can reproduce continuously every 30 minutes while others can take 24 hours or longer to multiply.

Cancer cells continue to reproduce by splitting (including the nucleus) into two daughter cells which themselves split and grow into adult cancer cells and then split again, on and on continually for the life of the malignancy. This process of cell splitting, called mitosis, only produces daughter cells, which enlarges into a massive collection of cells, which is referred to as a tumor. Designated cancer cells on the outer edges of the tumor can be released and travel to other distant sites by a process called metastasis. Once this metastatic process proceeds, the cancer spreads to critical body parts and usually heralds a poor overall outcome for the patient. Cancer cells are typically unregulated, disorganized, and engage in extremely rapid rates of mitosis. When enough cancer cells are made, they form larger tumors, which interfere with the duties and nutrition of nearby normal cells.

Cancer does its damage in complex ways that include strangling or distorting organs, blood vessels, and nerves as well as working its way into bones, brain, and muscles. Cancer cells perform no function that contributes to the homeostasis (life equilibrium) of the body in any way.

As described in the '835 patent, cancer cells have developed ways to repel or block the human body immune system by several means including erecting an electrical shield on the outer surface of the plasma membrane, which is produced by the cancer cell itself. Such a thin electrical shield is called the glycocalyx and generates a negative charge to oppose the animal or human immune system, which is also negatively charged. Two negative bodies repel each other, which in the case of cancer mean that the immune system cannot engage the tumor to destroy it. The body's natural immune system is not effective in attacking cancer as it does in attacking invading bacteria or viruses or even malfunctioning cells that have been injured, which are usually positively charged. Positively charged microbes or ill cells are susceptible to killer T-cell and other immune system attacks because the negatively charged immune defenses can approach its target successfully.

Additionally, there is a programmed cell death called apoptosis. Apoptosis as a bio-medical term that indicates that there is a state of natural or induced reprogramming of a cell to enter a suicide mode whereby the cell dies without any inflammatory process. Thereafter, the lifeless cell is phagocytized and removed by macrophages of the immune system. Apoptosis can occur in many kinds of cells such as erythrocytes as a method to rid the body of non-performing or defective cells. In general, cancer cells are thought to not have much opportunity to have preprogrammed cell death because those cells have an immortal ability to continue to reproduce and reorganize their cellular electrochemical system in a way that suits the purpose of the cancerized cell.

Some 200 ion channels or more populate all sides of the cell plasma membrane which encompasses and shelters the interior operations of the cancer cell. Cells, including malignant ones, are considered to have an internal signaling mechanism in order for them to operate the cell and remain alive as well as participating in tumor life processes of continuous reproduction of more cancer cells.

Signaling between cells of a tumor is also believed to make it possible to know when to release adult cells so that they can metastasize to other areas and begin a new tumor colony. The metastatic cancer cells travel within the blood vessels or the lymphatic system or propel themselves across an organ, nerve, gland or muscle to seed a new tumor site. For the individual cancer cells to communicate among themselves, they seemingly have to establish links to neighboring cells. These connections between the individual adjacent cancer cells are specifically tied to one another to allow for the sharing of signals. Ordinarily, cancer cells do not communicate with normal cells and are unable to affect the healthy normal cell in any way, therefore, sparing the unaffected normal cell from any direct operational assault.

An initiating cancer cell starts out as a normal cell, but develops a chromosomal and/or a genetic chaos that drives a transformation to malignancy. Prevailing cancer theory blames mutations in important regulatory genes for disturbing the normal controls on cells that are destined to become malignant. Such theory does not give credit to the damaging changes to actual chromosomes that are seen in all cancer cells. The distorted, broken or bent chromosomes can unbalance thousands of genes and are believed to be sufficient to trigger cellular instability that can lead to serious genetic disruption, transforming so-called normal cells into malignant ones. While the cancer cells may retain their electrochemical signaling and operating systems which existed when it was a normal cell, changes seemingly occur to rearrange its cellular mechanisms in new ways to eventually disconnect its communication ability from adjacent normal cells and to start rapid reproduction of more cancerous cells.

As described in the '835 patent, the first cancer cells that are adjacent to normal unaffected cells are sometimes not “wired” into the rest of the tumor. Perhaps these first cells are only a demarcation line from malignant to normal and do not have to participate in the cellular communication system. Later cells do develop the desmosome interconnection communication system that allows a way for each cell to speak to its adjacent neighbor cells. Other means of communicating between cancer cells beside desmosomes are gap junctions, direct cell connections, and tight junctions. The various junctions are connected with the intermediate filaments so as to provide the pathway to transmit messages between the various cancer cells.

It is believed that neither the normal cell nor the malignant cell can live without a functioning electrical signaling mechanism to operate the electro-chemical processes that are shelved on the cytoskeleton shelving. The cytoskeleton is the framework within the cell that provides a somewhat flexible geodesic-like framework to maintain cell shape, provide shelves for chemical or electrochemical process, and allow space for the organelles, nucleus, and protein manufacturing elements within the cell. The liquid within the cell is called cytoplasm. There is a cytoplasmic streaming process that causes directional movement of the liquid cytoplasm as a means of local transport for the semi-floating organelles (functional cell components). Likely this allows these floating structures some sort of communication between the cellular membrane and the nucleus as they come into close proximity.

As described in the '835 patent, individual cells operate themselves by electrical and chemical processes to maintain life and to perform the function for which a given cell has been constructed. Cancer cells are considered to have different electrical signals than normal cells.

Cells generate their electrical energy and communication signals within the plasma membrane. The plasma membrane may also have electrical connections to adjacent cells of the same type. The nucleus is considered in communication with activities occurring in the plasma membrane, for that matter all other activities of the cell.

Cell signaling may be accomplished by a combination of electrical and chemical interactions. Different types of cells should require a varied level of signaling qualities. The creation or generation of a given cell signal is believed to begin in the plasma membrane where raw material and chemical ions are taken in from the extracellular matrix to both generate electricity and establish the signal format. The plasma membrane is a sort-of cell wall that takes in the required raw material via its ion channels. Ion channels open and close to allow passage into and from the cell interior. Electrical signals are likely generated in the plasma membrane before they are sent via the cytoskeleton, all about the cell to go and participate and contribute to cell operations.

The cytoskeleton also serves as a geodesic-style dome providing a framework to shape and support the cell. In addition, the cytoskeleton serves as the pathway by which cell signals generated in their plasma membrane travel within and around the cell to do its work. In addition, communication to adjacent cancer cells could happen through connections such as desmosomes, which are extensions that bridge and allow communication between adjacent cells of a tumor.

Necrosis, apoptosis, autophagy, stasis, macroautophagy, cell starvation, tumor reduction, shut-down of mitochondria production of ATP, consuming contents of cytoplasm, incipient starvation, blebbing, cell shrinkage, nuclear fragmentation, chromatin condensation, chromosomal DNA fragmentation, pyknosis, karyolysis, karyorrhexis. Human bodies have complex daily cellular maintenance duties to dispose of some 50 million worn out cells every day. Average adult humans operate an ever-busy apoptosis and repair system. Key elements are briefed below.

Necrosis is a form of traumatic cell death that results from acute cellular injury. Necrosis death of cells can happen because of infection or fever that result in the premature death of cells in living tissue. Untreated necrosis results in a buildup of dead and decomposing cell debris in the region of actual cell death. A classic example would be gangrene. Cells dying from necrosis don't follow the usual apoptosis transduction pathways.

Apoptosis is the original programmed cell death technology that helps repair and model the body beginning with birth and continuing on throughout life. Some 50 billion cells die every day due to apoptosis. For example, the lining of the digestive tract from the stomach lining on to the colon undergoes apoptosis every 3 to 5 days to replace the entire inner lining of the digestive tubular structures. Red blood cells are programmed to replace themselves every 90 days by undergoing killing by the spleen and the bone marrow manufacturing new blood cells and releasing them back into the blood vessels to do their work of carrying oxygen and carbon dioxide.

Technical events that appear during an apoptosis event include characteristic changes that include cell shrinkage, generating heat, hypoxic events and an increase in calcium concentration which causes snappy signaling in the nucleus that triggers and orchestrates the imminent apoptotic event.

Autophagy is from the Greek definition as “self-eating.” Inside a living cell's cytoplasm are organelles identified as autophagosomes which move around the cell to sweep up viruses, bacteria, and worn out materials from the cell itself. The autophagosomes bag up or concentrates the cell sludge and worn out protein and other debris to be handled by recycling organelles that float in the cytoplasm. Some of the unusable waste is forced out of designated cell ion ports by pumping it through the plasma membrane into the extra-cellular fluid surrounding the cells. Since some neurons live as long as the body they have to use autophagy to maintain the quality of the overall cell health. Autophagy and mitochondrion can work together to cause apoptosis to trigger programmed cell death to rid the cell of unwanted cell component that can't be rehabilitated. Unlike necrosis, apoptosis produces cell fragments called apoptotic bodies that phagocytic cells are able to engulf, eat, digest, and then dispose of in league with the autophagy process in a well-established method to keep the overall cellular system in order.

Pyknosis is the irreversible concentration of chromatin in the nucleus of a cell involved in necrosis or apoptosis. This is followed by condensing its nucleus before expelling it to become a reticulocyte. The maturing neutrophil will be involved in forming blebs that stay in the cell until the end of its life. Blebs are distortion of the nucleus and the cancer cell shape. It is the formation of protrusion or pimple structures of what was previously a symmetrical nucleus and overall cell shape. It is followed by fragmentation of the changing nucleus on its way to experiencing karyorrhexis. During bleb formation of the nucleus, a sort of pimple formation gives the nucleus an unhealthy appearance, which does not improve.

Karyorrhexis is the ultimate bursting of the cellular nucleus into multiple pieces that cannot be repaired. The nucleus of a cell represents and is equivalent to the brain of any creature, once it is broken into pieces it is finished.

Karyorrhexis is an important cancer killing skill, which is accomplished by fragmentation of the cancer cell nucleus into apoptotic bodies, which are then engulfed and ingested by phagocyte(s). A phagocyte is a special cell that locates and surrounds broken cellular components and then eats them. There are fixed phagocytes that live in the liver, bone marrow, and spleen. Such phagocytes are represented by neutrophils and macrophages. Also, there are freely moving phagocytes such as leukocytes (white blood cells) that circulate in the blood stream to do their clean-up work. The job of the nucleus is to control all cellular operations and to participate in communication and coordination with nearby cells. If a nucleus is fragmented, it is like fragmenting the brain of a human or animal, life cannot go on with such as injury.

Electrical signaling can function to control and regulate chemical activities, autophagy, regulates the mitochondrial production of ATP which serves as an energy source for the cell, and controls the ribosome's protein manufacturing operations. In addition, the electrical codes can serve as communication means with the adjoining cells including when to release cells for metastasis operations among other duties.

Electrical signal flow traveling throughout the many cells of the tumor may allow for the generation of instructions to select cells that are destined to metastasize to distant sites to spread colonies for the malignancy. Such cells become soft and slightly puffy as they are released into the lymph or blood circulation system to travel to distant sites to start a new metastatic colony.

Electrical signals from the plasma membrane may travel on the surface of the intermediate filaments and reach chemical processes and likely ignite or stimulate a reaction that contributes to reproduction, protein manufacture or metabolic operations. Without electrical activity and the molecular devices that operate the cell plasma membrane, the cell could not function properly.

The charge of the outer wall takes on a protective negative charge, especially on the very thin outermost cell coating which is called the glycocalyx. This glycocalyx in cancers is considered to have a continuous negative charge protecting the malignant structure from the immune system which is also electrically charged in a negative format to repel the immune system from attacking the cancer, while non-cancerous glycocalyx coatings are positive in their protective electrical charge. All of this allows the positive protective charge to permit the negative charged immune system to embrace the positive cell protective elements and engage undesirable invaders like viruses or bacteria. Not so for the cancer glycocalyx with its negative shield which may repel the immune killer T-cells as they approach.

Many issues go wrong during the formation of tumorous mass including and not limited to uncontrolled proliferation, loss of apoptosis, tissue invasion and metastasis and Angiogenesis. Though the fundamental mechanisms are still unclear, it is safe to assume that there is information (regardless of its nature) that is originated from a group of cells, an individual cell or a sub-cellular component, this information is transmitted through some means (the transmission line) and then received by a group of cells, an individual cell or a sub-cellular components that have the ability to act on the received information. It is also safe to assume that the interruption of the information from one originator to the receptor would result in information loss and therefore interruption of the communication. The information during Cancer proliferation is more often than not viewed as chemical, genetic but not electro-magnetic. In addition to the chemical and genetic information carrier entities it has been demonstrated that electromagnetic transmission is taking place. The decoding of such information is yet to be achieved. Suffice it to say that all the fundamentals of electro-magnetic communication have been established. It is therefore useful to add the possibility of electromagnetic transmission to the existing understanding of the dynamics fueling cancer.

The membrane of biological cells and organelles act like platelike capacitors with the capacitance:

$$C = \frac{χεε0 p 2}{d}$$

Where χ is the portion of the plate-like capacitor, ε and ε₀ are the permittivity of the biological media and the permittivity of free space, d the distance or space in the inter-membrane and ρ is the radius of curvature of the platelet. The energy stored is related to the established voltage gradient divided by the distance.

The helical coils of bio-molecules result in an inductance represented by the equation:

$$L = μ {μ}_{0} d / 8 π$$

Where μ is the Permeability of the biological media.

Lastly the dynamic circuitry of highly nonlinear biological interconnections which contributes to charge storage as well as resistance between the various molecules as gated by hoping of electrons or conductance of heavier charged species (ions, anions, low molecular weight species) leads to an impedance described by

$$Z = {( {R}^{2} + ( ω L - 1 / ω C ) )}^{1 / 2}$$

The impedance Z is the smallest when Z=R where ωL=1/ωC. Under this condition and a constant electric field (as established in the metabolic pump and as exemplified by the mitochondria ions build up in the interlayer, which therefore represents a power supply condition with a well-established voltage gradient, a variety of oscillatory conditions can be established. These oscillatory conditions do take into account the inter-connectivity of the biological media with many constituents each sharing boundary conditions and contributing to an overall energy continuum of the collective. These biological oscillatory systems are complex, and many fundamental electromagnetic laws and thermodynamic principles need to be applied, simulated, verified gaged for their predictive effectiveness. This aside, the establishment of conditions intrinsic to the biological system leading to the charge up and storage of electrical energy and subsequently discharge and decay of the stored energy under the form of electro-magnetic energy is empirically well established.

FIG. 4A-1 is a depiction of on the left a) a conventional LRC circuit capable of resonating and releasing stored energy E in the electromagnetic energy emission at well defined frequencies and on the right (b) an equivalent type biological circuit with a metabolic pump (MP), coiled molecules (CM) with a representative inductance (L), a capacitive layer (CL) from a phospho-lipid bi-layer, and the highly interconnected biological media (BM) completing the electrical circuit. This biological circuit exhibits similar characteristics as a low energy storage (low Q) LRC and can resonate in the range of 10¹⁴-10¹⁵ Hz range which encompasses the visible and the UV range. Low Q typically translates into a broad emission frequencies.

Furthermore, taking values of g, E and x that pertain to biological media, one can calculate the frequency of the resonance oscillators. These calculations yield frequencies in the range of 10¹⁴-10¹⁵ Hz range which encompasses the visible and the UV range. These findings were the subject of publications including:
Chwirot, W. B., Dygdala, R. S. and Chwirot, S. (1985) optical coherence of white light induced photon emission form microsporocytes of Larix and Europeas Mill. Cytobios, 44, 239-249.
Frohlich, H. and Kramer, F. (1983) Cohemet exciation in biological systems. Springer Verlag, Heidelberg.
Smith, C. W., Jafary Asl, A. H., Choy, R. Y. S. and Monro, J. A., (1987) the emission of low intensity electromagnetic radiation form multiple allergy patients and other biological systems. Photon Emission from Biological Systems, Jezowska-Trsebiatowska, B. Kochel, B. Slawinski, J. and Strek, W. (eds). World Scientific, Singapore, pp. 110-126.
Tiblury, R. N. (1992) the effect of stress factors on the spontaneous photon emission from microorganisms. Eperientia, 48, 1030-1041.

It is therefore useful, in view of the empirically well-established bio-photonic energy and the sound theoretical understanding, to view the classical cancer proliferation steps and identify the presence and the possible fit and play of the of electromagnetic energy in the various proliferation steps including cell proliferation, loss of apoptosis, tissue invasion and metastasis and angiogenesis.

Receptors consist of three domains and extracellular Ligand binding domain a transmembrane domain and an intracellular domain as illustrated in the figure:

Binding of a ligand to the extracellular domain activates the receptor tyrosine kinase which activates other proteins by phosphorylation of adding a phosphate to the amino acid tyrosine on a protein inside the cell.

The binding of the ligand to the extracellular domain could be accompanied by the emission of light in view of the Gibbs free energy reduction that accompany a favorable chemical reaction. When a ligand binds to the receptor a signal goes to the intracellular domain activating the associated enzyme and initiating a cascade of signals to the nucleus that tells the cell to grow and divide or to stop growing. These signals can in fact be electromagnetic in nature.

A protein kinase is a kinase enzyme that modifies other proteins by chemically adding phosphate groups to them (phosphorylation). Phosphorylation usually results in a functional change of the target protein (substrate) by changing enzyme activity, cellular location, or association with other proteins. The human genome contains about 560 protein kinase genes and they constitute about 2% of all human genes. Up to 30% of all human proteins may be modified by kinase activity, and kinases are known to regulate the majority of cellular pathways, especially those involved in signal transduction.

### Autocrine Stimulation:

Malignant cells generate many of their own growth signals which allows them to divide with reduced external growth stimulation some cells are able to produce their own growth factors and stimulate their own growth. These growth factors are then driven or diffused to the cell membrane and release to the environment outside of the cell which stimulates certain ligand.

It is possible that the autocrine process is the results of electromagnetic radiation that results from within the cell or group of cells when under stress. The stress signal stimulates biophotons which favor the over production of certain molecules (growth factors in this case) and keeling the system off balance. For example: Glioblastomas express platelet derived growth factor or PDGF and sarcomas express tumor growth factor alpha or TGF-alpha & epidermal growth factor receptors or E-GFR. In one embodiment, the present invention can interfere with the transmission of information related to proliferation by having energy modulators that get excited by X-Ray energy and emit UV energy tuned to denature such growth factors as EGFR and PDGF described in sarcomas and glioblastomas. The growth factors are targeted by UV energy to halt the growth factor inside and outside the cell. It is conceivable to have energy modulators (of small enough size) to migrate into the cytoplasm and emit UV radiation selective to the full or partial denaturization of the growth factor.

In normal cells, the production of cell surface receptors is limited by cellular restraints on gene expression and protein translation. In tumor cells, however, mutations in the genes and coding for the receptors disrupts this finely tuned regulation and too many copies of the gene are produced in a phenomenon called gene amplification.

Excessive transcription and production of receptors leads to the fact that the more receptors expressed, the more binding sites are available for the ligands. This is a sort of runway condition. The off balance of finely tuned dynamics, results in tumor cells that have increased potential to be triggered into a growth phase by the binding of ligands to the excess receptors that decorate the cell walls.

Gross overexpression of growth factor receptors can result in ligand independent signaling where receptors are active in the absence of stimulating molecules. Structural changes to a receptor can also lead to ligand independent activation. This structural change including a modified conformation could be triggered by light, such as truncated versions of the EGRF where much of the intracellular domain is missing or constitutively active.

EGF-Receptor (such as HER1 or ErB-1) is a member of a sub family of type one receptor tyrosine kinases. These receptors are found primarily in the membranes of normal epithelial cells from: skin, breast, colon and lungs (amongst others). EGF-Receptor and its ligand play a central role in the regulation of cell proliferation differentiation & survival. EGFR is overexpressed in tumors arising from the colon, rectum and head and neck to name a few.

When a specific ligand binds to its receptor this leads to changes in the receptor that transmit a specific signal into the cell. For example the receptor tyrosine kinase is activated and initiates a signaling pathway specific to that receptor. This phenomenon is called signal transduction.

Activation of a signal transduction pathway creates a complex chain of events in the cytoplasm or fluid intracellular space that eventually leads into the cell nucleus where the transcription of genes regulating cell cycle progression are stimulated resulting in cell proliferation.

One of the major cascades implicated in cancers is the Ras Raf Activated Protein MAP kinase pathway. Another interesting pathway is the phosphor type 3 kinase or PI 3K/Akt/mTOR pathway. These pathways are linked to each other and other signal transduction pathways in the cell de-regulation or loss of normal controls in any of these pathways is thought to be present in all human tumors.

Once the signal reaches the nucleus, transcription factors are activated. These factors transcribe the genes that are translated into proteins, such as growth factors, that are necessary to allow the cell to continue to proliferate. Therapies can target factors responsible for tumor growth include the ligand receptors, intracellular second messengers and nuclear transcription factors.

Ligands can be neutralized before they bind to the receptors: An example of this is Avastin which is a humanized monoclonal antibody that targets circulating vascular endothelial growth factor or VEGF.

Platelet derived growth factor or PDGF, fibroblast growth factor or FGF, and other examples of ligands can be targeted for different cells in the body. Light based therapies can target the denaturization of these chemistries as exemplified before.

The receptors on the surface of normal and tumor cells can be inhibited directly. Erbitux is an example of this. It is a chimeric antibody that binds directly to the epidermal growth factor receptor and competitively inhibits the binding of EGF and other ligands such as TGF-alpha. Another way to block the receptors function is through small molecule inhibitors of receptor phosphorylation associated with them. For example, EGF receptors have a tyrosine kinase that can be blocked by molecules such as Gefitinib (Iressa) or Erlotinib (Tarceva).

### Apoptosis:

Apoptosis is a mechanism by which organisms limit the growth and replication of cells. If apoptosis did not occur it would be hard to control growth and tissue homeostasis would be lost (in fact this is one of the key mechanisms behind cancer). The genetic alterations in the cancer cell not only lead to increased cellular proliferation and growth they also lead to loss of apoptosis (i.e., excessive cell growth and little cell death in malignant tissue). Apoptosis occurs in normal cells to allow for removal of damaged cells and maintaining a constant number of cells in regenerating tissues and is an important part of embryogenesis. In an average human adult 50 to 70 billion cells undergo apoptosis per day. Apoptosis is characterized by changes such as: cell shrinkage, mitochondrial cytochrome C release, and fragmentation of cell DNA into multiples of 180 base pairs. In the end, cells are broken into small apoptotic bodies which will be cleared through phagocytosis. Phagocytosis is a process where cells take in the cell fragments or microorganisms in membrane-bound vesicles. The vesicles fuse with lysosome containing proteases and the engulfed material is processed for recycling.

There are two pathways that can activate apoptosis:
1—The first is the death receptor or extrinsic pathway. It is triggered by activation of members of the tumor necrosis factor receptor superfamily.
2—The second is through the mitochondrial or intrinsic pathway. This is set in motion by DNA damage.

Both pathways ultimately stimulate a set of enzymes called caspases which interact with inhibitors of apoptosis proteins or IAP and a Bcl-2 family of proteins (which individually have either pro and anti-apoptotic properties).

In some malignant cells there is resistance to apoptosis due to overexpression of anti-apoptotic proteins. For example Bcl-2 is overexpressed in B-cell lymphoma as a result of the translocation of its gene. Conversely, deactivating mutations having a pro-apoptotic molecule like backs is seen in some gastrointestinal tumors and leukemias. Anticancer agents have been developed targeting anti-apoptotic molecules. For instance, short segments of DNA complementary to the RNA of Bcl-2 or antisense oligonucleotides have been designed to reduce the translation of this anti-Apoptosis protein.

Activation of transcription factors can lead to apoptotic resistance. This occurs for example when members of the nuclear factor kappa B or (NF-kB) family of transcription factors are over expressed in certain tumors which lead to increased transcription anti-Apoptotic members of the IAP and Bcl-2 families. Ubiquitin proteasome pathway regulates the expression of transcription factors and other cell cycle proteins. Certain molecules can suppress or reduce NF-kB and IAP one activation and inhibit tumor promotion. Bortezomib is a proteasome inhibitor that has shown promising results in multiple myeloma. It inhibits the proteasome which leads to increased levels of the NF-kB inhibitor and therefore less anti-apoptotic proteins.

### Tissue Invasion and Metastasis:

Normal cells grow in a controlled manner that form tissues that form organs with specific functions. Malignant cells are defined by their ability to invade adjacent structures and be disseminated or metastasize. Malignant tumors can metastasize at any point. They do so by having cells break off from the main to enter the bloodstream and/or lymphatic channels and travel to other parts of the body to initiate a new tumor. Their ability to invade eventually affects the function of the normal tissue into which they are growing. Metastasis is a multi-factorial process involving complex interactions between tumor cells.

The EGFR pathway activates and modulates metastasis. When the appropriate signals enter the cell, a complex chain of events within the cytoplasm is set in motion. These events eventually lead into the cell nucleus where the transcription of gene regulating cell cycle progression and cell growth are stimulated. One protein produced through the cell activation process is the enzyme matrix metalloproteinase or MMP. When a tumor cell metastasizes, it breaks off from the main tumor and enters the extracellular space. Tumor cells secrete MMP which degrade the collagenous extracellular matrix, or ECM, breaking through the basement membrane that surrounds the tumor allowing the tumor cells to migrate toward the blood or lymph vessels.

When the MMPs reach the vessel they break down the basement membrane surrounding the vessel through enzymatic action opening access to the epithelial cells lining the vessel. Tumor cells can then migrate into the blood and lymph by entering through the tight junctions of the epithelial cells. The tumor cells are then transported through the blood and lymph to other tissues. It is known that metastatic tumor cells tend to target some organs more than others although the reason why is poorly understood. The migration of tumor cells into the organs is very much like the recruitment of white blood cells to tissues after injury.

Initially there is weak adhesion of tumor cells to endothelial cells which allows the tumor cells to shelter along the vessel lining until stronger bonds are formed. Once the metastatic cells are securely attached to the endothelial lining, they leave the vessel and enter the tissue. They also leave an open pathway that allows less aggressive tumor cells to invade the tissue and grow.

### Angiogenesis:

As the tumor grows it will eventually reach a size where it will need to have additional vasculature to sustain continued growth. To achieve this the tumor cells excrete certain proteins to stimulate blood vessel growth into and around the tumor in a process called angiogenesis. One of the major pathways involved in angiogenesis involves vascular endothelial growth factor, or VGEF, and its family of receptors. There are seven subtypes of VEGF and three receptors that each bind differently. VGEF affects the endothelial cells that line the blood vessels in a number of ways. It can cause them to proliferate by activating the extracellular kinases and MAP kinase signal transduction pathways. It can induce proteins that can break down the basement membrane to allow endothelial cells to migrate and invade these proteins including matrix metalloproteinases or MMPs, euro kinase plasminogen activator uPA and its receptor uPAR, as well as the tissue type plasminogen activator. It makes vessels more permeable allowing molecules and fluids to leak out.

When MMP is secreted into the extracellular space it degrades the extracellular matrix to allow pro-angiogenic factors to reach the vasculature. With the extracellular matrix degraded pro-angiogenic factors including VGEF can reach receptors on the endothelial cells of blood vessels surrounding the tumor, thus stimulating the angiogenic signal in the vessel.

VGEF also helps the new endothelial cells survive by up regulating inhibitors of apoptosis. VEGF also activates the endothelial cells to express the proteins necessary to allow the new blood vessels to form. The end result is the growth of new blood vessels into the tumor. With this growth of new vessels into the tumor, additional nourishment can be delivered to the tumor. New blood vessels in the tumor thus facilitate further tumor growth. Strategies targeting VEGF and its receptors have been used successfully in clinical practice. Avastin is an antibody that binds VEGF and prevents its binding to its receptor. Another therapy is Sutint which is a small molecule inhibitor with high binding affinity for VEGF and PDGF receptors. With psoralen compounds and UV energy modulators, it is possible to achieve the same results by binding the affinity of VEGF and PDGF. Another strategy is to target the exact frequency (derived from a UV-VIS) to cause ionization or denaturization of the VEGF and PDGF.

While much is known about these various biological processes, and much is known about the phenomenon of cell-to-cell communication/signaling from laboratory in vitro experiments on cell cultures, there is a need for the ability to detect such cell-to cell communication/signaling in vivo in living organisms, and to use the information gathered from such detection in methods and techniques for harnessing that communication/signaling power of cells to affect and/or trigger various of these biological processes within a subject.

### SUMMARY OF THE INVENTION

Accordingly, one object of the present invention is to provide methods and/or devices/apparatus for detecting and/or monitoring communication within a cell or between cells.

A further object of the present invention is to provide methods and/or devices/apparatus for the ex vivo detection and monitoring of cell-to-cell communication/signaling in a living subject.

A further object of the present invention is to provide methods and/or devices/apparatus for causing or triggering cell-to-cell communication/signaling in order to cause or enhance one or more biological processes in a living subject.

A further object of the present invention is to provide a method of treating a subject having a disease, disorder, or condition, by detecting the presence of one or more biomarkers correlated with the disease, disorder, or condition in affected cells of the subject, determining a photon wavelength that triggers the cells to alter the one or more biomarkers in a desired direction, then treating affected cells of the subject with radiation at that wavelength to treat the disease, disorder, or condition.

A further object of the present invention is to provide for a way to monitor biomarkers (indicative a change in cell function) inside a living cell.

A further object of the present invention is to provide for a way to monitor biomarkers (indicative a change in cell function) inside a living cell exposed to a stimulant light.

A further object of the present invention is to provide for a way to monitor biomarkers (indicative a change in cell function) inside a living cell not exposed to a stimulant light but otherwise coupled to a living cell exposed to the stimulant light.

A further object of the present invention is to correlate cell function with biomarker production.

Still another object of the invention is to correlate cell function with both biomarker production and stimulant light exposure.

Still a further object of the invention is to customize a stimulant light treatment based on measured biomarker production occurring inside a living cell exposed to a stimulant light.

Still yet another object of the invention is to customize a subject treatment based on measured biomarker production occurring inside a living cell.

Still another object of the invention is to customize a subject treatment based on measured biomarker production occurring inside a living cell exposed to a stimulant light.

Still a further object of the invention is to generate and correlate a response at a non-treatment site in a patient in response to a stimulant light treatment at another site.

In one aspect of the invention, the change in the cellular environment of the cells in the first region is stimulated by light stimulation of the cells in the first region.

In one aspect of the invention, the change in the cellular environment of the cells in the first region is tracked or monitored by the monitoring of the biomarkers in the cells in the first region.

In another aspect of the invention, the induced biological change in the second region inside the subject can be monitored by detecting the biomarkers in cells in the second region and correlating the biomarkers with the subject treatment.

In another aspect of the invention, the induced biological change in the second region may be local changes in proximity to the first site and/or global changes throughout the patient.

In another aspect of the invention, the photonic stimulus of a local region burdened with a disease leads to immune system activation yielding a systemic response against the disease.

In another aspect of the invention, the photonic stimulus of a local region burdened with a disease leads to the presentation of neoantigens and becomes amenable to existing therapies.

In another aspect of the invention, the photonic stimulus of a local region burdened with a disease leads to the production of messengers that curb the proliferation of the disease leading to retardation, partial remission, complete remission or stabilization of the disease.

In another aspect of the invention, the photonic stimulus of a local region burdened with a disease prohibits the signals leading to angiogenesis and prevents the formation of new blood vessels.

In another aspect of the invention, the photonic stimulus of a local region burdened with a disease result in the reduction of ATP production in the mitochondria in the said treated region.

In another aspect of the invention, the broadband photonic stimulus of a local region burdened with a disease result in the reduction of ATP production in the mitochondria in the said treated region as well as prohibits the signals leading to angiogenesis.

In another aspect of the invention, the photonic stimulus of a local healthy region unburdened with a disease leads to the increase in the length of the telomeres.

In another aspect of the invention the exogenous light stimulus results in proliferation of specific biological tissue.

In another aspect of the invention, the photonic stimulus of a local healthy region unburdened with a disease leads to the growth of nerve tissue.

In another aspect of the invention, the photonic stimulus leads to the change in conformation of select proteins leading to a biological change at the cell level.

In another aspect of the invention, the photonic stimulus leads to the change in conformation of select proteins leading to closure of ion channels of the cell surface.

In another aspect of the invention, a bio-feedback loop is established between the light stimulus and gene expression.

In another aspect of the invention, an exogenous light stimulus leads to endogenous light production.

In another aspect of the invention, an exogenous light stimulus leads to expression signaling messengers.

In another aspect of the invention, the induced biological change in the second region may be by way of cell-to-cell communication from the first site treated with stimulant light to a remote site not stimulated.

In one embodiment of the invention, there is provided a method (and system) for detecting and/or monitoring communication within a cell or between cells, comprising:
placing, in a region of interest within a living organism, a detector configured to monitor one or more signals emitted within a cell or between cells in a plurality of cells, wherein the region of interest is adjacent to the cell or plurality of cells undergoing a biological change;
collecting the one or more signals emitted from said cell or plurality of cells; and
identifying one or more characteristics associated with the one or more signals and correlating the one or more characteristics to the biological change.

In another embodiment of the invention, there is provided method (and system) for stimulating communication within a cell or between cells, comprising:
inserting into a region of interest within a living organism a signal emitter emitting a signal having a predetermined characteristic or combination of characteristics; and
causing the signal emitter to emit the signal having a predetermined characteristic or combination of characteristics;
wherein the predetermined characteristic or combination of characteristics are correlated to trigger a desired biological change within the cell or cells, said desired biological change being communicated within a cell or between cells to propagate the desired biological change.

In one embodiment of the invention, there is provided a method (and system) of treating a subject comprising:
initiating a change in a cellular environment of cells in a first region of a biological material inside the subject; and
due to a change in biological or chemical activity of the cells in the first region, inducing a biological change in a second region inside the subject; and
monitoring biophoton emission from the first region, into the second region, or from the second region.

In one embodiment of the invention, there is provided a method (and system) of of treating a subject comprising:
initiating a change in a cellular environment of cells in a first region of a biological material inside the subject; and
due to a change in biological or chemical activity of the cells in the first region, inducing a biological change in a second region inside the subject by enhancing a coupling of the first region to the second region.

These and other objects of the present invention, which will become more apparent in conjunction with the following detailed description of the preferred embodiments, either alone or in combinations thereof, have been satisfied by the discovery of the embodiments enumerated and claimed below.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 is a schematic illustrating various cellular components of an example cell 100.

FIG. 1A is a schematic showing the cellular components of FIG. 1 along with the presence of biophotons and phosphors for emitting light to stimulate or mimic biophoton radiation.

FIG. 1B is a schematic showing the cellular components of FIG. 1 along with the presence of biophoton emission acting as a sensor of cell activity change at a distal site.

FIG. 1C is a schematic showing the cellular components of FIG. 1 along with the presence of biophoton emission produced in response to stimulation light.

FIG. 1D is a schematic showing various embodiments of the present invention where the biophoton emission from a treated cell is measured/detected by an in vivo biophoton detector in the vicinity of the cell(s) being treated.

FIG. 1E is a schematic showing one embodiment of the invention where simulated (replicated) biophoton light treats a target cell

FIG. 2 illustrates a schematic drawing of the structure of a plasma membrane 100 of the cell 100 shown in FIG. 1.

FIG. 3 illustrates a junction view 300 of the attachments between tumor cells.

FIG. 3A is a schematic showing the junction view of FIG. 3 along with the presence of biophotons and phosphors for emitting light to stimulate or mimic biophoton radiation.

FIG. 4 illustrates a pictorial drawing of the internal framework 400 of a cell, such as the cell 100 shown in FIG. 1.

FIG. 4A is a schematic showing the internal framework of FIG. 4 along with the presence of biophotons and phosphors for emitting light to stimulate or mimic biophoton radiation.

FIG. 4A-1 is a depiction of a conventional LRC circuit and an equivalent type biological circuit.

FIG. 5 is a depiction of a biophoton collector 500 according to one embodiment of the present invention.

FIGS. 6A-6C are depictions of an electromagnetic biophoton collector 600 according to one embodiment of the present invention.

FIG. 7 is a depiction of a fractal antenna according to one embodiment of the present invention.

FIG. 7-1 is a schematic showing a section of waveguide 710 according to one embodiment of the present invention, having a high-k dielectric material 712, a low-k dielectric material 714 and a central metal 716.

FIG. 7-2 is a schematic showing the antenna pickup area of one embodiment of the present invention having an open concentric polarization construction 720a.

FIG. 7-3 depicts an array 730 of antennae 732 according to one embodiment of the present invention.

FIG. 7-4 depicts a cross section of the stub configuration 730 shown in FIG. 7-3 with antennae 732 interconnected together according to one embodiment of the present invention.

FIG. 7-5 is a schematic of a multi-up arrayed antenna 750 according to one embodiment of the present invention.

FIG. 7-6 is another schematic of the multi-up arrayed antenna 750 shown in FIG. 7-5 showing a top-level interconnection network 762 under the top surface of multi-up arrayed antenna 750, according to one embodiment of the present invention.

FIG. 7-7 is a further schematic of the multi-up arrayed antenna 750 shown in FIG. 7-5 showing the full interconnection network including top-level interconnection network 762 and bottom-level interconnection network 764, according to a further embodiment of the present invention.

FIG. 7-8 is a depiction of antennae that can be arrayed in different manners including a square antenna 780a, a rectangular antenna 780b, and a diamond shaped antenna 780c, according to embodiments of the present invention.

FIG. 7-9 is a depiction of a spiral-type packing antenna arrangement 790 according to one embodiment of the present invention.

FIG. 7-10 is a depiction of a window chamber according to one embodiment of the present invention.

FIG. 7-11 is a depiction of a window 795 made of a quartz wafer that has different sections that are independent of each other, according to one embodiment of the present invention.

FIG. 8 is a depiction of a hollow optic biophoton bypass 800 according to one embodiment of the present invention.

FIGS. 9A and 9B are depictions of an electrically conducting biophoton bypass 900 according to one embodiment of the present invention.

FIGS. 10A and 10B are depictions of another electrically conducting biophoton bypass 1000 according to one embodiment of the present invention.

FIG. 11 is a depiction of a magnetic biophoton bypass 1100 according to one embodiment of the present invention.

FIG. 12 is a depiction of a DNA-based biophoton bypass 1200 according to one embodiment of the present invention.

FIGS. 13A and 13B are depictions of a living-cell biophoton radiator 1300 according to one embodiment of the present invention.

FIG. 14 is a depiction of a system of the present invention for application of microwave energy to a target region to locally heat the cells in the target region and thereby induce biophoton emission.

FIG. 15 is a depiction of an in vivo biophoton source 1500 according to one embodiment of the present invention.

FIG. 16 shows the spectral emission of the BP3, BP10, and BP6 phosphors.

FIG. 17 is a chart showing that photonic energy from BP3 tends to produce more MA than BP6 or BP10.

FIG. 18 is a chart showing MA formation under BP3 photonic energy as a function of distance from the X-ray source and time.

FIG. 19 is a chart showing XL under BP3 photonic energy as a function of distance from the X-ray source and time.

FIGS. 20-24 show results from other experiments corroborating MA formation and/or XL under photonic energy exposure.

FIG. 25 is a chart showing a non-linear effect on MA seen by mixing two phosphors.

FIG. 26 is a depiction of a helical “allosteric lever arm” as considered by Strickland et al. to be a mechanism for coupling the function of two proteins.

FIG. 27 is a depiction of a design of an allosteric, light activated repressor.

FIG. 28 is a depiction of the light-triggered dissociation of UVR8-tagged proteins.

FIG. 29 is a flowchart of one method for treating a subject according to an embodiment of the present invention.

FIG. 30 is a flowchart of another method for treating a subject according to a further embodiment of the present invention.

FIG. 31 is a side view of a cuvette construction according to one embodiment.

FIG. 32 is a top view of the cuvette construction of FIG. 31.

FIG. 33A is a depiction of a cuvette construction according to two different embodiments.

FIG. 33B is a depiction of a cuvette construction according to another embodiment.

FIG. 33C is a depiction of a cuvette construction according to yet another embodiment.

FIG. 33D is a depiction of a cuvette construction according to still another embodiment.

FIG. 33E is a depiction of a cuvette construction according to another embodiment.

FIG. 34 is a depiction of a cuvette construction of\for wavelength discrimination of cell to cell biophotons.

FIG. 35A is a depiction of fiber optic for in vivo biophoton detection.

FIG. 35B is a depiction of an organic in vivo biophoton detector.

FIG. 35C is a representation of a retina showing various components of a retina and retinal cells.

FIG. 36 is a depiction of near field scanning microscope for use in the present invention.

FIG. 37 is a schematic representation of a segment of a neuron, and an eigenmode of a cylindrical myelinated axon FIG. 38 is a schematic representation of an ultraviolet laser source useful in the present invention to stimulate or replicate biophotons.

FIG. 39A is a diagram of hydrogen atom emission states.

FIG. 39B is a molecular energy diagram for the transition metal manganese.

FIG. 40 is a diagram representative of a set of energy band diagrams with and without vibronic coupling.

FIG. 41 is a diagram representative of transient photoluminescence.

FIG. 42 is a schematic representation of a system for pulsed x-ray beam production for use in various embodiments of the invention.

FIG. 43 is a diagram representing the chemical conversion of luminol to a corresponding di-carboxylic acid.

FIG. 44 is schematic representation of a hydrogen peroxide sensor for use in various embodiments of the invention.

FIG. 45 is a diagram representing selective co-targeting of multiple pathways for cancer cell suppression.

FIGS. 46-65 show the experimental data from canine trials where bioimarkers were measured and tracked with cancer progression/regression.

FIGS. 66-71 show statistical analysis of the biomarkers measured and tracked in the canine trials.

### DETAILED DESCRIPTION OF THE INVENTION

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

While not limited to the following, the present invention with its natural sources of biophoton radiation and its artificial sources of biophoton radiation can alter the structures of the cells or the functions described above including the electrical signaling, can alter the chemical pumping and ion transport processes promoting cell growth (reproduction) or cell death, and can alter the “communication” or “coupling” between various cells to thereby provide a method for treatment of a condition, disorder or disease in a subject.

As used herein, biophoton radiation encompasses mitogenic radiation to any degree that the art considers these “radiations” or ultra weak emissions to be different. Indeed, the phenomenon of ultra weak emission from cellular systems has been a topic of various inquiries since the 1900s. This topic can be traced back to the early investigations of the Russian biologist Gurwitsch Alexander G. Gurwitsch more than seventy years ago, who speculated that ultraweak photon emission transmit information in cells [A. G. Gurwitsch, S. S. Grabje, and S. Salkind, “Die Natur des spezifischen Erregers der Zellteilung,” Arch. Entwicklungsmech. Org. 100, 11-40, 1923]. His research was an attempt to answer a question not responded in its full scale even now: “what are the causes of cell division?” Combining several observations, Gurwitsch concluded that this event required a coincidence of two factors: (1) internal cell “preparedness” to division, and (2) external impulse, i.e., a signal coming from the outside and “switching on” the (already prepared) mitosis. He suggested that the external impulse was non-chemical (i.e., a kind of radiation), and induced “collective excitation” of special molecular receptors located on a cell surface. This work and more recent work has shown that an “induction length” (that is a distance from cell emitting biophoton radiation and the cell reacting to the biophoton radiation) is extremely short, on the order of mm's, with some finding an optimal distance to be 1-10 mm.

Within the context of the present invention, the term “exogenous light” refers to light externally applied to a cell, a group of cells or a tissue in vivo or ex-vivo. The term “endogenous light” refers to light generated by or within a cell or a group of cells either spontaneously or as a result of said applied exogenous light.

The invention pertains to ways of applying an exogenous light as a stimulus and subsequently detecting its transmission to and impact on internal molecules or its transmission to and impact on neighboring cells. The invention further pertains to endogenous light generated at the molecular level, the cell level or the tissue level and then the transmission of said endogenous light to neighboring cells or neighboring tissue. The invention further includes the biological impact of exogenous and endogenous light to induce, affect, or monitor a biological change in a medium, particularly a living subject.

The invention in various embodiments encompasses methods and techniques for stimulating, detecting and/or identifying bio-photonic electromagnetic energy and stimulating the production of such naturally produced and transmitted electromagnetic energy inside a cell (intracellular) and amongst a group of short ranged neighboring cells (intercellular) and finally between two distinct group of cells as in the case between a group of diseased cells inside a tumor and a group of non-diseased cells in the Tumor Micro Environment (TME). Within the context of the present invention, unless specifically noted otherwise, the term cell-to-cell communication encompasses both intracellular communication (biophotonic or other types of communication between organelles within a single cell) and intercellular communication (biophotonic or other types of communication between separate cells, between groups of cells, etc.). In some cases the invention relates to the stimulation or interruption of the transmission of naturally occurring bio-photonic electromagnetic energy or simulation of naturally occurring bio-photonic electromagnetic energy (sometimes occurring as a form of cell-to-cell communication).

In various embodiments, the present invention relates to a method or methods for targeted light stimulus, detecting and/or monitoring communication within a cell or between cells. Preferably the communication is electromagnetic communication, but can take other forms as well, such as, for example, electrical or chemical. While investigators have been able to detect such electromagnetic communication, such as biophotons, between groups of cells in a laboratory in vitro setting, the detection of such electromagnetic communication in vivo in a living organism (plant or animal) has not been done. The present invention provides methods for doing just that.

The present invention provides a method for detecting and/or monitoring communication within a cell or between cells, comprising:
placing, in a region of interest within a living organism, a detector configured to monitor one or more signals within a cell or between cells in a plurality of cells, wherein the region of interest is adjacent to a cell or group of cells undergoing a biological change;
collecting the one or more signals from said cell or group of cells; and
identifying one or more characteristics associated with the signal and correlating the one or more characteristics to the biological change.

In a preferred embodiment, the detector is a biophoton detector and the one or more signals are electromagnetic signals, more preferably biophotons. In a further preferred embodiment, the electromagnetic signals are a wavelength or wavelength range. In a further embodiment, the detector is a combination of a biophoton emitter and a biophoton detector.

In this method, the biophoton detector can be any biophoton detector that is configured in such a manner to be placed within the living organism, preferably those biophoton detectors described below, most preferably the fractal antenna cannula or fractal antenna quartz wafer described below.

In further embodiments, the present invention provides a method for stimulating communication within a cell or between cells, comprising:
inserting into a region of interest within a living organism a signal emitter emitting a signal having a predetermined characteristic or combination of characteristics; and
causing the signal emitter to emit the signal having the predetermined characteristic or combination characteristics;
wherein the predetermined characteristic or combination of characteristics are correlated to trigger a desired biological change within the cell or cells, said desired biological change being communicated within a cell or between cells to propagate the desired biological change.

Preferably, the signal emitter is a biophoton emitter and the signal is emission at a predetermined wavelength or combination of wavelengths.

In these embodiments, the biophoton emitter may be any biophoton emitter configured to be used within the living organism. In preferred embodiments, the biophoton emitter is one or more optical fibers configured to transmit the predetermined wavelength or combination of wavelengths. Such optical fibers can be multimode fibers and can be constructed of any biocompatible material. For certain optical fibers, the material used to construct the fiber does not need to be inherently biocompatible but can be rendered biocompatible by surface treatments and/or coatings. An example is a multimode fiber using a polyamide coating for enhanced biocompatibility. Optical fibers are particularly attractive for biomedical applications because they are thin, flexible, dielectric (nonconductive), immune to electromagnetic interference, chemically inert, nontoxic, and lightweight. They can also be sterilized using the standard medical sterilization techniques: including, but not limited to, steam heat, radiation, or dry gas. The optical fibers are preferably formed from a material capable of transmitting the desired wavelength of electromagnetic radiation to be emitted. Suitable materials for the optical fibers include, but are not limited to, om silica, but some other materials, such as fluorozirconate, fluoroaluminate, and chalcogenide glasses as well as crystalline materials like sapphire, along with polymeric materials such as polymethyl methacrylate, amorphous fluoropolymer (poly(perfluoro-butenylvinyl ether), and polystyrene. In one embodiment, the biophoton emitter is a single optical fiber, which may be configured to transmit electromagnetic radiation from a single wavelength to broadband spectrum of wavelengths. Alternatively, the biophoton emitter can be a plurality of optical fibers, preferably in a unitary fiber optic bundle, wherein each individual fiber in the plurality of optical fibers can transmit any desired wavelength or range of wavelengths. In this manner, the plurality of optical fibers can be tuned to transmit a defined set of wavelengths to the site of interest to stimulate one or more biological processes simultaneously. Further, the plurality of single mode or multimode optical fibers can be configured to transmit their various emissions simultaneously, or can be programmed to transmit the wavelengths in a pre-programmed sequence, thus permitting complex treatment paradigms and regimens.

In a further embodiment, the biophoton emitter can be one or more of the below described energy modulation agents, energy converters, or phosphors, selected to provide emission at the desired wavelength or wavelength range.

In additional embodiments, the present invention provides methods for treating a disease, condition, or disorder in a living organism, by the in vivo triggering of electromagnetic stimulus to induce expression of chemical messengers and/or communication within a cell or between cells in the living organism, to cause cellular changes within the living organism to treat the disease, condition, or disorder. Further preferred embodiments of these methods are described in greater detail below.

In a further embodiment, the present invention provides a method of treating a subject having a disease, disorder, or condition, comprising:
testing affected cells of a subject for the presence of one or more biomarkers correlated to treatment of the disease, disorder, or condition;
determining a wavelength or wavelength range of photon that triggers the affected cells to communicate and alter a level of the one or more biomarkers in a desirable manner associated with treatment of the disease, disorder, or condition; and
treating the affected cells of the subject in vivo with radiation at the determined wavelength or wavelength range to cause a desirable change in the level of the one or more biomarkers in vivo, thus treating the disease, disorder, or condition.

In this embodiment, the one or more biomarkers can be any of those biomarkers identified below. The determining of the wavelength or wavelength range can be performed in a variety of ways, including, but not limited to, the use of the various cuvette arrangements described herein, which permit the identification of wavelength ranges for biophoton communication between groups of cells in the various cuvettes. The treating can be performed with any emitter that emits the desired wavelength or wavelength range, including, but not limited to, the various biophoton emitters and energy modulation agents identified herein.

In the 1970s, this area of research was investigated by a number of investigators. The presence of biological radiation from a variety of cells was later investigated by several research groups in Europe and Japan using low-noise, sensitive photon-counting detection systems [B. Ruth and F.-A. Popp, “Experimentelle Untersuchungen zur ultraschwachen Photonenemission biologischer Systeme,” Z. Naturforsch., A: Phys. Sci. 31c, 741-745, 1976; T. I. Quickenden and S. S. Que-Hee, “The spectral distribution of the luminescence emitted during growth of the yeast Saccharomyces cerevisiae and its relationship to mitogenetic radiation,” Photochem. Photobiol. 23, 201-204, 1976; H. Inaba, Y. Shimizu, Y. Tsuji, and A. Yamagishi, “Photon counting spectral analysing system of extra-weak chemi- and bioluminescence for biochemical applications,” Photochem. Photobiol. 30, 169-175, 1979]. Popp and coworkers suggested the evidence of some ‘informational character’ associated with the ultra-weak photon emission from biological systems, often referred by Popp as “bio-photons”. Other studies reported ultra-weak photon emission from various species including plant, and animals cells [H. J. Niggli, C. Scaletta, Y. Yan, F.-A. Popp, and L. A. Applegate, “Ultraweak photon emission in assessing bone growth factor efficiency using fibroblastic differentiation,” J. Photochem. Photobiol., B, 64, 62-68, 2001;]. Results of experiments of UV-irradiated skin fibroblasts indicated that repair deficient xeroderma pigmentosum cells show an efficient increase of ultraweak photon emission in contrast to normal cells. [H. J. Niggli, “Artificial sunlight irradiation induces ultraweak photon emission in human skin fibroblasts,” J Photochem. Photobiol., B 18, 281-285 (1993)].

A delayed luminescence emission was also observed in biological systems [F.-A. Popp and Y. Yan, “Delayed luminescence of biological systems in terms of coherent states,” Phys. Lett. A 293, 93-97 (2002); A. Scordino, A. Triglia, F. Musumeci, F. Grasso, and Z. Rajfur, “Influence of the presence of Atrazine in water on in-vivo delayed luminescence of acetabularium acetabulum,” J Photochem. Photobiol., B, 32, 11-17 (1996); This delayed luminescence was used in quality control of vegetable products [A. Triglia, G. La Malfa, F. Musumeci, C. Leonardi, and A. Scordino, “Delayed luminescence as an indicator of tomato fruit quality,” J. Food. Sci. 63, 512-515 (1998)] or for assessing the quality or quality changes of biological tissues [Yu Yan, Fritz-Albert Popp, Sibylle Sigrist, Daniel Schlesinger, Andreas Dolf, Zhongchen Yan, Sophie Cohen, Amodsen Chotia, “Further analysis of delayed luminescence of plants”, Journal of Photochemistry and Photobiology B: Biology 78, 235-244 (2005)].

It was reported that UV excitation can further enhance the ultra-weak emission and a method for detecting UV-A-laser-induced ultra-weak photon emission was used to evaluate differences between cancer and normal cells. [H. J. Niggli et al, Laser-ultraviolet-A-induced ultraweak photon emission in mammalian cells, Journal of Biomedical Optics 10(2), 024006 (2005)].

Further, the area of photobiology and the use of various types of electromagnetic radiation for therapeutic reasons has been known for many years (see, for example, Parrish, J. A., C. F. Rosen & R. W. Gange. 1985. Therapeutic uses of light. Ann. N.Y. Acad. Sci. 453: 354-364 which describes various photochemically based therapies and types of therapeutic photomedicine, the entire contents of which are incorporated herein by reference).

There are those that maintain that the health of the body depends on certain bioelectric vibrations that are susceptible to chemical or physical toxic factors. Fröhlich notes that there are coherent electric vibrations in the frequency range 100 GHz to 1 THz, excited in cells by metabolic processes (see Fröhlich H. Coherent electric vibrations in biological systems and the cancer problem, IEEE Transactions on Microwave Theory and Techniques, Vol. MTT-26, No. 8, August, 1978, pp 613-617). This idea is based on observation of the inhibition or stimulation of the growth of yeast and bacterias functions of the applied frequency, showing very stable and repetitive resonances. If such vibrational states are indeed metabolically excited, then they should be manifested in Raman spectroscopy. Actually, their existence has been demonstrated during periods of metabolic activity of lysozyme and E. coli (700 GHz to 5 THz). Emissions have also been observed at lower frequencies (150 GHz or less). These vibrations occur in the tissue of higher organisms and they have been hypothesized exercise some control on cellular growth (see also S. J. Webb et al, Nature, Vol. 218, Apr. 27, 1968, pp. 374-375; and S. J. Webb et al et al, Nature Vol. 222, Jun. 21, 1969, pp. 1199-1200).

Cancerization could result from a modification of these vibrations by the invasion of foreign molecules, e.g., the presence of free electrons in the condition bands of proteins. There is some evidence for the presence of double spectral lines at 1.5 and 6 THz in breast carcinoma, which may be an indication of an interaction between normal cellular vibrations and free electrons. In such coherent frequency communication between cells, it is believed that the medium through which the communication is transmitted is the water within and around the cells (see Smith, Coherent Frequencies, Consciousness and the Laws of Life, 9^{th} International Conference CASYS '09 on Computing Anticipatory Systems, Liege, Belgium, August 3-8, 2009).

Farhardi et al, in “Evidence for non-chemical, non-electrical intercellular signaling in intestinal epithelial cells” in Biochemistry 71 (2007) 142-148 in Science Direct (the entire contents of which are incorporated herein by reference) reported on a synchrony in which mechanically separated neighboring cells (which were not able to communicate via chemical or electrical mechanisms) nevertheless showed responses in the neighboring cells (untreated) to a treated cell undergoing apoptosis. Farhardi et al, found that “detector cells” as far as 4 cm away from the control cell (where H₂O₂ was added to induce cell death in an intestinal epithelial cell line) also showed cell death although not exposed to the hydrogen peroxide.

Matsuhashi et al, in “Bacillus carbibiphilis cells respond to growth-promoting physical signals from cells of homologous and heterologous bacgteris” in J. Gen. Appl. Microbiol. 42, 315-323 (1996) (the entire contents of which are incorporated herein by reference) reported that bacteria cells alone can emit signals that stimulate colony formation in neighboring cells as far away as 30 cm and even those separated by an iron plate. Matsuhashi et al concluded that sonic waves were the likely signals being propagated between cell cultures.

Attempts to measure the wavelength spectrum of biophoton radiation have reported spectra in the area from 190-250 nm in the 330-340 nm wavelength range where absorption in the natural medium would be expected, thereby limiting how far biophoton radiation would travel inside a subject. While also being weak and in the UV range, natural sources of biophoton radiation emit this radiation in short bursts of a duration of approximately 10-3 s at a frequency of 10 to 100 Hz.

Others have reported biophoton emission from skin cells with a spectra of photon emission detected from 500 to 700 nm, with primary and secondary emission peaks at 630-670 nm and 520-580 nm, respectively.

Shanei et al. in Detection of Ultraweak Photon Emission (UPE) from Cells as a Tool for Pathological Studies, 2016 published on line at www.jbpe.org (the entire contents of which are incorporated herein by reference) report that it is well-known that all living cells emit ultra-weak photon emission (UPE), which is considered due to byproducts of chemical reactions in cell metabolisms. Shanei et al. reported that it has been shown that Reactive Oxygen Species (ROS) in the cells enhances the UPE intensity. Shanei et al. reported that the magnitude of such UPE is extremely weak (i.e. a few to 10³ photons/(sec·cm²)), and the detection of such ultra-weak signals is hardly possible via sensitive instruments like photomultiplier tube (PMT) that can detect single photons. Shanei et al. also reported on earlier work where UPE from tumor tissue was observed to be higher than UPE from normal tissue.

In the experiments conducted by Shanei et al., they used 9235B as a 51 mm (2″) diameter, end window Photomultiplier (ET Enterprises Limited, United Kingdom) to measure photons emitted from HT-29 cells (a common cancer of digestive tract). Their detector had its maximum response at 350 nm with the quantum efficiency of 30% in detection range of 250 nm to 600 nm. Shanei et al. showed that the application of H₂O₂ to the HT-29 cells caused their death and a corresponding increase in the ultra-weak photon emission (UPE).

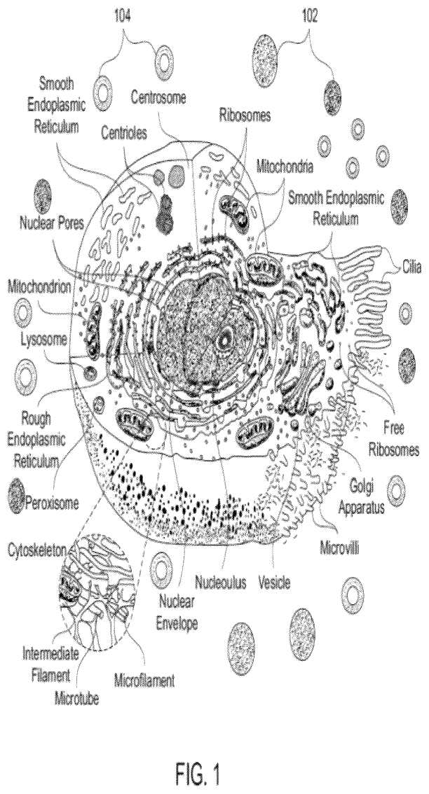

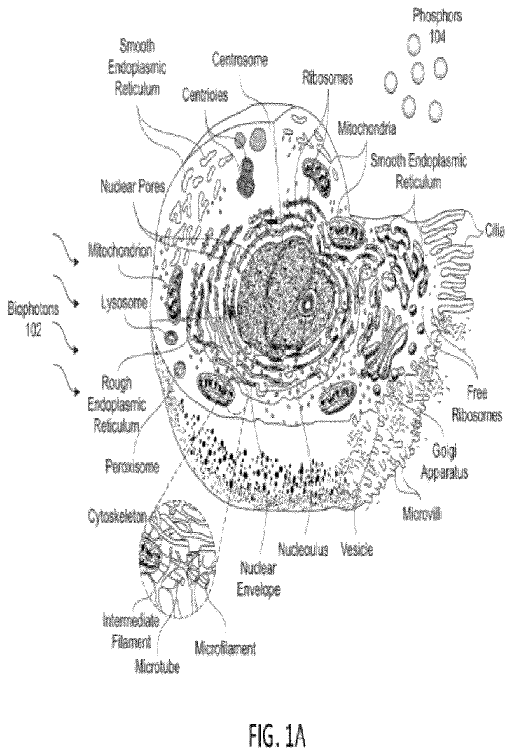

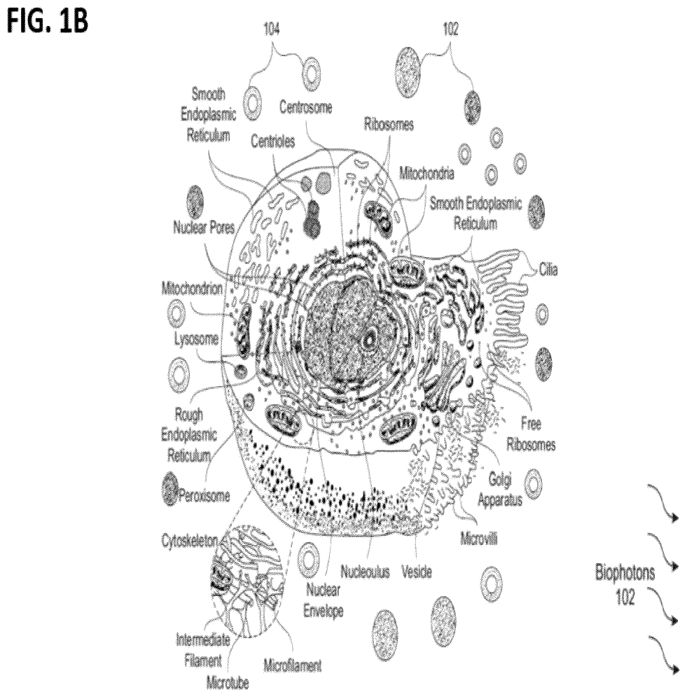

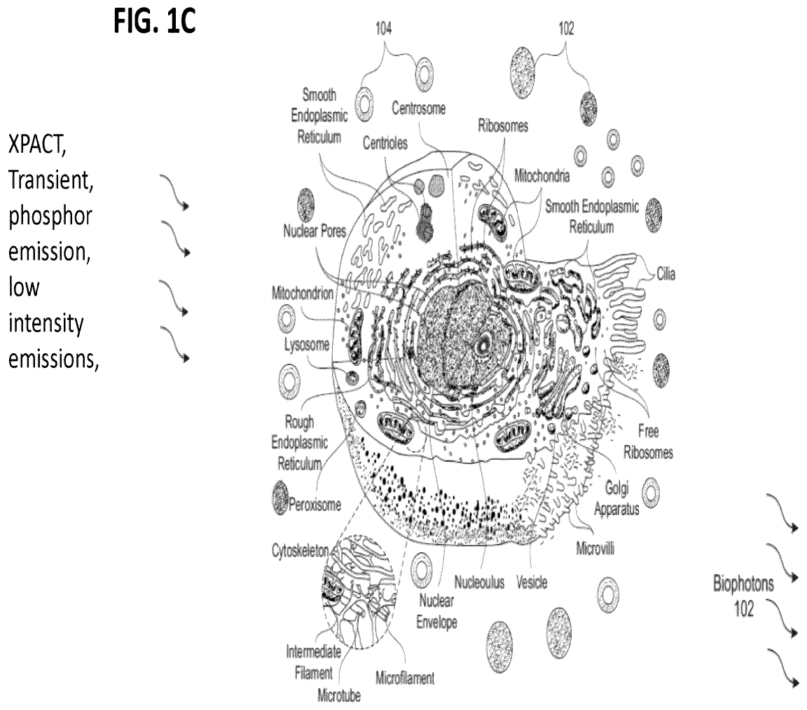

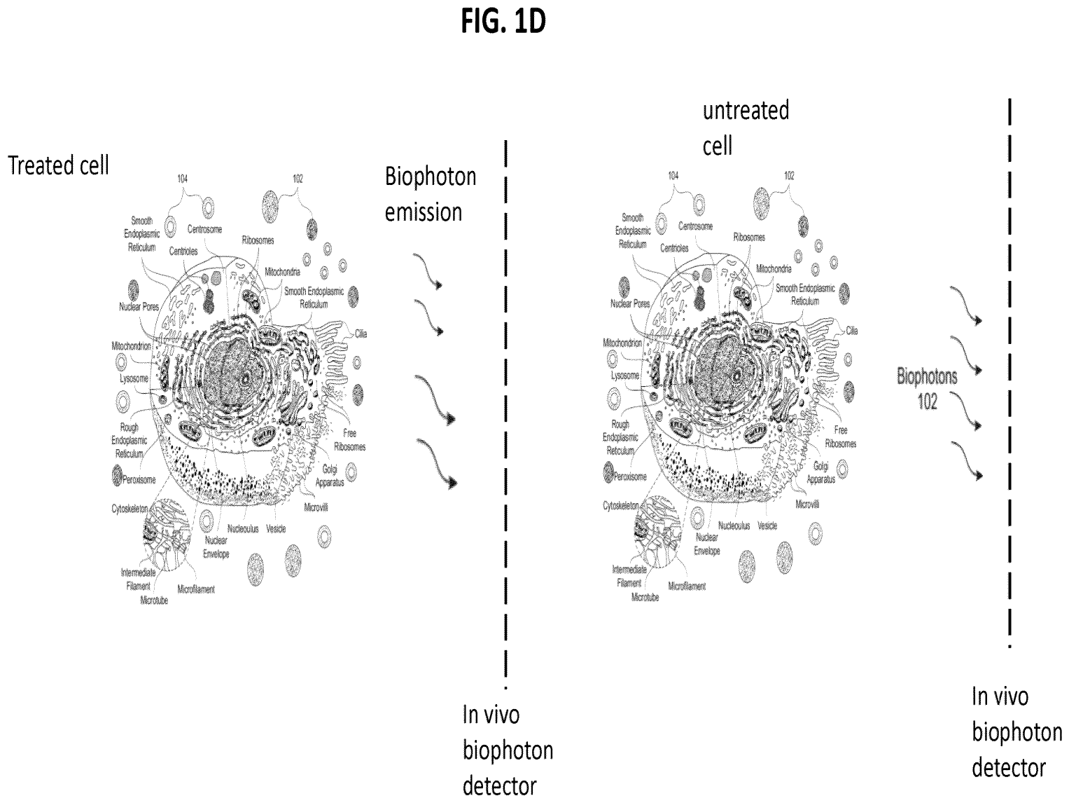

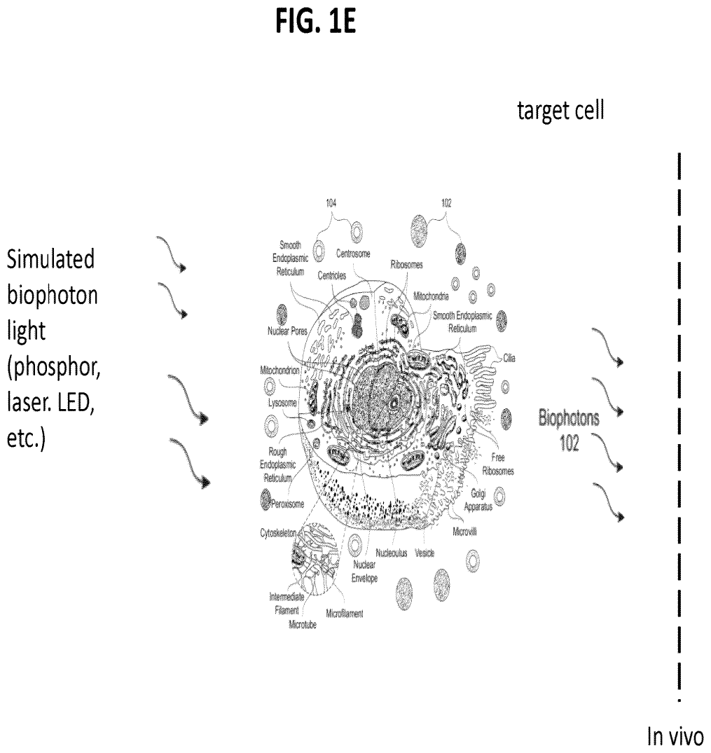

FIG. 1A illustrates the coupling of one region (not shown) into the region shown in FIG. 1A by way of for example “natural” biophoton radiation 102 (that is radiation from nearby living cells). FIG. 1A while depicting one cell represents a collection of cells and may well represent a tissue of an organ at a treatment site. Living cells have been observed to emit biophoton radiation which may play a significant role as a signal carrier for cell-to-cell communication and/or which may itself be a signal that cells at a distance from a treatment site are both a) receiving cell-to-cell communication from the cells at the treatment site and b) responding to the cell-to-cell communication. In one embodiment of the invention, with the coupling of these regions together, due to a change in biological or chemical activity of the cells in a first region, a biological change in a second region inside the subject will be induced.

Coupling as used herein refers to a number of ways that cells in one region induce a biological change in another region. This coupling can utilize mitogenic radiation, biophotonic radiation, electromagnetic radiation, ultraviolet radiation, visible radiation, and near infrared radiation. These radiation types can be a form of cell-to-cell communication or can be indicative of cell-to-cell communication especially when two regions are coupled together, or both. The present invention in various embodiments detects and replicates these radiation types for treatment to a target site. The present invention in various embodiments monitors directly or indirectly the effect of the coupling and/or the radiation (biophoton emissions) between the two coupled regions as feedback on a diagnostic treatment.

Meanwhile, this coupling between different regions can be via the quantum entanglement of associated states, magnetic coupling, coupling via electric field propagation, coupling via bioplasma states, coupling via sonic waves, coupling via single-photon-type non-classical optics, coupling via coherent light emissions, coupling through tunneling nanotubes, coupling through satellite DNA, coupling through biological waveguides, coupling via a biophoton bypass, coupling via stimulation or simulation of biophotonic radiation, and combinations of any of these mechanisms described above and in more detail below. Regardless of the coupling mechanism, according to one embodiment of the invention, there is provided a method of treating a subject comprising: providing a first region of biological material coupled to the subject; initiating a change in a cellular environment of the cells in the first region (for example using simulated biophoton emission determined in the present invention by measuring biophotons from known cell reactions); and optionally due to a change in biological or chemical activity of the cells in the first region, inducing a biological change in a second region inside the subject while detecting biophotonic radiation to or from the second region or by stimulating the second region with artificially produced biophotonic radiation (preferably correlated with a predetermined cell response).

In one embodiment, the phosphors 104 shown in FIGS. 1A, 3A, and 4A can mimic a “natural” biophoton radiation 102 and thus provide the aforementioned simulated biophoton emission, and therefore may induce the same or similar changes that would have been induced by the natural biophoton radiation. Light emission from phosphors 104 may also be used to stimulate the “natural” biophoton radiation 102 or may stimulate the generation of “naturally occurring” chemical messengers.

FIG. 1B is a schematic showing the cellular components of FIG. 1 along with the presence of biophoton emission acting as a sensor of cell activity change at a distal site. More specifically, FIG. 1B illustrates the emission from the region shown in FIG. 1 reflecting the “sensor effect” of the present invention where the coupling of the region shown to another region (not shown) being treated produces “natural” biophoton radiation 102 from the untreated region. In one embodiment of the invention, with the coupling of a treatment region to an activation region together, due to a change in biological or chemical activity of the cells in a first region (the activation region), a second region not directly treated (the treatment region) emits radiation.

FIG. 1C is a schematic showing the cellular components of FIG. 1 along with the presence of biophoton emission produced in response to stimulation light. More specifically, FIG. 1C shows prior art work from some of the inventors where XPACT (with psoralen), or in general phosphor emissions in vicinity of the cell(s) or low intensity UV phosphor emissions in the vicinity of the cell(s) is known to induce programmed cell death. The tissues undergoing programmed cell death would emit biophotons. Here, the biophoton emission from the region shown in FIG. 1 is activated by stimulation light (such as for example from nearly phosphors being driven by an activation source such an X-ray or laser). In one embodiment of the invention, with the coupling of these regions together, due to a change in biological or chemical activity of the cells in a first region, a second region (not directly treated) emits radiation. In one embodiment of the invention, the detection of biomarkers (indicative of a biological change) at an untreated site can provide an indication of cell-to-cell communication to the untreated site, and can provide a feedback for a patient's treatment schedule.

FIG. 1D shows various embodiments of the present invention where the biophoton emission from a treated cell is measured/detected by an in vivo biophoton detector in the vicinity of the cell(s) being treated. The in vivo biophoton detector may directly measure the biophoton emissions, may measure directly or indirectly cell progression or regression (as an indicator biophoton emissions or cell-to-cell communication between for example a treated and untreated site), with in one embodiment the coupling of the two regions together enhanced, or may measure biomarkers indicative of cell progression or regression (to be detailed later).

In FIG. 1D, an untreated cell remote from the treated cell (i.e., at a distance where it would not be exposed to the treatment protocol) reacts to cell-to-cell communication from the treated site, and for example experiences programmed cell death. In one embodiment, the biophoton emissions from treated cell is characterized in terms of its wavelength(s), intensity, modulation, etc. so that the biophoton emission can be replicated. In one embodiment, the biophoton emission from treated cell is compared to the biophoton emission from the untreated cell as an indicator that the reaction in the untreated cell is following the trends (death/growth/etc.) in the treated cell.

FIG. 1E shows one embodiment of the invention where simulated (replicated) biophoton light treats a target cell. In one embodiment, detection of biophotons emitted from the target cell by the in vivo biophoton detector may provide feedback for “tuning” the simulated biophoton light to improve the diagnostic treatment.

### The Biophoton Collector

As used herein, because the exact nature of the biophoton radiation is not known and, because it may well comprise many different kinds of radiation, the radiation collector of the present invention is a collector (or a series of different kinds of collectors) that can collect radiation from various spectra ranging from ultraviolet light through visible, infrared, and far infrared bands. Furthermore, in one embodiment, the radiation collector is designed to collect electric and/or magnetic field radiation emitted from the live biological cells. Moreover, in one embodiment, the radiation collector is designed to collect acoustic or sonic waves emitted from the live biological cells and to redirect and/or amplify those collected signals to a treatment region.

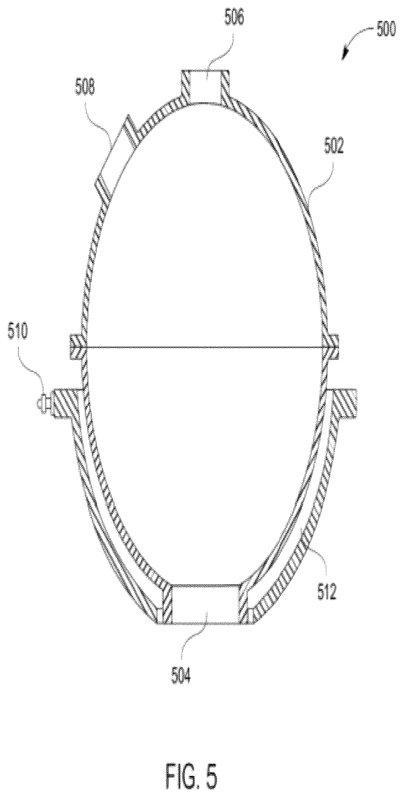

One optical device suitable for collecting biophotons would be an integrating sphere. US Pat. Application Publ. No. 2017/019867 (the entire contents of which are incorporated herein by reference) describes an integrated sphere configuration suitable for the present invention, except that the window element in the '867 application) would be replaced by a cell holding a sample of living tissue. FIG. 5 is a depiction of a biophoton collector 500 according to one embodiment of the invention.

As shown in FIG. 5, the biophoton collector 500 includes an integrating sphere 502 with a highly reflective inner surface as described below. The biophoton collector 500 includes living cell container 504 shown in FIG. 5 at the base of the sphere 502. The biophoton collector 500 includes an output window 506 for transmitting the biophotons from the sphere 502. The biophoton collector 500 includes optionally a stimulation window 508 which can be used to expose cells in container 504 to radiation which can stimulate biophoton radiation. The biophoton collector 500 includes nozzle 510 for supply cells or nutrients or effluent to the container 504. Channel 512 can be used for supply and removal of the effluents.

In one embodiment of the invention, the biophoton collector 500 would likely be disposed outside a patient with a transmission optic (not shown) for transmission of the light from window 506 into a patient. The integrating sphere 502 would have its interior surfaces made of and/or coated with a highly reflective material. For example, the integrating sphere 502 can be formed from a hollow sphere, with an inner wall of the sphere is coated with a material coating layer (e.g., a barium sulfate layer or titanium dioxide, etc.). Biophoton light emitted from container 504 would be reflected on the interior surfaces and directed to an output window 506. In one embodiment of the invention, having a thin layer of the biological material on container 504 would avoid self-absorption effects and provide a source of biophoton radiation to be transmitted to a diseased site. In one embodiment of the invention, nozzle 502 provides a way to add effluent to container 504 such as hydrogen peroxide to induce controlled cell death or nutrients to promote cell growth.

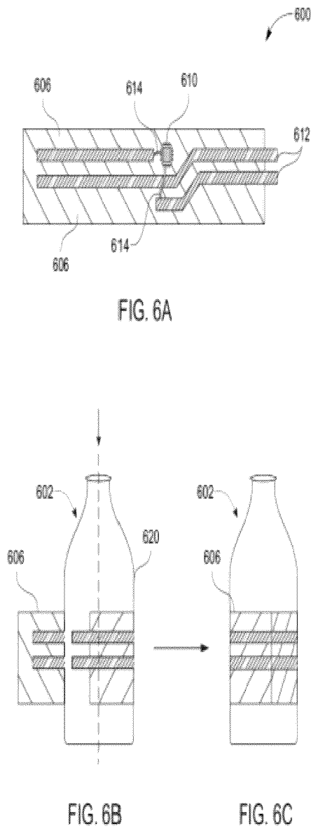

For biphoton emission of electromagnetic radiation in the radio wave or microwave spectrum, an antenna can be used. FIGS. 6A-6C are depictions of an electromagnetic biophoton collector 600 of one embodiment of the present invention.

Biophoton collector 600 is similar to that described in US Pat. Application Publ. No. 20010/0032437 (the entire contents of which are incorporated herein by reference). Biophoton collector 600 includes a container 602 for storing substances. The container 602 is provided with a radio frequency antenna 604. Circuitry 606 can include a chip 610, circuit paths 612 forming a coil of the antennae and wires 614 for connecting the circuit paths with chip 610.

As shown in FIGS. 6B and 6C, the circuitry is disposed on an exterior surface 620 of container 602 and in the embodiment shown encircles the container 602. Inside container 602 would be live cells. In one embodiment of the invention, an effluent can be added to container 602 such as hydrogen peroxide to induce controlled cell death or nutrients to promote cell growth. In one embodiment of the invention, biphoton emission as electromagnetic radiation would be collected and transmitted from circuitry 606 to a target treatment region.

In one embodiment of the invention, biphoton emission as electromagnetic radiation would be detected and its waveform characteristics would be stored by chip 610. In one embodiment of the invention, a radio wave or microwave generator (or another electromagnetic radiation broadcaster) could use the stored waveform characteristics to generate/simulate biphoton for transmission to a target treatment region.

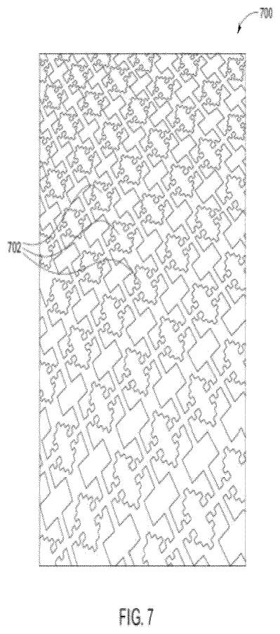

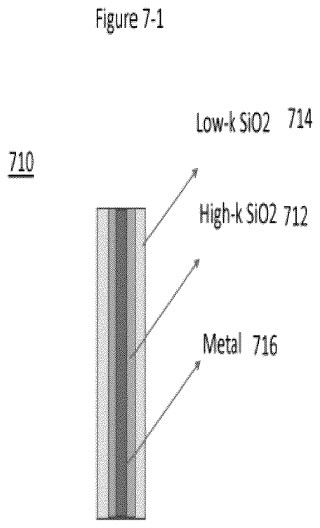

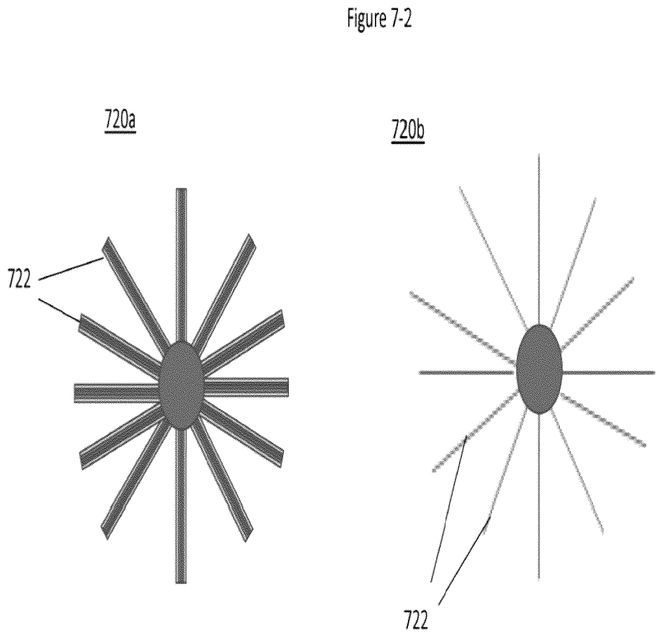

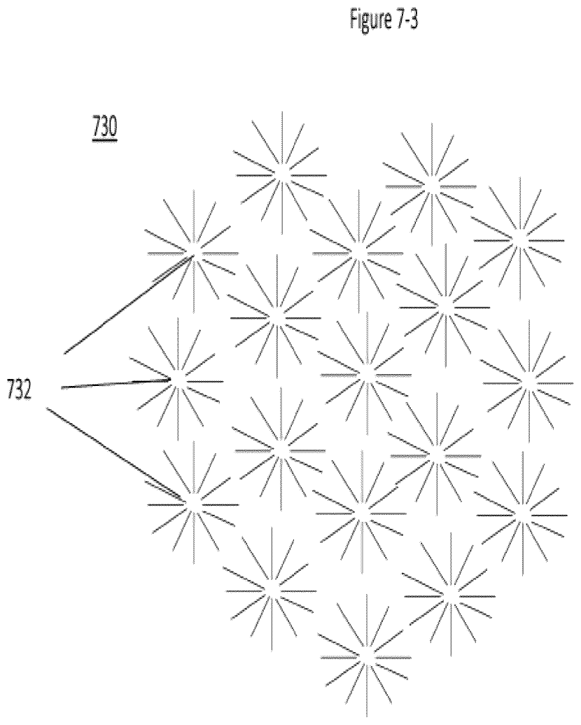

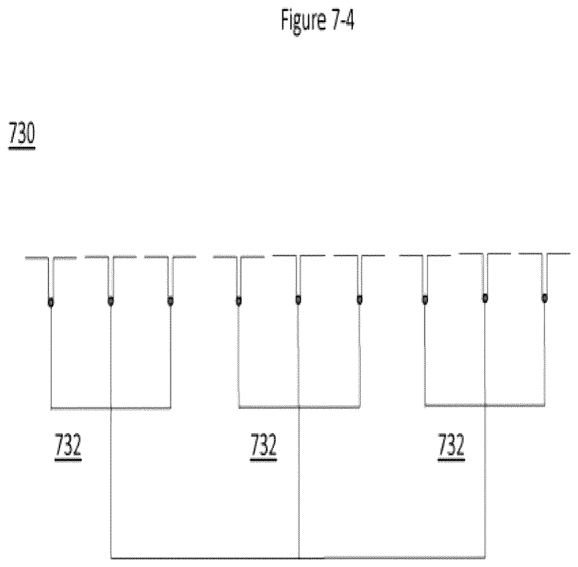

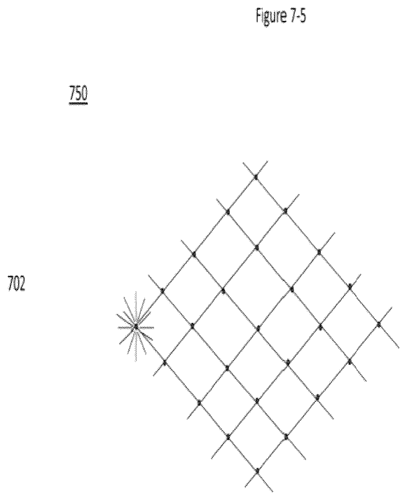

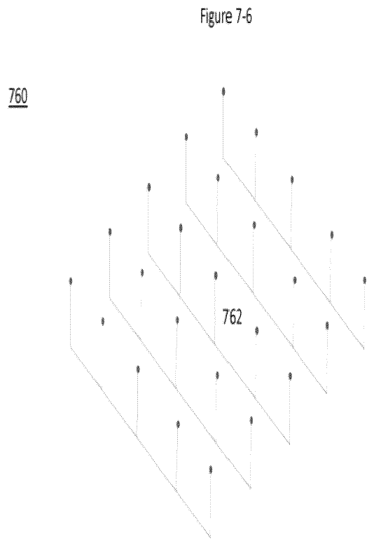

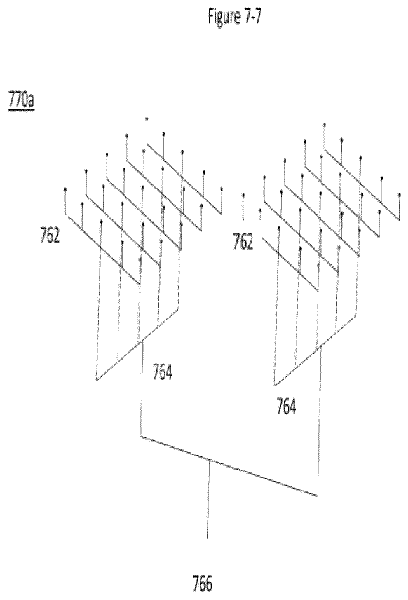

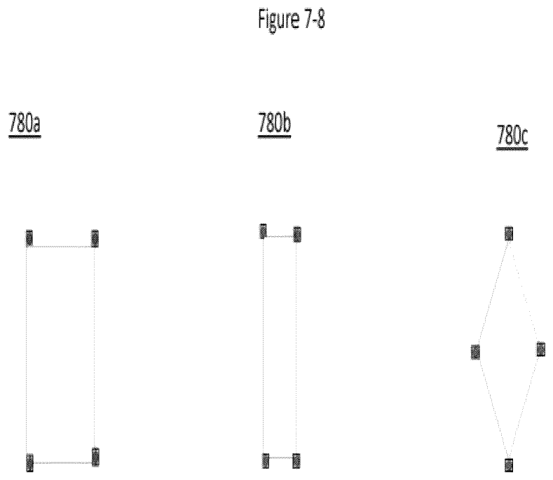

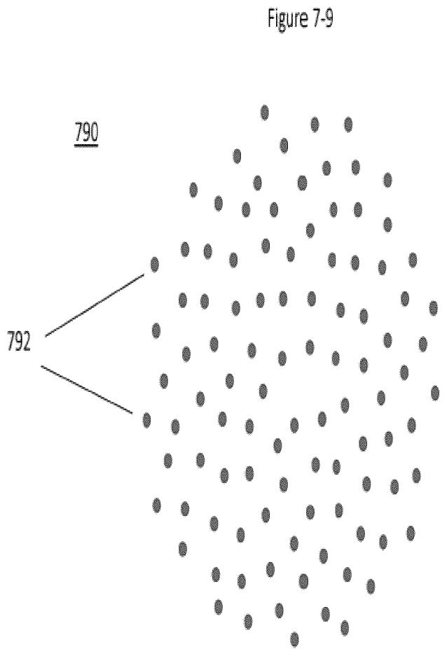

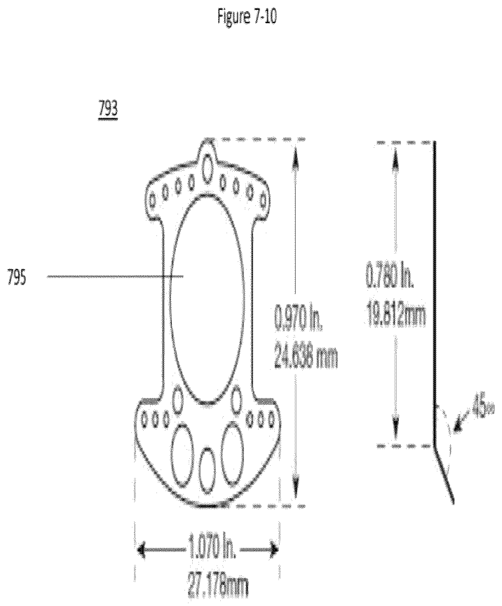

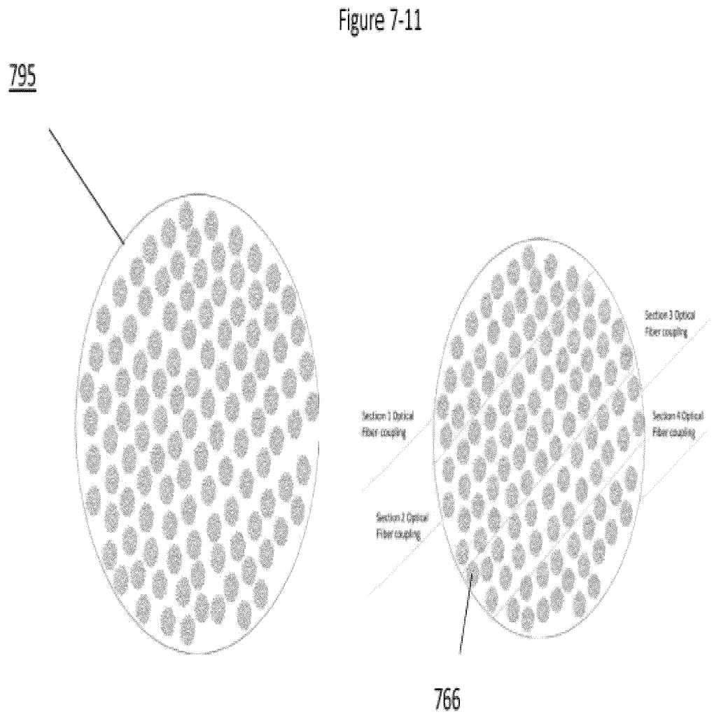

FIG. 7 is a depiction of a fractal antenna that can be used in one embodiment of the invention as the antenna for electromagnetic biophoton collector 600.

A fractal antenna uses a self-repeating design such as self-repeating design 702, or other fractal patterns. It can maximize the length of an antennae material in a total surface area. In general, fractal antennas are compact and have a wide band of operation because a fractal antenna resonates at many different resonances, meaning it can act as an antenna for many different electromagnetic frequencies. The different resonances arise because the fractal nature of the antenna acts as a virtual network of capacitors and inductors.

In one embodiment of the invention a fractal antenna could be printed (or otherwise formed) onto the external surface 620 of container 602. In one embodiment of the invention a fractal antenna could be printed (or otherwise formed) on a Petri dish. In one embodiment of the invention a fractal antenna could be printed onto a biocompatible polymer supporting living cells. These fractal antennae would be used to collect biophoton electromagnetic radiation.

Regardless of the antenna used, the antenna could be connected to a spectrum analyzer to evaluate the frequency characteristics of the electromagnetic radiation captured from the biological cells. Once measured, a rf or microwave generator could be used to replicate the measured spectrum.

### Antenna Design for Light Collection:

Due to the circular polarization of light, it is difficult to maximize optical fiber coupling to the source of light, especially if the source of light is small and/or the intensity of light is very weak, as is generally thought to be the case for naturally occurring sources of biophotonic radiation. In one embodiment of the present invention, the circular polarization of the electric field of non-polarized light is best captured by an optical waveguide having metallized stubs of different orientations. In one embodiment, a stub would be dimensioned about 14 wavelength wide by % wavelength long, and the stubs would be oriented in all possible concentric and spherical radiated orientations. In one embodiment of the invention, the biophotonic activity taking place is measured either in-vivo through a window chamber (described below) or in-vitro in a well plate or in a container. A planar array, multiple stub configuration provides a unique antenna for other purposes and one that is suited for collection of biophoton radiation.

In one embodiment of the invention, the collection of biophoton radiation including light can use a fractal antenna design, similar to that described above for collection of electromagnetic radiation collection at radio or microwave frequencies, but in this embodiment designed for the visible light range or frequencies about the visible light range and much shorter that the radio or microwave frequencies. In one embodiment, the repetitive patterns do not have stubs with lengths shorter than λ/8. Preferably, the antennae stubs have lengths that range from near λ/4 to near 3λ/4. Accordingly, if the intended light measurements are centered around 300 nm, for example, then the stub length of interest would be between 75 nm and 225 nm.

The fabrication of the antenna can be performed using well known semiconductor processes for build-up of small metallic features, including, but not limited to, low-k SiO₂ dielectric, and high-k SiO₂ dielectric. The growth of various layers could be done through a sequential build-up process. The metallized features can be achieved through metal atomic layer deposition (ALD) or through other metal deposition processes known in the art such as sputtering or evaporation, with photo-resist processing used to pattern the deposited metal layer(s) leaving the appropriate metallized patterns of interest. The metallic pattern in one embodiment would be surrounded by a high-k dielectric in contact with the metal, and that structure embedded inside a low k dielectric to a form a sensitive optical waveguide that is capable of detecting the stimulus of a weak electric field from the bio-photonic activity. The fabrication of the antenna may also be completed using 3D printing technology (i.e. conductive filaments and cell printing) to fabricate antennas both in vivo and ex-vivo.

Metallic features are considered in electromagnetic theory to have an infinite dielectric constant, and are therefore able to pick up an oscillating electric field of the biophotons. The electromagnetic energy propagates along the path of the highest dielectric constant which in this case is the metal. The light can and will propagate along a path with the the high k SiO₂ dielectric. However, due to internal scattering, the light will remain confined to the high-k SiO₂ dielectric and the metal. Any time the electromagnetic energy approaches the boundary interface between the high-k SiO₂ and the low-k SiO₂, it will bend back and confine itself to the intended waveguide area formed by the metallic path as surrounded by the high k dielectric material. FIG. 7-1 is a schematic showing a section of waveguide 710 with a high-k dielectric material 712, a low-k dielectric material 714, and a central metal 716.

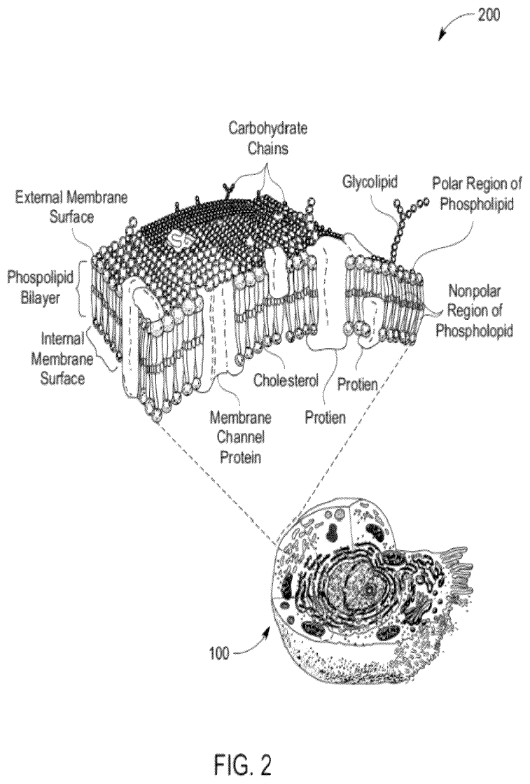

The patterning can be done on a quartz wafer of appropriate dimensions. The antenna pickup area is preferably of an open concentric polarization construction 720a as shown in FIG. 7-2. In one embodiment, the polarization orientation of the quartz wafer may be linear and/or circular.

Metal stubs 722 extend radially from a common center. Other patterns are possible and can be used, including the simpler representation of the open concentric polarization construction 720b also shown in FIG. 7-2.

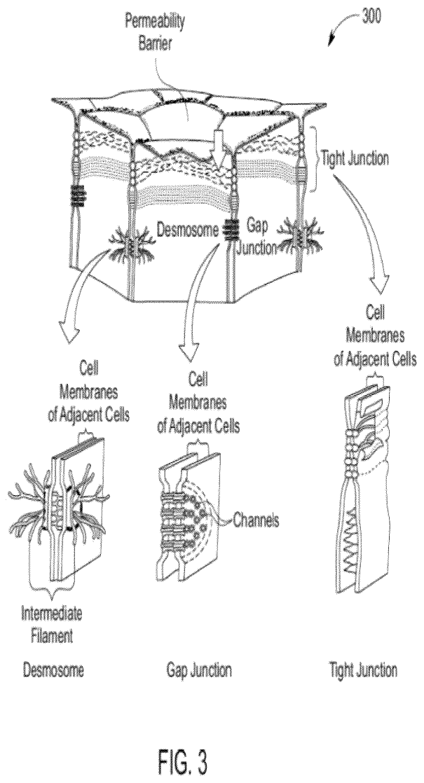

FIG. 7-3 depicts an array 730 of antennae 732 configured on a quartz wafer (not shown). Various patterns are possible depending on the intended use. As an example, the arrayed antennae each have pick up stubs that are concentric and planar as shown in FIG. 7-3.

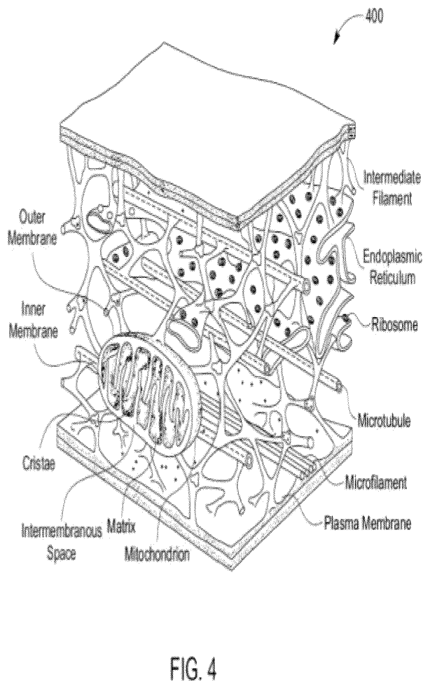

The antenna stubs connect with an internal column (see internal column 766 shown in FIG. 7-7) made of the same materials design as FIG. 7-1 having a configuration with a metal core, surrounded by a high k SiO₂ dielectric and a low k SiO₂ dielectric that forms the optical waveguide. The metal can be, but is not necessarily, made of a metal enabling a photoelectric effect. A cross section of the stub configuration 730 shown in FIG. 7-3 with antennae 732 interconnected together is shown in FIG. 7-4.

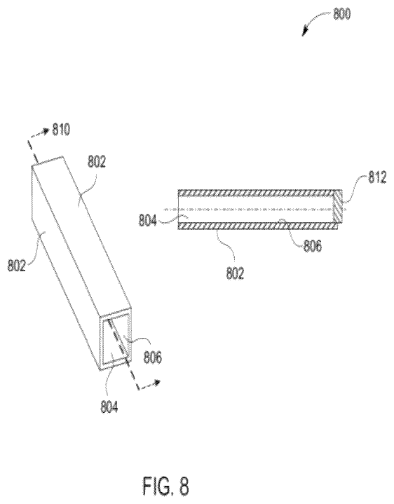

FIG. 7-5 is a schematic of multi-up arrayed antenna 750. FIG. 7-6 is another schematic of the multi-up arrayed antenna 750 shown in FIG. 7-5 showing a top-level interconnection network 762 under the top surface of multi-up arrayed antenna 750. FIG. 7-7 is another schematic of the multi-up arrayed antenna 750 shown in FIG. 7-5 showing the full interconnection network including top-level interconnection network 762 and bottom-level interconnection network 764. FIG. 7-8 is a depiction of antennae that can be arrayed in different manners including a square antenna 780a, a rectangular antenna 780b, and a diamond shaped antenna 780c. FIG. 7-9 is a depiction of a spiral-type packing arrangement 790 where each antenna petal 792 is placed at 0.618034 per turn (out of a 360° circle) allowing for the best possible exposure to cellular-light. This desirable spiral arrangement follows from what is commonly referred to as the Fibonacci sequence.

The resulting multi-up pattern has a high density and a spiral configuration similar to the one found in pine cones and sunflowers. This spiral pattern is desirable for the packing it enables.

This patterned antenna can be built on a quartz wafer of any size that can fit within semiconductor equipment capability. A quartz wafer hosting thousands of antennae (2,000 to 100,000 antennae) can be built. This quartz wafer can be used in accordance to the window chamber model. Similarly, the quartz wafer equipped with fractal antennae can be used inside a polycarbonate well plate. The cell plating can be performed on top of the quartz wafer with embedded fractal antennae. Various experiments can be envisaged to elucidate the light-based communication inside of a single cell or amongst multiple cells. The ability to conduct photonic measurement in-vivo using fractal antennae permits one to measure biophoton radiation from living tissue in vivo or in vitro. Similar to that noted above, the fabrication of the patterned antenna may also be completed using 3D printing technology (i.e. conductive filaments and cell printing) to fabricate antennas both in vivo and ex-vivo.

As in other embodiments discussed above, once measured, these signals can be transmitted from their source to a treatment site or could be duplicated to mimic biophoton radiation.

### In-Vivo Measurements of Bio-Photonics:

The window chamber mouse model has gained great acceptance for conducting medical research in-vivo while maintaining the ability to see through for direct observation and monitoring. FIG. 7-10 is a depiction of a window chamber according to one embodiment of the invention, where the window area 795 is constructed for transmission of biophoton radiation therethrough.

For example, window chamber 793 could be equipped with fractal antennae (of the same or different designs) to permit measurements of photonic activity as well as having the ability for direct observation and monitoring. For fractal antennae, the antenna patterning in at least one portion of the window 795 can be made with antenna elements dimensioned at subwavelengths of visible light so that observation of the biological region underneath window chamber 793 is possible. The fractal antenna can be as described herein above, or can be any desired fractal antenna configuration.

FIG. 7-11 is a depiction of a window 795 made of a quartz wafer that has different sections that are independent of each other. This design permits the photonic activity to be measured from different sectors of the subject. In one embodiment, window 795 of FIG. 7-11 could be used to answer the question if there is (ON) or if there is not (OFF) photonic activity. The wavelength or spectral information could be collected and stored regardless of whether the antenna was sectioned or not.

FIG. 7-11 also illustrates one embodiment of the invention where the antennae are sectioned such that photonic activity (or the absence thereof) can be monitored from each section. Each fractal antenna in window 795 could be connected to separate fiber optic columns 766, or all the fractal antennae in each section could be connected together to one common fiber optic column 766.

### The Biophoton Bypass

Complicating the literature recognized problem that biophoton radiation is weak is the further problem that these weak signals (naturally originating inside a subject) travel in a dispersive medium with scattering and absorption making it unlikely that the biophoton radiation can travel extended distances. Even the distances of mm in the in vitro test cells are remarkable. The inventive solution: bypass nature's dispersive optical pathway with an artificial conduit (hereinafter the “biophoton bypass”) having little if any scatter and low absorption.

The biophoton bypass might have physical characteristics of a fiber or fiber bundle if the bio-photons needed to be transmitted over significant distances, as from outside the body into the body or from one region of the body more accessible for the control than the target region.

The biophoton bypass might have physical characteristics of an optical sheet with evanescent waves from the sheet penetrating a shallow depth into a diseased organ.

The biophoton bypass might be a simple polymeric window separating a control region from the diseased organ made along the ways described in the Yevgeny patent application U.S. Pat. Appl. Publ. No. 2009 (discussed in more detail below).

The biophoton bypass might be capillary filled with a protein solution. In prior work, a narrow capillary was filled with a dilute protein solution and exposed to MGR (another name for biophoton radiation) on one end. No radiation was detected at the other end until the protein filed capillary was aligned with an electric field. Hence, in one embodiment, the biophoton bypass of the invention could be a protein-filled conduit wherein an applied electric field which can “gate” to either turn on or turn off the transmission of biophotons along the protein-filled conduit.

In one embodiment, the biophotons emitted from one cell induce photo-assisted reactions in a nearby or proximate cell that itself produces its own biophotonic emission, thereby leading to biophoton emission from one cell to another cell, appearing as a “communication” across many cells.

In one embodiment, the biophotons are emitted from excited states of luminescing species. The set of excited states can be considered a “bioplasma.” In this context, bioplasma is a term derived from bioelectronics, molecular biology and solid state plasma physics and refers to a state in which biomolecules in vivo are predominantly in a stable, collective, excited state. It is considered a “cold plasma” that forms an energetic and informational network throughout the organism involving a colloid of semi-conducting proteins (or natural/unnatural amino acids alone or in combination with the proteins) as the main constituent in a redox (oxidation-reduction) chemical oscillator displaying complex dynamics. This is analogous to a low-power laser that uses chemical, electrical or magnetic energy to pump it into an excited metastable state.

Coupling between the biochemical reactions of the living state takes place electromagnetically, with a wave-like internal coordination surrounded by an electromagnetic wave externally emitted. Biological effects of exogenous electromagnetic fields are ascribed to collective resonance properties of the whole bioplasma and not just to any of its individual parts.

Accordingly, in one embodiment of the invention, the collective state of this bioplasma can be influenced by localized changes. One candidate to influence local changes would be the application of an electric field, to change the polarization of the cells and turn off (or on) chemical reactions. Other candidates are described in more detail elsewhere but include providing ultrasonic, microwave, or localized cooling to selected portions of cells in an organ.

Regardless of the collection technique, an energy transmitting structure (as the biophoton bypass) could carry the biophotons to a target site. An optical fiber could be used if the biophoton light were in the UV to near IR range. In one embodiment, vacuum/air would be the most reliable medium for the biophoton bypass. Accordingly, a hollow optic could be used for a biophoton bypass of the invention for transmitting biophotons in the UV to near IR range inside the hollow optic while bypassing media of the subject to be treated. FIG. 8 is a depiction of a hollow optic biophoton bypass 800 according to one embodiment of the invention. U.S. Pat. No. 8,454,669 (the entire contents of which are incorporated herein by reference) describes a similar device for UV phototherapy. In the hollow optic biophoton bypass 800 of the invention, there are walls 802 which define a hollow cavity 804 filled with air, a gas, or possibly under a vacuum for transmitting UV light into a subject could be utilized in this invention. The interior surfaces 806 would be highly reflective surface. At the distal (or exit) end 810, there would be a light optic which could either disperse or concentrate the biophoton light flux into a treatment site If the biophotons are low frequency electric signals (including a DC field), then a wire or conductive trace (as the biophoton bypass) could be used to transmit the low frequency electric waves. The biophoton bypass while in one embodiment may be considered a light optic or optical waveguide in other embodiments may be an acoustic waveguide transmitting sound waves from one site to another.

In general, the present invention provides a method of treating a subject comprising: initiating a change in a cellular environment of cells in a first region of a biological material inside the subject; and due to a change in biological or chemical activity of the cells in the first region, inducing a biological change in a second region inside the subject by enhancing coupling of the first region to the second region. The coupling may be enhanced by an applied magnetic field extending from the first region to the second region. The coupling may be enhanced by an applied electric field extending from the first region to the second region. The coupling may be enhanced by a light pipe extending from the first region to the second region. The coupling may be enhanced by an acoustic waveguide extending from the first region to the second region. The coupling may be enhanced by growth of nanotubes connecting cells from the first region to the second region. The coupling may be enhanced by chemical transport of biomarkers from the first region to the second region.

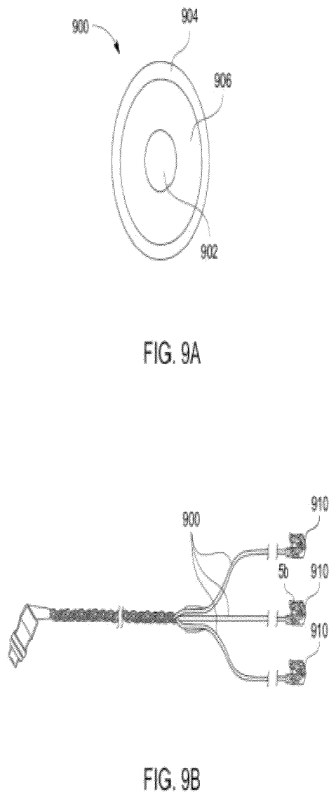

FIGS. 9A and 9B are depictions of an electrically conducting biophoton bypass 900 according to one embodiment of the invention where low frequency electric signals are transmitted therein while bypassing media of the subject to be treated.

The conductors 902 shown in FIG. 9A are similar to those described in U.S. Pat. No. 7,272,427 (the entire contents of which are incorporated herein by reference) where the conductors in the '427 patent were used to measure bio-electric signals from the heart muscle while a patient was in an MRI environment. Here, in the FIG. 9A/9B embodiment of this invention, the electrically conducting biophoton bypass 900 has an electrically conductive part 902 and a sheath part 904 arranged over conductive part 902. The conductive part 902 and sheath part 904 are separated by a dielectric 906. The electrically conducting biophoton bypass 900 can include multiple conductors 902 terminating on connectors 910 for attachment to a subject to be treated. Connectors 910 conductors can be attached to the living cells noted above and/or to a target region if the wire or conductive trace is being used as a biophoton bypass to deliver the low frequency electric signals from a source of the low frequency electric signals to a target site for treatment. In one embodiment, as shown in FIGS. 9A/9B, the multiple conductors 902 with multiple sheaths (not shown) are twisted together to reduce high frequency noise.

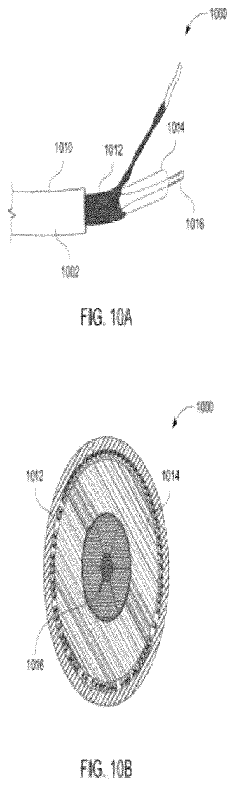

FIGS. 10A and 10B are depictions of another electrically conducting biophoton bypass 1000 according to one embodiment of the invention where the biophotons as high frequency electrical waves are transmitted therein while bypassing media of the subject to be treated.

In the example of FIG. 10A, a coaxial cable 1002 is used. Waveguides (as biophoton bypasses) can also be used to transmit high frequency electrical waves. These devices (coaxial cables and waveguides) are highly selective for delivery of specific frequencies of radiation. As shown in FIGS. 10A/10B, the electrically conducting biophoton bypass 1000 includes a coaxial cable 1002 having an outer plastic sheath 1010, a woven copper shield 1012, an inner dielectric insulator 1014, and a copper core 1016. The core 1016 could be made of other metals or alloys, but copper is commonly used. A coaxial cable differs from other shielded cables because the dimensions of the cable are controlled to give a precise, constant conductor spacing, which is needed for the coaxial cable to function efficiently as a transmission line.

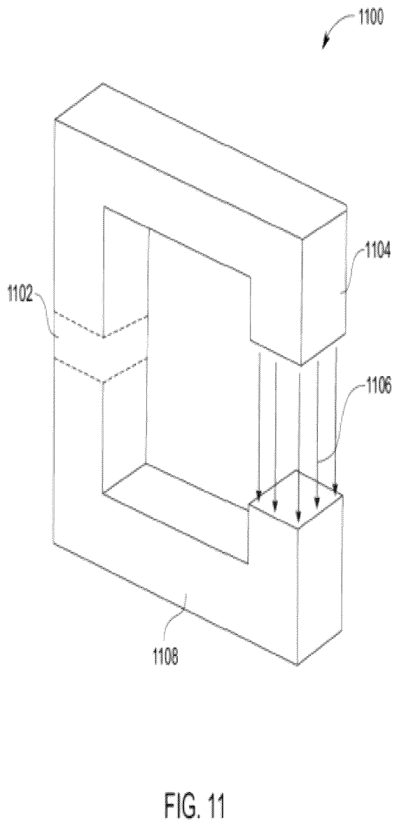

FIG. 11 is a depiction of a magnetic biophoton bypass 1100 according to one embodiment of the invention where the biophotons as time-varying or static magnetic fields are transmitted therein while bypassing media of the subject to be treated. As shown in FIG. 11, magnetically permeable materials form a magnetic circuit (as the biophoton bypass) carrying the time-varying or static magnetic field from a source to a target. The magnetic biophoton bypass 1100 utilizes a dual gap design. In one gap, there is a source of the magnetic fields. As shown in FIG. 11, in one gap, there is disposed a cell containing living tissue, that is living cell biophoton emitter 1102 which is a source of magnetic biophotons. The magnetic yokes 1104 and 1108 form a “circuit” carrying the magnetic field in the circuit from the living cell biophoton emitter 1102 through magnetic yoke 1104 to a target or treatment region 1106, and back by magnetic yoke 1108 to the living cell biophoton emitter 1102.

In a further embodiment of the present invention, the biophoton radiation applied to a first region is capable of triggering an altered metabolic activity in one or more cells, preferably in the 100 GHz to 10 THz region, which triggers the cell(s) to undergo altered metabolic activity, and optionally, to further trigger subsequent biophoton emissions from the cell(s). Microwave broadcasters or microwave waveguide structures can be used to apply these frequencies to a target structure.

In one embodiment, the spiral chains of DNA naturally present in biological materials are used to transmit radiation in the frequency range of 100 GHz to 5 THz or are used for charge transport or signaling along DNA traditionally thought to be “satellite” or “junk” DNA (hereinafter referred to as “signaling DNA”). This signaling DNA corresponds to approximately 98.5% of the DNA strand, with only about 1.5% of the DNA strand functioning genetically to code for proteins or RNA, etc. Traditionally believed to be merely composed of various repeating nucleotide base fragments having no function, this signaling DNA may function as one of the components of cell-to-cell communication or signaling within humans, as well as other animals. That the signaling DNA has some form of function has also been hypothesized by others (see, e.g., Jiin-Ju (Jinzhu), “PHYSICAL PROPERTIES OF BIOPHOTONS AND THEIR BIOLOGICAL FUNCTIONS”, Indian Journal of Experimental Biology, Vol. 46, May 2008).

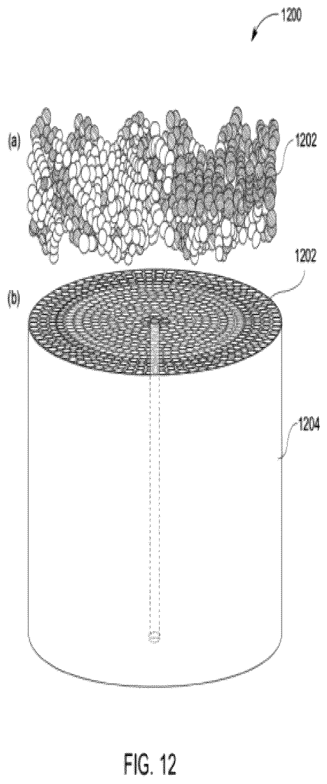

FIG. 12 is a depiction of a DNA-based biophoton bypass 1200 according to one embodiment of the invention where the biophotons in the frequency range of 100 GHz to 5 THz are transmitted therein while bypassing media of the subject to be treated.

In FIG. 12, the signaling DNA 1202 is included in a waveguide type outer structure 1204. The length and diameter of the outer structure 1204 is sized according to the frequency range to be transmitted. Lithographic and printing processes (including 3D printing) can be used to generate trenches in silicon substrates that could both hold the signaling DNA and form a waveguide structure for propagation of biophotons in the frequency range of 100 GHz to 5 THz across the surface of the silicon substrate and to a treatment site. Wafer thinning processes known in the art could be used to thin the silicon wafer making the DNA-based biophoton bypass 1200 potentially a flexible biophoton bypass.

Accordingly, in various embodiments of the invention, the application of biophoton radiation to a target structure may directly affect a diseased region or it may enhance biophoton emissions from a first region (where cell death is being artificially induced) to a second or treatment region). This biophoton emission may also act as a way of “communicating changes” in the first or control region which induce changes in the second or target region. This artificial biophoton emission may also act to enhance naturally occurring biophoton emission. This biophoton emission may also result in or be a consequence of quantum coupling (or quantum entanglement) between the control and the target regions. Such quantum coupling (or quantum entanglement) may be assisted by a magnetic field in there the control and the target regions or by a common magnetic field between control and the target regions.

Accordingly, in various embodiments of the present invention, the first and second regions are “coupled” to each other with a medium (whether artificial or natural or that intrinsically present in the biological materials of the first and second region) that transmits bio-photons to the target region as a way of “communicating changes” in the first or control region which may induce (or be representative of) changes in the second or target region.

### Living-Cell Biophoton Radiators

Accordingly, in one embodiment of the present invention, live biological cells in a container could be used as a source of biophoton radiation. A number of ways can be used to form this type of source. In one example, a Petri dish or container outside the subject could contain the live biological cells. See FIGS. 5 and 6. In one embodiment of the invention, the base of the Petri dish would contain a radiation collector in near direct contact with the living cells. The radiation collector would have (if needed) a thin passivation layer to insure that the materials of the radiation collector do not interact with the solutions in the petri dish.

Biophoton radiation emitted from the biological cells would be captured by the optical collector and then transmitted to a treatment site, for example inside the subject. For example, a cancer strain (the same or similar to that of a patient) could be treated in a container with hydrogen peroxide to induce cell death. The biophoton radiation would be collected from the container and transmitted in a biophoton bypass (bypassing intervening tissue of the patient) into the diseased region promoting cell death.

U.S. Pat. Appl. Publ. No. 2009/0203530 (the entire contents of which are incorporated herein by reference) describes a method for producing polymers having properties suitable for catalytic activity or binding activity, via evolutionary nucleic acid-mediated chemistry. As described in the '530 application and suitable for the present invention, non-biological polymers (e.g., polymers other than DNA, RNA, or protein) can be synthesized. Such polymers include, but are not limited to, peptide nucleic acid (PNA) polymers, polycarbamates, polyureas, polyesters, polyacrylate, polyalkylene (e.g., polyethylene, polypropylene), polycarbonates, polypeptides with unnatural stereochemistry, polypeptides with unnatural amino acids, and combination thereof. In certain embodiments, the polymers comprise at least 10, 25, 75, 100, 125, 150 monomer units or more. These polymers could be used to encapsulate the biological cells of the living-cell biophoton radiator.

In this embodiment, a living-cell biophoton radiator could exist outside the patient or be surgically disposed inside the patient at the diseased site. U.S. Pat. No. 8,999,376 (the entire contents of which are incorporated herein by reference) describes tissue patches comprising fibrinogen (and/or fibrin). This type of fibrin glue has been approved by the FDA and can be used to impart topical hemostasis, provide sealant properties that are suitable is some clinical applications, and promote tissue approximation. Fibrin glue mimics the final steps of the coagulation cascade. FIGS. 13A and 13B are depictions of a living-cell biophoton radiator 1300 according to one embodiment of the invention where living cells are added as a part of living cell layer 1320.

The matrix 1310 shown in FIGS. 13A/13B can be in the form of a cylindrical disc 1350 with a substantially circular cross-sectional geometry. In other embodiments, the matrix 1310 (or the entire tissue patch) can have other cross-sectional geometries such as, for example, substantially elliptical, polygonal (e.g., including any number of sides such as in the form of a triangle, a quadrilateral (e.g., rectangular or substantially square), etc.), irregularly-shaped, or any other suitable shape such as for example both organic and inorganic “microneedle” patches.

In the present invention, these types of patches 1310 can be applied to organ tissue. For example, matrix 1310 would be attached to an organ (not shown). Living cells of a kind similar to that to be treated using biophoton radiation would be contained in living cell layer 1320. An encapsulant layer 1330 would be applied over the living cell layer 1320. In one embodiment of the invention, encapsulant layer 1330 would contain either a substance to promote cell growth or a substance to promote cell death which would be controllably released into the living cell layer 1320.

In one embodiment of the invention, the matrix 1310 would be a porous or semi-porous structure having pores 1350 in a membrane 1355 permitting biological and fluid connections from living cell layer 1320 and the organ to be made.

As the cells in the living cell layer 1320 are affected by the substances released from the encapsulant layer 1330, biophoton radiation from living cell layer to the organ is achieved. Alternatively, encapsulant layer 1330 could contain phosphors or other elements such as metals significantly heavier than carbon for preferential absorption of x-rays (with the phosphors producing ultraviolet or visible light) or for preferential absorption of microwaves (with the metals locally heating). In one embodiment, the UV or visible light or the local heating would “stress” the cells in living cell layer 1320 to thereby produce biophoton radiation.

U.S. Pat. Appl. Publ. No. 2010/0120117 (the entire contents of which are incorporated herein by reference) describes polymers may be used to coat living cells in cell therapy applications, and thereby would be suitable as a container used in the present invention for holding biological cells of the living-cell biophoton radiator. The '117 publication describes a polymer-coated cell construction comprising a living cell and a polymer comprising at least one recurring unit represented by a formula selected from the group consisting of formula (I), formula (II), and formula (III)

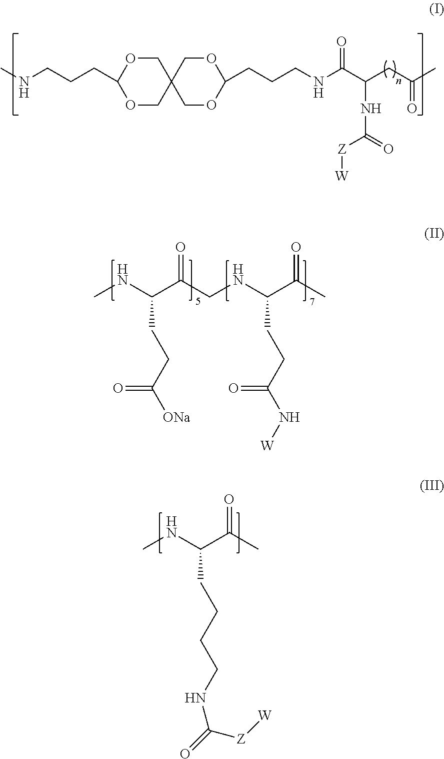

wherein n is 1 or 2; wherein x and y are each individually integers of from about 1 to about 500; wherein Z is an optional linker group comprising from about zero to about 20 carbon atoms, from about zero to about 5 oxygen atoms, from about zero to about five nitrogen atoms, from about zero to about 5 sulfur atoms, and from about zero to about five phosphorous atoms; and wherein each W is individually selected from the group consisting of biotin, a fatty acid, a fluorescent dye, an antibody, a peptide, a targeting ligand, a polysaccharide, and a negatively charged group, the polymer being non-covalently attached to at least a portion of the exterior of the living cell.

The '117 publication further describes a method for coating a living cell, comprising intermixing the living cell with a polymer which includes at least one recurring unit represented by a formula selected from formulas (I), (II), and (III) as described above, wherein the polymer is intermixed with the living cell in an amount effective to at least partially coat the exterior of the living cell.

Similar to that described in the '117 publication, in one embodiment of the present invention directed to the living cell biophoton radiator, a variety of diseased cells may be contained or carried by the polymer-coated cell construction noted above. These diseased cells may include cells exhibiting neurologic diseases (e.g. Parkinson's disease, multiple sclerosis), cardiovascular disease (myocardial ischemia, repair and regeneration of infarcted myocardium), hepatic disease (liver failure), diabetes, skin, and renal failure (chronic renal failure, acute renal failure), and cancer tissues.

In one embodiment of the invention, the target tissue to be treated with the living-cell biophoton radiator of this invention may be an organ such as heart, brain, kidney, skin, liver, muscle, spleen, lung, spinal cord and bone marrow. Tissues of this type or from these organs can be biopsied, cultured, and returned to the patient at the site of the disease. These cells may contain therapeutic agents to promote cell death or cell growth depending on the treatment under consideration. As these therapeutic agents work, biophoton emission radiates adjacent cells not contained in the polymeric coating, thereby inducing a change in the adjacent cells.

Conventionally, there are four basic issues for cell-based therapies. These are mobilization of the cells, homing to a target site, integration into the native tissue or organ and survival of the implanted cells. In one embodiment of this invention, the polymer coatings assist integration of cells of the living-cell biophoton radiator into native tissue and survival of implanted cells at least until biophoton radiation from the polymer encased cells can be used. By coating of the cells with the polymers, the cells may be protected in the blood for several hours. The polymer coated cells may also be protected from the immune response of the host. These coatings may protect the cell therapeutic while allowing passage of vital nutrients including oxygen.

The selection of cell type is a function of the disease which is being treated, the cell type being coated and forming part of the living-cell biophoton radiator. For example, skeletal myocytes would be injected into post-myocardial infarction scar tissue; neuronal cells would be administered to the brain of patients with Parkinson's Disease. Cell sources which may be used for the living-cell biophoton radiator of this invention include embryonic stem (ES) cells, adult stem cells, progenitor cells such as skeletal myoblasts, fetal and neonatal cariomyocytes, and chord blood.

As an example of cells contained in the above noted polymer for the living-cell biophoton radiator of this invention, cardiovascular and lung tissues may also contain progenitor or stem cells that under the correct conditions could be induced to proliferate and repair cellular damage. For instance, recent findings suggest that a sub-population of fetal proliferative alveolar epithelial stem cells is present in adult lung. In addition, other tissues such as skin, liver, brain, and muscle have progenitor or stem cell populations that may provide additional sources of cells for cellular therapies.

For neovascularization of ischemic myocardium, endothelial progenitor cells for the living-cell biophoton radiator of this invention may be injected into the target area to promote new vessel growth. The cells are isolated from the mononuclear cell fraction of bone marrow or peripheral blood. The cells may be whole isolated cells or the cells may first be expanded in culture. Other examples for the living-cell biophoton radiator of this invention include treatment of skin disease with replacement grafts. Skeletal stem cell implantation may be used for bone regeneration. Chondrocytes may be used to repair joint cartilage. Acute and chronic renal failure may be treated with stem/progenitor cells using the living-cell biophoton radiator of this invention.

The cell source for the living-cell biophoton radiator of this invention may be either an autologous source or a non-autologous source. In some embodiments, the cells may be genetically modified. In cases where an adequate supply of cells is not possible from the patient due to the disease or other condition, non-autologous sources may be used. Non-autologous cells include allogeneic and xenogeneic cells. Non-autologous sources must overcome the natural host immunologic rejection processes. The polymer coating according to the embodiments provides protection from the host immune response.

The use of autologous cells generally involves obtaining the patient's own cells, expanding the cells in vitro in large quantities over several weeks, and reintroducing the cells in a site-specific manner.

A variety of means for administering cells for the living-cell biophoton radiators of this invention will be apparent to those of skill in the art. Such methods include injection of the cells into a target site in a subject. Cells may be inserted into a delivery device which facilitates introduction by injection or implantation into the subjects. Such delivery devices may include tubes, e.g., catheters, for injecting cells and fluids into the body of a recipient subject. In a preferred embodiment, the tubes additionally have a needle, e.g., a syringe, through which the cells of the embodiments can be introduced into the subject at a desired location. In a preferred embodiment, cells are formulated for administration into a blood vessel via a catheter (where the term “catheter” is intended to include any of the various tube-like systems for delivery of substances to a blood vessel). The cells may be prepared for delivery in a variety of different forms. For example, the cells may be suspended in a solution or gel. Cells may be mixed with a pharmaceutically acceptable carrier or diluent in which the cells of the embodiments remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic. Conventional sterilization techniques may be used such as for example UV-C, gamma, heat, and/or e-beam exposure. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Modes of administration of the polymer coated cells include but are not limited to systemic intracardiac, intracoronary, intravenous or intra-arterial injection and injection directly into the tissue at the intended site of activity. The preparation can be administered by any convenient route, for example by infusion or bolus injection and can be administered together with other biologically active agents. Administration is preferably systemic. Most preferably, the site of administration is close to or nearest the intended site of activity. In some embodiments, the polymer coated cells will migrate or home to the tissue or organ in need of treatment in response to chemotactic factors produced due to the injury without specific modification of the polymer coated cells for targeting.

Modifications of the polymer coating can provide for homing of the cells for the living-cell biophoton radiators of this invention to the target site. Protein targeting agents such as antibodies or proteins that bind to specific membrane sites may be used to target the polymer coated cells to the target organ or tissue. In some embodiments of the methods described herein, the polymer coated cells are modified prior to implantation into the individual so as to promote their targeting to tissue or organ in need of treatment. For example, the polymer may include an antibody which binds an antigen that is abundant at the target site, that is, at the site of the tissue or organ which is diseased or in need of treatment.

For example, monoclonal antibodies are known that specifically target cancer cells. Antibody fragments and cell specific peptides can also be used to target cancer cells. Many of these are antibodies to growth factor receptors which are preferentially expressed on the surface of cancer cells. These include the humanized monoclonal antibody trastuzumab (Herceptin) which targets the HER-2/neu oncogene (Sato, et al. (2005) Int. J. Radiation Oncology Biol. Phys. vol. 61 (1): 203-211). The HER-2/neu oncogene is found in ovarian cancer, lung cancer, gastric cancer, oral squamous cell carcinoma, breast cancer, and esophageal cancer. BLCA-38 monoclonal antibody has been shown to target prostate and bladder cancer (Russell, et al. (2004) Cancer Immunol Immunother. vol. 53:995-1004). Other monoclonal antibodies are known and it is within the level of skill in the art to select a monoclonal antibody appropriate to the cancer or other disease or injury to be treated.

Migration of polymer coated cells for the living-cell biophoton radiators of this invention to target tissues may be enhanced by genetic modification, e.g., introduction of an exogenous nucleic acid encoding a homing molecule into the cells. Examples of homing molecules include receptors specific to the target tissue such as chemokine receptors, interleukin receptors, estrogen receptors, and integrin receptors.

In various embodiments, a receptor ligand such as transferrin or epidermal growth factor can be included in the polymer for homing to cancer cells. These ligands provide specific targeting to receptors on tumor cells. Thus, delivery of the coated cells is localized to the area in need of treatment for maximum effectiveness.

Another method of homing a cell such as a stem cell to an injured tissue is carried out by increasing the amount of an injury-associated polypeptide, e.g., a cytokine or adhesion protein, in the injured tissue. The method increases the number of stem cells in an area of injured tissue compared to the number of stem cells in the area in the absence of an exogenous injury-associated polypeptide or nucleic acid encoding such a polypeptide. For example, identification of injury-associated polypeptides, e.g., growth factors, activate endogenous mechanisms of repair in the heart such as proliferation and differentiation of cardiac progenitor cells. These effects can give rise to biophoton radiation supplementing healing in adjacent cells. The injured tissue is contacted with a nucleic acid encoding a protein such as a cytokine or adhesion protein. Alternatively, cells such as fibroblast cells expressing exogenous nucleic acid molecules encoding the cytokine or adhesion protein are introduced to the site of injury.

In one embodiment for the living-cell biophoton radiators of this invention, the cells optionally can contain an exogenous nucleic acid encoding a gene product, which increases endocrine action of the cell, e.g., a gene encoding a hormone, or a paracrine action of the cell. For example, stem cells are genetically modified to contain an exogenous nucleic acid encoding a bone morphogenetic factor and engrafted into bone, cartilage, or tooth tissue, e.g., to treat periodontitis.

The cells for the living-cell biophoton radiator of this invention, optionally also include nucleic acids encoding other biologically active or therapeutic proteins or polypeptides, e.g., angiogenic factors, extracellular matrix proteins, cytokines or growth factors. For example, cells to be engrafted into pancreatic tissue contain a nucleic acid(s) encoding insulin or insulin precursor molecules. The cells also optionally include nucleic acids encoding gene products that decrease transplant rejection, e.g., CTLA4Ig CD40 ligand, or decrease development of transplant arteriosclerosis, e.g., inducible nitric oxide synthase (iNOS).

Tissue specificity is a fundamental problem for gene therapy as proteins that are therapeutic in target cells also may be harmful to normal tissue. Thus, non cell-specific expression of a transgene has the potential for inducing metabolic and physiologic mechanisms that could result in pathology over the long term. Localized injections can provide certain degree of localized expression of the targeting vector, however, there may still be a spill over into the circulation which will affect other cells and organs. In some embodiments, transcriptionally targeted vectors may be used that can restrict the expression of the therapeutic proteins primarily to the target cells by the use of tissue-specific promoters.

Once the cells for the living-cell biophoton radiator of this invention are implanted, maintenance of the cells is dependent upon adequate nutrient and oxygen delivery to the implanted cells. The polymer cell coating according to the embodiments can allow for entry of oxygen and other nutrients into the coated cell.

In one embodiment of the present invention, a selected portion of cells in an organ can ne be subjected to stress. Accordingly, in this embodiment, a number of sources of stress can be used to introduce at least one of chemical and physical stresses on the selected portion of cells in the organ. For example, ultrasonic waves concentrated on a particular region of the organ could induce mechanical stresses (e.g., compression and/or elongation of the cell membranes) changing the transport of nutrients across the membrane, thereby stressing those cells to induce biophoton emission.

In another example, localized cooling of tissues in one part of an organ would produce stress in the cells to induce biophoton emission. In another example, localized heating of tissues in one part of an organ would produce stress in the cells undergoing the local heating to induce biophoton emission.

In this example, microwave hyperthermia treatment systems such as those described in U.S. Pat. No. 9,079,011 (the entire contents of which are incorporated herein by reference) could be used to locally heat tissues in one part of an organ, producing stress in those cells to induce biophoton emission. Conventionally, hyperthermia has been used to elevate the temperature of tissues for a variety of purposes including: (i) destroying tissues such as tumors by the application of heat, (ii) increasing the susceptibility of heated tissue to chemical or radiation therapy, and (iii) triggering heat activated or released drugs. It is generally known to use microwave electromagnetic radiation for hyperthermia treatment.

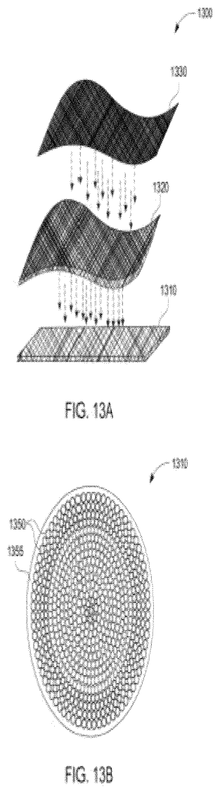

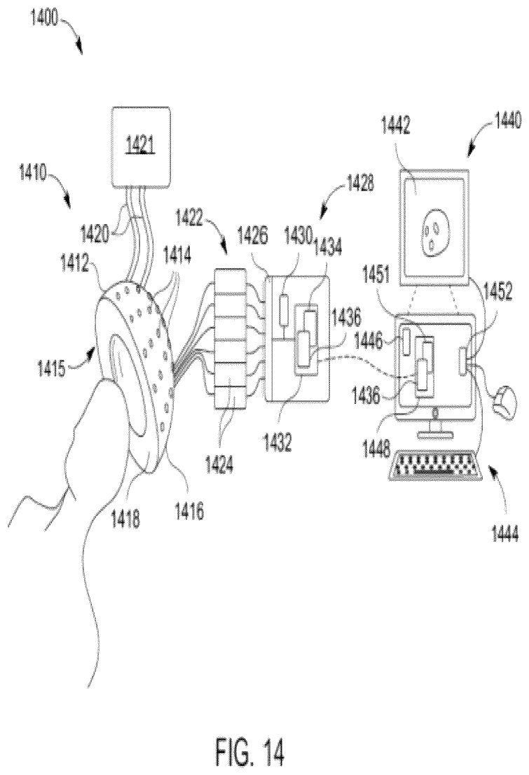

FIG. 14 is a depiction of a system 1400 of the present invention for application of microwave energy to a target region to locally heat the cells in the target region and thereby induce biophoton emission.

System 1400 of the present invention can have an antenna fixture 1412 supporting a plurality of antennas 1414 about a treatment volume 1415. In one embodiment, the treatment volume may be defined by a substantially hemispherical shell 1416 whose inner surface may contain a collar 1418 receiving and supporting the top of the patient's head. The collar may be filled with de-ionized water that may be circulated through connecting hoses 1420 with a cooler/pump 1421 providing skin cooling of at approximately 15 degrees centigrade of the patient's head to minimize surface heating of the skin by microwave energy from the antennas 14.

The antennas 1414 preferably direct microwave energy inward toward the treatment volume 1415 and may, for example, be microwave horns or patch antennas or other antennas of a type known in the art and are spaced to provide for substantially uniform separation of less than six centimeters.

Each antenna 1414 may be connected to a radiofrequency power source 1422 providing independent phase (phi) and amplitude (A) control of the radiofrequency power applied to the antenna. The radiofrequency power source 1422 may provide a separate radiofrequency amplifier/synthesizer 1424 for each antenna 1414 or may use a single radiofrequency power source with separate amplitude and phase shifters. In one embodiment, a set of discrete phases and amplitudes may be implemented in a switching fashion.

The radiofrequency power source 1422 may be controlled by a treatment controller 1428 via an interface board 1426, for example, providing a multiplexed A/D converter outputting phase and amplitude values from the treatment controller 1428. The treatment controller 1428 may include a processor 1430 communicating with a memory 1432 holding a stored program 1434 and treatment plan data 1436 describing a treatment schedule of changing phases and amplitudes of microwave frequency to be applied to the antennas 14 during treatment.

The treatment plan data 1436 may be developed on the treatment controller 1428 but also can be developed off-line on a separate workstation 1440 having a display 1442 for displaying treatment maps for physician input, as will be described, generated by a communicating standard desktop computer 1444 also having a processor 1446, a stored memory 1448 holding a treatment planning program 1451 and the treatment plan data 1436, the latter which may be transferred to treatment controller 1428. The desktop computer 1444 may also communicate with input devices 1450 by interface 1452 according to well understood techniques for physician input as will be described. It will be appreciated that the processing and data storage required by the present invention may be freely distributed among one or more processors and different types of computers according to well-understood techniques.

Microwaves provide a number of advantages including an ability to pass though some body structures such as the skull for treatment of the brain, and an ability to be focused to permit, for example, localized treatment of a tumor surrounded by tissue with reduced damage to the surrounding tissue. However, in this embodiment of the invention, localized and focused heating of selected portion of cells in an organ preferentially stops short of cell death, as dead cells would not emit biophoton radiation. Rather, the treatment plan stresses living cells in the targeted region to emit biophoton radiation.

In another example, stress could be applied by UV light at a non-lethal dose level using external sources of UV light “piped” into the subject (using for example the hollow cavity waveguide described above), or using phosphors under high energy or x-ray irradiation to produce internally within an organ localized stress.

### Artificial Ex-Vivo Biophoton Radiators

On the market today is at least one commercial biophoton source, the BEP-AN15 made by Biolight, a Korean company. The Biolight source is reported to radiate ultra-weak photon emission, generating energy through modulation of visible light, and delivers the energy at a frequency similar to “biophotons by voluntary absorption.”

Joohyeong Lee et al in “Oocyte maturation under a biophoton generator improves preimplantation development of pig embryos derived by parthenogenesis and somatic cell nuclear transfer.” Korean J Vet Res (2017) 57 (2), pp. 89-95 (the entire contents of which are incorporated herein by reference) report the use of the artificial source of biophoton radiation noted above, the BEP-AN15 made by Biolight. Their work reported to shown that biophoton treatments during in vitro maturation improved the “developmental competence” of parthenogenesis and somatic cell nuclear transfer derived embryos. In their paper, Lee et al described that, in prior work, “leakage of a very small amount of photons from external sources has been shown to alter ultraweak photon emissions and cell-to-cell communication.”

Accordingly, in one embodiment of the invention, an artificial ex vivo (or in vivo) biophoton generator is used to produce biophoton radiation or to affect ultraweak photon emissions and cell-to-cell communication.

One possible artificial source for biophoton radiation includes the device(s) described in U.S. Pat. No. 5,800,479 (the entire contents of which are incorporated herein by reference) owned by Biolight Patent Holding AB (Danderyd, SE). The '479 patent describes a device for an external medical treatment with the aid of light, including a light emitting element which is intended to lie against or be held close to a wound or sore on the body of an individual. The light emitting element included light emitting diodes or like devices and was constructed to (1) to emit infrared light in a first stage for a first predetermined length of time and thereafter to emit visible light in a second stage for a second predetermined length of time.

Another possible artificial source for biophoton radiation includes the device(s) described in U.S. Pat. No. 6,238,424 (the entire contents of which are incorporated herein by reference) owned by Biolight Patent Holding AB (Danderyd, SE). The '424 patent describes an apparatus for external medical treatment with light. A light-emitting device in the '424 patent is provided in close proximity to the body of an individual and that includes light-emitting diodes or corresponding elements that are adapted to emit monochromatic light of a first wavelength. The light emitting device is driven by a drive arrangement for causing the light-emitting device to emit the monochromatic light over a first predetermined time period in a first state, and thereafter emit selectively monochromatic light of a different wavelength than the first wavelength and over a second predetermined time period in a possible second state. The drive arrangement causes the light-emitting device to pulsate the emitted light in accordance with a predetermined pulse frequency or series of pulse frequencies over the respective time periods, and causes the light-emitting device to emit the pulsating light with a pulse length that lies within an interval of about 60% to about 90% of the time between respective start edges of two mutually sequential pulse.

Another possible artificial source for biophoton radiation includes the device(s) described in U.S. Pat. No. 6,537,303 (the entire contents of which are incorporated herein by reference) owned by Biolight Patent Holding AB (Danderyd, SE). The '303 patent describes a method for treatment of mammals by draining lymph along a lymph pathway within a mammal's body. In the '303 patent, an infrared-light-emitting device is used to emit pulsating infrared light at a low pulse repetition frequency. The light-emitting device is brought into contact with the body and is moved along a lymph pathway in a direction toward the lymphatic gland to which the pathway of the lymph vessel in question leads.

In one embodiment of the present invention, these artificial sources would be attenuated to produce weak or ultraweak light emissions with duty cycles and wavelengths that mimic natural biophoton radiators. For example, UV emitting light emitting diodes could be used along with the visible and infrared light emitting diodes described above. UV light emitting diode are described in U.S. Pat. No. 8,907,320 (the entire contents of which are incorporated herein by reference) as including an n-type semiconductor layer, an active layer disposed on the n-type semiconductor layer, a p-type semiconductor layer disposed on the active layer and formed of p-type AlGaN, and a p-type graphene layer disposed on the p-type semiconductor layer and formed of graphene doped with a p-type dopant.

In one embodiment of the invention, a target cell to be treated is analyzed first to ascertain its biophoton emission characteristics. If the target cell is a known cancer strain, representative cancer lines could be analyzed. Alternatively, biopsies could remove small regions of the cancerous tumor. These representative or biopsied samples could be subject to cell death and the natural biophoton radiation could be observed. Once characteristics (e.g., wavelengths, duty cycle, total emittance) are known or inferred or estimated, the configuration and driving of the LED array elements can be used to mimic the natural biophoton spectra.

From the literature results noted above, in one embodiment of the invention, the mimic spectra could have one or more of the following characteristics:
emissions in 190-250 nm wavelength range;
emissions in the 330-340 nm wavelength range;
a combination of emissions in the 190-250 nm and in the 330-340 nm wavelength ranges;
emissions across the range of 250 nm to 600 nm;
emissions in the infrared range;
a duration of emission in short bursts of approximately a millisecond at a repetition frequency of 10 to 100 Hz; and
a range of photon flux from a few to a 1000 photons/(sec·cm²) or higher.

These characteristics are merely exemplary and would be designed in one embodiment as discussed above to mimic the natural biophoton spectra of a target cell to be treated.

Light from the external biophoton radiators would be coupled to the diseased or malignant site using the biophoton bypass noted above.

### In Vivo Point of Use Biophoton Generator

The present invention can use any desired energy converter, including, but not limited to, organic fluorescent molecules or inorganic particles capable of fluorescence and/or phosphorescence having crystalline, polycrystalline or amorphous micro-structures.

Organic fluorescent compounds with high quantum yield include, but are not limited to:
naphthalene, pyrene, perylene, anthracene, phenanthrene, p-terphenyl, p-quaterphenyl, trans-stilbene, tetraphenylbutadiene, distyrylbenzene, 2,5-diphenyloxazole, 4-methyl-7-diethylaminocoumarin, 2-phenyl-5-(4-biphenyl)-1,3,4-oxadiazole, 3-phenylcarbostyryl, 1,3,5-triphenyl-2-pyrazoline, 1,8-naphthoylene-1′,2′-benzimidazole, 4-amino-n-phenyl-naphthalimide.

Inorganic fluorescent and/or phosphorescent materials span a wide variety of materials. Furthermore, these materials can be doped with specific ions (activators or a combination of activators) that occupy a site in the lattice structure in the case of crystalline or polycrystalline materials and could occupy a network forming site or a bridging and/or non-bridging site in amorphous materials. These compounds include, but are not limited to, (not ranked by order of preference or utility):
CaF₂, ZnF₂, KMgF₃, ZnGa₂O₄, ZnAl₂O₄, Zn₂SiO₄, Zn₂GeO₄, Ca₅(PO₄)₃F, Sr₅(PO₄)₃F, CaSiO₃, MgSiO₃, ZnS, MgGa₂O₄, LaAl₁₁O₁₈, Zn₂SiO₄, Ca₅(PO₄)₃F, Mg₄Ta₂O₉, CaF₂, LiAl₅O₈, LiAlO₂, CaPO₃, AlF₃, and LuPO₄:Pr³⁺. Examples further include the alkali earth chalcogenide phosphors which are in turn exemplified by the following non-inclusive list: MgS:Eu³⁺, CaS:Mn²⁺, CaS:Cu, CaS:Sb, CaS:Ce³⁺, CaS:Eu²⁺, CaS:Eu²⁺Ce³⁺, CaS:Sm³⁺, CaS:Pb²⁺, CaO:Mn²⁺, CaO:Pb²⁺, Ca₃(PO₄)₂:Tl⁺, (Ca, Zn)₃(PO₄)₂:Tl⁺.

Further examples include the ZnS type phosphors that encompass various derivatives: ZnS:Cu,Al(Cl), ZnS:Cl(Al), ZnS:Cu,I(Cl), ZnS:Cu, ZnS:Cu,In.

Also included are the compound IIIb-Vb phosphors which include the group IIIb and Vb elements of the periodic table. These semiconductors include BN, BP, BSb, AlN, AlP, AlAs, AlSb, GaN, GaP, GaAs, GaSb, InN, InP, InAs, InSb and these materials may include donors and acceptors that work together to induce light emission diodes. These donors include, but are not limited to, Li, Sn, Si, Li, Te, Se, S, O and acceptors include, but are not limited to, C, Be, Mg, Zn, Cd, Si, Ge. Further included are the major GaP light emitting diodes which include, but are not limited to, GaP:Zn,O, GaP:NN, Gap:N and GaP, which emit colors Red, Yellow, Green and Pure Green respectively.

The materials can further include such materials as GaAs with compositional variation of the following sort: In_{1-y}(Ga₁₋ₓAlₓ)_{y}P.

Also included is silicon carbide SiC, which has commercial relevancy as a luminescent platform in blue light emitting diodes. These include the polytypes 3C-SiC, 6H-SiC, 4H-SiC with donors such as N and Al and acceptors such as Ga and B.

Further examples include multiband luminescent materials include, but not limited to, the following compositions (Sr, Ca, Ba)₅(PO₄)₃Cl:Eu²⁺, BaMg₂Al₁₆O₂₇:Eu²⁺, CeMgAl₁₁O₁₉:Ce³⁺:Tb³⁺, LaPO₄:Ce³⁺:Tb³⁺, GdMgB₅O₁₀:Ce₃:Tb³⁺, Y₂O₃:Eu³⁺, (Ba,Ca,Mg)₅(PO₄)₃Cl:Eu²⁺, 2SrO_{0.84}P2O50.16B2O3:Eu²⁺, Sr₄Al₁₄O₂₅:Eu²⁺.

Materials typically used for fluorescent high pressure mercury discharge lamps are also included. These can be excited with X-Ray and are exemplified by way of family designation as follows: Phosphates (Sr, M)(PO₄)₂:Sn²⁺, Mg or Zn activator, Germanate 4MgO·GeO₂:Mn⁴⁺, 4(MgO, MgF₂)GeO₂:Mn⁴⁺, Yttrate Y₂O₃:Eu³⁺, Vanadate YVO₄:Eu³⁺, Y(P,V)O₄:Eu³⁺, Y(P,V)O₄:In⁺, Halo-Silicate Sr₂Si₃O₈₂SrCl₂:Eu²⁺, Aluminate (Ba,Mg)₂Al₁₆O₂₄:Eu²⁺, (Ba, Mg)₂Al₁₆O₂₄:Eu²⁺,Mn²⁺, Y₂O₃Al₂O₃:Tb³⁺.

Another grouping by host compound includes chemical compositions in the halophosphates phosphors, phosphate phosphors, silicate phosphors, aluminate phosphors, borate phosphors, tungstate phosphors, and other phosphors. The halophosphates include, but are not limited to: 3Ca₃(PO₄)₂·Ca(F,Cl)₂:Sb³⁺, 3Ca₃(PO₄)₂·Ca(F,Cl)₂:Sb³⁺/Mn²⁺, Sr₁₀(PO₄)₆Cl₂:Eu²⁺, (Sr,Ca)₁₀(PO₄)₆Cl₂:Eu²⁺, (Sr,Ca)₁₀(PO₄)₆·nB₂O₃:Eu³⁺, (Sr, Ca,Mg)₁₀(PO₄)₆Cl₂:Eu²⁺. The phosphate phosphors include, but are not limited to: Sr₂P₂O₇:Sn²⁺, (Sr,Mg)₃(PO₄)₂:Sn²⁺, Ca₃(PO₄)₂·Sn²⁺, Ca₃(PO₄)₂:Tl⁺, (Ca,Zn)₃(PO₄)₂:Tl⁺, Sr₂P₂O₇:Eu²⁺, SrMgP₂O₇:Eu²⁺, Sr₃(PO₄)₂:Eu²⁺, LaPO₄:Ce³⁺, Tb³⁺, La₂O₃·0.2SiO₂·0.9P₂O₅:Ce³⁺·Tb³⁺, BaO·TiO₂·P₂O₅. The silicate phosphors Zn₂SiO₄:Mn²⁺, CaSiO₃:Pb²⁺/Mn²⁺, (Ba, Sr, Mg)·3Si₂O₇:Pb²⁺, BaSi₂O₅:Pb²⁺, Sr₂Si₃O₈·2SrCl₂:Eu²⁺, Ba₃MgSi₂O₈:Eu²⁺, (Sr,Ba)Al₂Si₂O₈:Eu²⁺.

The aluminate phosphors include, but are not limited to: LiAlO₂:Fe³⁺, BaAl₈O₁₃:Eu²⁺, BaMg₂Al₁₆O₂₇:Eu²⁺, BaMg₂Al₁₆O₂₇:Eu²⁺/Mn²⁺, Sr₄Al₁₄O₂₅:Eu²⁺, CeMgAl₁₁O₁₉:Ce³⁺/Tb³⁺.

The borate phosphors include: Cd₂B₂O₅:Mn²⁺, SrB₄O₇F:Eu²⁺, GdMgB₅O₁₀:Ce³⁺/Tb³⁺, GdMgB₅O₁₀:Ce³⁺/Mn³⁺, GdMgB₅O₁₀:Ce³⁺/Tb³⁺Mn²⁺.

The tungstate phosphors include, but are not limited to: CaWO₄, (Ca,Pb)WO₄, MgWO₄. Other phosphors Y₂O₃:Eu³⁺, Y(V,P)O₄:Eu²⁺, YVO₄:Dy³⁺, MgGa₂O₄:Mn²⁺, 6MgO·As₂O₅:Mn²⁺, 3.5MgO·0.5MgF₂·GeO₂:Mn⁴⁺.

The activators to the various doped phosphors include, but are not limited to: Tl⁺, Pb²⁺, Ce³⁺, Eu²⁺, WO₄²⁻, Sn²⁺, Sb³⁺, Mn²⁺, Tb³⁺, Eu³⁺, Mn⁴⁺, Fe³⁺. The luminescence center Tl⁺ is used with a chemical composition such as: (Ca,Zn)₃(PO₄)₂:Tl⁺, Ca₃(PO₄)₂:Tl⁺. The luminescence center Mn²⁺ is used with chemical compositions such as MgGa₂O₄:Mn²⁺, BaMg₂Al₁₆O₂₇:Eu/Mn²⁺, Zn₂SiO₄:Mn²⁺, 3Ca₃(PO₄)₂·Ca(F,Cl)₂:Sb²⁺/Mn²⁺, CaSiO₃:Pb²⁺/Mn²⁺, Cd₂B₂O₅:Mn²⁺, CdB₂O₅:Mn²⁺, GdMgB₅O₁₀:Ce³⁺/Mn²⁺, GdMgB₅O₁₀:Ce³⁺/Tb³⁺Mn²⁺. The luminescence center Sn2+ is used with chemical compositions such as: Sr₂P₂O₇:Sn²⁺, (Sr,Mg)₃(PO₄)₂:Sn²⁺. The luminescence center Eu²⁺ is used with chemical compositions such as: SrB₄O₇F:Eu²⁺, (Sr,Ba)Al₂Si₂O₈:Eu²⁺, Sr₃(PO₄)₂:Eu²⁺, Sr₂P₂O₇:Eu²⁺, Ba₃MgSi₂O₈:Eu²⁺, Sr₁₀(PO₄)₆Cl₂:Eu²⁺, BaMg₂Al₁₆O₂₇:Eu²⁺/Mn²⁺, (Sr,Ca)₁₀(PO₄)₆Cl₂:Eu²⁺. The luminescence center Pb²⁺ is used with chemical compositions such as: (Ba,Mg,Zn)₃Si₂O₇:Pb²⁺, BaSi₂O₅:Pb²⁺, (Ba,Sr)₃Si₂O₇:Pb²⁺.

The luminescence center Sb²⁺ is used with chemical compositions such as: 3Ca₃(PO₄)₂·Ca(F,Cl)₂:Sb³⁺, 3Ca₃(PO₄)₂·Ca(F,Cl)₂:Sb³⁺Mn²⁺.

The luminescence center Tb³⁺ is used with chemical compositions such as: CeMgAl₁₁O₁₉:Ce³⁺/Tb³⁺, LaPO₄:Ce³⁺/Tb³⁺, Y₂SiO₅:Ce³⁺/Tb³⁺, GdMgB₅O₁₀:Ce³⁺/Tb³⁺. The luminescence center Eu³⁺ is used with chemical compositions such as: Y₂O₃:Eu³⁺, Y(V,P)O₄:Eu³⁺. The luminescence center Dy³⁺ is used with chemical compositions such as: YVO₄:Dy³⁺. The luminescence center Fe³⁺ is used with chemical compositions such as: LiAlO₂:Fe³⁺. The luminescence center Mn⁴⁺ is used with chemical compositions such as: 6MgO·As₂O₅:Mn⁴⁺, 3.5MgO0.5MgF₂·GeO₂:Mn⁴⁺. The luminescence center Ce³⁺ is used with chemical compositions such as: Ca₂MgSi₂O₇:Ce³⁺ and Y₂SiO₅:Ce³⁺. The luminescence center WO₄²⁻ is used with chemical compositions such as: CaWO₄, (Ca,Pb)WO₄, MgWO₄. The luminescence center TiO₄⁴⁻ is used with chemical compositions such as: BaO·TiO₂·P₂O₅. Additional phosphor chemistries of interest using X-Ray excitations include, but are not limited to, the k-edge of these phosphors. Low energy excitation can lead to intense luminescence in materials with low k-edge. Some of these chemistries and the corresponding k-edge are listed below:

| | | |
|---|---|---|
| BaFCl:Eu²⁺ | 37.38 | keV |
| BaSO₄:Eu²⁺ | 37.38 | keV |
| CaWO₄ | 69.48 | keV |
| Gd₂O₂S:Tb³⁺ | 50.22 | keV |
| LaOBr:Tb³⁺ | 38.92 | keV |
| LaOBr:Tm³⁺ | 38.92 | keV |
| La₂O₂S:Tb³⁺ | 38.92 | keV |
| Y₂O₂S:Tb³⁺ | 17.04 | keV |
| YTaO₄ | 67.42 | keV |
| YTaO₄:Nb | 67.42 | keV |
| ZnS:Ag | 9.66 | keV |
| (Zn, Cd)S:Ag | 9.66/26.7 | keV |

These materials can be used alone or in combinations of two or more. A variety of compositions can be prepared to obtain the desired output wavelength or spectrum of wavelengths.

In the present invention, the phosphor selection could be chosen such that under x-ray or other high energy source irradiation, the light emitted from the phosphors would mimic the natural biophoton spectra of a target cell to be treated, similar to that described above where exemplary characteristics could include:
emissions in 190-250 nm wavelength range;
emissions in the 330-340 nm wavelength range;
a combination of emissions in the 190-250 nm and in the 330-340 nm wavelength ranges;
emissions across the range of 250 nm to 600 nm;
emissions in the infrared range;
a duration of emission in short bursts of approximately a millisecond at a repetition frequency of 10 to 100 Hz; and
a range of photon flux from a few to a 1000 photons/(sec·cm2) or higher.

Thus, in one embodiment of the invention, ultraviolet and visible emissions can be used for the inventive in vivo biophoton source.

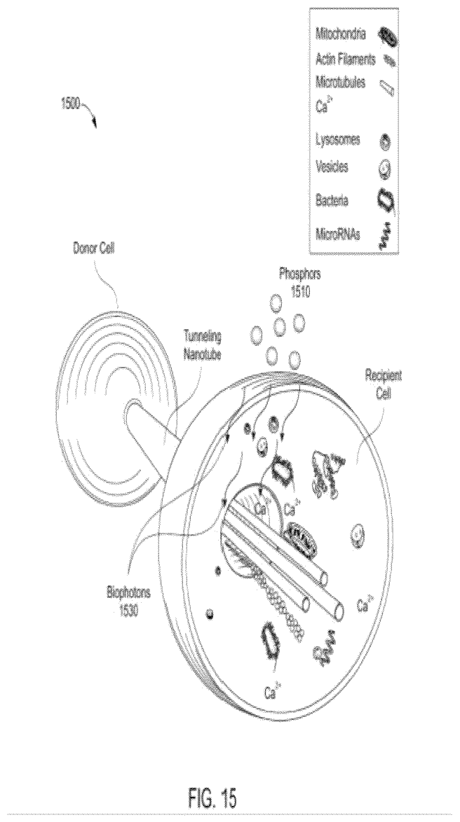

FIG. 15 is a depiction of an in vivo biophoton source 1500 where phosphors 1510 in proximity to the cells are excited by high energy such as x-rays or e-beams to generate biophoton radiation 1530 mimicking the characteristics known or measured from the target cells for their biophoton radiation.

In the depiction of FIG. 15, the biophotons 1530 can penetrate the cell and interact with the interior components of the cell such as the mitochondria and bacteria in the cell. In one embodiment of the invention, the biophotons 1530 can be transmitted to the donor cell by transmission through the tunneling nanotube joining the cells. A more thorough discussion of tunneling nanotubes is given later. In one embodiment of the invention, the biophoton radiation may change the chemical and charge transport along the tunneling nanotubes by photoionization events which place charge on the interior walls of the tunneling nanotubes.

Accordingly, in one embodiment of the invention, the photon flux from the inventive biophoton sources can be, but is not necessarily, a low photon flux source (in the range of single photons and therefore not operating as a classical light wavefront subject to scattering and absorption). Higher flux may be used with the expectation that beneficial results would still follow, especially under conditions where the natural absorption/scatter in the subject would result in appropriate photon fluxes within the treatment region.

With the in vivo point of use biophoton generator, the duty cycle of the x-ray unit would determine the duty cycle of the biophoton radiation produced, the phosphor selection or combination of phosphors would determine the wavelength emission characteristics, and external coatings on the phosphors would serve to attenuate the level of light emitted at the target site.

Moreover, since the level of light emission for biophotons is low, the x-ray dose to the patient for a biophoton radiation treatment can be significantly lower than that for other radiation treatments.

In this embodiment, a downconverting energy modulation agent (e.g., a down converting phosphor) can comprise inorganic particulates selected from the group consisting of: metal oxides; metal sulfides; doped metal oxides; and mixed metal chalcogenides. In one aspect of the invention, the downconverting material can comprise at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, ZnS; ZnSe; MgS; CaS and alkali lead silicate including compositions of SiO₂, B₂O₃, Na₂O, K₂O, PbO, MgO, or Ag, and combinations or alloys or layers thereof. In one aspect of the invention, the downconverting material can include a dopant including at least one of Er, Eu, Yb, Tm, Nd, Mn Tb, Ce, Y, U, Pr, La, Gd and other rare-earth species or a combination thereof. The dopant can be included at a concentration of 0.01%-50% by mol concentration. U.S. Pat. Appl. Publ. Nos. 2017/0157418 and 2017/0239489 (the entire contents of both are incorporated herein by reference) provided details of these and other suitable phosphors.

In one aspect of the invention, the downconverting energy modulation agent can comprise materials such as ZnSeS:Cu, Ag, Ce, Tb; CaS: Ce,Sm; La₂O₂S:Tb; Y₂O₂S:Tb; Gd₂O₂S:Pr, Ce, F; LaPO₄. In other aspects of the invention, the downconverting material can comprise phosphors such as ZnS:Ag and ZnS:Cu, Pb. In other aspects of the invention, the downconverting material can be alloys of the ZnSeS family doped with other metals. For example, suitable materials include ZnSexSy:Cu, Ag, Ce, Tb, where the following x, y values and intermediate values are acceptable: x:y; respectively 0:1; 0.1:0.9; 0.2:0.8; 0.3:0.7; 0.4:0.6; 0.5:0.5; 0.6:0.4; 0.7:0.3; 0.8:0.2; 0.9:0.1; and 1.0:0.0.

In other aspects of the invention, the downconverting energy modulation agent can be materials such as sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide:europium (Gd₂O₂S:Eu), lanthanum oxysulphide:europium (La₂O₂S:Eu), and gadolinium oxysulphide:promethium, cerium, fluorine (Gd₂O₂S:Pr,Ce,F), YPO₄:Nd, LaPO₄:Pr, (Ca,Mg)SO₄:Pb, YBO₃:Pr, Y₂SiO₅:Pr, Y₂Si₂O₇:Pr, SrLi₂SiO₄:Pr,Na, and CaLi₂SiO₄:Pr.

In other aspects of the invention, the downconverting energy modulation agent can be near-infrared (NIR) downconversion (DC) phosphors such as KSrPO₄:Eu²⁺, Pr³⁺, or NaGdF₄:Eu or Zn₂SiO₄:Tb³⁺, Yb³⁺ or β-NaGdF₄ co-doped with Ce³⁺ and Tb³⁺ ions or Gd₂O₂S:Tm or BaYF₅:Eu³⁺ or other down converters which emit NIR from visible or UV light exposure (as in a cascade from x-ray to UV to NIR) or which emit NIR directly after x-ray or e-beam exposure.

In one aspect of the invention, an up converting energy modulation agent can also be used such as at least one of Y₂O₃, Y₂O₂S, NaYF₄, NaYbF₄, YAG, YAP, Nd₂O₃, LaF₃, LaCl₃, La₂O₃, TiO₂, LuPO₄, YVO₄, YbF₃, YF₃, Na-doped YbF₃, or SiO₂ or alloys or layers thereof.

In one aspect of the invention, the energy modulation agents can be used singly or in combination with other down converting or up converting materials.

TABLE 1 shows a list of other suitable phosphors:

TABLE 1

| | | Emission | X-Ray Absorption | | | | | |
|---|---|---|---|---|---|---|---|---|
| | | Spectrum | Emiss | | | Microstructure | | |
| | | Peak Emission | Eff | Eff | K-edge | Specific | Crystal | |
| # | Phosphor | (nm) | % | (Z) | (keV) | Gravity | Structure | Hygroscopic |
| 1 | BaFCl:Eu²⁺ | 380 | 13 | 49.3 | 37.38 | 4.7 | Tetragonal | N |
| 2 | BaSO₄—:Eu²⁺— | 390 | 6 | 45.5 | 37.38 | 4.5 | Rhombic | N |
| 3 | LaOBr:Tm⁺³ | 360, 460 | 14 | 49.3 | 38.92 | 6.3 | Tetragonal | N |
| 4 | YTaO₄ | 337 | | 59.8 | 67.42 | 7.5 | Monolithic | N |
| 5 | YTaO₄:Nb (*) | 410 | 11 | 59.8 | 67.42 | 7.5 | Monolithic | N |
| 6 | CaWO₄ | 420 | 3 | 61.8 | 69.48 | 6.1 | Tetragonal | N |
| 7 | LaOBr:Tb³⁺ | 420 | 20 | 49.3 | 38.92 | 6.3 | Tetragonal | N |
| 8 | Y₂O₂S:Tb²⁺ | 420 | 18 | 34.9 | 17.04 | 4.9 | Hexgonal | N |
| 9 | ZnS:Ag | 450 | 17 | 26.7 | 9.66 | 3.9 | Hexgonal | N |
| 10 | (Zn,Cd)S:Ag | 530 | 19 | 38.4 | 9.66/26.7 | 4.8 | Hexgonal | N |
| 11 | Gd₂O₂S:Tb³⁺ | 545 | 13 | 59.5 | 50.22 | 7.3 | Hexgonal | N |
| 12 | La₂O₂S:Tb³⁺ | 545 | 12.5 | 52.6 | 38.92 | 6.5 | Hexgonal | N |

In one embodiment of the invention, besides the YTaO₄, noted above, other energy modulation agents can include phosphors were obtained from the following sources. “Ruby Red” obtained from Voltarc, Masonlite & Kulka, Orange, Conn., and referred to as “Neo Ruby”; “Flamingo Red” obtained from EGL Lighting. Berkeley Heights, N.J, and referred to as “Flamingo”; “Green” obtained from EGL Lighting, Berkeley Heights, N.J. and referred to as “Tropic Green”; “Orange” obtained from Voltarc, Masonlite & Kulka. Orange, Conn, and referred to as “Majestic Orange”; “Yellow” obtained from Voltarc. Masonlite & Kulka, Orange. Conn., and referred to as “Clear Bright Yellow.” The “BP” phosphors are shown in detail below in TABLE 2:

TABLE 2

| | | Emission | | | | | | |
|---|---|---|---|---|---|---|---|---|
| | | Spectrum | X-Ray Absorption | | | Density | | |
| | Phosphor | Peak | Emiss | | | g/cc | Xtal | |
| | Material | Emission | Eff | Eff | K-edge | Specific | Crystal | |
| Code | Color | (nm) | (%) | (Z) | (keV) | Gravity | Structure | Hygroscopic |
| BP1 | CaWO4:Pb | 425 | | | | | | N |
| BP2 | Y2SiO5:Ce | 410 | | | | | | N |
| BP3 | YTaO4 | 337 | 10 | 59.8 | 67.42 | 7.5 | Monolithic | N |
| BP3-C | YTaO4 | 337 | 10 | 59.8 | 67.42 | | Monolithic | N |
| BP4 | BASF-1 | 460 | | | | | | |
| BP5 | BASF-2 | 490 | | | | | | |
| BP6 | YTaO4:Nb (*) | 410 | 11 | 59.8 | 67.42 | 7.5 | Monolithic | N |
| BP6-C | YTaO4:Nb (*) | | | | | | | |
| BP7-C | LaOBr:Tm3+ | 360, 460 | 14 | 49.3 | 38.92 | 6.3 | Tetragonal | N |
| | (coated) | | | | | | | |
| BP8-C | LaF3:Ce | 280 | | | | | | |
| BP9 | Y2O3 | 365 | | | | | | |
| BP-10 | BaSO4——:Eu2+ | 390 | 6 | 45.5 | 37.38 | 4.5 | Rhombic | N |
| | (coated) | | | | | | | |
| BP10-C | BaSO4—:Eu2+ | 390 | 6 | 45.5 | 37.38 | 4.5 | Rhombic | N |
| | (coated) | | | | | | | |
| BP11 | LaOCl:Tm | | | | | | | |
| BP12 | Y2O2S:Tm | | | | | | | |
| BP13 | BaSi2O5:Pb2+ | 350 | | | | | | N |
| | StB6O10:Pb | 360 | | | | | | N |
| | CaI:Na (Coated) | 338 | | | | | | Y |
| | Gd₂O₂S:Tm | Blue to | | | | | | Y |
| | | Green | | | | | | |

The “BP” phosphors are available from PhosphorTech Corporation of Kennesaw, Ga., from BASF Corporation, or from Phosphor Technology Ltd, Norton Park, Norton Road Stevenage, Herts, SG1 2BB, England.

Other useful energy modulation agents include semiconductor materials including for example TiO₂, ZnO, and Fe₂O₃ which are biocompatible, and CdTe and CdSe which would preferably be encapsulated because of their expected toxicity. Other useful energy modulation agents include ZnS, CaS, BaS, SrS and Y₂O₃ which are less toxic. Other suitable energy modulation agents which would seem the most biocompatible are zinc sulfide, ZnS:Mn²⁺, ferric oxide, titanium oxide, zinc oxide, zinc oxide containing small amounts of Al₂O₃ and AgI nanoclusters encapsulated in zeolite. For non-medical applications, where toxicity may not be as critical a concern, the following materials (as well as those listed elsewhere) are considered suitable: lanthanum and gadolinium oxyhalides activated with thulium; Er³⁺ doped BaTiO₃ nanoparticles. Yb³⁺ doped CsMnCl₃ and RbMnCl₃. BaFBr:Eu²⁺ nanoparticles, cesium iodide, bismuth germanate, cadmium tungstate, and CsBr doped with divalent Eu. Table 3 below provides a list of various useful energy modulation agents

In various embodiments of the invention, the following luminescent polymers are also suitable as energy modulation agents: poly(phenylene ethynylene), poly(phenylene vinylene), poly(p-phenylene), poly(thiophene), poly(pyridyl vinylene), poly(pyrrole), poly(acetylene), poly(vinyl carbazole), poly(fluorenes), and the like, as well as copolymers and/or derivatives thereof.

As a non-limiting list, the following are also suitable energy modulation agents: Y₂O₃ ZnS; ZnSe; MgS; CaS; Mn, Er ZnSe; Mn, Er MgS; Mn, Er CaS; Mn, Er ZnS; Mn, Yb ZnSe; Mn, Yb MgS; Mn, Yb CaS; Mn, Yb ZnS:Tb³⁺, Er³⁺; ZnS:Tb³⁺; Y₂O₃:Tb³⁺; Y₂O₃:Tb³⁺, Er³⁺; ZnS:Mn²⁺; ZnS:Mn,Er³⁺; CaWO₄, YaTO₄, YaTO₄:Nb, BaSO₄:Eu, La₂O₂S:Tb, BaSi₂O₅:Pb, NaI(Tl), CsI(Tl), CsI(Na), CsI(pure), CsF, KI(Tl), LiI(Eu), BaF₂, CaF, CaF₂(Eu), ZnS(Ag), CaWO₄, CdWO₄, YAG(Ce) (Y₃Al₅O₁₂(Ce)), BGO bismuth germanate, GSO gadolinium oxyorthosilicate, LSO lutetium oxyorthosilicate. LaCl₃(Ce). LaBr₃(Ce). LaPO₄; Ce, Tb (doped). Zn₂SiO₄:Mn with Mn doped between 0.05-10%, and YTaO₄.

TABLE 3

| | Emission | | | X-Ray | | | |
|---|---|---|---|---|---|---|---|
| | Spectrum | | | Absorp- | | | |
| | Peak | Emiss | | tion | | | |
| Phosphor | Emission | Eff | Eff | K-edge | Specific | Crystal | |
| Color | (nm) | (%) | (z) | (keV) | Gravity | Structure | Hygroscopic |
| Zn3(PO4)2:Tl+ | 310 | | | | | | N |
| BaF2 | 310 | | | | | | Slightly |
| CsI | 315 | | | | | | N |
| Ca3(PO4)2:Tl+ | 330 | | | | | | N |
| YTaO4 | 337 | | 59.8 | 67.42 | | 7.5 Monolithic | N |
| CsI:Na | 338 | | | | | | Y |
| BaSi2O5:Pb2+ | 350 | | | | | | N |
| Borosilicate | 350 | | | | | | N |
| LaCl3(Ce) | 350 | | | | | | Y |
| SrB4O7F:Eu2+ | 360 | | | | | | N |
| RbBr:Tl+ | 360 | | | | | | ? |
| (Ba,Sr,Mg,)3Si2O7:Pb2+ | 370 | | | | | | N |
| YAlO3:Ce3+ | 370 | | | | | | N |
| BC-422 | 370 | | | | | Organic | ? |
| BaFCl:Eu2+ | 380 | 13 | 49.3 | 37.38 | 4.7 | Tetragonal | N |
| BaSO4——:Eu2+ | 390 | 6 | 45.5 | 37.38 | 4.5 | Rhombic | N |
| BaFBr:Eu2+ | 390 | | | | | | ? |
| BC-420 | 391 | | | | | Organic | ? |
| BC-414 | 392 | | | | | Organic | ? |
| SrMgP207:Eu2+ | 394 | | | | | | N |
| BaBr2:Eu2+ | 400 | | | | | | N |
| (Sr,Ba)Al2Si2O8:Eu2+ | 400 | | | | | | N |
| YTaO4:Nb(*) | 410 | 11 | 59.8 | 67.42 | 7.5 | Monolithic | N |
| Y2SiO5:Ce3+ | 410 | | | | | | N |
| CaWO4 | 420 | 5 | 61.8 | 69.48 | 6.1 | Tetragonal | N |
| LaOBr:Tb3+ | 420 | 20 | 49.3 | 38.92 | 6.3 | Tetragonal | N |
| Y2O2S:Tb3+ | 420 | 18 | 34.9 | 17.04 | 4.9 | Hexgonal | N |
| Lu2SiO5:Ce3+ | 420 | | | | | | N |
| Lu1.8Y0.2SiO5:Ce | 420 | | | | | | N |
| ZnS:Ag | 450 | 17 | 26.7 | 9.66 | 3.9 | Hexgonal | N |
| CdWO4 | 475 | | | | | | Slightly |
| Bi4Ge3O12(BGO) | 480 | | | | | | N |
| (Zn,Cd)S:Ag | 530 | 19 | 38.4 | 9.66/26.7 | 4.8 | Hexgonal | N |
| Gd2O2S:Tb3+ | 545 | 13 | 59.5 | 50.22 | 7.3 | Hexgonal | N |
| La2O2S:Tb3+ | 545 | 12.5 | 52.6 | 38.92 | 6.5 | Hexgonal | N |
| Y3Al5O12(Ce) | 550 | | | | | | N |
| LaOBr:Tm3+ | 360, 460 | 14 | 49.3 | 38.92 | 6.3 | Tetragonal | N |
| CaF2(Eu) | 435/300 | | | | | | N |

In one embodiment, phosphors used in the invention as energy modulation agents can include phosphor particles, ionic doped phosphor particles, single crystal or poly-crystalline powders, single crystal or poly-crystalline monoliths, scintillator particles, a metallic shell encapsulating at least a fraction of a surface of the phosphors, a semiconductor shell encapsulating at least a fraction of a surface of the phosphors, and an insulator shell encapsulating at least a fraction of a surface of the phosphors, and phosphors of a distributed particle size.

In one embodiment of this invention, the phosphors for the in vivo point of use biophoton generator can be coated with the '117 publication polymers noted above for homing of the phosphors for the in vivo point of use biophoton generator to the target site.

With the capability to produce in vivo or deliver in vivo, specified wavelengths of light, the present invention may utilize a hybrid process in which both biophoton radiation and “activation” radiation are available for treatment. An activation radiation would be radiation of a specific wavelength to activate a photoactivatable drug such as psoralen or coumarin.

The selection of activatable pharmaceutical agents depends on a number of factors such as the desired cellular change, the desired form of activation, as well as the physical and biochemical constraints that may apply. Exemplary activatable pharmaceutical agents may include, but are not limited to, agents that may be activated by photonic energy, electromagnetic energy, acoustic energy, chemical or enzymatic reactions, thermal energy, or any other suitable activation mechanisms. An activatable agent may be a small molecule; a biological molecule such as a protein, a nucleic acid or lipid; a supramolecular assembly; a nanoparticle; or any other molecular entity having a pharmaceutical activity once activated.

When activated, the activatable pharmaceutical agent may effect cellular changes that include, but are not limited to, apoptosis, redirection of metabolic pathways, up-regulation of certain genes, down-regulation of certain genes, secretion of cytokines, alteration of cytokine receptor responses, or combinations thereof.

The mechanisms by which an activatable pharmaceutical agent may achieve its desired effect are not particularly limited. Such mechanisms may include direct action on a predetermined target as well as indirect actions via alterations to the biochemical pathways. A preferred direct action mechanism is by binding the agent to a critical cellular structure such as nuclear DNA, mRNA, rRNA, ribosome, mitochondrial DNA, or any other functionally important structures. Indirect mechanisms may include releasing metabolites upon activation to interfere with normal metabolic pathways, releasing chemical signals (e.g. agonists or antagonists) upon activation to alter the targeted cellular response, and other suitable biochemical or metabolic alterations.

In one embodiment, the activatable pharmaceutical agent is capable of chemically binding to the DNA or mitochondria at a therapeutically effective amount. In this embodiment, the activatable pharmaceutical agent, preferably a photoactivatable agent, is exposed to an activating energy emitted from an energy modulation agent (e.g. a phosphor), which, in turn receives energy from an initiation energy source (e.g. an x-ray source).

The activatable agent may be derived from a natural or synthetic origin. Any such molecular entity that may be activated by a suitable activation signal source to effect a predetermined cellular change may be advantageously employed in the present invention. Suitable photoactive agents include, but are not limited to: psoralens and psoralen derivatives, pyrene cholesteryloleate, acridine, porphyrin, fluorescein, rhodamine, 16-diazorcortisone, ethidium, transition metal complexes of bleomycin, transition metal complexes of deglycobleomycin, organoplatinum complexes, alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations, porphyrins, dyes such as neutral red, methylene blue, acridine, toluidines, flavine (acriflavine hydrochloride) and phenothiazine derivatives, coumarins, quinolones, quinones, and anthroquinones, aluminum (111) phthalocyanine tetrasulfonate, hematoporphyrin, and phthalocyanine, and compounds which preferentially adsorb to nucleic acids with little or no effect on proteins. The term “alloxazine” includes isoalloxazines.

Endogenously-based derivatives include synthetically derived analogs and homologs of endogenous photoactivated molecules, which may have or lack lower (1 to 5 carbons) alkyl or halogen substituents of the photosensitizers from which they are derived, and which preserve the function and substantial non-toxicity. Endogenous molecules are inherently non-toxic and may not yield toxic photoproducts after photoradiation.

In one embodiment of the invention, a hybrid treatment is used. In one embodiment of the hybrid treatment, a control region inside a patient containing phosphors is exposed to x-rays, from which ultraviolet light and visible light of a spectrum to activate one of the activatable agents noted above. The photoactivated agents induce apoptosis, causing the cancer cells to emit naturally biophoton radiation. Simultaneously, phosphors mimicking the natural biophoton radiation are exposed with the same x-rays and emit also biophoton radiation.

In one embodiment of the invention, since cell death induced by the photoactivated agents occurs over a longer duration than the x-ray exposure, the simultaneous generation in situ of the biophoton radiation can be viewed as “signaling” adjacent cells not affected by the photoactivated agent of the cell death event.

In one embodiment of the invention, the photoactivated x-ray treatment can precede the generation of biophotons in vivo by first dosing the diseased site with the phosphors for photoactivation and then later dosing the diseased site with phosphors for biophoton generation. Since the level of light for biophotons is low, the x-ray dose to the patient for biophoton radiation can be significantly lower than that for activation of the photoactivated agents.

### Biophoton Stimulator

In one embodiment of the invention, a light source is used, not to mimic the natural biophoton spectra of a target cell to be treated, but rather to stimulate natural biophoton radiation. It is known that the entire range of visible light can stimulate a living system to emit a biophoton signal. It is also known that non-damaging ultraviolet radiation also stimulates living systems to emit biophoton signals. For example, it has been observed that light in the 300 to 450 nm wavelength range can induce ultraweak photon emission. The strongest emission observed occurred when the living cells were stimulated at 350 nm. In another example, “white light” also induced biophoton emission.

Thus, in this embodiment, the phosphors and combinations noted above for the in vivo biphoton generator embodiment can be remixed/reselected such the phosphor selection under x-ray or other high energy source irradiation, would emit light from the phosphors which would stimulate living tissue in a subject to generate its own natural biphoton radiation.

In one embodiment of the present invention, stimulated emission coherence is achieved because the living cells themselves (under stimulation) as in nature will generate coherent emissions. For example, without chemical toxins or high energy radiation, one can induce cancer cells (by exposure to “white light” or 350 nm light) to emit biophotons as if they themselves are undergoing apoptosis. The neighboring cancer cells would then respond to this “signaling” and die, and during the stress leading to death rebroadcast actual biophoton signals associated with cell death to their neighbors. Since the “rebroadcast” is from living cells, natural coherence would be obtained.

Coherence is considered advantageous if, at a distance from the coherent emission, constructive interference could promote a biological, physical, or chemical reaction. Coherence is considered advantageous if, at a distance from the coherent emission, long-range dynamic order is to be promoted and/or controlled. For example, electrically polar structures of biomolecules that contain electric charges can generate electromagnetic fields when they vibrate, thereby producing an endogenous electromagnetic field of the organism with coherent modes. In relation to this, the majority of proteins are electrically polar structures typically immersed in water, a highly polar liquid. When metabolic energy exceeds a critical level, these polar structures engage in a steady state of nonlinear vibration, and energy is stored in a highly ordered manner, as a coherent excitation. This order expresses itself as a long range phase correlation. The order in biological systems is considered not just spatial, but dynamic, and can include long-range coherence within the entire organism.

The cytoskeleton of living cells include microtubules, tree-like structures, throughout the cytoplasm. These microtubules are electrically polar structures that can be excited and are expected to generate an endogenous coherent electric field that could have a dominant effect directing the transport of molecules and electrons throughout the cell. Moreover, connective tissue with an extracellular matrix composed of collagen that interconnects cells throughout the body is another possible network for the collective bioplasma state.

Others have predicted resonant frequencies of the biological field in the microwave region of the electromagnetic spectrum between 100-1000 GHz. Thus, in another embodiment, the biophoton stimulator of the invention is a microwave source operating in this frequency range to “drive resonance” or otherwise influence the behavior of this bioplasma collective system.

### Target Treatments

Exemplary conditions, disorders or diseases which may be treated with the present invention can include, but are not limited to, cancer, autoimmune diseases, cardiac ablation (e.g., cardiac arrhythmia and atrial fibrillation), photoangioplastic conditions (e.g., de novo atherosclerosis, restinosis), intimal hyperplasia, arteriovenous fistula, macular degeneration, psoriasis, acne, hopeciareata, portwine spots, hair removal, rheumatoid and inflammatory arthrisis, joint conditions, lymph node conditions, and cognitive and behavioral conditions.

Although not intending to be bound by any particular theory or be otherwise limited in any way, the following theoretical discussion of scientific principles and definitions are provided to help the reader gain an understanding and appreciation of the present invention.

As used here, the term “subject” is not intended to be limited to humans, but may also include animals, plants, or any suitable biological organism.

As used herein, the phrase “a disease or condition” refers to a condition, disorder or disease that may include, but are not limited to, cancer, soft and bone tissue injury, chronic pain, wound healing, nerve regeneration, viral and bacterial infections, fat deposits (liposuction), varicose veins, enlarged prostate, retinal injuries and other ocular diseases, Parkinson's disease, and behavioral, perceptional and cognitive disorders. Exemplary conditions also may include nerve (brain) imaging and stimulation, a direct control of brain cell activity with light, control of cell death (apoptosis), and alteration of cell growth and division. Yet other exemplary a condition, disorder or disease may include, but are not limited to, cardiac ablation (e.g., cardiac arrhythmia and atrial fibrillation), photoangioplastic conditions (e.g., de novo atherosclerosis, restinosis), intimal hyperplasia, arteriovenous fistula, macular degeneration, psoriasis, acne, hopeciareata, portwine spots, hair removal, rheumatoid and inflammatory arthritis, joint conditions, and lymph node conditions.

The nature of the predetermined cellular change will depend on the desired pharmaceutical outcome. Exemplary cellular changes may include, but are not limited to, apoptosis, necrosis, up-regulation of certain genes, down-regulation of certain genes, secretion of cytokines, alteration of cytokine receptor responses, regulation of cytochrome c oxidase and flavoproteins, activation of mitochondria, stimulation antioxidant protective pathway, modulation of cell growth and division, alteration of firing pattern of nerves, alteration of redox properties, generation of reactive oxygen species, modulation of the activity, quantity, or number of intracellular components in a cell, modulation of the activity, quantity, or number of extracellular components produced by, excreted by, or associated with a cell, or a combination thereof. Predetermined cellular changes may or may not result in destruction or inactivation of the target structure.

The inventive treatments may be used in one embodiment to induce an auto vaccine effect for malignant cells, including those in solid tumors. To the extent that any rapidly dividing cells or stem cells may be damaged by a systemic treatment, then it may be preferable to direct any signals, chemical agents, biological agents, or blocking agents directly into the first region, preventing damage directly to normal, healthy cells or stem cells in the second (or treatment) region can be induced by activating a chemiluminescent, phosphorescent or bioluminescent compound with an appropriate activation energy, either outside the subject or inside the subject.

Candidates might be 1) in vivo stimulated regrowth of organ tissue, 2) generation of alternative pathways for nerve cell to nerve cell communication perhaps by promotion of TNTs, and 3) anti-inflammatory responses.

### Assisted Photobiomodulation

Photobiomodulation, which is also traditionally known as low level laser therapy (LLLT), cold laser therapy, and laser biostimulation, is an emerging medical and veterinary technique in which exposure to low-level laser light can stimulate or inhibit cellular function leading to beneficial clinical effects. The “best” combination of wavelength, intensity, duration and treatment interval is complex and sometimes controversial with different diseases, injuries and dysfunctions needing different treatment parameters and techniques.

In one embodiment of this invention, wavelengths of biophoton radiation can be applied to or emitted from within a first region can for example, aid tissue regeneration, resolve inflammation, relieve pain and boost the immune system. Observed biological and physiological effects to be expected include changes in cell membrane permeability, and up-regulation and down-regulation of adenosine triphosphate and nitric oxide. All of these changes in the biological material of the first region can, according to one embodiment of the invention, be responsible for inducing corresponding changes in a second or treatment region.

Clinical applications of photobiomodulation suitable for causing or initiating changes in the biological material of the first or target region of this invention include, for example, treating soft tissue and bone injuries, chronic pain, wound healing and nerve and sensory regeneration/restoration, and possibly even resolving viral and bacterial infections, treating neurological and phychiatric diseases (e.g., epilepsy and Parkinson's disease) (e.g., Zhang F., et al., Nature, 446:617-9 (Apr. 5, 2007; Han X., et al., PloS ONE, 2(3):e299 (Mar. 21, 2007); Arany P R, et al., Wound Repair Regen., 15(6):866-74 (2007); Lopes C B, et al., Photomed. Laser Surg., 25(2):96-101 (2007)). One other suitable clinical application is the treatment of inflammation, where the anti-inflammatory effect of location-and-dose-specific laser irradiation produces similar outcomes as NSAIDs, but without the potentially harmful side-effects (Bjordal J M, Couppe C, Chow R T, Tuner J, Ljunggren E A (2003). “A systematic review of low level laser therapy with location-specific doses for pain from chronic joint disorders”. The Australian journal of physiotherapy 49(2):107-16). Accordingly, in one embodiment of the present invention, biophoton irradiation from the biophoton radiation sources noted above can be applied to the biological material of the first or target region, and thereby inducing changes in the second or target region which may treat in the second region soft tissue and bone injuries, chronic pain, wound healing and nerve and sensory regeneration/restoration, and possibly even resolve viral and bacterial infections, and treat neurological and phychiatric diseases.

An NIR light treatment has been shown to prevent cell death (apoptosis) in cultured neurons (brain) cells (Wong-Reiley M T, et al., J B C, 280(6):4761-71 (2005)). Specific wavelengths of light can promote cellular proliferation to the activation of mitochondria, the energy-producing organelles within the cell via cytochrome c oxidase. An NIR treatment can augment mitochondrial function and stimulate antioxidant protective pathways. The evidence that the NIR treatment can augment mitochondrial function and stimulate antioxidant protective pathways comes from photobiomodulation experiments carried out using a laboratory model of Parkinson's disease (PD) (cultures of human dopaminergic neuronal cells) (Whelan H., et. al., SPIE, Newsroom, pages 1-3 (2008)). Accordingly, in one embodiment of the present invention, biophoton radiation from the biophoton sources noted above and NIR light can be applied or internally generated in the biological material of the first or target region, and thereby inducing changes in the second or target region to address the disorders noted above.

When the excitable cells (e.g., neurons, cardiomyocites) are irradiated with monochromatic visible light, the photoacceptors are also believed to be components of respiratory chain. It is clear from experimental data (Karu, T. I., (2002). Low-power laser therapy. In: CRC Biomedical Photonics Handbook, T. Vo-Dinh, Editor-in-Chief, CRC Press, Boca Raton (USA)) that irradiation can cause physiological and morphological changes in nonpigmental excitable cells viabsorption in mitochondria. Later, similar irradiation experiments were performed with neurons in connection with low-power laser therapy. It was shown in 80's that He-Ne laser radiation alters the firing pattern of nerves; it was also found that transcutaneous irradiation with HeNe laser mimicked the effect of peripheral stimulation of a behavioral reflex. These findings were found to be connected with pain therapy (Karu T I, et al., (2002)). Accordingly, in one embodiment of the present invention, low power laser therapy along with biophoton radiation from the biophoton sources noted above can be applied or internally generated in the biological material of the first or target region, and thereby inducing changes in the second or target region to address the disorders noted above.

When photoacceptors absorb photons, electronic excitation followed by photochemical reactions occurring from lower excitation states (first singlet and triplet) takes place. It is also known that electronic excitation of absorbing centers alters their redox properties. Until yet, five primary reactions have been discussed in literature (Karu T I, et al., (2002)). Two of them are connected with alteration of redox properties and two mechanisms involve generation of reactive oxygen species (ROE). Also, induction of local transient (very short time) heating of absorbing chromophores is possible. Details of these mechanisms can be found in (Karu T I, et. al., (2002); Karu T I, et al., (1998). The Science of Low Power Laser Therapy. Gordon and Breach Sci. Publ., London). Accordingly, in one embodiment of the present invention, the absorption of photons in the biological material of the first or target region (e.g., from the biophoton sources noted above) can contribute to changes in the first region, thereby inducing changes in the second or target region to alter the pathways noted above.

Photobiological action via activation of respiratory chain is believed to be a general mechanism occurring in cells. Crucial events of this type of cell metabolism activation are occurring due to a shift of cellular redox potential into more oxidized direction as well as due to ATP extrasynthesis. Susceptibility to irradiation and capability for activation depend on physiological status of irradiated cells: the cells, which overall redox potential is shifted to more reduced state (example: some pathological conditions) are more sensitive to the irradiation. The specificity of final photobiological response is determined not at the level of primary reactions in the respiratory chain but at the transcription level during cellular signaling cascades. In some cells, only partial activation of cell metabolism happens by this mechanism (example: redox priming of lymphocytes). Accordingly, in one embodiment of the present invention, the absorption of photons in the biological material of the first or target region (e.g., from the biophoton sources noted above) can induce changes in the first region, thereby inducing changes in the second or target region to affect the respiratory chain as noted above Far red and NIR radiation have been shown to promote wound healing, e.g., infected, ischemic, and hypoxic wounds (Wong-Reley, W T T, JBC, 280(6):4761-4771 (2005)). Red-to-NIR radiation also protects the retina against the toxic actions of methanol-derived formic acid in a rodent model of methanol toxicity and may enhance recovery from retinal injury and other ocular diseases in which mitochondrial dysfunction is postulated to play a role (Eells J T., PNAS, 100(6):3439-44 (2003)). Another clinical application of photobiomodulation is repair of soft and bone tissues by IR laser irradiation (Martinez M E, et al., Laser in Med. Sci., 2007). Invasive laser assisted liposuction is a recently developed method, wherein a laser fiber is introduced through a tube into the skin and directly to the fat cells causing the cells to rapture and drain away as liquid (Kim K H, Dermatol. Surg., 32(2):241-48 (2006)). Tissue around the area is coagulated. Yet, another application of photobiomodulation is a non-surgical varicose vein treatment (an endovenous laser therapy), wherein a laser is threaded through an incision and the full length of the varicose vein (Kim H S, J. Vasc. Interv. Radiol., 18(6):811 (2007)). When the laser is slowly withdrawn, heat is applied to the vein walls, causing the vein to permanently close and disappear. Accordingly, in one embodiment of the present invention, the absorption of red and IR photons in the biological material of the first or target region along with biophoton radiation can cause changes in the first region, thereby inducing changes in the second or target region to promote wound healing, e.g., infected, ischemic, and hypoxic wounds and/or help repair soft tissue, noted above.

Yet, another area of application of photobiomodulation is a direct control of brain cell activity with light. The technique is based upon NIR spectroscopy and is simpler to use and less expensive than other methods such as functional magnetic resonance imaging and positron emission tomography.

Whenever a region of the brain is activated, that part of the brain uses more oxygen. This technique works by measuring the blood flow and oxygen consumption in the brain. The light emitted by NIR laser diodes is carried through optical fibers to a person's head. The light penetrates the skull where it assesses the brain's oxygen level and blood volume. The scattered light is then collected by optical fibers, sent to detectors and analyzed by a computer. The scattered light is affected also by activation of heavy isotopes of O2 showing increased emission times. By examining how much of the light is scattered and how much is absorbed, portions of the brain and extract information about brain activity can be mapped. By measuring the scattering, it is determined where the neurons are firing. This means that scientists can simultaneously detect both blood profusion and neural activity. The technique could be used in many diagnostic, prognostic and clinical applications. For example, it could be used to find hematomas in children, to study blood flow in the brain during sleep apnea, and to monitor recovering stroke patients on a daily, or even hourly, basis (that would be impractical to do with MRI). To validate the technique, hemoglobin oxygen concentrations in the brain obtained simultaneously by NIR spectroscopy and by functional MRI, the current “gold standard” in brain studies, was compared. Both methods were used to generate functional maps of the brain's motor cortex during a periodic sequence of stimulation by finger motion and rest. Spatial congruence between the hemoglobin signal and the MRI signal in the motor cortex related to finger movement was demonstrated. The researchers also demonstrated collocation between hemoglobin oxygen levels and changes in scattering due to brain activities. The changes in scattering associated with fast neuron signals came from exactly the same locations. Accordingly, in one embodiment of the present invention, the absorption of NIR in the biological material of the first or target region coupled to brain tissue along with biophoton radiation the biophoton sources noted above can directly cause changes in the first region, thereby inducing changes in the second or target region in the actual brain tissue for control of brain cell activity, as noted above.

A low-intensity laser light-oxygen cancer therapy is another application of photobiomodulation. The light-oxygen effect (LOE), which involves activation of or damage to biosystems by optical radiation at low optical doses by direct photoexcitation of molecular oxygen dissolved in a biosystem so that it is converted to the singlet state, i.e., by photogeneration of molecular singlet oxygen from O₂ dissolved in cells, similar to photodynamic effect (Zakharov S D, et al., Quantum Electronics, 29(12):1031-53 (1999)). It was shown that the He-Ne laser radiation destroys tumor cells in the presence or absence of the photosensitiser. The LOE can be activated by small optical doses, which are 4-5 orders of magnitude lower that those found if a comparison is made with the familiar analogue in the form of the photodynamic effect (PDE). Accordingly, in one embodiment of the present invention, the absorption of He-Ne laser radiation in the biological material of the first or target region coupled to cancerous tissue along with biophoton radiation the biophoton sources noted above can cause changes in the first region, thereby inducing changes in the second or target region in the actual cancerous tissue.

### Assisted Photostimulation

One photostimulation technique involves chemical modification of ion channels and receptors to render them light-responsive. Some of the most fundamental signaling mechanisms in a cell involve the release and uptake of Ca²⁺ ions. Ca²⁺ is involved in controlling fertilization, differentiation, proliferation, apoptosis, synaptic plasticity, memory, and developing axons. It has been shown that Ca²⁺ waves can be induced by UV irradiation (single-photon absorption) and NIR irradiation (two-photon absorption) by releasing caged Ca2+, an extracellular purinergic messenger InsP3 (Braet K., et al., Cell Calcium, 33:37-48 (2003)), or ion channel ligands (Zhang F., et al., 2006).

Directly controlling a brain cell activity with light is a novel means for experimenting with neural circuits and could lead to therapies for some disorders. This accomplishment is a step toward the goal of mapping neural circuit dynamics on a millisecond timescale to see if impairments in these dynamics underlie severe psychiatric symptoms. Knowing the effects that different neurons have could ultimately help researchers figure out the workings of healthy and unhealthy brain circuits. If use of the technique can show that altered activity in a particular kind of neuron underlies symptoms, for example, this insight will allow development of targeted genetic or pharmaceutical treatments to fix those neurons. Conceivably, direct control of neuronal activity with light could someday become a therapy in itself. Here, the phosphor configurations of the invention can be programmed or instructed or configured to deliver light for direct control of neuronal activity.

In living organisms, scientists have been able to cause worms, C. elegans, to stop swimming while their genetically altered motor neurons were exposed to pulses of yellow light intensified through a microscope. In some experiments, exposure to blue light caused the worms to wiggle in ways they weren't moving while unperturbed. When the lights were turned off, the worms resumed their normal behavior.

Meanwhile, in experiments in living brain tissues extracted from mice, the researchers were able to use the technique to cause neurons to signal or stop on the millisecond timescale, just as they do naturally. Other experiments showed that cells appear to suffer no ill effects from exposure to the light. The mice resume their normal function once the exposure ends.

The most direct application of an optical neuron control is experimenting with neural circuits to determine why unhealthy ones fail and how healthy ones work.

In patients with Parkinson's disease, for example, researchers have shown that electrical “deep brain stimulation” of cells can help patients, but they don't know precisely why. By allowing researchers to selectively stimulate or dampen different neurons in the brain, the light stimulation techniques could help in determining which particular neurons are benefiting from deep brain stimulation. That could lead to making the electrical treatment, which has some unwanted side effects, more targeted.

Another embodiment of the present invention is the stimulation of neural communications. Because neurons communicate by generating patterns of signals-sometimes on and sometimes off like the 0s and 1s of binary computer code-flashing blue and yellow lights in these patterns could compel neurons to emit messages that correspond to real neural instructions. The present invention can be used to test and tune sophisticated neuron behaviors. The ability to artificially stimulate neural signals, such as movement instructions using the present invention may allow doctors to bridge blockages in damaged spinal columns, perhaps restoring some function to the limbs of paralyzed patients.

Accordingly, in one embodiment of the present invention, the absorption of photons designed for photostimulation in the biological material of the first or target region along with biophoton radiation from one of the biophoton sources noted above can cause or induce changes in the first region via photostimulation, thereby inducing changes in the second or target region for stimulation and/or control of neural communication and other neuron activities.

### In Vivo or In Vitro Internal Light Sources

In one embodiment, sources of internal light can be used in this invention to stimulate bioactivity (as discussed above and elsewhere) and or to simulate natural biophoton sources. In one embodiment, the sources of internal light for use in this invention can include persistent after-glow phosphor materials emitting light in the visible to near ultraviolet and ultraviolet range. These sources of internal light can be either sources inside a patient or inside an artificial construct containing biological material to be exposed to the light where the sources comprise up converting or down converting phosphors or fluorescent agents, and preferably down converting phosphors or fluorescent agents which, upon exposure to x-rays (or other high energy waves or particles) emit ultraviolet and/or visible light at the known emission bands of these phosphors and fluorescent agents. These sources of internal light can be those described above for the in vivo point of use biophoton generator and the biophoton stimulator.

In one embodiment, Eu-doped strontium aluminate is used as an internal light source in which deep UV light or x-ray or electron beams “charge” the photoluminescence such that these phosphors can, for example, be charged outside a patient and then injected into a target or diseased site where UV photons would be emitted. In another embodiment, gadolinium strontium magnesium aluminate is used as an internal light source in which deep UV light or x-ray or electron beams “charge” the photoluminescence such that these phosphors can, for example, be charged outside a patient and then injected into a target or diseased site where UV photons would be emitted. U.S. Pat. Appl. Publ. No. 20070221883 (the entire contents of which are incorporated herein by reference) describes specifically gadolinium-activated strontium magnesium aluminate having an excitation maximum at about 172 nm, and which emits in a narrow-band UV emission at about 310 nm. The '883 publication also describes other useful internal light sources for this invention, making note of emission spectra between 300 nm and 320 nm for a Sr(Ai,Mg)₁₂O₁₉:Gd phosphor and two 312 nm line emitting phosphors, YMgB₅O₁₀:Gd, Ce and YMgB₅O₁₀:Gd, Ce, Pr. WO2016200349 (the entire contents of which are incorporated herein by reference) describes long lasting yellowish-green emitting phosphorescent pigments in the strontium aluminate (SrAl2O4) system, which could serve as internal light sources in the present invention. WO 2016200348 (the entire contents of which are incorporated herein by reference) describes long lasting bluish-green emitting phosphorescent pigments in the strontium aluminate (Sr4Al14O25) system, which could serve as internal light sources in the present invention. Xiong et al in “Recent advances in ultraviolet persistent phosphors,” Optical Materials X 2 (2019) (the entire contents of which are incorporated herein by reference) describes a number of ultraviolet persistent phosphors that could serve as internal light sources in the present invention. Table 4 below provides a listing of such persistent phosphors:

TABLE 4

| SrO:Pb²⁺ | 390 | >1 | h |
|---|---|---|---|
| CaAl₂O₄:Ce³⁺ Tb³⁺ | 400 | >10 | h |
| CaAl₂O₄:Ce³⁺ Tb³⁺ | 413 | >10 | h |
| Sr₂Al₂SiO [symbol] :Ce³⁺ | 400 | several minutes | |
| SrZrO [symbol] | 395 | <1000 | s |
| BaZrO [symbol] :Mg²⁺ | 400 | >2400 | s |
| SrZrO [symbol] :Pr [symbol] ⁺ | 356 | | |
| CdSiO₃:Bi³⁺ | 360 | | |
| CdSiO₃:Bi³⁺ Dy³⁺ | 360 | | |
| CdSiO₃:Bi³⁺ Gd [symbol] | 344 | >6 | h |
| Sr₂MgGe₂O₇:Pb²⁺ | 370 | >12 | h |
| NaLaGeO [symbol] :Bi³⁺ [symbol] | 400 | >63 | h |
| CaZnGe₂O₈:Bi³⁺ | 300-700 | >12 | h |
| C [symbol] Na [symbol] :Pr³⁺ | 250 | >2 | h |

[symbol]

indicates data missing or illegible when filed

In one embodiment, the phosphor described by Xiong et al as CaAl₂O₄:Ce³⁺ having an emission peak of 400 nm and a persistent time of more than 10 h could be used, where it would be charged by x-ray irradiation outside a patient and then injected at a diseased site to provide internally generated UV light.

In one embodiment, the persistent phosphors noted could be activated ex vivo and introduced along with psoralen (or other photoactivatable drug) into the patient by exchange of a bodily fluid or for example by supplying the persistent phosphors and the photoactivatable drug into a patient's blood stream.

In one embodiment, the persistent phosphors noted could be activated in vivo by injection of the phosphors into a diseased site (or at a site to be treated) and then exposed to x-rays producing a persistent internal light source.

In another embodiment, a combined electromagnetic energy harvester molecule could be used as an internal light source, such as the combined light harvester disclosed in J. Am. Chem. Soc. 2005, 127, 9760-9768, the entire contents of which are hereby incorporated by reference. By combining a group of fluorescent molecules in a molecular structure, a resonance energy transfer cascade may be used to harvest a wide band of electromagnetic radiation resulting in emission of a narrow band of fluorescent energy. In another embodiment, a Stokes shift of an emitting source or a series of emitting sources arranged in a cascade is selected to convert a shorter wavelength energy, such as X-rays, to a longer wavelength fluorescence emission such an optical or UV-A.

In one embodiment, a lanthanide chelate capable of intense luminescence is used as an internal light source. In another embodiment, a biocompatible, endogenous fluorophore emitter can be used as an internal light source.

In one embodiment, the internal light source of this invention can include visible and UV-light emitting bioluminescent materials. In one embodiment, bioluminescent materials such as coelenterate-type luciferin analogues could be used including amide monoanion known to emit at 480 nm and oxyluciferin known to emit at 395 nm.

In another embodiment of the invention, mechano-luminescent materials can be used as internal light sources.

Mechano-luminescent materials convert ultrasonic or mechanical energy (such as vibrations naturally existing on an article such as motor or vibrations from driven by transducers) into visible light. Here, for example, the mechano-luminescent materials would be placed in a vicinity of a diseased site or at a site or sites to be treated with internally generated light.

Within the context of the present invention, the phrase “in a vicinity of”, and variations thereof, includes near, adjacent, or within/inside a diseased site or site or sites to be treated.

Various mechano-luminescent materials suitable for the present invention include ZnS:Mn²⁺, SrAl₂O₄:Eu²⁺, ZnS:Cu, SrAMgSi₂O₇:Eu²⁺ (A=Ca, Sr, Ba), KCl, KI, KBr, NaF, NaCl, LiF, RbCl, RbBr, RbI, MgO, SrAl₂O₄, CaAl₂O₄, Sr₁₋ₓBaₓAl₂O₄(x=0, 0.1, 0.2, 0.4), Sr_{0.9}Ca_{0.1}Al₂O₄, Zn₂Ge_{0.9}Si_{0.1}O₄, MgGa₂O₄, ZnGa₂O₄, ZnAl₂O₄, ZnS, ZnTe, (ZnS)₁₋ₓ(MnTe)ₓ (x<¼), CaZnOS, BaZnOS, Ca₂MgSi₂O₇, Sr₂MgSi₂O₇, Ba₂MgSi₂O₇, SrCaMgSi₂O₇, SrBaMgSi₂O₇, SrₙMgSi₂O₅₊ₙ (1≤n≤2), Ca₂Al₂SiO₇, Sr₂Al₂SiO₇, CaYAl₃O₇, CaAl₂Si₂O₈, Ca₁₋ₓSrₓAl₂Si₂O (x<0.8), SrMg₂(PO₄)₂, Ba₁₋ₓCaₓTiO₃ (0.25<x<0.8), Ba₁₋ₓCaₓTiO₃, LiNbO₃, Sr₂SnO₄, (Ca, Sr, Ba)₂SnO₄, Sr₃Sn₂O₇, Sr₃(Sn, Si)₂O₇, Sr₃(Sn, Ge)₂O₇, Ca₃Ti₂O₇, CaNb₂O₆, Ca₂Nb₂O₇, Ca₃Nb₂O₈, BaSi₂O₂N₂, SrSi₂O₂N₂, CaZr(PO₄)₂, ZrO₂.

In one embodiment, a europium-holmium co-doped strontium aluminate can be used as a mechano-luminescent material (i.e., an internal light source). The europium-holmium co-doped strontium aluminate and the other mechano-luminescent materials convert sonic or acoustic energy into photon emissions which may be placed in a vicinity of a diseased site or at a site or sites to be treated with internally generated light.

Yanim Jia, in “Novel Mechano-Luminescent Sensors Based on Piezoelectric/Electroluminescent Composites,” Sensors (Basel). 2011; 11(4): 3962-396, the entire contents of which are incorporated by reference, describes a mechanoluminescent composite made of a piezoelectric material and an electroluminescent material. In this composite device, when a stress is applied to the piezoelectric layer, electrical charges will be induced at both the top and bottom faces of piezoelectric layer due to the piezoelectric effect. These induced electrical charges will result in a light output from the electroluminescent layer due to the electroluminescent effect.

Here, in one embodiment of the present invention, such composites made of a piezoelectric material and an electroluminescent material, hereinafter “composite mechano-luminescent emitters,” provides a structure that, upon stimulation with mechanical or vibrational energy such as from an acoustic or ultrasonic transducer, emit light to a diseased site or at a site or sites to be treated with internally generated light.

The present invention in various embodiments can utilize organic fluorescent molecules or inorganic particles capable or fluorescence and phosphorescence having crystalline, polycrystalline or amorphous micro-structures for the internal light sources of this invention generating light at a diseased site or at a site or sites to be treated with internally generated light.

The list of inorganic molecules that can be used for the electroluminescence and phosphorescent materials described below include but is not limited to the following inorganic electroluminescent phosphor materials:
SrS:Ce³⁺
CaGa₂S₄:Ce³⁺
SrS:Cu⁺
CaS:Pb²⁺
BaAl₂S₄:Eu²⁺
ZnS:Tb³⁺
ZnMgS:Mn²⁺
SrGa₂S₄:Eu²⁺
CaAl₂S₄:Eu²⁺
BaAl₂S₄:Eu²⁺
ZnS:Mn²⁺
MgGa₂O₄:Eu³⁺
(Ca, Sr)Y₂S₄:Eu²⁺
BaAl₂S₄:Eu²⁺

Organic molecules that can phosphoresce under the influence of an electric field are also of interest in the present application. The organic fluorescent compounds with high quantum yield include by way of illustration:
Naphthalene,
Pyrene,
Perylene,
Anthracene,
Phenanthrene,
p-Terphenyl,
p-Quartphenyl,
Trans-stilbene,
Tetraphenylbutadiene,
Distyrylbenzene,
2,5-Diphenyloxazole,
4-Methyl-7-diethylaminocoumarin,
2-Phenyl-5-(4-biphenyl)-1,3,4-oxadiazole,
3-Phenylcarbostyryl,
1,3,5-Triphenyl-2-pyrazoline,
1,8-Naphthoylene-1′,2′-bezimidazole,
4-Amino-N-phenyl-naphthalimide.

The inorganic fluorescent and phosphorescent materials detailed here are numerous, and various examples are given by way of illustration rather than limitation and can be used for the internal light sources of this invention generating light at a diseased site or at a site or sites to be treated with internally generated light.

Furthermore, these materials can be doped with specific ions (activators or a combination of activators) that occupy a site in the lattice structure in the case of crystalline or polycrystalline materials and could occupy a network forming site or a bridging and/or non-bridging site in amorphous materials. These compounds could include (not ranked by order of preference or utility) the following material examples:
CaF₂, ZnF₂, KMgF₃, ZnGa₂O₄, ZnAl₂O₄, Zn₂SiO₄, Zn₂GeO₄, Ca₅(PO₄)₃F, Sr₅(PO₄)₃F, CaSiO₃, MgSiO₃, ZnS, MgGa₂O₄, LaAl₁₁O₁₈, Zn₂SiO₄, Ca₅(PO₄)₃F, Mg₄Ta₂O₉, CaF₂, LiAl₅O₈, LiAlO₂, CaPO₃, AlF₃.

Further included are alkali earth chalcogenide phosphors which are in turn exemplified by the following non-inclusive list:
MgS:Eu³⁺, CaS:Mn²⁺, CaS:Cu, CaS:Sb, CaS:Ce³⁺, CaS:Eu²⁺, CaS:Eu²⁺Ce³⁺, CaS:Sm³⁺, CaS:Pb²⁺, CaO:Mn²⁺, CaO:Pb²⁺.

The examples include the ZnS type phosphors that encompass various derivatives:
ZnS:Cu,Al(Cl), ZnS:Cl(Al), ZnS:Cu,I(Cl), ZnS:Cu, ZnS:Cu,In.

Compound IIIb-Vb phosphors which include the group IIb and Vb elements of the periodic table are suitable phosphors. These semiconductors include BN, BP, BSb, AlN, AlP, AlAs, AlSb, GaN, GaP, GaAs, GaSb, InN, InP, InAs, InSb and these materials have donors and acceptors that work in together to induce light emission diodes. The donors include Li, Sn, Si, Li, Te, Se, S, O, and acceptors include C, Be, Mg, Zn, Cd, Si, Ge. As an example, GaP light emitting diodes include GaP:Zn, O, GaP:NN, Gap:N and GaP which emit colors Red, Yellow, Green and Pure Green respectively.

The compounded materials further include such materials as GaAs with compositional variation of the following sort: In1-y(Ga1-xAlx)yP (provides a simple example).

Silicon Carbide SiC as a luminescent platform has commercial relevancy for blue light emitting diodes and could be used as an internal light source if appropriately powered from the outside. The SiC luminescent platform could include the polytypes 3C-SiC, 6H-SiC, 4H-SiC with donors such as N and Al and acceptors such as Ga and B.

Multiband luminescent materials suitable for converter materials include for example the following compositions:
(Sr, Ca, Ba)₅(PO₄)₃Cl:Eu²⁺, BaMg₂Al₁₆O₂₇:Eu²⁺, CeMgAl₁₁O₁₉:Ce³⁺:Tb³⁺, LaPO₄:Ce³⁺:Tb³⁺, GdMgB₅O₁₀:Ce³⁺:Tb³⁺, Y₂O₃:Eu³⁺, (Ba,Ca,Mg)₅(PO₄)₃Cl:Eu²⁺, 2SrO_{0.84}P₂O₅·0.16B₂O₃:Eu²⁺, Sr₄Al₁₄O₂₅:Eu²⁺.

Other materials suitable for the internal light sources of this invention generating light at a diseased site or at a site or sites to be treated with internally generated light include those materials typically used for fluorescent high pressure mercury discharge lamps but which can be excited with X-Ray and are exemplified by way of family designation as follows:
Phosphates (Sr, M)(PO₄)₂:Sn²⁺, Mg or Zn activator, Germanate 4MgO·GeO₂:Mn⁴⁺, 4(MgO, MgF₂)GeO₂:Mn⁴⁺, Yttrate Y₂O₃:Eu³⁺, Vanadate YVO₄:Eu³⁺, Y(P,V)O₄:Eu³⁺, Y(P,V)O₄:In⁺, Halo-Silicate Sr2Si3O₈·2SrCl₂:Eu²⁺, Aluminate (Ba,Mg)₂Al₁₆O₂₄:Eu²⁺, (Ba, Mg)₂Al₁₆O₂₄:Eu²⁺,Mn²⁺, Y₂O₃Al₂O₃:Tb³⁺.

Another grouping of materials suitable for converter materials for the internal light source include chemical compositions in the Halophosphates phosphors, Phosphate phosphors, Silicate phosphors, Aluminate phosphors, Borate phosphors, Tungstate phosphors, and other phosphors.

The halophosphates include by way of illustration:
3Ca₃(PO₄)₂·Ca(F,Cl)₂:Sb³⁺, 3Ca₃(PO₄)₂·Ca(F,Cl)₂:Sb³⁺Mn²⁺, Sr₁₀(PO₄)₆Cl₂:Eu²⁺, (Sr,Ca)₁₀(PO₄)₆Cl₂:Eu²⁺, (Sr,Ca)₁₀(PO₄)₆·nB₂O₃:Eu³⁺, (Sr, Ca,Mg)₁₀(PO₄)₆Cl₂:Eu²⁺. The phosphate phosphors include by way of illustration Sr₂P₂O₇:Sn²⁺, (Sr,Mg)₃(PO₄)₂:Sn²⁺, Ca₃(PO₄)₂·Sn²⁺, Ca₃(PO₄)₂:Tl⁺, (Ca,Zn)₃(PO₄)₂:Tl⁺, Sr₂P₂O₇:Eu²⁺, SrMgP₂O₇:Eu²⁺, Sr₃(PO₄)₂:Eu²⁺, LaPO₄:Ce³⁺, Tb³⁺, La₂O₃·0.2SiO₂·0.9P₂O₅:Ce³⁺·Tb³⁺, BaO·TiO₂·P₂O₅. The silicate phosphors Zn₂SiO₄:Mn²⁺, CaSiO₃:Pb²⁺/Mn²⁺, (Ba, Sr, Mg)·3Si₂O₇:Pb²⁺, BaSi₂O₅:Pb²⁺, Sr₂Si₃O₈·2SrCl₂:Eu²⁺, Ba₃MgSi₂O₈:Eu²⁺, (Sr,Ba)Al₂Si₂O₈:Eu²⁺.

The aluminate phosphors include:
LiAlO₂:Fe³⁺, BaAl₈O₁₃:Eu²⁺, BaMg₂Al₁₆O₂₇:Eu²⁺, BaMg₂Al₁₆O₂₇:Eu²⁺/Mn²⁺, Sr₄Al₁₄O₂₅:Eu²⁺, CeMgAl₁₁O₁₉:Ce³⁺/Tb³⁺.

The borate phosphors include:
Cd₂B₂O₅:Mn²⁺, SrB₄O₇F:Eu²⁺, GdMgB₅O₁₀:Ce³⁺/Tb³⁺, GdMgB₅O₁₀:Ce³⁺/Mn³⁺, GdMgB₅O₁₀:Ce³⁺/Tb³⁺/Mn²⁺.

The tungstate phosphors include:
CaWO₄, (Ca,Pb)WO₄, MgWO₄. Other phosphors Y₂O₃:Eu³⁺, Y(V,P)O₄:Eu²⁺, YVO₄:Dy³⁺, MgGa₂O₄:Mn²⁺, 6MgO·As₂O₅:Mn²⁺, 3.5MgO·0.5MgF₂·GeO₂:Mn⁴⁺.

Activators of relevance to the various doped phosphors include the following list:
Tl⁺, Pb²⁺, Ce³⁺, Eu²⁺, WO₄²⁻, Sn²⁺, Sb³⁺, Mn²⁺, Tb³⁺, Eu³⁺, Mn⁴⁺, Fe³⁺.

In various embodiments, the luminescence center Tl+ can be used with a chemical composition such as:
(Ca,Zn)₃(PO₄)₂:Tl⁺, Ca₃(PO₄)₂:Tl⁺.

Similarly, the luminescence center Mn2+ can be used with chemical compositions such as
MgGa₂O₄:Mn2+, BaMg₂Al₁₆O₂₇:Eu²⁺/Mn²⁺, Zn₂SiO₄:Mn²⁺, 3Ca₃(PO₄)₂·Ca(F,Cl)₂:Sb²⁺/Mn²⁺, CaSiO₃:Pb²⁺/Mn²⁺, Cd₂B₂O₅:Mn²⁺, CdB₂O₅:Mn²⁺, GdMgB₅O₁₀:Ce³⁺Mn²⁺, GdMgB₅O₁₀:Ce³⁺/Tb³⁺/Mn²⁺.

Further, the luminescence center Sn²⁺ can be used with chemical compositions such as:
Sr₂P₂O₇:Sn²⁺, (Sr,Mg)₃(PO₄)₂:Sn²⁺.

The luminescence center Eu²⁺ can also be used with chemical compositions such as: SrB₄O₇F:Eu²⁺, (Sr,Ba)Al₂Si₂O₈:Eu²⁺, Sr₃(PO₄)₂:Eu²⁺, Sr₂P₂O₇:Eu²⁺, Ba₃MgSi₂O₈:Eu²⁺, Sr₁₀(PO₄)₆Cl₂:Eu²⁺, BaMg₂Al₁₆O₂₇:Eu²⁺/Mn²⁺, (Sr,Ca)₁₀(PO₄)₆Cl₂:Eu²⁺.

The luminescence center Pb²⁺ can be used with chemical compositions such as:
(Ba,Mg,Zn)₃Si₂O₇:Pb²⁺, BaSi₂O₅:Pb²⁺, (Ba,Sr)₃Si₂O₇:Pb²⁺.

The luminescence center Sb²⁺ can be used with chemical compositions such as:
3Ca₃(PO₄)₂·Ca(F,Cl)₂:Sb³⁺, 3Ca₃(PO₄)₂·Ca(F,Cl)₂:Sb³⁺/Mn²⁺.

The luminescence center Tb3+ can be used with chemical compositions such as:
CeMgAl₁₁O₁₉:Ce³⁺/Tb³⁺, LaPO₄:Ce³⁺/Tb³⁺, Y₂SiO₅:Ce³⁺/Tb³⁺, GdMgB₅O₁₀:Ce³⁺/Tb³⁺.

The luminescence center Eu³⁺ can be used with chemical compositions such as:
Y₂O₃:Eu³⁺, Y(V,P)O₄:Eu³⁺.

The luminescence center Dy³⁺ can be used with chemical compositions such as:
YVO₄:Dy³⁺.

The luminescence center Fe³⁺ can be used with chemical compositions such as:
LiAlO₂:Fe³⁺.

The luminescence center Mn⁴⁺ can be used with chemical compositions such as:
6MgO·As₂O₅:Mn⁴⁺, 3.5MgO·0.5MgF₂·GeO₂:Mn⁴⁺.

The luminescence center Ce³⁺ can be used with chemical compositions such as:
Ca₂MgSi₂O₇:Ce³⁺ and Y₂SiO₅:Ce³⁺.

The luminescence center WO₄²⁻ can be used with chemical compositions such as:
CaWO₄, (Ca,Pb)WO₄, MgWO₄.

The luminescence center TiO₄⁴⁻ can be used with chemical compositions such as:
BaO·TiO₂·P₂O₅.

In various embodiments of this invention, the phosphor chemistry utilized in x-ray excitations can be used for the internal light sources of this invention generating light at a diseased site or at a site or sites to be treated with internally generated light.

Of particular interest is the k-edge of these phosphors. Low energy excitation can lead to intense luminescence in materials with low k-edge. Some of these chemistries and the corresponding k-edge are included as follows:

| | | |
|---|---|---|
| BaFCl:Eu²⁺ | 37.38 | keV |
| BaSO₄:Eu²⁺ | 37.38 | keV |
| CaWO₄ | 69.48 | keV |
| Gd₂O₂S:Tb³⁺ | 50.22 | keV |
| LaOBr:Tb³⁺ | 38.92 | keV |
| LaOBr:Tm³⁺ | 38.92 | keV |
| La₂O₂S:Tb³⁺ | 38.92 | keV |
| Y₂O₂S:Tb³⁺ | 17.04 | keV |
| YTaO₄ | 67.42 | keV |
| YTaO₄:Nb | 67.42 | keV |
| ZnS:Ag | 9.66 | keV |
| (Zn, Cd)S:Ag | 9.66/26.7 | keV |

In one embodiment of this invention, light from these materials (excited for example by high energy particles including x-rays, gamma rays, protons, and electrons) can have their emissions act as the internal light sources of this invention generating light at a diseased site or at a site or sites to be treated with internally generated light.

Various materials used for the electro-luminescence can be used for the internal light sources of this invention generating light at a diseased site or at a site or sites to be treated with internally generated light. The electro-luminescence materials can include but are not limited to:
4,4′,4″-Tris[phenyl(m-tolyl)amino]triphenylamine (m-MTDATA)
N,N′-Bis(3-methylphenyl)-N,N′-diphenylbenzidine (TPD)
4,4′,4″-Tris[phenyl(m-tolyl)amino]triphenylamine (m-MTDATA)
N,N′-Bis(3-methylphenyl)-N,N′-diphenylbenzidine (TPD)
Tris-(8-hydroxyquinoline)aluminum
2,4,6-Tris(2-pyridyl)-s-triazine (TPT)

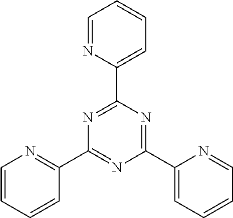 [SMILES: c1ccc(-c2nc(-c3ccccn3)nc(-c3ccccn3)n2)nc1]

2,2′,2″-(1,3,5-Benzinetriyl)-tris(1-phenyl-1-H-benzimidazole) Alq

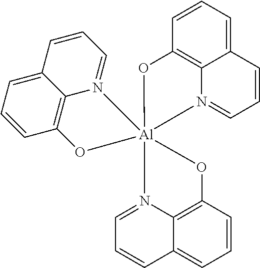

2,2′,2″-(1,3,5-Benzinetriyl)-tris(1-phenyl-1-H-benzimidazole) TPBI

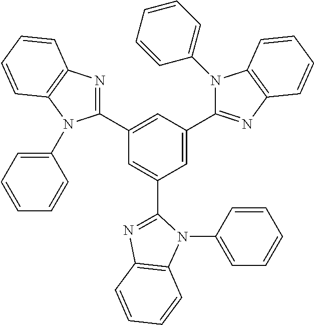 [SMILES: c1ccc(-n2c(-c3cc(-c4nc5ccccc5n4-c4ccccc4)cc(-c4nc5ccccc5n4-c4ccccc4)c3)nc3ccccc32)cc1]

2,9-Dimethyl-4,7-diphenyl-1,10-phenanthroline, BCP2,9-Dimethyl-4,7-diphenyl-1,10-phenanthroline, BCP

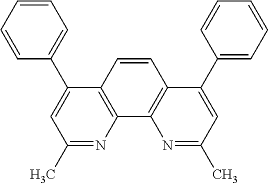 [SMILES: Cc1cc(-c2ccccc2)c2ccc3c(-c4ccccc4)cc(C)nc3c2n1]

### Stimulated Regeneration and Phototreatment

In one embodiment of this invention, the photon radiation generated by the sources described above such as the in vivo point of use biophoton generator, the biophoton stimulator, and the in vivo and in vitro internal light sources described above (and the fluorescing materials and phosphors described herein) can be used as a source of light to stimulate bioactivity (as discussed above and elsewhere) and/or to simulate or stimulate natural biophoton sources.

In prior work entitled “Conjugated polymers optically regulate the fate of endothelial colony-forming cells, conjugated polymers were used with visible light excitation to gain optical control of cell fate” by Lodola et al. in Science Advances 27 Sep. 2019: Vol. 5, no. 9, the entire contents of which are incorporated herein by reference, endothelial progenitor cells (EPCs) and, in particular, endothelial colony-forming cells (ECFCs) were evaluated for optical regulation. ECFCs can be mobilized from the bone marrow and vascular stem cell niche to reconstruct a vascular network destroyed by an ischemic insult and to restore local blood perfusion. ECFCs can be harvested from peripheral blood, and are known to display robust clonogenic potential, exhibit tube-forming capacity in vitro, and generate vessel-like structures in vivo.

This work by Lodola et al. demonstrated that polymer-mediated optical excitation during the first steps of ECFC growth could lead to a robust enhancement of both proliferation and tubulogenesis through the optical modulation of the Ca²⁺-permeable transient receptor potential vanilloid 1 (TRPV1) channel and NF-κB-mediated gene expression. The material used for light absorption and phototransduction was regioregular poly(3-hexyl-thiophene) (P3HT), a thiophene-based conjugated polymer which acted as an exogenous, light-responsive actuator. This work by Lodola et al. used polymer thin films (approximate thickness, 150 nm) deposited by spin coating on top of polished glass substrates. Both polymer-coated and glass substrates have been thermally sterilized (120° C., 2 hours), coated with fibronectin, and, lastly, used as light-sensitive and control cell culturing substrates, respectively. ECFCs were seeded on top of polymer coated glass substrates.

This work by Lodola et al. provided optical excitation by a light-emitting diode (LED) source, with maximum emission wavelength at 525 nm, incident from the substrate side. A protocol based on 30-ms excitation pulses, followed by a 70-ms dark condition, at a photoexcitation density of 40 mW/cm² was used to minimize heating. The whole protocol is continuously repeated for a minimum of 4 up to 36 hours, depending on the type of functional assay, at controlled temperature (37° C.) and CO₂ levels (5%). This work found that optical excitation, properly mediated by biocompatible polymer substrates, positively affects ECFC fate by spatially and temporally selective activation of the TRPV1 channel which has been shown to be expressed and drive angiogenesis in human ECFCs.

More significantly, this work postulated that the P3HT polymer upon interaction with light induced an excited state of P3HT resulting in charged oxygen state O₂⁻, subsequently producing hydrogen peroxide, triggering intracellular reactive oxygen species (ROS) enhancement.

Lesions have been treated with target light-sensitive molecules called photosensitizers (PSs). When irradiated with light, PSs generate reactive oxygen species (ROS) which very rapidly react with any nearby biomolecule and can eventually kill cells through apoptosis or necrosis. The technique, called chromophore-assisted light inactivation (CALI), has been used for the treatment of precancerous lesions and superficial tumors.

In one embodiment of this invention, the stimulated activity generated by the light internally generated in the medium to be treated promotes the formation of new blood vessels using at least one of ultraviolet and/or visible light emission into the medium to be treated. Here, the internal light sources generate the ultraviolet and/or the visible light which exposes a photosensitive material (for example the P3HT polymer noted above) contained within or in a vicinity of natural or artificial tissue cells containing endothelial progenitor cells. In one embodiment, the ultraviolet and/or the visible light generated within the photosensitive material generates reactive oxygen species which can promote an angiogenesis process within the natural or artificial tissue cells containing the endothelial progenitor cells. In one embodiment, the light internally generated in the medium is generated by phosphorescence or fluorescence of light emitting materials disposed within the photosensitive material (for example the P3HT polymer noted above) when the light emitting materials are exposed to x-rays.

In one embodiment, the phosphorescence or fluorescence light emitting materials are disposed in a biocompatible polymer that is not necessarily photosensitive. The biocompatible material is coated or else is to be located in vicinity to endothelial progenitor cells. X-ray exposure of this composite biocompatible polymer generates UV light emission from the phosphorescence or fluorescence light emitting materials which exits the composite biocompatible polymer and generates ROS in the medium about the endothelial progenitor cells, thereby stimulating blood vessel growth.

In other prior work, Andres Garcia of the Georgia Institute of Technology and his team have made blood vessels grow by shining light on skin. In this prior work, entitled “Light-triggered in vivo activation of adhesive peptides regulates cell adhesion, inflammation and vascularization of biomaterials: published in Nature Materials volume 14, pages 352-360 (2015), the entire contents of which are incorporated herein by reference, a RGB peptide (used to signal cells to grow on new tissues) and a photo-responsive blocker were impregnated into a water-based gel, or hydrogel, which was later activated by UV light from an external source. The UV light released the blocker and cell growth was observed. However, the depth of penetration of UV light from an external source limits the utility of this approach.

In one embodiment of the present invention, a water-based gel, or hydrogel, is impregnated with a RGB peptide and a material of one of the internal light sources described above such that activation for example by x-ray exposure generates within the hydrogel the ultraviolet and/or the visible light. When UV light from the internal light source in the hydrogel is generated, the UV light causes the blocker to be released, and the RGB peptide to become active.

In one embodiment, the hydrogel with the impregnated RGB peptide, the blocker, and the internal light source material is implanted into a patient and exposed to x-ray flux which generates within the hydrogel UV light which causes the blocker to be released, and the RGB peptide to become active within the patient.

In another embodiment, the hydrogel with the impregnated RGB peptide, the blocker, the internal light source material, and a vascular endothelial growth factor protein that stimulates the growth of new blood vessels is implanted into a patient and exposed to x-ray flux which generates within the hydrogel UV light which causes the blocker to be released, and the RGB peptide and the vascular endothelial growth factor protein to become active.

Thus, in one embodiment of the invention, there is provided a method for regenerative medicine using internal light sources within artificial or in vivo living cells to regrow cells of an organ in a patient in which light for the internal light sources stimulates or otherwise promote s the regrowth/regeneration of cells of the organ, for example where angiogenesis (blood vessel regrowth occurs as an example due to generation of reactive oxygen species or for example the removal of blocking proteins preventing endothelial progenitor cells from generating new cells.

In other prior work, Berkowitz et al. and his research team at John Hopkins in an article entitled “Melanopsin mediates light-dependent relaxation in blood vessels,” in Proceedings of the National Academy of Sciences in North America, first published Nov. 17, 2014, vol. 11, no. 50 pp 17977-17982 (the entire contents of which are incorporated herein by reference) have found that delivering light to blood vessels can deter vascular disease. Accordingly, in another embodiment of the present invention, light from internal light sources inside blood vessels can stimulate blood vessels. It was learned by Berkowitz that melanopsin (opsin 4) is one group of nonimage-forming light receptors that are present in blood vessels elsewhere in the human body which help set the circadian rhythms that affect the body's daily cycle of physical, mental and behavioral changes. Berkowitz et al. reported a physiological role for Opn4 in regulating blood vessel function, particularly in the context of photorelaxation.

Berkowitz et al. further reported that opsin 4 (a classic G protein-coupled receptor) is expressed in blood vessels. Vasorelaxation was reported by Berkowitz et al. to be wavelength-specific, with a maximal response at vessels at low-intensity blue light (380-495 nm), which was reported by Berkowitz et al. to correspond to the optimal absorption wavelength for the mouse Opn4 receptor. In short, Berkowitz et al. found that exposure of the blood vessels to blue light increased blood flow.

In general, a variety of different microbial opsins and genetically modified opsins have been used and developed to date for optogenetic manipulations. In the art, the term opsin describes a light-responsive protein, independent of its chromophore type (e.g., retinal, flavin), mode of action (e.g., phosphorylation, ion conductance's), or function (e.g., phototaxis, vision).

Typically, two superfamilies are distinguished: (1) microbial opsins (type I), including opsins from prokaryotes, fungi, and algae and (2) animal opsins (type II), which are found in eumetazoans.

Although both opsin types are transmembrane proteins and may share a common origin, they differ significantly from each other. Microbial opsins are mainly light-activated ion pumps or channels, which directly transduce electromagnetic signals into electrical currents. On the other hand, all type II opsins belong to the family of G protein-coupled receptors (GPCRs), which initiates protein-protein interaction and subsequent intracellular signaling cascades.

Microbial opsins, of type I, utilize all-trans as a chromophor, which stays covalently bound to the opsins after photoisomerization, whereas type II opsins use cis to trans isomerization of retinal (retinaldehyde) to transmit light stimuli. All vertebrate tissues investigated so far already contain sufficient amounts of retinal to constitute the protein, so that no additional retinal has to be supplied.

After the establishment of channelrhodopsin 2 (ChR2), a blue light-gated cation-selective ion channel from green algae ChR2, as an excitatory optogenetic tool, the first inhibitory tool was described. NpHR, a chloride pump from Natronomonas pharaonis, was used to silence neurons in vitro and in vivo. NpHR has its excitation maximum around 600 nm. In addition to opsins that regulate chloride pumps, opsins that control outward proton pumps, i.e., bacteriorhodopsins, such as eBR, Arch, and Mac, have also demonstrated their ability to inhibit neuronal firing. These capabilities have raised the possibility that optogenetic therapies can treat degenerative diseases of the eyes, hearing loss, and spinal cord injuries, as well as play a role in deep brain stimulation therapies.

In general, light-gated actuators have been known to control neuronal activity. Specific light-sensitive elements in phototransduction machineries underlying animal vision were found to be membrane-embedded photopigments called rhodopsins, each rhodopsin molecule consisting of a protein called opsin (belonging to the family of G-protein-coupled receptors or GPCRs) covalently bound to a chromophore (a vitamin A-related compound called retinal or one of its derivatives). Upon illumination, the bound retinal molecule undergoes isomerization, which induces conformational changes in the opsin backbone and activates a G-protein signaling pathway. Indeed, the first light-actuated control systems were designed to modulate neuronal firing.

In this invention, the light from the internal light source materials noted above (e.g., phosphors, fluorescent agents, etc.) can be used to treat different types of diseases and disorders such as those described above. In one embodiment, the light from the internal light source materials noted above could be used to treat degenerative diseases of the eyes, hearing loss, and spinal cord injuries, as well as play a role in deep brain stimulation therapies. In one embodiment, the light from the internal light source materials noted above could be used to treat vascular disease including peripheral artery disease, aneurysms and Raynaud's disease (a condition causing people to feel numbness and cold in their fingers and toes due to the narrowing of the small arteries that supply blood to the skin) by emission of characteristic wavelengths of light which triggers the light receptors in blood vessels. Specifically, in one embodiment, the endothelial cells that line blood vessels can be exposed to blue light (380-495 nm) generated from the internal light sources noted above such that, upon patient exposure to x-rays, blue light emitted from the internal light source would affect blood flow.

In one embodiment of the invention, a phosphorescent or fluorescent or light emitting material such as those described above (e.g., x-ray induced persistent phosphors) would be encased with a biocompatible coating transparent to blue light and introduced into the blood stream or into the body of the blood vessel or nearby a blood vessel. Upon exposure to x-rays, the phosphorescent or fluorescent or light emitting material would emit blue light which would be absorbed in the walls of the blood vessel to affect a change in blood flow for example by way of triggering a response in melanopsin (opsin 4) in the blood vessel walls.

In other work, workers have sought to optically control Ca²⁺ signals. Ca²⁺ acts as a messenger to regulate a myriad of cellular activities, ranging from short-term reactions occur-ring within seconds (e.g., muscle contraction and neurotransmitter release) to long-term processes that last for hours or even days (e.g., gene transcription). The location, amplitude and frequency of Ca²⁺ signals in mammalian cells undergo constant changes to maintain Ca²⁺ homeostasis while meeting the diverse requirements of different Ca²⁺-modulated events. Activation of cell-surface receptors, such as G protein-coupled receptors (GPCRs) and receptor tyrosine kinases (RTKs), results in mobilization of Ca²⁺ release from internal Ca^{2+ stores. Upon ligand binding to these receptors, PLC is activated to hydrolyzethe PM-bound lipid, phosphatidylinositol} 4,5-bisphosphate (PIP2), generating two second messengers: inositol 1,4,5-trisphosphate(IP3) and diacylglycerol (DAG). DAG is an activator of protein kinase C (PKC) and may directly activate certain types of transient release potential (TRP) channels, resulting in Ca²⁺ influx from the extracellular space. Photo-switchable DAG and its analogs based on the azobenzene photo-switch have been developed to modulate PKC dependent pathways.

In one embodiment of the present invention, the examples given above are but illustrative of the present invention's capability for use in optogenetics. More specifically, the present invention provides the capability to provide light to opsins and other light-driven actuator proteins in order to impact a number of physiological parameters ranging from membrane voltage and calcium concentration to metabolism.

### Tunneling Nanotubes

Tunneling nanotubes TNTs have been found to exist between adjacent cells. Moreover, recent studies have found TNTs to be dynamic connections between cells, providing a route for cell-to-cell communication. TNTs are considered to play a role in intercellular exchanges of signals, molecules, organelles, and pathogens. TNTs can from in a number so cell types, including neuronal cells, epithelial cells, and almost all immune cells.

In myeloid cells (e.g., macrophages, dendritic cells, and osteoclasts), intercellular communication via TNT is believed to contribute to their differentiation and immune functions. TNTs are believed to be one way for myeloid cells to communicate with a targeted neighboring or distant cell, as well as with other cell types, therefore creating a complex variety of cellular exchanges. TNTs may also contribute to pathogen spread as they are believed to serve as “corridors” from a cell to another.

Vignas et al have described in “Cell Connections by Tunneling Nanotubes: Effects of Mitochondrial Trafficking on Target Cell Metabolism, Homeostasis, and Response to Therapy,” in Stem Cells Int. 2017; 2017: 6917941. (the entire contents of which are incorporated herein by reference) that TNTs can be a means of communication between cells devised to allow long-distance cell-to-cell contacts. This paper reported that the formation of tunneling nanotubes (TNTs) between these cells was initially reported in the rat pheochromocytoma- (PC12-) derived cells and in immune cells. These TNTS were long tubular structures, with diameters between 50 and 1500 nm, that could span several tens to hundreds of microns, connecting two cells together. In a characteristic manner, in 2D cultures, TNTs were not tethered to the extracellular matrix, rather floating in the culture medium. This paper reported that the tunneling nanotubes allowed a continuity in plasma membrane and cytoplasm between the connecting cells, thus allowing trafficking of a number of cellular components from one cell to the other.

Furthermore, this paper reported that cells of the immune system, notably macrophages, dendritic cells (DCs), NK, and B cells, extensively use TNTs to communicate. According to this paper, the transfer of antigenic information from migratory DCs to lymph node-residing DCs through TNTs has been shown to be critical for the induction of immune responses. TNT formation was also described in neural CAD cells (mouse cell line of catecholaminergic origin) and from bone marrow-derived dendritic cells to primary neurons.

Rustom in “The missing link: does tunneling nanotube-based supercellularity provide a new understanding of chronic and lifestyle diseases?,” from http://rsob.royalsocietypublishing.org/ on Sep. 3, 2018 (the entire contents of which are incorporated herein by reference) describes a number of ways for TNT formation. The paper notes that, in general, oxidative stress is defined as an imbalance between the production of free radicals and reactive metabolites, such as H₂O₂ or superoxide anions, and their elimination by the antioxidative cell defense system. The list of severe diseases that have been linked to oxidative stress is long, including neurodegenerative disorders, such as Alzheimer's and Parkinson's, chronic inflammation, diabetes and cancer. The paper notes that it is well accepted that most reactive oxygen species (ROS) are generated in cells by the mitochondrial respiratory chain.

The paper noted that to counter stress, “stressed cells” will distribute “‘call-for-help’ signals to determine the position of unstressed cells in their surrounding.” The paper describes that, while the nature of these signals is still under debate, candidate molecules are advanced glycation end products (AGEs).

In this paper, local stress leads to increasing ROS levels and AGE distribution from the stressed cell (a-1). AGE and receptor for AGE (RAGE) interaction at the target cells leads to cROS increase (a-2) and AC-TNT formation via actin-based, filopodia-like cell protrusions in order to restore redox/metabolic homeostasis by intercellular material exchange (a-3). Further increasing ROS levels lead to MT-TNT formation (b-1), allowing for efficient redox/metabolic rescue of stressed cells, e.g. via motor protein-mediated intercellular transfer of mitochondria along microtubules (b-2). Finally, exaggerated ROS levels induce apoptosis (c-1). Note that prior to apoptosis, remaining TNT connections are severed in order to isolate and remove ‘degenerated’ cells from the collective (c-2)—probably controlled by altered cholesterol/oxysterol homeostasis.

Accordingly, in one embodiment of the present invention, the biophotonic sources described above and/or the biophotoic bypasses could be used to stimulate formation of TNT growth. For example, the live biophotonic sources described above could be stressed (in a number of conventional ways) or selected portions of organs could be stressed as noted elsewhere. The stressed cells would then emit “call-for-help signals” (which regardless of their origin and nature would stimulate formation of TNTs. For example, the artificial biophotonic sources or the above-noted biophoton stimulator would emit light at a frequency and dose level which could stimulate formation of TNTs. For example, Wang et al in “Transfer of mitochondria via tunneling nanotubes rescues apoptotic PC12 cells,” in Cell Death Differ. 2015 July; 22(7): 1181-1191 (the entire contents of which are incorporated herein by reference) show that UV light induced TNT formation presumably through the stress induced on the cells by the UV light.

In one embodiment of the present invention, the network of TNTs induced by the cell communication would permit healthy cells to strengthen their interconnection with other healthy cells, thus providing resistance to infection from other diseased cells.

In one embodiment of the present invention, the network of TNTs induced would permit cancerous cells undergoing apoptosis to experience cell death at a higher rate, thus controlling tumor growth.

In one embodiment of the present invention, the network of TNTs induced would permit organs subject to inflammation to build there interconnection to other nearby cells, thus providing a mechanism for the inflammation to be reduced by permitting mesenchymal stem cells (MSCs) to be transferred. Such cells are known to contributes to tissue repair and immunosuppressive properties. Once at the inflammation site, MSCs prevent cellular destruction and damage to surrounding tissues. MSC immuno-suppression is mediated by the secretion of soluble factors like indoleamine 2,3-dioxygenase (IDO), IL-10, TSG-6 (TNF-α-stimulated gene/protein 6), prostaglandin E2 (PGE2), TGF-β-1, inducible nitric oxide synthase (iNOS) and human leukocyte antigen (HLA-G).

### Building Blocks of the Invention

The present invention takes advantage of several fundamental building blocks by which a physical, chemical, and/or therapeutic change can be implemented to a treatment area. Below is a non-limiting and non-exclusive discussion of these building blocks provided as guidance on implementing the procedures and tools described above.

One building block involves the phenomenon of cell-to-cell communication discussed above in which different cells in different regions “communicate” with each other even without necessarily being in physical or fluid contact with one another. As discussed above, there are a number of mechanisms in the literature for how the cell-to-cell communication can work. One mechanism discussed above and utilized in the present invention is by omission of a biophoton also known as mitogenic radiation. Other mechanisms discussed above and utilized in the present invention is by emission of electromagnetic radiation or sonic radiation. Another mechanism discussed above and utilized in the present invention is by coupling through coherent quantum states, where the change in the state at one location produces a concomitant change in the quantum state at another location. Another mechanism discussed above and utilized in the present invention is by coupling of excited states in a cellular bioplasma.

Closely related to the last two effects is that of quantum entanglement. Experiments with green fluorescent proteins in a biological living medium have shown the photons emitted from separated molecules to be related as “entangled pairs” with the photons' polarizations entangled such that, by determining the polarization of one emitter, the polarization of the quantum entangled other emitter was known a priori. Here, the cell-to-cell communication utilized in the present invention to induce effects in neighboring cells may well be a quantum entanglement phenomenon.

Another building block involves the capability to affect the quantum or physical states of the biological structures of a cell. For example, as discussed above, cellular processes associated with membranes in a cell are controlled by factors such as pore size, the thickness of the membrane, and the polarity of the membrane. These pore sizes and thicknesses are on the nanometer scales, and therefore are susceptible to being influenced by applied radiation, by applied electromagnetic fields, and/or by applied localized electric fields which the physics of diffusion and transport even at the quantum scale can influence the transport of materials through the membranes or the attachment of antibodies to the cell membrane.

Another building block used by the present invention is the realization that photosynthesis-type reactions (occurring in the realm of plants) are also a mechanism at play inside living cells of animals. Here, light can induce not only the generation of biophotons as discussed above but also can promote reactions in the cells such as increased metabolism of a cell, cell division, or cell death

Another building block used by the present invention is the realization that there are many pathways before communication between cells including those of physically connected pathways such as the tunneling nanotubes (TNTs) discussed above. These pathways can be used for both productive and detrimental uses. In the present invention, mechanisms to shut down selected pathways can be used to control/restrict the spread of viruses, bacteria, or cancer from one region of the body to another. In the present invention, mechanisms to promote certain pathways can be used to promote cell regeneration, for example, in the regrowth of healthy heart tissue inside a diseased heart.

Yet another building block used by the present invention is the realization of the impact of outside stimulus, such as a biophoton, on the epigenome. For example, it has been shown that identical twins having identical DNA at birth can have their DNA changed by environmental factors. Here, in vivo light or light delivered in situ such as for example biophotons can be used to interact directly with the DNA encoded in the cells to implement a therapeutic change.

### Quantized Biology:

In one embodiment of the invention, and in other embodiments described below, photonic energy can participate and control the various metabolic processes in an individual cell or a group of cells or may act as a sensor where an untreated control cell emits the photonic energy in response to an event in a distal cell. Control of the metabolic processes in one region (a control region) may be coupled to another region (e.g., a treatment site inside the patient, where the coupling can induce a biological, chemical, physical, or therapeutic change in the subject at the other region or the treatment site). Alternatively, in one embodiment of the invention, the photonic energy (as described for example in the following) can directly cause a biological, chemical, physical, or therapeutic change at a treatment site.

In one embodiment of the invention, hvᵢ is a photonic energy that is ionizing and can therefore be responsible for catalyzing a chemical reaction. Other energies (hvⱼ) generated through energy converters (such as UV or other energy generated from the phosphors described herein) could create a free radical hence inducing a charge-build up in a protein of low molecular weight or on a side group of a long molecular weight protein. Once the ionization of a protein takes place, this ionization could result in a dysfunctional behavior of the protein, and subsequently the failure of the protein to achieve the intended process. For example, in one embodiment of the invention, hvᵢ or hvⱼ induced ionization of an epidermal growth factor receptor (EGFR) protein can denature or render the EGFR protein dysfunctional. EGFR is considered a transmembrane protein that is a receptor for members of the epidermal growth factor family (EGF family) of extracellular protein ligands. Deficient signaling of the EGFR and other receptor tyrosine kinases in humans is associated with diseases such as Alzheimer's, while over-expression is associated with the development of a wide variety of tumors. Interruption of EGFR signaling, either by blocking EGFR binding sites on the extracellular domain of the receptor or by inhibiting intracellular tyrosine kinase activity, may prevent the growth of EGFR-expressing tumors and might improve a patient's condition.

In one embodiment of the invention, hvₖ is a photonic energy responsible for signaling an aspect of a protein conformation. This photonic energy hvₖ would typically not be ionizing. In another embodiment of the invention, hv_{z} is a photonic energy responsible for signaling an aspect of a protein conformation that closes an ion channel or multiple ion channels. In a further embodiment of the invention, hvₓ is a photonic energy responsible for signaling an aspect of a protein conformation that opens an ion channel or multiple ion channels.

Hence, in one aspect of this invention, photonic energy can be used to promote reactions in some cases (hvᵢ), promote ionization and denaturing of certain proteins (hvⱼ), change protein conformation (hvₖ), and/or signal the closure and the opening of ion channels (hv_{z}, hvₓ). Energy converters in one embodiment (such as the phosphors described elsewhere) can be used to convert high energy incident radiation such as x-ray into one or more of hvᵢ, hvⱼ, hvₖ, hv_{z}, and/or hvₓ which can interact within the cell environment to promote or prohibit the functions of those cells. The use of such high energy incident radiation (such as x-ray), which can penetrate completely through the subject body, permits the implementation of the invention deep within the body in a non-invasive manner, requiring at most only injection of the desired energy converters to the desired site.

The following examples performed using poly(deoxyadenylic-deoxythymidylic) acid sodium salt (poly-dAdT) and 8-methoxypsoralen (8-MOP) demonstrates this aspect of the invention and shows the effects of photonic energy to promote biologically driven reactions via quantized effects.

### Monoadduct (MA) Formation and Di-Adduct Formation or Cross-Linking (XL)

In the examples below, the energy promoters (i.e., the phosphors designated below as BP3, BP10, and BP6) absorb X-Ray energy and emit photonic energy from the UVA to the visible range as listed below (name, emission peak):

| | (BP3, 327 nm) | (BP10, 355 nm) | (BP6, 410 nm) |
|---|---|---|---|
| Properties | BP3 | BP10 | BP6 |
| Peak Emission (nm) | 327 | 390 | 410 |
| hν = Photonic | 6.075E−19 | 5.093E−19 | 4.845E−19 |
| Energy (J) | | | |
| Intensity (AU) | 15,000 | 5,000 | 22,000 |
| Composition | YTaO₄ | BaSO₄₋:Eu²⁺ | YTaO₄:Nb |

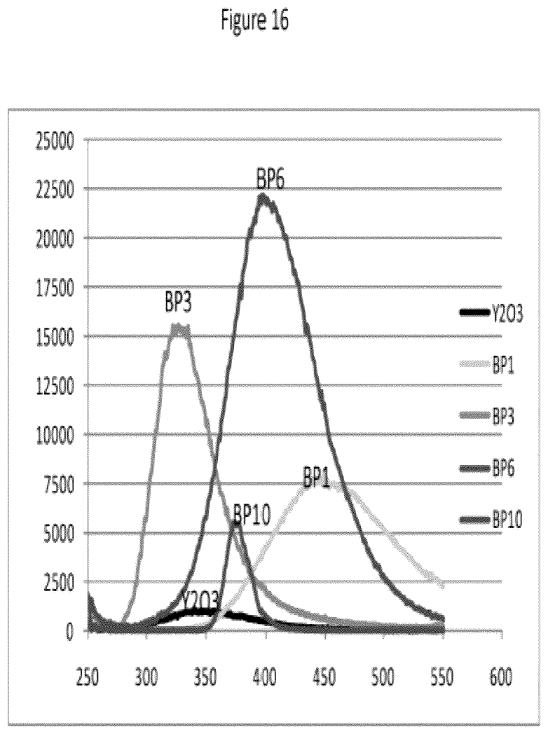

FIG. 16 shows the spectral emission of the BP3, BP10, and BP6 phosphors Phosphors BP3, BP6, and BP10 were added to a solution of 8-MOP and Poly-dAdT, and exposed to various X-ray conditions. The plates were placed at the following distances from the X-ray source: 100 mm and 200 mm. This had the effect of changing the dose rate. The X-Ray parameters included 320 kV and 10 mA for a fixed time period. This example shows that monoadduct (MA) formation can be promoted for example by UV light with the higher energy light promoting more MA formation even at a lower flux or intensity.

### Photonic Energy Comparison:

$${hv}_{( BP 3 )} > {hv}_{( BP 10 )} > {hv}_{( BP 6 )}$$

### Intensity Comparison:

$${I}_{( BP 6 )} > {I}_{( BP 3 )} > {I}_{( BP 10 )}$$

Comparison of the phosphors in terms of Mono-Adduct (MA) formation as demonstrated in detail below showed that:

$${MA}_{( BP 3 )} > {MA}_{( BP 10 )} > {MA}_{( BP 6 )}$$

Moreover, the observed MA formation tends to follow the photonic energy ranking rather than the intensity of the energy conversion from X-Ray to UV or visible light.

Similarly, other sets of experiments were performed further demonstrating the effect of photonic energy on both MA and di-Adduct formation (e.g. cross linking (XL)). Measurements of the MA and di-adduct formation (XL) were performed using high performance liquid chromatography (HPLC) to identify the presence of these compounds after exposure to photonic energy.

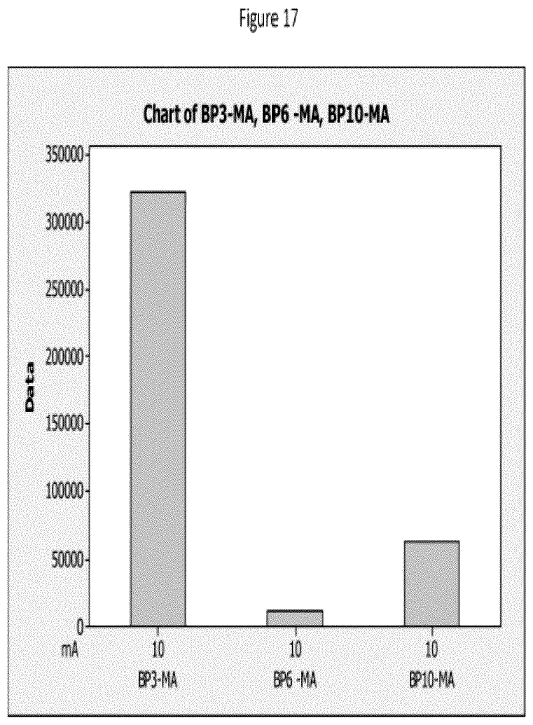

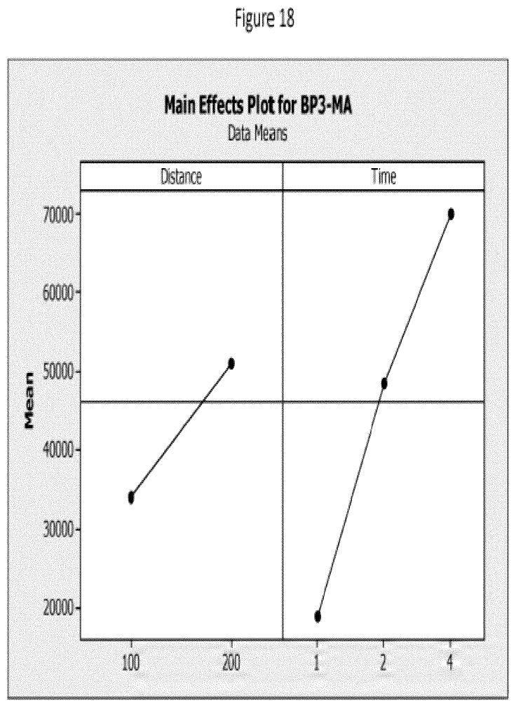

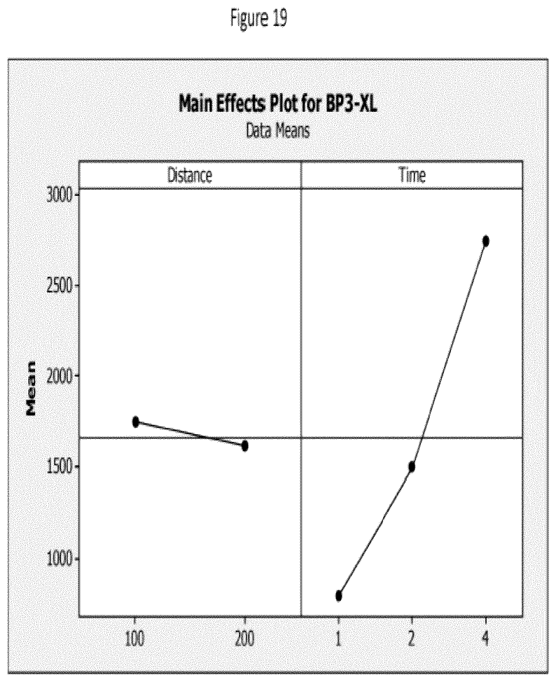

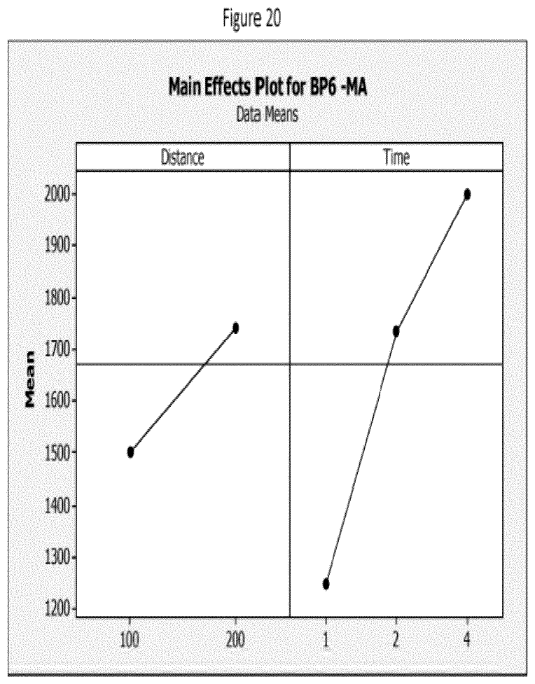

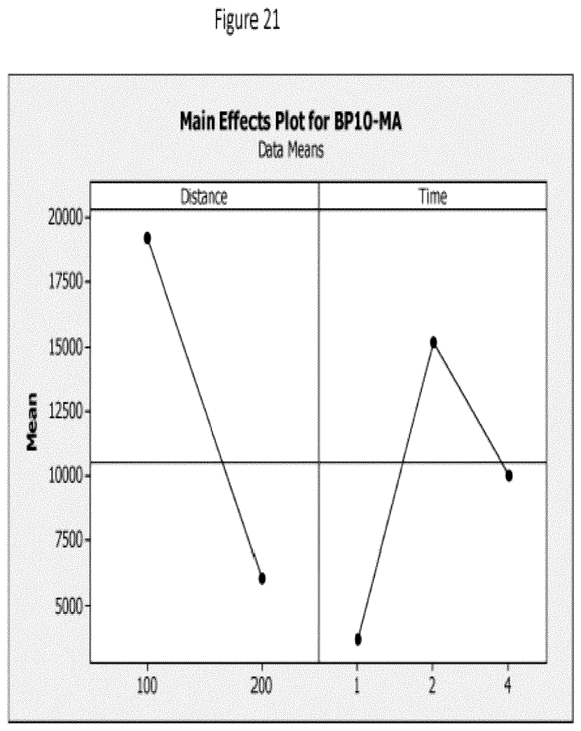

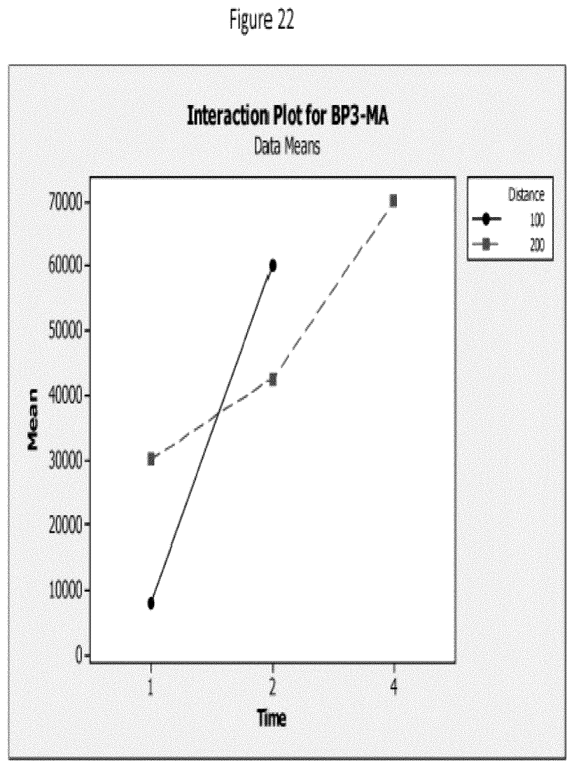

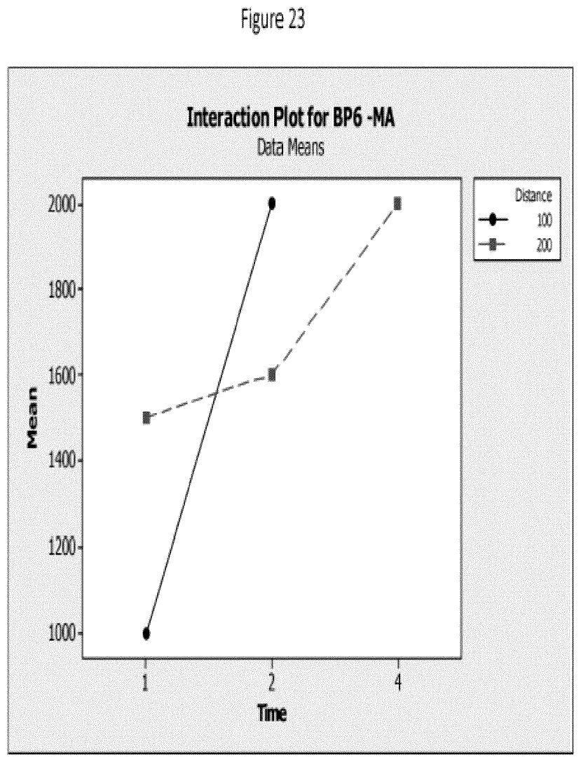

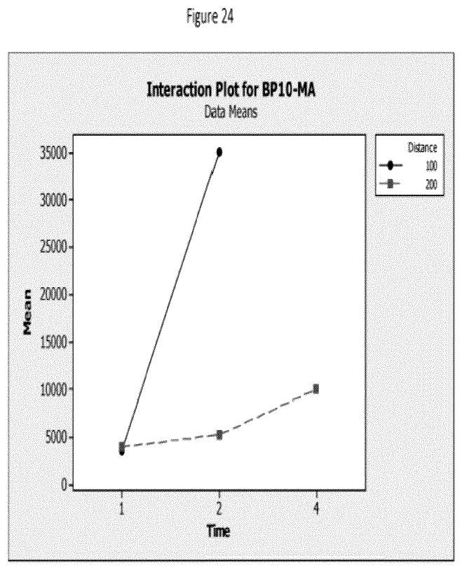

FIG. 17 is a chart showing that photonic energy from BP3 tends to produce more MA than BP6 or BP10. FIG. 18 is a chart showing MA formation under BP3 photonic energy as a function of distance from the X-ray source and time. Somewhat surprisingly, MA formation increases as the distance from the X-ray source increases. This points out that the right reaction is sensitive to dose rate. Lower dose rates in this case could be more beneficial. Regardless, the results show MA formation under BP3 photonic energy. FIG. 19 is a chart showing XL under BP3 photonic energy as a function of distance from the X-ray source and time. Here, XL decreases as the distance from the X-ray source increases and increases with time. It is worth noting that the XL reaction is reversible. FIGS. 20-24 show results from other experiments corroborating MA formation and/or XL under photonic energy exposure.

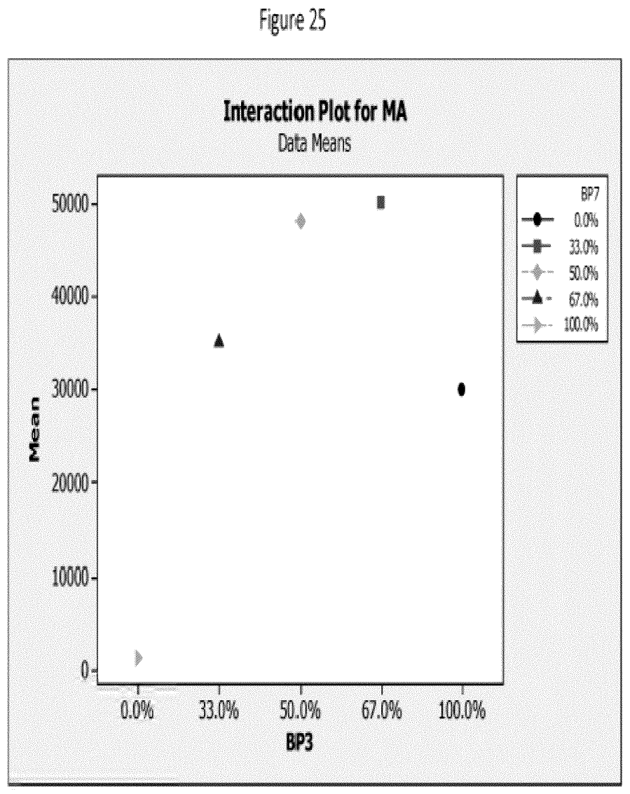

FIG. 25 is a chart showing a non-linear effect on MA seen by mixing two phosphors. Indeed, the mixtures of BP7 from at least 33% to 67% show higher MA formation than observed when using only BP3 or only BP7. The tables below (tables 5, 6, and 7) summarize the results:

### Tables 5, 6, and 7

BP6

| | kvp | mA | Distance mm | Time (min) | MA1 | XL |
|---|---|---|---|---|---|---|
| S2 | 220 | 10 | 200 | 1 | 1,500 | |
| S6 | 320 | 10 | 200 | 2 | 1,700 | |
| S10 | 320 | 10 | 200 | 2 | 1,500 | |
| S14 | 320 | 10 | 200 | 4 | 2,000 | |
| S18 | 320 | 10 | 200 | 4 | 2,000 | |
| S22 | 320 | 10 | 100 | 2 | 2,000 | |
| S26 | 320 | 10 | 100 | 1 | 1,000 | |

BP8

| | kvp | mA | Distance mm | Time (min) | MA1 | XL |
|---|---|---|---|---|---|---|
| S2 | 220 | 10 | 200 | 2 | 30,000 | 600 |
| S5 | 320 | 10 | 200 | 2 | 40,000 | 1,000 |
| S9 | 320 | 10 | 200 | 2 | 45,000 | 2,000 |
| S13 | 320 | 10 | 200 | 4 | 70,000 | 2,500 |
| S17 | 320 | 10 | 200 | 4 | 70,000 | 3,000 |
| S21 | 320 | 10 | 100 | 2 | 60,000 | 2,500 |
| S25 | 320 | 10 | 100 | 1 | 8,000 | 1,000 |

BP10

| | kvp | mA | Distance mm | Time (min) | MA1 | XL |
|---|---|---|---|---|---|---|
| S2 | 220 | 10 | 200 | 1 | 4,000 | |
| S8 | 320 | 10 | 200 | 2 | 4,000 | |
| S16 | 320 | 10 | 200 | 4 | 6,500 | |
| S20 | 320 | 10 | 200 | 4 | 10,000 | |
| S24 | 320 | 10 | 100 | 2 | 35,000 | |
| S28 | 320 | 10 | 100 | 1 | 3,500 | |

Similarly, experiments have shown that phosphor combinations can promote higher XL than the individual phosphors alone. See Table 9 below.

TABLE 9

| BP3 (g) | BP7 (g) | BP3 | BP7 | MA | XL |
|---|---|---|---|---|---|
| 0.3 | 0 | 100.0% | 0.0% | 3.00E+04 | 5.00E+02 |
| 0.2 | 0.1 | 67.0% | 33.0% | 5.00E+04 | 6.00E+02 |
| 0.15 | 0.15 | 50.0% | 50 | 4.80E+04 | 5.00E+02 |
| 0.1 | 0.2 | 33.0% | 67.0% | 3.50E+04 | 4.50E+02 |
| 0 | 0.3 | 0.0% | 100.0% | 1.40E+03 | 0.00E+00 |

### Activation and Deactivation of a Signaling Protein

Living cells possess sophisticated molecular machinery and control systems. Living cells convert food into energy, such as ATP, which drives the millions of biochemical processes necessary for keeping us alive. The pathways used to convert substrates such as glucose into products are collectively referred to as metabolic pathways. The drivers of these metabolic pathways are enzymes that work to assist chemical reactions by building or breaking down molecules. The enzymatic protein does not drive reactions at a constant rate. The reaction rate can in fact speed up or slow down or even stop completely according to the cell's needs. The cell is considered to be self-regulated, and the supply of products does not exceed demand. If products are being created at a rate that is faster than they can be used, a slower rate or complete stoppage can take place through a process called feedback inhibition, which is part of Allosteric regulation. Allosteric regulation plays a role in many metabolic pathways and is considered to keep everything running smoothly and efficiently while maintaining homeostasis.

Allosteric Regulation: There are enzymatic and non-enzymatic proteins. Enzymes catalyze reactions, for example, such as in the case of DNA polymerase and Amylase. Non-enzymatic proteins play a large number of functions and roles, including, but not limited to, receptors/ion channels, transport, motor and antibodies.

Enzymes have active sites where substrates combine as well as allosteric sites where enzyme regulator can bind. There are two types of regulators: allosteric activators which increase enzymatic activity and allosteric inhibitors which decrease enzymatic activity. A feedback loop gets established whereby the downstream products regulate upstream reactions. An increase or decrease of enzymatic activity is therefore tailored to the specific needs of the cell.

Mutated enzymes that do not respond to allosteric regulation have been linked to disease states, such as cancer. Many processes in our bodies rely on molecular feedback inhibition to maintain homeostasis.

In the present invention, photonic energy can be used to promote allosteric activators which increase enzymatic activity and/or promote allosteric inhibitors which decrease enzymatic activity, therefore targeting diseased cells to curtail runaway growth conditions

In one embodiment, as noted above, a signaling protein can be activated and deactivated using photonic energy. In this embodiment, an energy converter such as BP3, BP6, and/or BP10 would be located nearby or inside a cell to generate photonic energy, such as UV or visible light, to promote the function or the suppression of a signaling protein.

Light-activated DNA binding in a designed allosteric protein has been reported by Devin Strickland, Keith Moffat, and Tobin R. Sosnick, Department of Biochemistry and Molecular Biology and Institute for Biophysical Dynamics, University of Chicago, 929 East 57th Street, Chicago, IL 60637, edited by David Baker, University of Washington, Seattle, WA, and approved May 12, 2008 (received for review Oct. 9, 2007) in “Light-activated DNA binding in a designed allosteric protein,” the entire contents of which are incorporated herein by reference.

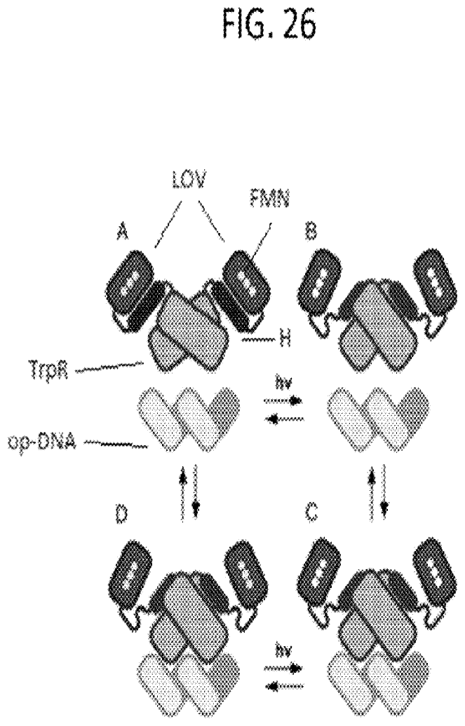

An understanding of how allostery, the conformational coupling of distant functional sites, arises in highly evolvable systems is of considerable interest in areas ranging from cell biology to protein design and signaling networks. The rigidity and defined geometry of α-helical domain linker was reasoned to make it effective as a conduit for allosteric signals. The idea was tested by designing twelve fusions between the naturally photoactive light-oxygen-voltage-sensing domain LOV2 domain from Avena Sativa phototropin 1 and the Escherichia coli trp repressor were investigated by Strickland et al. When illuminated with photonic energy, one of the fusions selectively binds to operator DNA and protects it from nuclease digestion. The helical “allosteric lever arm” was considered by Strickland et al. to be a mechanism for coupling the function of two proteins. This is illustrated in FIG. 26.

In FIG. 26, the LOV domain (containing the three dots representing the three ring FMN chromophore), the TrpR domain in orange, and the operator DNA are depicted above in various states. The shared helix, H, is shown contacting the LOV domain in (A) and contacting the TrpR domain in (B)-(D). The three-ring FMN chromophore is in the ground state in (A) and (D) above and when photoexcited in (B) and (C). (A) In the dark DNA-dissociated state, the shared helix H contacts the LOV domain, populating an inactive conformation of the TrpR domain. (B) Photoexcitation disrupts contacts between the shared helix H and the LOV domain, populating an active conformation of the TrpR domain. (C) LovTAP binds DNA. (D) The LOV domains return to the dark state. LovTAP dissociates from the DNA, contacts between the shared helix H and the LOV domain are restored, and the system returns to the initial state.

Strickland et al. concluded that a successful design of an allosteric lever arm and a bistable energy surface, along with the observation of a natural analogue, suggesting the existence of a general but largely unrecognized mode of connecting modular domains into a functionally integrated whole. The α-helical structure of the linker distinguishes this mode from others in which allostery results from intramolecular binding between domains connected by linkers of undefined structure. Because a regular helix resists bending and twisting, it can function as an allosteric lever arm to transmit forces created by interdomain contacts to generate bistable systems.

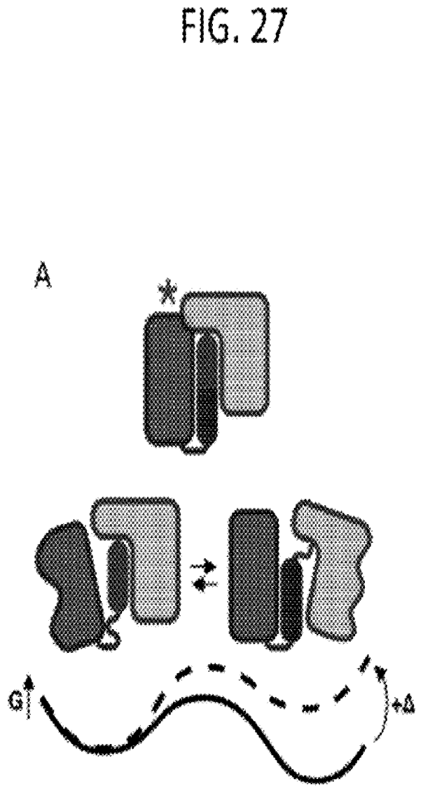

FIG. 27 provides a depiction of a design of an allosteric, light activated repressor. As shown in FIG. 27, (A) represents a conceptual model of an allosteric lever arm. Joining two domains across terminal α-helices creates a bi-stable system in which steric overlap (star) is relieved by the disruption of contacts between the shared helix and one or the other of the domains. A perturbation (Δ) such as ligand binding or photo-excitation alters the energy surface of the system (blackline) to favor a new conformational ensemble (dashed line) with different functional properties.

Here, in the present invention, photonic energy, for example from the energy converters noted above, can be used to photoexcite these types of reactions to promote light-activated DNA binding.

Furthermore, in the present invention, photonic energy can be used to activate a repressor. The following references (all of which are incorporated herein in their entirety by reference) describe repressors:
Freeman, S. Hamilton, H., Hoot, S., Podgorski, G., Ryan, J. M., Smtill, S. S., & Weigle, D. S (2002). Bilogical Science (Vol 1.). Upper Saddle River, NJ: Prentice Hall.
Gerhart, J. C., & Pardee, A. B. (1962). The enzymology of control by feedback inhibition. J Biol Chem, 237, 391-896.
Tansey, J. T., Baird, T., Cox, M. M., Fox, K. M., Knight, J., Sears, D., Bell, E. (2013). Foundational concepts and underlying theories for majors in “biochemistry and molecular biology”. Biochemisty and molecular biology education, 41 (5), 289-296.
Webb, B. A., Forouhar, F., Szu, F. E., Seetharaman, J., Tong, L., Barber, D. L. (2015). Structures of huma phosphofructokinase-1 and atomic basis of cancer associated mutations. Nature 523 (7558). 111-114.
Oana I Lungu, Ryan A Hallett, Eun Jung Choi, Mary J. Aiken, Klaus M Hahn, and Brian Kuhlman, Department of Biochemistry and Biophysics, Department of Pharmacology, University of North Carolina Chapel Hill, NC 27599, USA, published an article in Chemistry & Biology 19, 507-517, Apr. 20, 2012 entitled “Designing Photoswitchable Peptides using the AsLOV2 Domain,”
   the entire contents of each of which are incorporated herein by reference.

Lungu et al. describes that peptides can regulate a variety of biological processes by acting as competitive inhibitors, allosteric regulators and localization signals. Photo-control of peptide activity represents a tool for precise spatial and temporal control of cellular functions.

Lungu et al. showed that genetically encoded light-oxygen-voltage-sensing domain LOV2 domain of Avena Sativa phototropin 1 (AsLOV2) can be used to reversibly photo-modulate the affinity of peptides for their binding partners. Sequence analysis and molecular modeling were used to embed tow peptides into the Ja helix of the AsLOV2 domain while maintaining AsLOV2 structure in the dark but allowing for binding to effector proteins when the Ja helix unfolds in the light. Caged versions of the ipaA and SsrA peptides, LOV-ipaA and LOV-SsrA, bind their targets with 49- and 8-fold enhanced affinity in the light, respectively. These switched can be used as general tools for light-dependent colocalization, which Lungu et al. demonstrated with photo-activable gene transcription in yeast.

In another reference entitled A light-triggered protein secretion system, the entire contents of which are incorporated herein by reference, by Daniel Chen, Emily S. Gibson, and Matthew J. Kennedy, Department of Pharmacology, University of Colorado Denver School of Medicine, Aurora, CO 80045, Chen et al. confirmed the importance of light in various cellular functions.

Chen et al. used UVR8, a plant photoreceptor protein that forms photolabile homodimers, to engineer the first light-triggered protein secretion system. UVR8 fusion proteins were conditionally sequestered in the endoplasmic reticulum, and a brief pulse of light triggered robust forward trafficking through the secretory pathway to the plasma membrane. UVR8 was not responsive to excitation light used to image cyan, green, or red fluorescent protein variants, allowing multicolor visualization of cellular markers and secreted protein cargo as it traverses the cellular secretory pathway. Chen et al. showed that this could be used, as a tool in neurons, to demonstrate restricted, local trafficking of secretory cargo near dendritic branch points.

In general, the use of light to control basic cellular functions has transformed experimental biology. Some of the first approaches relied on photolabile small molecule analogues of second messengers, second messenger chelators, or neurotransmitters to control cellular physiology and signaling pathways with ultraviolet (UV) light. These “caged” compounds have been used for dissecting numerous molecular pathways governing cellular physiology with unprecedented spatial and temporal control. More recently, exogenously expressed photoreceptors from plants have been used to control cellular biochemistry by conditionally gating protein-protein interactions with light. This approach has emerged as a new and powerful way to control cellular processes on fast timescales with fine spatial precision without the need for small molecules. Some of the first studies describing engineered optical control of cellular functions used the plant photoreceptor phytochromeB. PhyB binds to members of the phytochrome-interacting family (PIF) of basic helix-loop-helix transcription factors when photoexcited with red (660 nm) light. Remarkably, PhyB/PIF interactions can be reversed by near-infrared (730 nm) excitation, allowing fast and local toggling of PIF binding.

However, PhyB-based systems require addition of an exogenous phycocyanobilin chromophore that is not normally present in yeast, flies, worms, or mammals, making it more difficult to implement than more recently developed systems that are entirely genetically encoded. These systems rely on blue light photoreceptor cryptochrome2 (Cry2), which binds to cryptochrome interacting basic-helix-loop-helix 1 (CIB1) in response to blue light, and the light, oxygen, voltage (LOV) domain photoreceptors, which undergo a large conformational change when photoexcited UVR8 has many unique properties that including constitutive formation of photolabile homodimers, slow reversal kinetics, and a UV-B absorption profile, which enables multicolor imaging of widely used fluorescent proteins without activating the photoreceptor. UVR8 can be used to conditionally sequester secretory cargo in the ER. Moreover, light triggers robust forward trafficking to the plasma membrane.

FIG. 28 shows the light-triggered dissociation of UVR8-tagged proteins. UVR8 fused to prenylated GFP (UVR8-memGFP) localizes to the plasma membrane where it recruits UVR8-mCh. Dissociation of the UVR8 dimer by UV-B light releases UVR8-mCh to the cytosol.

### Mitochondrial Metabolic Theory of Cancer and Cellular ATP Production

One theory on the origins of cancer, the Somatic Mutation Theory (SMT), holds that cancer is a complex genetic disease that arises from inherited or random somatic mutations in proto-oncogenes or in tumor suppressor genes. While many mutations have been found in various tumors, the so-called driver gene mutations are considered most responsible for causing the disease. Although the SMT is held out as the dominant scientific explanation for the origin of cancer, there are inconsistencies in the facts that seriously challenge the credibility of this theory. When viewed collectively, these inconsistencies imply that the nuclear somatic mutations found in many cancers cannot be the primary cause of the disease.

In a recent paper (Seyfried, T. N.; Chinopoulos, C. Can the Mitochondrial Metabolic Theory Explain Better the Origin and Management of Cancer than Can the Somatic Mutation Theory? Metabolites 2021, 11, 572. https://doi.org/10.3390/metabo11090572), Seyfried et al set forth a Mitochondrial Metabolic Theory (MMT) of cancer. According to Seyfried et al, cancer arises from a gradual disruption of ATP synthesis through oxidative phosphorylation (OxPhos) leading to compensatory ATP synthesis through substrate level phosphorylation. Accordingly, it is defective OxPhos that ultimately causes most of the genomic changes in cancer, not the reverse.

While aerobic fermentation (Warburg effect) is considered another emerging hallmark of cancer, the replacement of abnormal mitochondria with normal mitochondria will also reverse this hallmark. In other words, OxPhos sufficiency will reverse the Warburg effect (see Fu, A. et al., “Healthy mitochondria inhibit the metastatic melanoma in lungs” Int. J. Biol. Sci. 2019, 15, 2707-2718, and Hall, A. et al., “Dysfunctional oxidative phosphorylation makes malignant melanoma cells addicted to glycolysis driven by the V600EBRAF Oncogene” Oncotarget 2013, 4, 584-599). Thus, the energy transition from respiration to fermentation can explain the major hallmarks of cancer.

This is not intended to assert that genetic factors have no effect on cancer or its causation. In fact, there are genetic conditions (such as Lynch Syndrome, familial adenomatous polyposis (FAP), BRCA1 and BRCA2 gene mutations, etc.) that are known to increase the likelihood of a person getting cancers of various types. However, these genetic conditions are not the same as the genetic mutations set forth in the SMT, which as noted above are inherited or random somatic mutations in proto-oncogenes or in tumor suppressor genes. The MMT does not deny the existence of these genetic conditions that predispose certain individuals to increased likelihood of cancer, not is it inconsistent with such genetic conditions, since the MMT would hold that those predispositions caused by the genetic conditions likely increase the likelihood that the cells of the person affected go into fermentative ATP synthesis instead of the healthy Ox-Phos ATP synthesis.

The glutaminolysis pathway can produce high-energy phosphates through the sequential metabolism of glutamine->glutamate->alpha-ketoglutarate->succinyl CoA->succinate. Glutamine is the only amino acid that can generate significant ATP synthesis through mSLP in the glutaminolysis pathway [182]. With the exception of glutamate and glutamine, the catabolism of most other amino acids would expend high-energy phosphates during metabolic inter-conversions before becoming succinyl-CoA and cannot therefore effectively replace glutamine for ATP synthesis.

Seyfried et al describe glutamine-driven ATP synthesis in cancer cells as the Q effect (with Q being the symbol given to glutamine) to distinguish it from that involving the aerobic fermentation of glucose, i.e., the Warburg effect. Both the Warburg effect and the Q effect are downstream effects of compromised OxPhos function.

If a capability is necessary for tumor growth, then the inhibition of this capability should be essential for an effective management of cancer (see Seyfried, T. N. et al. “Press-pulse: A novel therapeutic strategy for the metabolic management of cancer” Nutr. Metab. 2017, 14, 19, and Hanahan, D. et al. “Hallmarks of cancer: The next generation” Cell 2011, 144, 646-674). A necessary capability for cancer cells, based on the MMT, is the fermentation metabolism needed for the synthesis of growth metabolites and ATP through the glycolytic and glutaminolysis pathways (see Seyfried, T. N. et al. “On the Origin of ATP Synthesis in Cancer” iScience 2020, 23, 101761). Accordingly, Seyfried et al view cancer as a relatively simple disease dependent almost exclusively on the availability of glucose and glutamine for survival. In short, no cancer cell can survive for very long without growth metabolites and ATP.

Seyfried et al propose that the simultaneous restriction of glucose and glutamine for the metabolic management of cancer can be best achieved using a “press-pulse” therapeutic strategy (Seyfried, T. N. et al. “Press-pulse: A novel therapeutic strategy for the metabolic management of cancer” Nutr. Metab. 2017, 14, 19). In adapting this concept to cancer management, “press” disturbances would eliminate the weakest cancer cells, while growth-restricting the heartiest cancer cells. In contrast, a “pulse” disturbance is an acute treatment that would kill most, but not all cancer cells. It is only when both the press and the pulse disturbances are used simultaneously that mass extinction of all cancer cells becomes possible. As one example of such a strategy, Seyfried et al mention ketogenic metabolic therapy (KMT) as a press and administration of the pan glutaminase inhibitor DON (6-diazo-5-oxo-L-norleucine) as a pulse. KMT restricts glucose availability while elevating ketone bodies and thus induces a competition between normal cells and tumor cells for glucose (Seyfried, T. N. “Metabolic management of cancer” Chapter 17. “In Cancer as a Metabolic Disease: On the Origin, Management, and Prevention of Cancer” John Wiley & Sons: Hoboken, NJ, USA, 2012; pp. 291-354). Ketone bodies and fatty acids are non-fermentable and cannot replace glucose in cells with defective mitochondria. Glucose restriction will inhibit tumor cell growth, as the glucose carbons are needed for the synthesis of growth metabolites through the pentose phosphate and glycolysis pathways. DON restricts availability of the glutamine nitrogen and the glutamine carbons that are necessary for the synthesis of nitrogen-containing metabolites and the synthesis ATP through mSLP in the glutaminolysis pathway. Hence, the simultaneous restriction of glucose and glutamine, while under KMT, will reduce acidification in the tumor microenvironment and will target both the glycolysis and glutaminolysis pathways that are essential for tumor cell growth and survival.

It is believed that this press-pulse therapeutic strategy can be used and taken a step further by combining such a KMT-DON or similar type press-pulse regimen with Immunolight therapy (also called X-PACT treatments) (as detailed, for example, in U.S. Pat. Nos. 9,352,040; 9,358,292; 9,682,146; 9,682,250; 10,300,299; 10,398,777; and 10,441,810, the contents of which are hereby incorporated by reference in their entireties) to result in a press-pulse² regimen, using the ketogenic diet to put the patient into a state of ketosis (the “press”), administration of a glutaminase inhibitor (pulse 1), then administration of a photoactivatable pharmaceutical agent (such as 8-methoxypsoralen or 8-MOP) and an energy modulation agent, followed by applying an energy to be converted by the energy modulation agent into an emitted energy to activate the photoactivatable pharmaceutical agent, which then attacks the cancer cells, sending them into apoptosis (pulse 2). Optionally, as detailed, for example, in U.S. Pat. No. 11,207,409 (the contents of which are hereby incorporated by reference in its entirety), this regimen could have a fourth step comprising either a booster treatment similar to the pulse 2 step, or palliative type radiation to highly activate the immune system and further destroy cancer cells within the body (a pulse 3 as it were).

The methods described in various embodiments of the present invention can be used to detect the Ox-Phos and fermentative processes and assist in treatments to return the patient's cells back to the Ox-Phos process. For monitoring the emissions associated with fermentative and Ox-Phos processes, the cuvette structures described herein can be used. Such cuvette structures could permit monitoring of emissions from both healthy cells and cancer cells simultaneously in separate chambers of the cuvette structures.

In particular, embodiments of the present invention provide a closed loop feedback process for returning the mitochondrial energy production in a patient from a fermentation based process to Ox-Phos based process, comprising:
monitoring mitochondria in cancer cells for light emissions and/or chemical signals (if any) during the fermentation process (these are the signals indicative of an aberrant energy production process);
monitoring mitochondria in healthy cells for light emissions and/or chemical signals (if any) during the Ox-Phos process (these are the signals indicative of a healthy energy production process); in order to distinguish the cell-to-cell communication signals emitted by cancer cells and healthy cells;
imparting an external light/radiation at the cellular level either directly using a fiber optic or indirectly using an energy modulation agent having an emission to trigger return of the cancer cells to an Ox-Phos process (the energy modulation agent can be a downconverter or an upconverter) and monitoring the mitochondrial energy production signals at different time points; and
changing the frequency, wavelength, or both of the imparted energy until the signals produced during the mitochondrial energy production are representative of a healthy Ox-Phos energy production process.

This process can be combined with the above described X-PACT treatment process, as well as a process by which the patient is first treated by fostering the dietary conditions necessary to place the patient into ketosis and/or administration of a glutaminase inhibitor. This process can also be used to personalize the treatment by having the cancer cells in the first monitoring step be from a biopsy of the actual cancer cells of the patient, and the healthy cells in the second monitoring step be from a biopsy of the health cells of the patient. This will permit fine tuning of the energy signals applied to change the ATP production from a fermentative process to an Ox-Phos process, and account for any differences that may occur with each patient's individual biological processes.

Here, in the present invention, photonic energy from for example the energy converters noted above can be used to photoexcite these types of reactions.

In stacking up amino-acids in the right sequence, the various units constituting a given protein enter into an energetically favorable and stable configuration. The presence of water is reported to enable the correct folding through the influence of the various water molecules in the vicinity of the amino-acids to be stacked. The proper staking and folding yields biologically compatible and functional molecules. If a solvent other than water is used the folding of proteins is derailed to biologically incompatible molecules. The energetically favorable stacking and folding is accompanied by remitting of any excess energy to the microenvironment hosting the staking process (the cell in this case). The excess energy release can be in various forms including electromagnetic radiation. This could in fact explain the presence of some of the low intensity photons in the cell environment. These photons are conceivably specific to the amino-acid being staked and the configuration to which they are folded. Hence the synthesis of the various proteins could be accompanied by the emission of electromagnetic radiation. In turn this electromagnetic radiation could play a role in guiding the conformational change of other existing (already made) protein.

### In-Vivo Photosynthesis (Human Photosynthesis):

It is well understood that cells, their proteins and genes are sensitive to light. A review of this area has been provided by Neves-Petersen, M. T., et al. (2012). “UV Light Effects on Proteins: From Photochemistry to Nanomedicine”, Molecular Photochemistry—Various Aspects, Dr. Satyen Saha (Ed.), ISBN: 978-953-51-0446-9, InTech, the entire contents of which are incorporated herein by reference.

Just a couple of examples of photoinitiated processes in human cells include (1) the vision process, which is initiated when photoreceptor cells are activated by light (photo-isomerization); and (2) near UV (290 nm) exposed prion protein fails to form amyloid fibrils (Thakur, A. K. & Mohan Rao Ch. (2008). “UV-Light Exposed Prion Protein Fails to Form Amyloid Fibrils, Plos one, Vol 3, No. 7, (July 2008), pp. E2688, eISSN 1932-6203).

Sunlight can activate the formation of vitamin D3. Interestingly, the precursor to vitamin D3 is cholesterol. Cells also produce an abundance of cholesterol sulfate the important precursor to vitamin D3. Due to the lack of depth of penetration of sunlight into the human body, the photo-induced bio-synthesis of vitamin D3 is confined to the skin area.

The benefits of vitamin D3 are actually stemming from cholesterol sulfate and lead to protection against diabetes, cardiovascular disease and certain cancers.

In view of the ability to convert X-Ray into UV light, the technology now exists to perform biosynthesis of vitamin D3 in-vivo anywhere in the human or animal body. Energy converting particles can be placed (through injection) in proximity to a cholesterol rich area and help convert such cholesterol into water soluble vitamin D3.

In one embodiment of the present invention, photonic energy from the energy converters noted above can be used to photo-excite these types of reactions to promote light-activated bio-synthesis. This can be used as a method of increasing vitamin D3 levels in a subject, lowering total cholesterol levels in a subject, or both simultaneously. Of course, the level of vitamin D3 production could be controlled by controlling the amount of incident high energy radiation (such as x-ray) which in turn would control the amount of UV production in vivo.

In another embodiment of the present invention, the photonic energy need not come from down-converting phosphors. Other means for generating ultraviolet or visible light in vivo may be used by injecting into the body in target regions upconverting phosphors, UV or visible light emitting diodes, light-emitting plasma capsules, etc. to photo-excite reactions promoting vitamin D3 production and/or other light-activated bio-synthesis.

Nucleic acids in living cells are associated with a large variety of proteins. Ultraviolet (UV) irradiation of cells is thought to lead to reactions between DNA and the proteins that are in contact with it, such as cross-linking between the amino acids in these associated proteins and the bases in DNA, which appears to be an important process that photoexcited DNA and proteins undergo in vivo, as well as in DNA-protein complexes in vitro. Twenty two (22) common amino acids are known to bind photochemically (upon 254 nm excitation) to uracil, with the most reactive being phenylalanine, tyrosine and cysteine. The three amino acid residues having side chains that absorb in the UV range are the aromatic residues tryptophan (Trp), tyrosine (Tyr) and phenylalanine (Phe).

One photochemical mechanism in proteins involves reduction of disulphide bridges upon UV excitation of Trp and Tyr side chains. As discussed by Neves-Petersen et al (2012), UV-excitation of tryptophan or tyrosine can result in their photoionization and to the generation of solvated electrons. The generated solvated electrons can subsequently undergo fast geminate recombination with their parent molecule, or they can be captured by electrophillic species like molecular oxygen, H3O+ (at low pH), and cystines (name given to each bridged cysteine in a disulphide bridge), which can also result in the breakage of the disulphide bridge. The free thiol radicals/groups thus formed can then react with other free thiol groups to create a new disulphide bridge. As detailed by Neves-Petersen et al (2012), this phenomenon has led to a new technology for protein immobilization (LAMI, light assisted molecular immobilization) since the created thiol groups can bind thiol reactive surfaces leading to oriented covalent protein immobilization.

There are many potential pathways for the breakage of intramolecular disulphide bridges in proteins upon UV excitation of aromatic residues, even in the absence of molecular oxygen. Breakage of the disulphide bridge can lead to conformational changes in the protein, not necessarily resulting in inactivation of the protein.

Neves-Petersen et al (2012) report that the solvated electron average lifetime is shorter at acidic pH values, which is correlated with the fact that H30+captures the solvated electron. Furthermore, the solvated electron lifetime is significantly shorter in protein systems as compared to from Trp alone in solution, thus indicating that a protein offers other pathways involving capture of the solvated electron. Neves-Petersen et al (2012) also report that data has shown that the higher the pH the longer time it takes for the solvated electron to recombine with the parent molecule (geminate recombination) or another electron scavenger molecule, such as H3O+. The observed lifetime increase with pH can be explained since the lower the pH, the higher the concentration of H30+ and therefore the larger the probability of recombination of the solvated electron with the hydronium ion. Furthermore, for proteins, the higher the pH of the solution, the larger the number of basic titratable residues that have lost their positive charge and became neutral (His, Lys, Arg) and the larger the number of acidic titratable residues that have acquired a negative charge (Asp, Gly, Tyr, Cys not bridged). This means that an increase of pH leads to a loss of positive charge in the protein and a gain of neutral and negative charged residues in the protein. This can lead to an increase of the areas in the protein that carry a negative electrostatic potential. Therefore, an increase in pH will decrease the efficiency of electron recombination with the molecule due to electrostatic repulsion. This can lead to an increase of the solvated electron lifetime.

The recent development of DNA microarrays has demonstrated the importance of immobilization technology, where multiple oligonucleotide or cDNA samples are immobilized on a solid surface in a spatially addressable manner. These arrays have revolutionized genetic studies by making the global analysis of gene expression in living organisms more readily possible. Similar approaches have been developed for protein analysis. The proteins bound to the microarrays can be assayed for functional or structural properties, making screening possible on a scale and with a speed previously unachievable. The simplest type of protein immobilization uses the high inherent binding affinity of surfaces to proteins in general, such as through the use of numerous weak contacts, including van der Waals and hydrogen bonding interactions. Molecules can also be immobilized on a carrier or solid surface passively through hydrophobic or ionic interactions, or covalently by attachment to surface groups. Due to the importance of immobilization for solid phase chemistry and biological screening, the analytical uses of the technology have been widely explored. The technology has found particularly broad application in different areas of biotechnology, including, but not limited to, diagnostics, biosensors, affinity chromatography and immobilization of molecules in assays such as ELISA assays.

Light-induced immobilization techniques have also been explored, leading to the use of quinone compounds for photochemical linking to a carbon-containing support (see, e.g., EP0820483). Activation occurs after irradiation with non-ionizing UV and visible light. Masks can be used to activate certain areas of the support for subsequent attachment of biomolecules. Following illumination, the photochemically active compound anthraquinone will react as a free radical and form a stable ether bond with a polymer surface. Because anthraquinone is not found in native biomolecules, appropriate ligands have to be introduced into the biomolecule. A further development of light-induced immobilization technology is disclosed in U.S. Pat. Nos. 5,412,087 and 6,406,844, each of which is incorporated herein by reference in its entirety.

Most of the known immobilization methods use one or more thermochemical/chemical steps, sometimes with hazardous chemicals, some of which are likely to have a deleterious effect on the structure and/or function of the bound protein. The available methods are often invasive, whereby foreign groups are introduced into a protein to act as functional groups, which can cause protein denaturation, as well as lower its biological activity and substrate specificity. Neves-Petersen et al (2012) have suggested this can be addressed by Light Assisted Molecular Immobilization technology (LAMI). This technology provides a photonic method for coupling a protein or a peptide on a carrier by way of stable bonds (covalent bond or thiol-Au bond) while preserving the native structural and functional properties of the coupled protein or peptide.

LAMI technology uses an inherent natural property of proteins and peptides, whereby a disulphide bridge in a protein or peptide, located in close proximity to an aromatic amino acid residue, is disrupted following excitation of aromatic amino acids. The thiol groups created by light induced disulphide bridge breakage in a protein or peptide are then used to immobilise the protein or peptide to a carrier. The formed free thiol groups in the protein can then attach the protein onto a thiol reactive surface, such as gold, thiol derivatized glass and quartz, or even plastics. The new protein immobilization technology has led to the development of microarrays of active biosensors and biofunctionalization of thiol reactive nanoparticles, aiming at engineering drug delivery systems.

### EXEMPLARY METHODS OF THE INVENTION

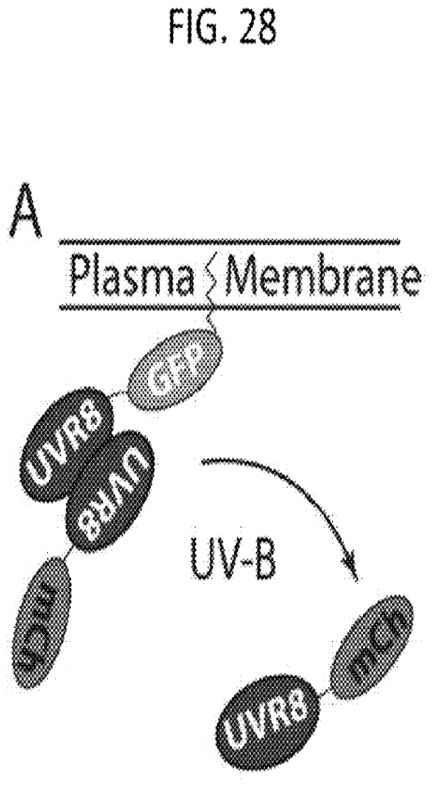

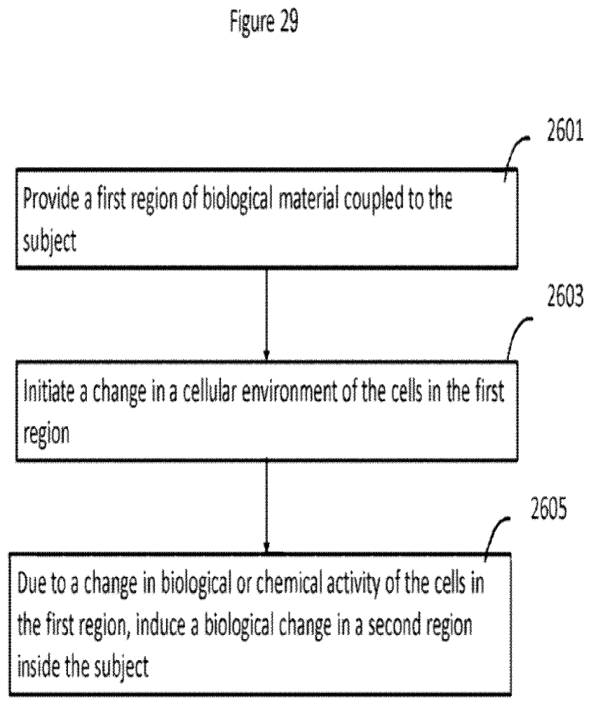

FIG. 29 is a flowchart of one method of the invention for treating a subject. At 2601, this method provides a first region of biological material coupled to the subject. At 2603, this method initiates a change in a cellular environment of the cells in the first region. At 2605, due to a change in biological or chemical activity of the cells in the first region, this method induces a biological change in a second region inside the subject.

According to various embodiments of the invention, the first region can be a region inside the subject proximate the second region or it can be a region inside the subject remote from the second region. In one embodiment, the first region can be a region outside the subject coupled physically to the second region or it can be a region inside the subject overlapping the second region.

Furthermore, at 2601, the biological material of the first region can be segregated from the second region by an artificial material. The artificial material may comprise a permeable material capable of transmission of chemical agents produced by the biological material from the first region into the second region. The artificial material may comprise a material capable of transmission of biophotons therethrough. The artificial material may comprise a material capable of transmission of sonic waves therethrough. The artificial material may comprise a material capable of transmission of ultraviolet light or visible light therethrough. The artificial material may comprise a material capable of transmission of infrared light therethrough. The artificial material may comprise a material capable of transmission of electrical signals therethrough. At 2605, the first region and the second region can be quantum entangled regions, permitting the coupling to occur.

Furthermore, at 2603, initiating a change can cause cell death of the biological material of the first region or initiating a change can cause cell growth of the biological material of the first region. The changes initialed can be caused by imposing an electric field in the first region to promote ion pumping through cells in the biological material of the first region, or by imposing an electric field in the first region to retard ion pumping through cells in the biological material of the first region. Furthermore, the changes initialed can be caused by changing a rate of transport of reagents through cell membranes cells in the biological material of the first region, for example by changing a probability of tunneling of the reagents through cell membranes. In one example, the probability of tunneling is changed by applying an electric field to promote or retard transmission of the reagents through the cell membranes in the biological material of the first region. In another example, the probability of tunneling is changed by applying a photon flux to the reagents to increase an energy of the reagents. In another example, the probability of tunneling is changed by applying a drug which thickens the cell membranes. In another example, the probability of tunneling is changed by applying a drug which dilates or constricts pores in the cell membranes. In a specialized example, the drugs affecting the cell membranes can be isolated only to the first region so that toxicity of the drug does not affect the subject.

Furthermore, at 2603, initiating a change can change a rate of enzymatic reactions occurring in the biological material or can change a rate of catalysis reactions occurring in the biological material. At 2603, initiating a change can change a rate of photosynthesis occurring in the biological material. At 2603, initiating a change can change the genomics of the biological material in the first region. This change in genomics in the first region can induce a therapeutic change in the second region.

Furthermore, at 2605, inducing a biological change in a second region inside the subject occurs by coupling to the second region via interactions of DNA molecules along a pathway from the first region to the second region. In this embodiment, the pathway may comprise as part or all the pathway signaling DNA. In one embodiment, coupling is provided by transporting charge along the signaling DNA. In one embodiment, inducing a biological change occurs by removing a protein that normally binds to signaling DNA in the biological material of the first region.

In these steps and actions above, biophotonic or mitogenic radiation from the first regions is transmitted (or otherwise coupled) to the second regions to thereby induce the change in the second region. When this coupling is via ultraviolet or visible light, the photon flux in a specialized case is that of a single photon emission and transmission, and possibly emitted and transmitted coherently with other sources of the biophoton radiation.

Optionally, in the steps and actions above, biophoton emission is stimulated by artificial sources such that living tissues in the first region produce biophoton radiation.

Optionally, in the steps and actions above, artificial or simulated biophoton emission is produced and coupled to the second or treatment region.

In these steps and actions above, a change in the viability of the cells in the first region produces a similar change in the second region of the subject.

Furthermore, at 2601, the biological material of the first region is surgically defined (isolated, separated, partially removed) from a diseased organ in the subject, a treatment is applied to the first region that had been surgically defined to promote cell death (or alternatively cell growth), thereby inducing cell death (or cell growth) as the biological change in the second region (or treatment region) of the subject. With this approach, the surgically defined first region can be selectively treated to induce cell death for example by chemically inducing cell death in the surgically defined first region or by chemically inducing cell death in the surgically defined first region by radiation. One example of such radiation can include ultraviolet light. Other examples include x-rays, gamma rays, protons, or other high energy sources.

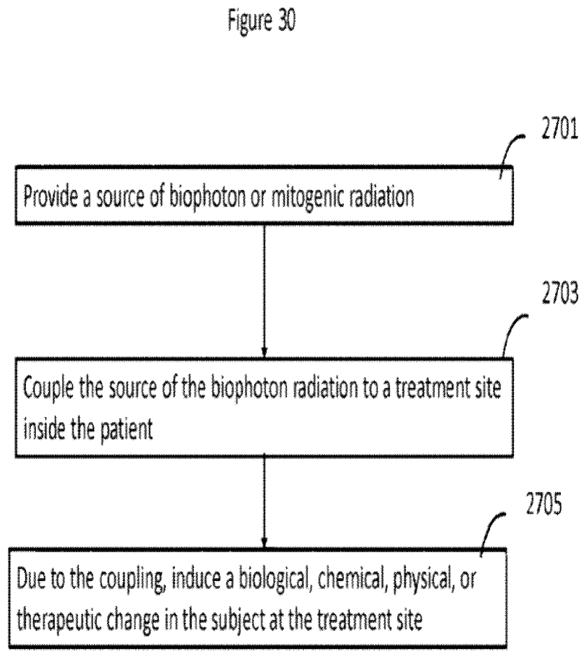

FIG. 30 is a flowchart of another method of the invention for treating a subject. At 2701, this method provides a source of biophoton or mitogenic radiation. At 2703, this method couples the source of the biophoton radiation to a treatment site inside the patient.

These steps or actions in FIG. 30 may occur with any of the other steps and actions set forth above with regard to FIG. 29.

### Strategies for Photonic Coupling to Diseased Tissue Via Cellular Communication:

One can identify a photonic energy hvₘₚ₊ that promotes the activity of the metabolic pump (referred to as mp+ energy). Conversely, it is possible to find photonic energy that diminishes the function of the metabolic pump called hvₘₚ₋ (referred to mp− energy). This makes it possible to target tumor cells and irradiate them with the appropriate “mp− energy” to limit their function and energy production. This approach is the deconstructive approach with the strategy of impeding the undesirable behavior (uncontrolled growth). This deconstructive approach has significant limitations in that insofar as apoptosis is not triggered in the tumor environment, even a small percentage of cancerous or mutated cells that are left behind can result in the formation of metastasis and relaunch of the disease. In fact, this is the problem of all of the state-of-the-art therapies. The eradication of disease is never complete and cancerous cells find a way to invade adjacent tissue and to recolonize mutated cells in different organs. The metastasis problem is one of the biggest issues facing all therapies.

Another strategy is referred to as the photonically constructive approach and comprises stimulating the biological circuitry to generate healthy bio-photonic signatures that in-turn communicate with the tumor micro environment (TME) to stay on a healthy course rather than to engage in undesirable mutations leading to cancer propagation. The ability to collect the bio-photonic signature from the healthy tissue and to compare it with a cancerous tissue would thus provide a feedback loop necessary to activate the biological circuitry to promote the right photonic signature. The biological circuitry can be stimulated by having an increase in the metabolic pump function.

By distinguishing diseased tissue from heathy tissue in any organ of the body, a regimen of photonic stimulus can be implemented periodically until the diseased cells go back to normal behavior and subsequently become regulated by the immune system. This constructive approach should be done first to limit the evasion of mutated cells from the surveillance of the immune system. Once the cells no longer have countermeasures to evade the immune system, then the disease is corrected quickly and efficiently by the existing (and complex) chain of events enabled by the immune system.

Since the biological circuitry can be stimulated by having an increase in the metabolic pump function, this makes it possible to stimulate the proteins gating the doorways to ion channels and to cause an increase in the uptake of ions ever so slightly to build up more voltage (hence more energy storage) which results in the decay and dissipation of said stored energy via photonic energy.

The photonic energy at the cellular level coupled with the ability to measure ultraweak photons is one preferred embodiment of the present invention. It is also recognized that photonic signatures can carry information of types not described herein. However, the ability to interact at the cellular level using photons opens a myriad of medical possibilities and novel therapies based on cellular light communication.

### Cell-to-Cell Communication

In various embodiments of the present invention, stimulation, detection, monitoring and/or measurement of cell-to-cell communication can lead to the replication of biophoton emissions for treatment of a target site or for a feedback on a diagnostic treatment of a target site. In one example, the determination of the light characteristics of biophoton emission from target cells in cell-to-cell communication can be used to replicate stimulant light which would be used to treat a target site. Determination of those light characteristics need not occur in vivo, but according to one embodiment of the invention can occur in a controlled in vitro apparatus where the optical configuration to measure biophoton radiation can be designed to detect such emissions, including, but not limited to, as detailed in the cuvette formats described below.

### 1—Cuvettes Formats

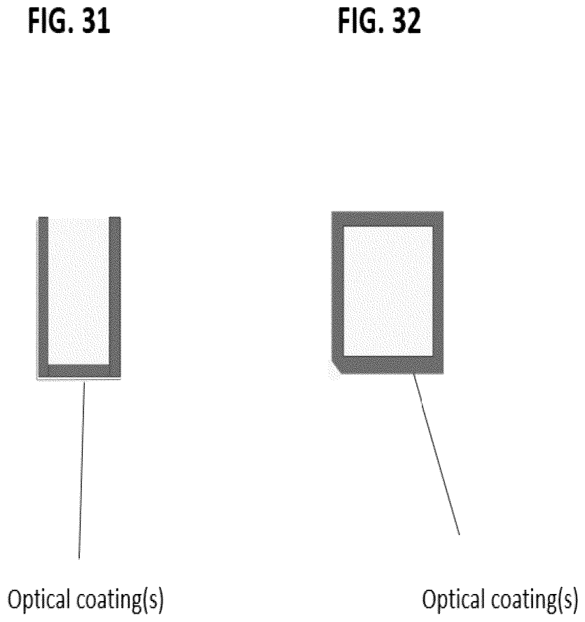

Cuvettes such as those detailed below, in one embodiment of the invention can monitor optical communication between cells or between organisms that are in suspension inside the cuvettes. FIGS. 31 and 32 are views of cuvettes suitable for the present invention. More specifically, FIGS. 31 and 32 show cross sectional views of cuvettes with outer walls coated with a low index of refraction optical coating. The cuvette in FIG. 32 represents a sealed cuvette structure.

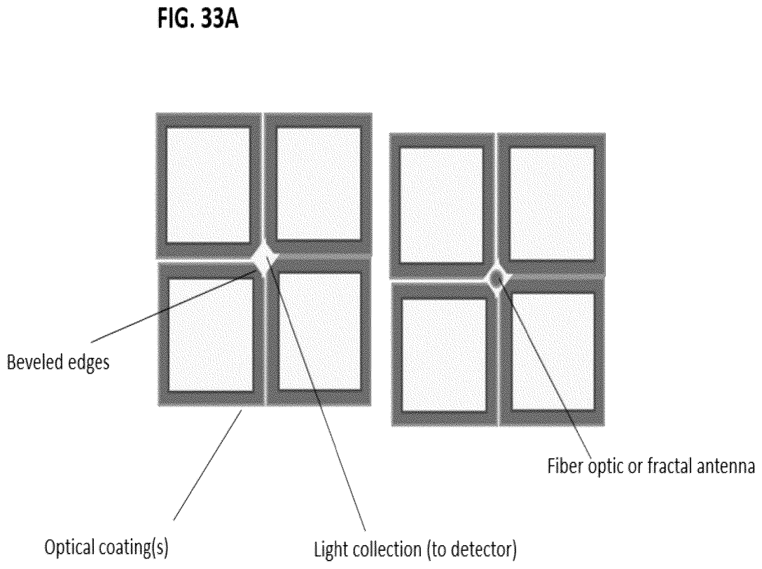

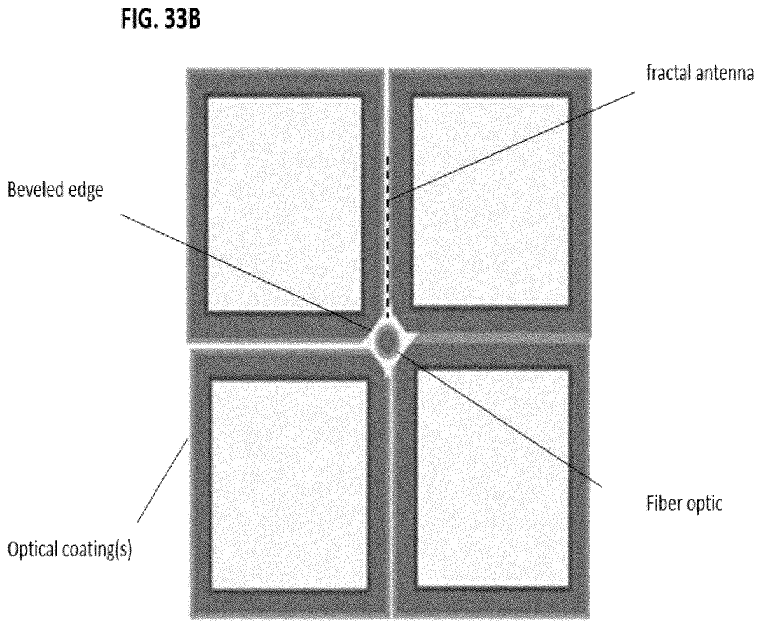

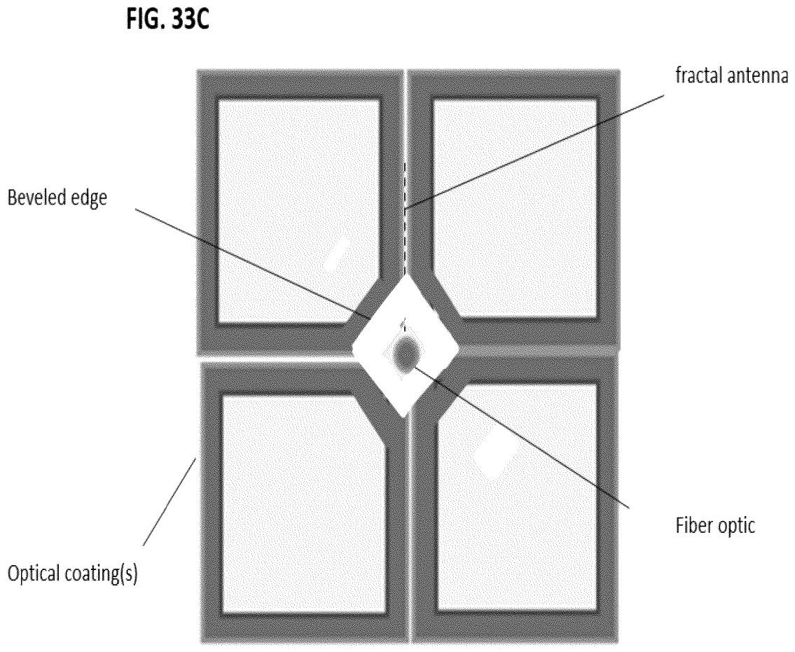

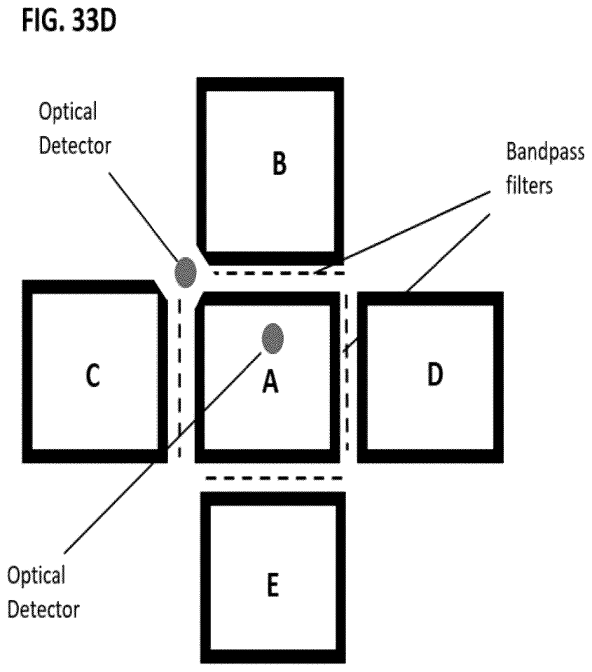

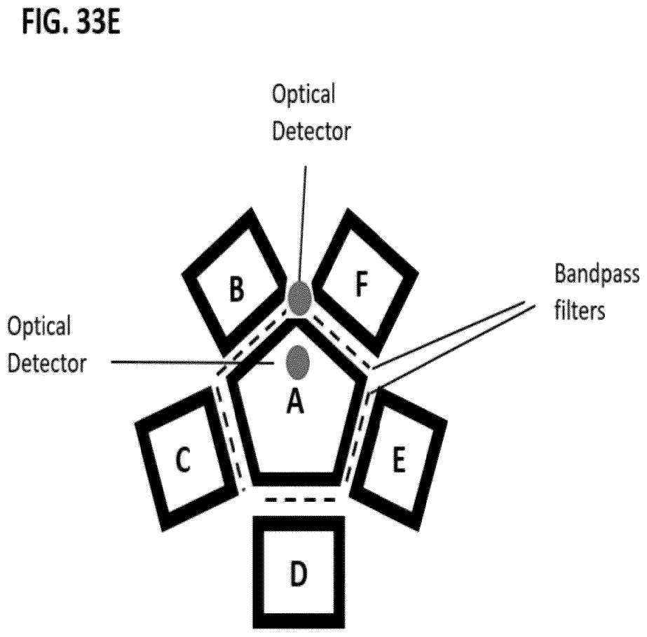

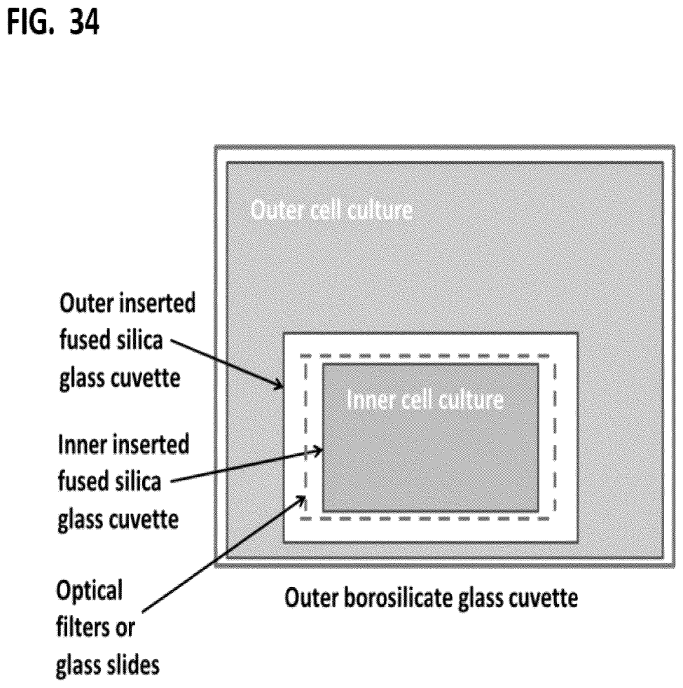

FIG. 33A shows top views of cuvettes in cuvette stack of the present invention with the beveled edges of the cuvettes placed in a juxtaposed position to monitor light communication between various organisms placed in the cuvettes.

The preferred cuvettes used in the present invention differ from standard cuvettes in two aspects: First, the outer walls are coated with a low index of refraction coating (an optical coating), and one edge is beveled to allow waveguiding to take place, thereby permitting the positioning of an optical fiber in close proximity to the wall. Second, a beveled edge of the cuvette allows the placement of an optical fiber in the intersection of four (4) cuvettes.

In one embodiment of the present invention, an optical fiber coated with a thin metallic coating is used to couple weak signals into the waveguide core. Additionally, a single cuvette with multiple internal barriers may be used to facilitate cell-to-cell communication via photonics. It is also envisioned that single cuvettes with semipermeable membranes may be of value to allow the diffusion of molecules modulated via photonic stimulation. Diffusion rate and molecule size can be modulated via different pore sizes on the semipermeable membrane allowing molecules but not cells to be transported across the membranes.

In one embodiment of the present invention, all the cells in the cuvette stack contain a biological material such as a biopsy material from a patient. By inducing cell death in all the cells, biophotons from all the cells are collected in the bevel-center region where the optical fiber resides.

In another embodiment of the present invention, one cell in the cuvette stack may contain the biopsy material to be treated, while the other cells in the four cuvette stack may contain untreated biopsy material. By inducing cell death in the one cell, biophotons from one cell can be collected in the bevel-center region where the optical fiber resides, and subsequent cell death in the remaining cells can be monitored by subsequent staining techniques and also by detecting subsequent biophoton emission.

In another embodiment of the present invention, one cell in the cuvette array may contain the biopsy material to be treated, while the other cells in the cuvette array may contain untreated biopsy material. The adjacent walls of adjacent cells would have a bandpass filter permitting transmission of a defined wavelength range placed between the adjacent walls, with each set of adjacent walls having different bandpass filters each with differing defined wavelength transmission ranges. By inducing cell death or another desired cellular change in the one cell, biophotons generated from the cellular change will be transmitted through the bandpass filter having a wavelength transmission range that corresponds to the generated biophotons from the cellular change. This transmission of biophotons will trigger a corresponding cellular change in the adjacent cuvette which has the corresponding bandpass filter transmission range, thus permitting identification of the wavelength range of biophoton emissions which cause the desired cellular change. This information can then be used in treatment methods for a living subject by generating corresponding simulated biophotonic emissions within the living subject using biophoton emitters such as those described elsewhere in this application, thus triggering the desired cellular change within the living subject.

In another embodiment of the present invention, as shown in FIG. 33B, a fractal antenna can be placed between adjacent cuvettes to monitor for biophotons emitted from the one cell being treated to an adjacent cell. In another embodiment of the present invention, the fractal antennae may be placed around the fiber optic (as detailed later in an example of an in vivo detector).

FIG. 33C is a depiction of another cuvette stack of the present invention where the beveled edge is cut effectively deeper into the corner of each cuvette to provide a larger surface for light coupling to the fiber optic (or optical detector) at the center of the cuvette stack.

FIG. 33D is a depiction of another cuvette configuration. In this configuration, the treated cells would be placed in the center cuvette A with bandpass filters between each wall between A and each of the surrounding cuvettes. Complete blocking filters could be used between each of the surrounding cuvettes if needed. While shown to have bevels on adjacent cuvettes at the top, the beveled configuration could be at every corner of every cuvette. For example, the “A” cuvette could have every corner beveled while each surrounding cuvette would have a bevel of 2 corners) and each corresponding position at the corners of cuvette A could have a fiber optic. In one embodiment of the invention, different wavelength band pass filters could be positioned allowing detection at a larger number of wavelength ranges. When cells are placed in the “A” central cuvette and caused to undergo a desired change (such as apoptosis), untreated cells can be placed in each of the surrounding cuvettes and those untreated cells monitored to detect the desired change in those untreated cells. Based on which cuvette contains cells that undergo the desired change, the wavelength range of biophoton emission communicating and potentially causing that change can be readily determined. Optional optical detectors may be disposed inside each of the cuvettes or in selected cuvettes.

FIG. 33E is a depiction of another cuvette configuration. In this embodiment, a central cuvette has a pentagonal shape. Cuvettes B, C, D, E., and F are disposed adjacent the central cuvette. In one embodiment of the invention, different wavelength band pass filters could be positioned allowing detection at a larger number of wavelength ranges. Optional optical detectors may be disposed inside each of the cuvettes or in selected cuvettes.

The configurations of FIGS. 33D and 33E can be extended such that the central cuvette can have any desired number of sides (any polygon shape), with each side having an adjacent cuvette B, C, D, etc. with bandpass filters of different wavelength ranges being placed between each external cuvette and the central cuvette side. In this manner, a single experiment can explore a wide variety of wavelength ranges to determine the wavelength range of biophotons being emitted from the central cuvette “A” and communicating/causing the desired change in one of the surrounding cuvettes.

### 2—Biophoton Wavelength Determination:

In another embodiment of the present invention, an outer cell container is made from borosilicate glass (Pyrex) for containing an outer cell culture. Within the outer cell culture, two sealed cuvettes made from fused silica are placed, one fully inside the other. The inner cuvette is fully contained inside the other sealed cuvette. The inner cell culture is fully contained inside the inner cuvette. Since both inserted cuvettes are sealed, there is no exchange of liquid or gas between the inner cell culture and the outer cell culture is possible.

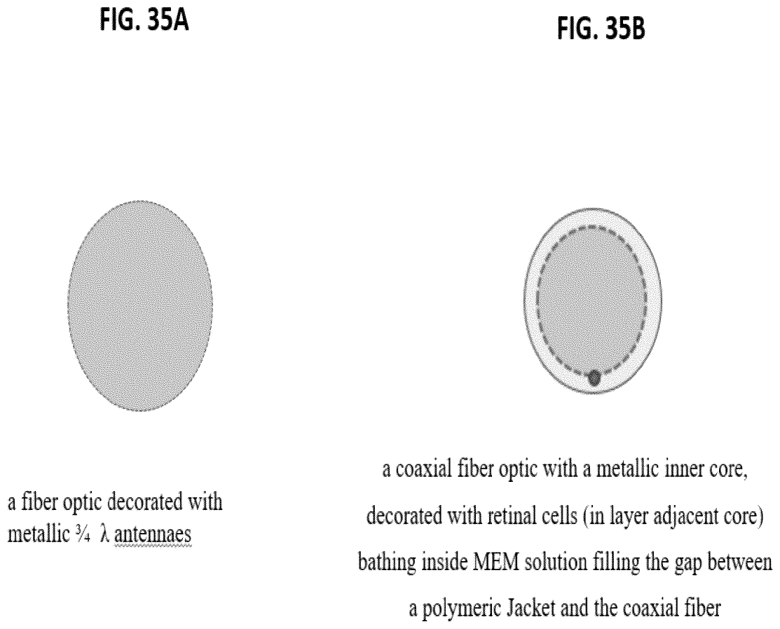

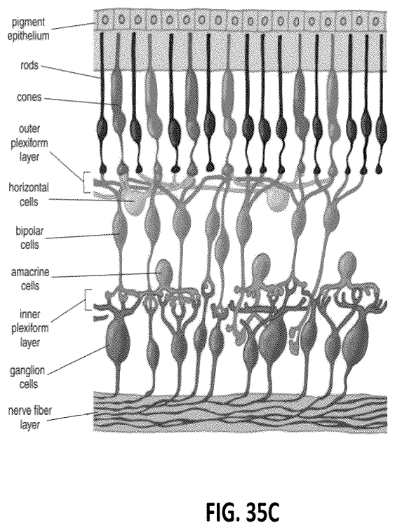

Since the inserted cuvettes are made of silica glass, there is no electrical communications between the inner cell culture and the outer cell culture. The gas inside both cuvettes is pure nitrogen gas. FIG. 35A shows a design for this structure.

### Example 1: Determination of a UV Wavelength Biophoton

If the outer cell culture is distressed to promote cell death for example by hydrogen peroxide injection, the inner cell culture will respond according to the following. If the cuvette containing the inner cell culture is made of borosilicate glass with a UV edge near 360 nm, then, there is no optical communications between the two cultures, and the inner culture would show no sign of distress. However, if the cuvette containing the inner cell culture is made from fused silica glass with a UV edge neat 180 nm, then the inner cell culture is expected to show the same kind of distress response as the outer cell culture. The reason seems to come from optical communications between the two cultures using light with wavelength between 180 nm and 360 nm, because the borosilicate glass absorbs the signal, and the fused silica glass passes the signal.

### Example 2: Determination of a Different Wavelengths Biophotons

In one embodiment of the invention, two inserted cuvettes with different optical absorbers can be placed one inside the outer inserted cuvette and the second outside the inner inserted cuvette. This allows one to reduce the transmission range between the two cell cultures with a choice of bandpass filters and/or glass slides with different UV edge absorptions. The filters and glass slide absorbers are designed to fully fill the area of all the sides of the inner cuvette. By placing the filters and/or glass absorbers between the two inserted cuvettes, any chemical contamination of the cell cultures by the filters or the absorbing glass slides is avoided.

For absorbing glass slides, the transmission ranges are as follows:

| Short wavelength limit | Inserted absorbing glass plates |
|---|---|
| 180 nm | Fused silica |
| 200 nm | Sapphire |
| 240 nm | Hafnia glass |
| 270 nm | Zirconia glass |
| 310 nm | Alluxa bandpass filter |
| 330 nm | Alluxa bandpass filter |
| 369 nm | Alluxa bandpass filter |
| 375 nm | Alluxa bandpass filter |
| 385 nm | Alluxa bandpass filter |

Should it be necessary to investigate shorter wavelengths, cuvettes made from Aluminum Fluoride with a UV edge of 150 nm and from Magnesium Fluoride with a UV edge of 140 nm can be used for the inserted cuvettes to replace fused silica.

### 3—Detector Optimization

In one embodiment of the present invention, once the wavelength range for biophoton emission has been established for a particular biological change, a detector with an optimized gain in that wavelength range is selected to more precisely measure the optical characteristics such as wavelength, intensity, modulation frequency, etc.

In one embodiment of the present invention, from these characteristics, light can be artificially produced with those characteristics and applied to a patient or two a cell culture of the patient to ascertain the biological change.

In one embodiment of the present invention, biomarkers indicative of the biological change can be measured from the patient or the cell culture.

### 4—In Vivo Detectors

a—the Inorganic In Vivo Detection System

In one embodiment of the present invention, an inorganic in vivo detection system is provided which has a fiber optic (as shown in FIG. 35A) decorated with % X antennae on the periphery. This fiber is then inserted in an area of interest such as the tumor boundary or the tumor microenvironment. (Other antennae designs as described earlier may be placed on the periphery of the fiber.) Indeed, the fractal antennae designs discussed above may be disposed on a substrate made of an artificially grown organism such as described in U.S. Pat. No. 6,428,802 entitled “Preparing artificial organs by forming polylayers of different cell populations on a substrate” (the entire contents of which are incorporated herein by reference) and as described in U.S. Pat. No. 6,372,495 entitled “Bio-artificial organ containing a matrix having hollow fibers for supplying gaseous oxygen” (the entire contents of which are incorporated herein by reference).

In this case, biophoton emission from the artificially grown organism may be coupled to a treatment site in a subject. Additionally, biophoton emission can be detected by the electrical conductors and used as feedback for treating the treatment site. In one embodiment, electrical conductors can be disposed in vivo nearby a treatment site to measure biophoton emission from the treatment site, and/or the electrical conductors can be disposed in vivo nearby an untreated site to monitor biophoton emission from a treated site to the untreated site.

b—the Organic In Vivo Detection System:

FIG. 35 B shows another design with a coaxial axial fiber optic having a metallic inner core decorated with retinal cells. This detection system has a coaxial fiber optic with a thin metallic inner core. The outer surface of the coaxial fiber is decorated with retinal cells. The retinal cells can be selected from the appropriated animal and/or insects described below. The cells are connected to the thin metallic inner core of the fiber to conduct electrical impulses. An outer jacket is placed on the coaxial fiber optic and sealed. The gap between the outer jacket and the coaxial line is filled with Minimal Essential Media to keep the cell alive.

The retina and retinal cells are described in detail in “How the Retina Works”, a paper by Helga Kolb, published January 2003, American Scientist 91(1); DOI:10.1511/2003.11.841, the entire contents of which are incorporated herein by reference. FIG. 35C depicts the structure of a typical retina. Intricately wired neurons in the retina allow a good deal of image assembly to take place in the eye itself. Scientists understand about half of the interactions among the cells in this delicate piece of tissue.

In FIG. 35C, light enters the eye from the top. The photons travel through the vitreous fluid of the eyeball and penetrate the entire retina, which is about half a millimeter thick, before reaching the photoreceptors—the cones and rods that respond to light (the colored and black cells attached to the epithelium at top). Signals then pass from the photoreceptors through a series of neural connections toward the surface of the retina, where the ganglion-cell nerve-fiber layer relays the processed information to the optic nerve and into the brain. Cells in the retina are arrayed in discrete layers. The photoreceptors are at the top of this FIG. 35C rendering, close to the pigment epithelium. The bodies of horizontal cells and bipolar cells compose the inner nuclear layer. Amacrine cells lie close to ganglion cells near the surface of the retina. Axon-to-dendrite neural connections make up the plexiform layers separating rows of cell bodies.

In a detector based on retinal cells as described herein, the nerve fiber layer of the retinal cells would connect with the metallic core to conduct electrical impulses. The fibers would be dedicated per retinal cell and cone types. Preferably, the fibers would be specific for a particular wavelength range. For example, an exemplary embodiment of retinal cell based detector would have the following:
Fibers with organic antennas for detecting UVA
Fibers with organic antennas for detecting blue
Fibers with organic antennas for detecting green
Fibers with organic antennas for detecting red
Fibers with organic antennas for detecting IR

Such an arrangement would permit detection and monitoring of biophoton emission throughout the electromagnetic spectrum, with the signal being represented in various current levels of electrical impulse corresponding to the various wavelengths detected.

Retinal cells for the visible and UV: Retinal receptors have been developed through evolution to allow animals to see colors. The retina of humans is made up of two types of cells that receive light and produces electrical impulses in response. The two types are rods and cones. Humans detect color in visible light using only three types of detectors (cones) that cover the visible range. These cones are sensitive over the following wavelength ranges: the short-wavelength cones (S) range from 390 to 480 nm in sensitivity, with a peak near 440 nm; these are called the blue cones; the mid-wavelength cones (M) range from 450 to 650 nm in sensitivity with a peak near 540 nm; these are called the green cones; the long-wavelength cones (L) range from 480 to 780 nm in sensitivity with a peak near 580 nm; these are called the red cones. The cones are located in the retina and are highly concentrated in the fovea. The second type of retinal light detectors are called rods and are black and white detectors. They have a slightly wider wavelength detection range than all three types of cones together and they are more sensitive, and thus able to detect weaker signals, so that all objects look gray in very low light levels.

In looking at an eye response, the different types of cones are not present in the eye in equal numbers. The red cones are most abundant (63%), and there are many green cones (31%). The eye contains a relatively small number of blue cones (6%). One consequence of the low abundance of short wavelength cones is the relatively poor ability of human eyes to discern the color blue. The human eye is, therefore, most responsive to green, yellow, orange and red light.

However, there are different types of retinal cells in other species. Birds, for example, have five different types of visual pigment cells and seven different types of photoreceptor cells consisting of rods, double or uneven twin cones, and four types of single cones. The wavelengths of maximum sensitivity of the different photoreceptors vary according to habitat and species. Hart (2001) investigated the short-wavelength sensitivity of birds' eyes by the examination of spectral sensitivities of avian retinal photoreceptors through micro-spectrophotometric measurements of single cells, spectrophotometric measurements of extracted or in-vitro regenerated visual pigments, and molecular genetic analyses of visual pigment opsin protein sequences. The author identified peak rod sensitivity at 505 nm, while the cones are as follows: long wavelengths—555 to 570 nm; mid wavelengths—500 to 506 nm; short wavelengths—446 to 454 nm; and a fourth set of cones that are violet or UV sensitive—362 to 415 nm, with some bird species slightly outside of these ranges. While green-sensitive cones are not found to be helpful for visualizing leaf contrast, the UV-sensitive cones in many birds' eyes seem to give the bird species that possess them the ability to distinguish between the top and bottom sides of leaves in a forest or jungle environment [Tidore & Nilsson] thus improving leaf edges differentiation and allowing birds to more easily navigate through the trees.

For avian cells, the visual pigments consist of an opsin protein bound via a Schiff base to a chromophore, either 11-cis-retinal or 11-cis-3,4-dehydroretinal. Spectral tuning in all but one class of pigment is achieved by replacement of one of the chromophores for the other [1](11-cis-retinal blueshifts compared to 11-cis-3,4-dehydroretinal [2,3]), long-pass filtering by pigmented cone oil-droplets [4] or substitutions of key amino acids (aa) in the opsin protein. The most sensitive shortwave cone is called the SWS1 cone in VS (violet sensitive) birds and has maximum absorption in the range 402 to 426 nm. In comparison, the true UV retinal cone cell is called the UVS cone in ultraviolet sensitive birds with a maximum absorption range 355-380 nm.

Retinal Cells for the Infrared side of the spectrum: Snakes such as pit vipers like pythons, boas, and rattlesnakes, have infrared vision. They have pits lined with heat sensors along their upper and lower jaws. Some blood sucking insects including mosquitoes also have infrared vision. Additionally, fish such as goldfish, salmon, piranha and cichlid can see infrared light. Of particular interest is the goldfish which is believed to see both infrared and ultraviolet light. One additional species of interest is the bullfrog who can see infrared. Bullfrogs use Cyp27cl, an enzyme linked to vitamin A to boost their infrared vision making them highly adaptable to both land and aquatic life. The retinal cells from these animals, insects and fish can be used to detect infrared light.

### Biomarkers

As background, X-RAY psoralen activated cancer therapy (X-PACT) as described in U.S. Pat. No. 11,207,409 (the entire contents of which are incorporated herein by reference) has proven to retard tumor growth, resulting in stabilization of disease, partial remissions, and complete remissions. Studies of XPACT by some of the present inventors have measured the concentration of key cytokines biomarkers in various patients and have observed a correlation with the clinical outcome. In one embodiment of the present invention, important biomarkers were identified including TGF-Beta, MCP1 and IFG.

In one embodiment of the invention, the present invention augments and/or replaces the conventional treatment of a patient based on averages with wide standard deviations with a closed loop feedback methodology (with potentially a statistic of N=1) by the gathering of real time data as needed for patient treatment where the concentration of biomarkers are measured real time (or frequently on a schedule). For instance, the tissue from a patient's biopsy could be subjected to various wavelengths of light to identify what upregulates certain mRNA messenger molecules, leading to a favorable clinical benefit. In one example of this approach, phosphors with appropriate wavelengths can be administered followed by low dose X-Ray irradiation to deliver the correct wavelengths to a treatment site in a patient. In another example of this approach, a laser source could be used through a fiber optic cable to deliver the correct wavelength of light to the treatment site. Once a treatment site has been treated, in one embodiment of the invention, cell-to-cell communication causes a similar cell response at an untreated site (e.g., a site removed from the treatment site). While the body may itself may provide its own mechanisms for cell-to-cell communication, in one embodiment of the invention, systems are provided for improving or channeling cell-to-cell communication.

Historically, in the study of light on cell function, there have been two approaches: direct interaction of the light with cellular components and activation of an added photosensitive agent that acts on the cellular components. Most of this work has been done with the latter approach. Here, in one embodiment of the present invention, DNA are modified with photosensitive or photoactive receptors that turn on when illuminated at the proper wavelength. This “on state” can activate a variety of processes in the cell, such as protein kinase activity, turning on or off peptide activity, controlling enzymatic activity and inducing DNA cleavage for cell multiplication.

In another embodiment of the present invention, light activates a compound (e.g., a photoactivable or photosensitive agent) added to the cellular network, and that compound controls cellular behavior. In some cases, the induced behavior, such as turning on the human body immune response, can have significant treatment value such as in XPACT noted above,

In one embodiment of the present invention, light stimulation or other types of non XPACT treatments (such as anti-inflammatories) may be initially applied to a treatment site, biomarkers measured, and an XPACT treatment applied if the biomarkers are trending in the expected direction.

The direct activation of cellular components by monochromatic and/or polychromatic light is less clearly understood. Most recent publications are of clinical studies in which the mechanisms are not elucidated nor investigated.

Animal cells are complex constructs consisting of many parts, as shown in FIG. 1 Light has been observed or speculated to have interacted with the cell nucleus, the DNA and its telomeres, with the mitochondria, with the endoplasmic reticulum and with ribosomes to name a few. This has been observed in a variety of cells from among the over 200 types of cells in the human body.

TABLE 5
Cell Classification

| Types of cells | Cells in the grouping |
|---|---|
| Stem cells | Embryonic and adult, differentiated |
| Red blood cells | Erythrocytes |
| White blood cells | Monocytes, lymphocytes |
| | Neutrophils, eosinophils, basophils |
| Platelets | Fragments of megakaryocytes |
| Nerve cells | Neurons |
| Muscle cells | Smooth, cardiac |
| Skeletal, bone cells | Osteoblasts, osteoclasts, osteocytes |
| Cartilage cells | Chondrocytes, |
| Skin cells | Keratinocytes, melanocytes, Merkel, |
| | Langerhans |
| Endothelial cells | |
| Epithelial cells | |
| Fat cells | Adipocytes |
| Sex cells | |

In various embodiments of the invention, light modulation for controlling specific cellular function(s) in the human body may affect disease pr cancer progression as illustrated by the stages of cancer progression below. In one embodiment of the present invention, light modulation which stops the progression of cancer to the escape stage (detailed below) may assist in stopping the spread cancer throughout the body. Moreover, morphological changes in the shape, porosity, interconnections (for example by nanotube formation), vesiculation, etc. can be an “optical biomarker” of the present invention serving as an (indirect) indicator that cell-to-cell communication at an untreated site was effective.

### 1. Stage 1—Single Cell Stage

The first stage is the effect of light modulation on single cell behavior. For example, in one embodiment, the shining light on stem cells may accelerate differentiation and cell division. Other effects of light modulation may be to affect cell division, angiogenesis, etc. The single cells in this stage may be types of human-derived cell lines such as for example undifferentiated stem cells, osteoblasts, dermal cells, neuron cells, brain cells, muscle cells, etc. In one embodiment of the invention, the effect of light modulation influences not only a single cell but groups of cells by affecting the collective behavior and cell communications pathways.

### 2. Stage 2—Groups of Cells/Organoids

The second stage is the effect of light modulation on groups of cells. Various pathways for cell-to-cell communication may be affected. In one embodiment of the invention, near-field optics permits the pathways to be identified and correlated. Near-field optics is a method by which the spatial resolution of light illumination is constricted to illuminate single cells in groups of cells. U.S. Pat. Nos. 6,388,239 and 6,064,060 and 8,393,010 (the entire contents of each patent are incorporated herein) describe near-field scanning optical microscopes.

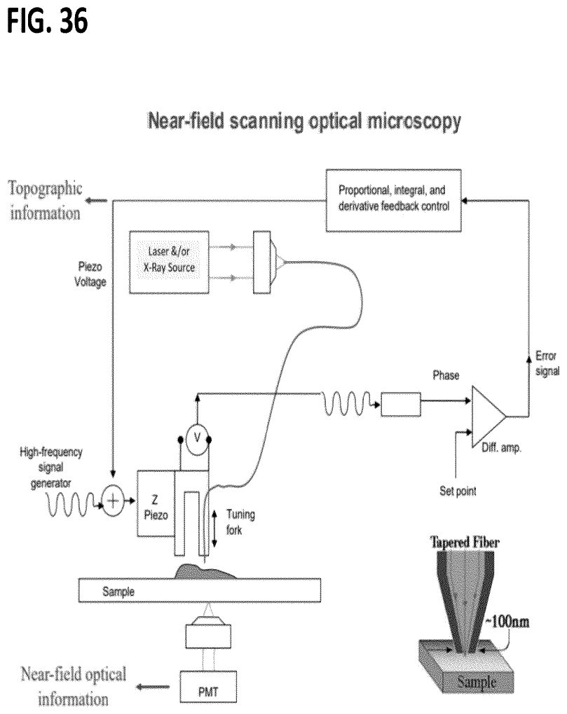

In one example as shown in FIG. 36, a metal-coated tapered fiber delivery method can achieve subwavelength resolution down to tens of nanometers. In FIG. 36, an additional fiber with its controller can be added to the set up. One fiber can deliver the light stimulus and the other fiber can monitor the light production away from the stimulus site. Two additional fibers with their controllers can be envisaged in this experiment for a total of 4 fibers. In one embodiment, at this spatial resolution, the effects of light on a single cell or cells in groups of cells can be resolved, and subsequently the effects of light modulation of selected regions within a single cell can be monitored at the dimensions of living cells. In various embodiments, the light signal is time resolved so that the responses can be correlated to the illumination, permitting the time of a response to be measured. In one embodiment of the invention, the antennas described above and shown in FIGS. 7-1 through 7-11 may be used in conjunction with Near-Field Optical Microscopy (NSOM) and Near-Field Raman spectroscopy for studies of cellular response and communications. In one embodiment, at this spatial resolution, morphological changes including the growth of nanotubes between cells can be monitored as a biomarker at a treatment site or at an intreated site as an indicator of cell-to-cell communication to the untreated site.

In one embodiment a X-ray fiber delivers X-Ray to a treatment site to cause UV emission from energy modulators embedded with the cell.

In one embodiment a suspension of minimal essential media and phosphors is delivered to the treatment site and mapped with the AF microscopy and then irradiated with X-Ray.

### 3. Stage 3—Cell Tissue

The third stage is the effect of light modulation on large groups of cells in tissue. In various embodiments, sources of stimulation light (such as for example from phosphors to produce the desired light wavelengths in the tissue upon low-dose x-ray exposure or from lasers or other sources of light) can stimulate/produce cell responses, which can be monitored using the spectroscopic tools noted above. In one embodiment, broader communications pathways can be correlated with the light amount and the light wavelength.

### 4. Stage 4—Subcellular and Molecular Levels

The fourth stage is the effect of light modulation on cellular components and the effect of light on photosensitive agents added to the cellular environment. In one embodiment, stimulant light which caused physical, chemical or biological changes in the cellular function(s) in stage 1 are applied to different subcellular components of the cell, such as the mitochondria, the nucleus, the cell membrane and various components of the cytoplasm such as proteins, and RNA molecules. In one embodiment, both eukaryotic cells (cells with a nucleus and sub-cellular mitochondria) as well as prokaryotic (cells lacking a nucleus and mitochondria) can be stimulated by light at least at the appropriate wavelengths and intensities of stage 1 exposure. In one embodiment, in either the structures of prokaryotic and eukaryotic cells, light is directed to specific parts of the cell(s) to assist in a cell's ability to synthesize/degrade ATP via oxidative phosphorylation. In one embodiment of the present invention, resonance Raman and FT Infrared absorption are tools for tracking light-induced cellular processes (including possible photon-electron processes. As before in the above embodiments, light guidance may use near-field optical microscopy as well as near-field optical spectroscopy.

In one specific example, the production/quantification and turnover of ATP from exposed mitochondrion cells (exposed to red and near IR light) is monitored, using near-field Raman spectroscopy. In one embodiment of the present invention, the measurement of telomere lengths upon exposure to various wavelengths of light will provide a direct measure of the stimulant light effect.

A telomere is a region of repetitive DNA sequences at the end of a chromosome. Telomeres protect the ends of chromosomes from becoming frayed or tangled. Each time a cell divides, the telomeres become slightly shorter. Telomere length is suggested to be a proxy for biological ageing. Telomere length is dynamic and shortens at every cell division as well as under oxidative stress. In one embodiment of the present invention, the telomere length is a biomarker which can be measured by known techniques, such as DNA sequencing.

Mitochondria—The structure of mitochondria is highly dynamic. Mitochondrial shape is cell-type specific and can be modified to meet changing requirements in energy production, calcium homeostasis, lipid biogenesis, fatty acid synthesis and other mitochondrial activities. This is achieved in one embodiment of the invention by modulating with the stimulant light the dynamic properties of mitochondria including fusion, division, movement, and positional tethering using the stimulant light.
a. Mitochondria—The mitochondria provide the energy that operates and drives cellular function. Unlike chromosomes in the nucleus, the mitochondria have a structure and energy production that are specific to the types of cells in which the mitochondria reside. By stimulant light, fusion, division, positional placement in the cell are dynamics which modify the structure of the mitochondria inside the cells. In one embodiment of the invention, mitochondria may play a prominent role in local cell to cell communications. In another embodiment of the invention, the localized effect on the mitochondria inside the cell can induce a biological change in a second region inside the patient remote from the stimulant light treatment site. The health of the mitochondria and its energy production capacity can determine the health of the cells, the tissues and the organs.
b. Of interest in this stage is the production/quantification and turnover of ATP from exposed mitochondria cells, exposed for example with red and near IR light, using near-field Raman spectroscopy and the measurement of telomere lengths upon exposure to various wavelengths of light. Since a reduction in telomere length has been linked to oxidative stress of the mitochondria, by observing the telomere length using the techniques above, both the local and non-local effects can be monitored by the near field optics noted above.

Gene expression. Here, in one embodiment of the invention, mRNA production can be monitored using for example near-field Raman spectroscopy or other clinical analysis to determine the excited states at the cell level or at the molecular level and subsequently correlate these excitation states with changes in gene expression and the flow of energy in the cell cytoplasm. In one embodiment of the present invention, the modulation of mRNA to induce the expression of specific molecules can be measured at a treated site or at an untreated site as an indicator of cell-to-cell communication to the untreated site.

### 5. Stage 5—Patient Treatment

The fifth stage is the use of light modulation for patient treatment. Light induced healing has been reported in the literature for accelerated wound healing, increased stem cell differentiation and growth, stimulation of the immune system, cancer cell identification and destruction, and various cell growth mechanisms.

Here, in the present invention, the stimulant light can initiate a change in a cellular environment of the cells in a first (treatment) region of a patient. The change is tracked or monitored by the monitoring of biomarkers in the cells in the first region. Due to a change in biological or chemical activity of the cells in the first region of the patient, a biological change in a second region inside the patient is induced. The biological change in the second region inside the patient can also be monitored by detecting biomarkers in cells in the second region. The mechanism of the induction may be local changes and/or more global changes throughout the patient induced by cell-to-cell communication from a test site exposed to the stimulant light and a remote site not treated. Regardless, in one embodiment, monitoring for biomarkers at the untreated site may serve as an early indication of cell response at the untreated site, and therefore more suitable for a closed loop feedback determining patient treatment.

### Photo-Bio-Modulation Using Energy Conversion and Using Low-Level-LASER

In one embodiment of the present invention, photo bio modulation by the stimulant light may use down-converted energy from X-Ray (energy conversion photo bio modulation EC-PBM) to affect cellular behavior. However, other forms of energy conversions are possible such as up-conversion from IR and microwave and RF. Additionally, low level light from laser sources can also be used for the stimulant light to induce photo bio modulation (low light level photo bio modulation LLL-PBM).

The penetration depth of (EC-PBM) is superior to that of LLL-PBM. This is so because the excitation energy X-Ray, RF and Microwave can penetrate deep into tissue compared to LASERs operating in the IR regime. Down conversion of energy can be mediated by energy converting particles comprising hosphor materials. These materials absorb X-Ray and emit UV and visible light. Such energy converting particles can be mixed with a liquid such as saline solution or minimal essential media to form a suspension that can be injected at the requisite (target treatment) site in the human body. The suspension can be injected for example in a targeted tissue at the requisite site followed by precise X-ray delivery to the area of interest. The injections can be done directly into a targeted tissue such as a tumor. In patients the injections can be guided by ultrasound energy or by fluoroscope. The delivery of suspensions containing phosphors can alternatively be done using microscopy as detailed in FIG. 36. Remote activation of EC particles using low level Xray makes EC-PBM an on-demand and targeted therapy.

Further, the X-Ray energy can be pulsed to minimize any collateral damage to the surrounding tissue. The pulsation approach can be further optimized by selecting phosphors with long decay time so that UV energy can be delivered even though X-Ray is delivered on a duty cycle.

The energy converters (EC) can emit light of different wavelengths. Some EC particles can emit a primary wavelength. A combination of EC particles can emit a broad spectrum of wavelengths. Therefore, the treatment modalities of EC-PBM which can be used in the present invention include using one to multiple wavelengths (forming a broad spectrum type emission). The range of wavelengths made possible by EC-PBM can span from 180 nm to 810 nm. This is not possible using laser light sources since laser have to be selected to emit a specific wavelength of light.

Furthermore, when exposed to X-ray, EC particles undergo an electron-hole disassociation process followed by recombination process of the e-h pairs. The electrons excited by X-Ray energy are emitted with sufficiently high kinetic energy that they can travel to the surface of the particles. Therefore, in addition to light emissions, some particles can supply electrons on their surfaces under X-Ray exposures making the EC particles both photonically and electrically active. These surface electrons can migrate through different mechanisms including electron-hopping to the surrounding biological matrices leading to constructive or deconstructive interference of biological reactions.

### Energy Converting Materials:

The present invention can use any one or more desired energy converters, including, but not limited to, organic fluorescent molecules or inorganic particles capable of fluorescence and/or phosphorescence having crystalline, polycrystalline or amorphous micro-structures. Also included in these energy converters are the various embodiments of energy modulation agents noted above.

Organic fluorescent compounds with high quantum yield include, but are not limited to: naphthalene, pyrene, perylene, anthracene, phenanthrene, p-terphenyl, p-quaterphenyl, trans-stilbene, tetraphenylbutadiene, distyrylbenzene, 2,5-diphenyloxazole, 4-methyl-7-diethylaminocoumarin, 2-phenyl-5-(4-biphenyl)-1,3,4-oxadiazole, 3-phenylcarbostyryl, 1,3,5-triphenyl-2-pyrazoline, 1,8-naphthoylene-1′,2′-benzimidazole, 4-amino-n-phenyl-naphthalimide.

Inorganic fluorescent and/or phosphorescent materials span a wide variety of materials. Furthermore, these materials can be doped with specific ions (activators or a combination of activators) that occupy a site in the lattice structure in the case of crystalline or polycrystalline materials and could occupy a network forming site or a bridging and/or non-bridging site in amorphous materials.

These compounds include, but are not limited to, the phosphors cited in the background of the invention to enable UV and Visible wavelengths of different kinds and intensities to be delivered to the treatment site, as well as the following (not ranked by order of preference or utility):
CaF2, ZnF2, KMgF3, ZnGa2O4, ZnAl2O4, Zn2SiO4, Zn2GeO4, Ca5(PO4)3F, Sr5(PO4)3F, CaSiO3, MgSiO3, ZnS, MgGa2O4, LaAl11O18, Zn2SiO4, Ca5(PO4)3F, Mg4Ta2O9, CaF2, LiA15O8, LiAlO2, CaPO3, AlF3, and LuPO4:Pr3+, Ca3(PO₄)₂:Tl⁺, (Ca, Zn)₃(PO₄)₂:Tl⁺.

Examples further include the alkali earth chalcogenide phosphors which are in turn exemplified by the following non-inclusive list: MgS:Eu3+, CaS:Mn2+, CaS:Cu, CaS:Sb, CaS:Ce3+, CaS:Eu2+, CaS:Eu2+Ce3+, CaS:Sm3+, CaS:Pb2+, CaO:Mn2+, CaO:Pb2+. Further examples include the ZnS type phosphors that encompass various derivatives: ZnS:Cu,Al(Cl), ZnS:Cl(Al), ZnS:Cu,I(Cl), ZnS:Cu, ZnS:Cu,In.

Also included are the compound IIIb-Vb phosphors which include the group IIb and Vb elements of the periodic table. These semiconductors include BN, BP, BSb, AlN, AlP, AlAs, AlSb, GaN, GaP, GaAs, GaSb, InN, InP, InAs, InSb and these materials may include donors and acceptors that work together to induce light emission diodes. These donors include, but are not limited to, Li, Sn, Si, Li, Te, Se, S, O and acceptors include, but are not limited to, C, Be, Mg, Zn, Cd, Si, Ge. Further included are the major GaP light emitting diodes which include, but are not limited to, GaP:Zn,O, GaP:NN, Gap:N and GaP, which emit colors Red, Yellow, Green and Pure Green respectively.

The materials can further include such materials as GaAs with compositional variation of the following sort: In1-y(Ga1-xAlx)yP. Also included is silicon carbide SiC, which has commercial relevancy as a luminescent platform in blue light emitting diodes. These include the polytypes 3C-SiC, 6H-SiC, 4H-SiC with donors such as N and Al and acceptors such as Ga and B.

Further examples include multiband luminescent materials include, but not limited to, the following compositions (Sr, Ca, Ba)5(PO4)3Cl:Eu2+, BaMg2Al16O27:Eu2+, CeMgAl11O19:Ce3+:Tb3+, LaPO4:Ce3+:Tb3+, GdMgB5O10:Ce3:Tb3+, Y2O3:Eu3+, (Ba,Ca,Mg)5(PO4)3Cl:Eu2+, 2SrO0.84P2O50.16B2O3:Eu2+, Sr4Al14O25:Eu2+.

Materials typically used for fluorescent high pressure mercury discharge lamps are also included. These can be excited with X-Ray and are exemplified by way of family designation as follows: Phosphates (Sr, M)(PO4)2:Sn2+, Mg or Zn activator, Germanate 4MgO·GeO2:Mn4+, 4(MgO, MgF2)GeO2:Mn4+, Yttrate Y2O3:Eu3+, Vanadate YVO4:Eu3+, Y(P,V)O4:Eu3+, Y(P,V)O4:In+, Halo-Silicate Sr2Si3O82SrCl2:Eu2+, Aluminate (Ba,Mg)2Al16O24:Eu2+, (Ba, Mg)2Al16O24:Eu2+, Mn2+, Y2O3Al2O3:Tb3+.

Another grouping by host compound includes chemical compositions in the halophosphates phosphors, phosphate phosphors, silicate phosphors, aluminate phosphors, borate phosphors, tungstate phosphors, and other phosphors. The halophosphates include, but are not limited to: 3Ca3(PO4)2]Ca(F,Cl)2:Sb3+, 3Ca3(PO4)2·Ca(F,Cl)2:Sb3+/Mn2+, Sr10(PO4)6Cl2:Eu2+, (Sr,Ca)10(PO4)6Cl2:Eu2+, (Sr,Ca)10(PO4)6·nB2O3:Eu3+, (Sr, Ca,Mg)10(PO4)6Cl2:Eu2+.

The phosphate phosphors include, but are not limited to: Sr2P2O7:Sn2+, (Sr,Mg)3(PO4)2:Sn2+, Ca3(PO4)2·Sn2+, Ca3(PO4)2:Tl+, (Ca,Zn)3(PO4)2:Tl+, Sr2P2O7:Eu2+, SrMgP2O7:Eu2+, Sr3(PO4)2:Eu2+, LaPO4:Ce3+, Tb3+, La2O3·0.2SiO2·0.9P2O5:Ce3+·Tb3+, BaO·TiO2·P2O5. The silicate phosphors Zn2SiO4:Mn2+, CaSiO3:Pb2+/Mn2+, (Ba, Sr, Mg)·3Si2O7:Pb2+, BaSi2O5:Pb2+, Sr2Si3O8.2SrCl2:Eu2+, Ba3MgSi2O8:Eu2+, (Sr,Ba)Al2Si2O8:Eu2+.

The aluminate phosphors include but are not limited to: LiAlO2:Fe3+, BaAl8O13:Eu2+, BaMg2Al16O27:Eu2+, BaMg2Al16O27:Eu2+/Mn2+, Sr4Al14O25:Eu2+, CeMgAl11O19:Ce3+/Tb3+.

The borate phosphors include: Cd2B2O5:Mn2+, SrB4O7F:Eu2+, GdMgB5O10:Ce3+/Tb3+, GdMgB5O10:Ce3+/Mn3+, GdMgB5O10:Ce3+/Tb3+/Mn2+.

The tungstate phosphors include but are not limited to: CaWO4, (Ca,Pb)WO4, MgWO4. Other phosphors Y2O3:Eu3+, Y(V,P)O4:Eu2+, YVO4:Dy3+, MgGa2O4:Mn2+, 6MgO·As2O5:Mn2+, 3.5MgO·0.5MgF2·GeO2:Mn4+.

Of particular interest are phosphors having deep UV emissions such as Ca₃(PO₄)₂:Tl⁺, (Ca, Zn)₃(PO₄)₂:Tl⁺ having peak emissions at 328 nm and 310 nm respectively. An increase in the Zn amount, the emission peak shifts to shorter wavelengths. The composition in practical use is (Ca_{0.9}, Zn_{0.1})₃(PO₄)₂:Tl⁺.

### Activators:

Activators are used to “dope” phosphors to generate specific emissions. Activators for the present invention include, but are not limited to: Tl+, Pb2+, Ce3+, Eu2+, WO42−, Sn2+, Sb3+, Mn2+, Tb3+, Eu3+, Mn4+, Fe3+. The luminescence center Tl+ is used with a chemical composition such as: (Ca,Zn)3(PO4)2:Tl+, Ca3(PO4)2:Tl+. The luminescence center Mn2+ is used with chemical compositions such as MgGa2O4:Mn2+, BaMg2Al16O27:Eu2+/Mn2+, Zn2SiO4:Mn2+, 3Ca3(PO4)2·Ca(F,Cl)2:Sb2+/Mn2+, CaSiO3:Pb2+/Mn2+, Cd2B2O5:Mn2+, CdB2O5:Mn2+, GdMgB5O10:Ce3+/Mn2+, GdMgB5O10:Ce3+/Tb3+/Mn2+. The luminescence center Sn2+ is used with chemical compositions such as: Sr2P2O7:Sn2+, (Sr,Mg)3(PO4)2:Sn2+. The luminescence center Eu2+ is used with chemical compositions such as: SrB4O7F:Eu2+, (Sr,Ba)Al2Si2O8:Eu2+, Sr3(PO4)2:Eu2+, Sr2P2O7:Eu2+, Ba3MgSi2O8:Eu2+, Sr10(PO4)6Cl2:Eu2+, BaMg2Al16O27:Eu2+/Mn2+, (Sr,Ca)10(PO4)6Cl2:Eu2+. The luminescence center Pb2+ is used with chemical compositions such as: (Ba,Mg,Zn)3Si2O7:Pb2+, BaSi2O5:Pb2+, (Ba,Sr)3Si2O7:Pb2+.

The luminescence center Sb2+ is used with chemical compositions such as: 3Ca3(PO4)2·Ca(F,Cl)2:Sb3+, 3Ca3(PO4)2·Ca(F,Cl)2:Sb3+/Mn2+.

The luminescence center Tb3+ is used with chemical compositions such as: CeMgAl11O19:Ce3+/Tb3+, LaPO4:Ce3+/Tb3+, Y2SiO5:Ce3+/Tb3+, GdMgB5O10:Ce3+/Tb3+. The luminescence center Eu3+ is used with chemical compositions such as: Y2O3:Eu3+, Y(V,P)O4:Eu3+. The luminescence center Dy3+ is used with chemical compositions such as: YVO4:Dy3+. The luminescence center Fe3+ is used with chemical compositions such as: LiAlO2:Fe3+. The luminescence center Mn4+ is used with chemical compositions such as: 6MgO.As2O5:Mn4+, 3.5MgO0.5MgF2≮GeO2:Mn4+. The luminescence center Ce3+ is used with chemical compositions such as: Ca2MgSi2O7:Ce3+ and Y2SiO5:Ce3+. The luminescence center WO42− is used with chemical compositions such as: CaWO4, (Ca,Pb)WO4, MgWO4. The luminescence center TiO44− is used with chemical compositions such as: BaO·TiO2·P2O5.

### K-Edge:

Additional phosphor chemistries of interest using X-Ray excitations include, but are not limited to, the k-edge of these phosphors. Low energy excitation can lead to intense luminescence in materials with low k-edge. Some of these chemistries and the corresponding k-edge are listed below:

| | | |
|---|---|---|
| BaFCl:Eu2+ | 37.38 | keV |
| BaSO4:Eu2+ | 37.38 | keV |
| CaWO4 | 69.48 | keV |
| Gd2O2S:Tb3+ | 50.22 | keV |
| LaOBr:Tb3+ | 38.92 | keV |
| LaOBr:Tm3+ | 38.92 | keV |
| La2O2S:Tb3+ | 38.92 | keV |
| Y2O2S:Tb3+ | 17.04 | keV |
| YTaO4 | 67.42 | keV |
| YTaO4:Nb | 67.42 | keV |
| ZnS:Ag | 9.66 | keV |
| (Zn, Cd)S:Ag | 9.66/26.7 | keV |

These materials can be used alone or in combinations of two or more. A variety of compositions can be prepared to obtain the desired output wavelength or spectrum of wavelengths.

### Output Spectra:

In one embodiment the present invention, the phosphor selection is chosen such that under x-ray or other high energy source irradiation, the light emitted from the phosphors would mimic natural biophoton spectra of a target cell being treated in order to simulate a particular biophoton emission (similar to that described above), where exemplary characteristics could include:
emissions in 190-250 nm wavelength range;
emissions in the 250-330 nm wavelength range;
emissions in the 330-340 nm wavelength range;
a combination of emissions in the 190-250 nm, in th 250-330 nm, and in the 330-340 nm wavelength ranges;
emissions across the range of 250 nm to 600 nm;
emissions in the infrared range;
a duration of emission in short bursts mimicking the pulsation of the excitation light
a pulsation that could vary from the nano second range, millisecond range and all the way to continuous wave
the pulsation and light intensity of the light stimulus would be studied to optimize the desired biological outcome;
and/or
a range of photon flux from a few to a 1000 photons/(sec*cm2) or higher.

With an in vivo point of use biophoton generator, the duty cycle of the x-ray unit or x-ray source would determine the duty cycle of the biophoton radiation produced, the phosphor selection or combination of phosphors would determine the wavelength emission characteristics, and external coatings on the phosphors would serve to attenuate the level of light emitted at a target site.

Moreover, in one embodiment of the present invention, since the level of light emission for biophotons is considered low in intensity, the x-ray dose to a patient for a biophoton radiation treatment can be significantly lower than that for other radiation treatments.

In one embodiment of the present invention, a downconverting energy modulation agent (e.g., a down converting phosphor) can comprise inorganic particulates selected from the group consisting of: metal oxides; metal sulfides; doped metal oxides; and mixed metal chalcogenides. In one aspect of the invention, the downconverting material can comprise at least one of Y2O3, Y2O2S, NaYF4, NaYbF4, YAG, YAP, Nd2O3, LaF3, LaCl3, La2O3, TiO2, LuPO4, YVO4, YbF3, YF3, Na-doped YbF3, ZnS; ZnSe; MgS; CaS and alkali lead silicate including compositions of SiO2, B2O3, Na2O, K2O, PbO, MgO, or Ag, and combinations or alloys or layers thereof. In one aspect of the invention, the downconverting material can include a dopant including at least one of Er, Eu, Yb, Tm, Nd, Mn Tb, Ce, Y, U, Pr, La, Gd and other rare-earth species or a combination thereof. The dopant can be included at a concentration of 0.01%-50% by mol concentration. U.S. Pat. Appl. Publ. Nos. 2017/0157418 and 2017/0239489 (the entire contents of both are incorporated herein by reference) provided details of these and other suitable phosphors.

In one aspect of the invention, the downconverting energy modulation agent can comprise materials such as ZnSeS:Cu, Ag, Ce, Tb; CaS: Ce,Sm; La2O2S:Tb; Y2O2S:Tb; Gd2O2S:Pr, Ce, F; LaPO4. In other aspects of the invention, the downconverting material can comprise phosphors such as ZnS:Ag and ZnS:Cu, Pb. In other aspects of the invention, the downconverting material can be alloys of the ZnSeS family doped with other metals. For example, suitable materials include ZnSexSy:Cu, Ag, Ce, Tb, where the following x, y values and intermediate values are acceptable: x:y; respectively 0:1; 0.1:0.9; 0.2:0.8; 0.3:0.7; 0.4:0.6; 0.5:0.5; 0.6:0.4; 0.7:0.3; 0.8:0.2; 0.9:0.1; and 1.0:0.0.

In other aspects of the invention, the downconverting energy modulation agent can be materials such as sodium yttrium fluoride (NaYF4), lanthanum fluoride (LaF3), lanthanum oxysulfide (La2O2S), yttrium oxysulfide (Y2O2S), yttrium fluoride (YF3), yttrium gallate, yttrium aluminum garnet (YAG), gadolinium fluoride (GdF3), barium yttrium fluoride (BaYF5, BaY2F8), gadolinium oxysulfide (Gd2O2S), calcium tungstate (CaWO4), yttrium oxide:terbium (Yt2O3Tb), gadolinium oxysulphide:europium (Gd2O2S:Eu), lanthanum oxysulphide:europium (La2O2S:Eu), and gadolinium oxysulphide:promethium, cerium, fluorine (Gd2O2S:Pr,Ce,F), YPO4:Nd, LaPO4:Pr, (Ca,Mg)SO4:Pb, YBO3:Pr, Y2SiO5:Pr, Y2Si2O7:Pr, SrLi2SiO4:Pr,Na, and CaLi2SiO4:Pr.

In other aspects of the invention, the downconverting energy modulation agent can be near-infrared (NIR) downconversion (DC) phosphors such as KSrPO4:Eu2+, Pr3+, or NaGdF4:Eu or Zn2SiO4:Tb3+, Yb3+ or β-NaGdF4 co-doped with Ce3+ and Tb3+ ions or Gd2O2S:Tm or BaYF5:Eu3+ or other down converters which emit NIR from visible or UV light exposure (as in a cascade from x-ray to UV to NIR) or which emit NIR directly after x-ray or e-beam exposure.

In one aspect of the invention, an up converting energy modulation agent can also be used such as at least one of Y2O3, Y2O2S, NaYF4, NaYbF4, YAG, YAP, Nd2O3, LaF3, LaCl3, La2O3, TiO2, LuPO4, YVO4, YbF3, YF3, Na-doped YbF3, or SiO2 or alloys or layers thereof.

In one aspect of the invention, the energy modulation agents can be used singly or in combination with other down converting or up converting materials.

TABLE 1 shows a list of other suitable phosphors:

TABLE 1

| | | Emission | X-Ray Absorption | | | | | |
|---|---|---|---|---|---|---|---|---|
| | | Spectrum | Emiss | | | Microstructure | | |
| | | Peak Emission | Eff | Eff | K-edge | Specific | Crystal | |
| # | Phosphor | (nm) | % | (Z) | (keV) | Gravity | Structure | Hygroscopic |
| 1 | BaFCl:Ba²⁺ | 380 | 13 | 49.3 | 37.38 | 4.7 | Tetragonal | N |
| 2 | BaSO₄—:Eu²⁺— | 390 | 6 | 45.5 | 37.38 | 4.5 | Rhombic | N |
| 3 | LaOBr:Tu⁺³ | 360, 460 | 14 | 49.3 | 38.92 | 6.3 | Tetragonal | N |
| 4 | YTaO₄ | 337 | | 59.8 | 67.42 | 7.5 | Monolithic | N |
| 5 | YTaO₄:Nb (*) | 410 | 11 | 59.8 | 67.42 | 7.5 | Monolithic | N |
| 6 | CaWO₄ | 420 | 3 | 61.8 | 69.48 | 6.1 | Tetragonal | N |
| 7 | LaOBr:Tb³⁺ | 420 | 20 | 49.3 | 38.92 | 6.3 | Tetragonal | N |
| 8 | Y₂O₂S:Tb²⁺ | 420 | 18 | 34.9 | 17.04 | 4.9 | Hexgonal | N |
| 9 | ZnS:Ag | 450 | 17 | 26.7 | 9.66 | 3.9 | Hexgonal | N |
| 10 | (Zn,Cd)S:Ag | 530 | 19 | 38.4 | 9.66/26.7 | 4.8 | Hexgonal | N |
| 11 | Gd₂O₂S:Tb³⁺ | 545 | 13 | 59.5 | 50.22 | 7.3 | Hexgonal | N |
| 12 | La₂O₂S:Tb³⁺ | 545 | 12.5 | 52.6 | 38.92 | 6.5 | Hexgonal | N |

In one embodiment of the invention, besides the YTaO4, noted above, other energy modulation agents can include phosphors were obtained from the following sources. “Ruby Red” obtained from Voltarc, Masonlite & Kulka, Orange, Conn., and referred to as “Neo Ruby”; “Flamingo Red” obtained from EGL Lighting. Berkeley Heights, N.J, and referred to as “Flamingo”; “Green” obtained from EGL Lighting, Berkeley Heights, N.J. and referred to as “Tropic Green”; “Orange” obtained from Voltarc, Masonlite & Kulka. Orange, Conn, and referred to as “Majestic Orange”; “Yellow” obtained from Voltarc. Masonlite & Kulka, Orange. Conn., and referred to as “Clear Bright Yellow.” The “BP” phosphors are shown in detail below in TABLE 10:

TABLE 10

| | | Emission | | | | | | |
|---|---|---|---|---|---|---|---|---|
| | | Spectrum | X-Ray Absorption | | | Density | | |
| | Phosphor | Peak | Emiss | | | g/cc | Xtal | |
| | Material | Emission | Eff | Eff | K-edge | Specific | Crystal | |
| Code | Color | (nm) | % | (Z) | (keV) | Gravity | Structure | Hygroscopic |
| BP1 | CaWO4:Pb | 425 | | | | | | N |
| BP2 | Y2SiO5:Ce | 410 | | | | | | N |
| BP3 | YTaO4 | 337 | 10 | 59.8 | 67.42 | 7.5 | Monolithic | N |
| BP3-C | YTaO4 | 337 | 10 | 59.8 | 67.42 | | Monolithic | N |
| BP4 | BASF-1 | 460 | | | | | | |
| BP5 | BASF-2 | 490 | | | | | | |
| BP6 | YTaO4:Nb (*) | 410 | 11 | 59.8 | 67.42 | 7.5 | Monolithic | N |
| BP6-C | YTaO4:Nb (*) | | | | | | | |
| BP7-C | LaOBr:Tm3+ | 360, 460 | 14 | 49.3 | 38.92 | 6.3 | Tetragonal | N |
| | (coated) | | | | | | | |
| BP8-C | LaF3:Co | 280 | | | | | | |
| BP9 | Y2O3 | 365 | | | | | | |
| BP-10 | BaSO4:Eu2+ | 390 | 6 | 45.5 | 37.38 | 4.5 | Rhombic | N |
| | (coated) | | | | | | | |
| BP10-C | BaSO4—:Eu2+ | 390 | 6 | 45.5 | 37.38 | 4.5 | Rhombic | N |
| | (coated) | | | | | | | |
| BP11 | LaOCl:Tm | | | | | | | |
| BP12 | Y2O2S:Tm | | | | | | | |
| BP13 | BaSi2O5:Pb2+ | 350 | | | | | | N |
| | StB6O10:Pb | 360 | | | | | | N |
| | CaI:Na (Coated) | 338 | | | | | | Y |
| | Gd₂O₂S:Tm | Blue to | | | | | | Y |
| | | Green | | | | | | |

The “BP” phosphors are available from PhosphorTech Corporation of Kennesaw, Ga., from BASF Corporation, or from Phosphor Technology Ltd, Norton Park, Norton Road Stevenage, Herts, SG1 2BB, England.

Other useful energy modulation agents include semiconductor materials including for example TiO2, ZnO, and Fe₂O₃ which are biocompatible, and CdTe and CdSe which would preferably be encapsulated because of their expected toxicity. Other useful energy modulation agents include ZnS, CaS, BaS, SrS and Y₂O₃ which are less toxic. Other suitable energy modulation agents which would seem the most biocompatible are zinc sulfide, ZnS:Mn2+/Mn+3 and Mn+5 complexes, ferric oxide, titanium oxide, zine oxide, zine oxide containing small amounts of Al2O3 and AgI nanoclusters encapsulated in zeolite. For non-medical applications, where toxicity may not be as critical a concern, the following materials (as well as those listed elsewhere) are considered suitable: lanthanum and gadolinium oxyhalides activated with thulium; Er3+ doped BaTiO3 nanoparticles. Yb3+ doped CsMnCl3 and RbMnCl3. BaFBr:Eu2+ nanoparticles, cesium iodide, bismuth germanate, cadmium tungstate, and CsBr doped with divalent Eu. Table 11 below provides a list of various useful energy modulation agents

In various embodiments of the invention, the following luminescent polymers are also suitable as energy modulation agents: poly(phenylene ethynylene), poly(phenylene vinylene), poly(p-phenylene), poly(thiophene), poly(pyridyl vinylene), poly(pyrrole), poly(acetylene), poly(vinyl carbazole), poly(fluorenes), and the like, as well as copolymers and/or derivatives thereof.

As a non-limiting list, the following are also suitable energy modulation agents: Y2O3 ZnS; ZnSe; MgS; CaS; Mn, Er ZnSe; Mn, Er MgS; Mn, Er CaS; Mn, Er ZnS; Mn, Yb ZnSe; Mn, Yb MgS; Mn, Yb CaS; Mn, Yb ZnS:Tb3+, Er3+; ZnS:Tb3+; Y2O3:Tb3+; Y2O3:Tb3+, Er3+; ZnS:Mn2+; ZnS:Mn, Er3+; CaWO4, YaTO4, YaTO4:Nb, BaSO4:Eu, La2O2S:Tb, BaSi2O5:Pb, NaI(Tl), CsI(Tl), CsI(Na), CsI(pure), CsF, KI(Tl), LiI(Eu), BaF2, CaF, CaF2(Eu), ZnS(Ag), CaWO4, CdWO4, YAG(Ce) (Y3Al5O12(Ce)), BGO bismuth germanate, GSO gadolinium oxyorthosilicate, LSO lutetium oxyorthosilicate. LaCl3(Ce). LaBr3(Ce). LaPO4; Ce, Tb (doped). Zn2SiO4:Mn with Mn doped between 0.05-10%, and YTaO4.

In one embodiment, phosphors used in the invention as energy modulation agents can include phosphor particles, ionic doped phosphor particles, single crystal or poly-crystalline powders, single crystal or poly-crystalline monoliths, scintillator particles, a metallic shell encapsulating at least a fraction of a surface of the phosphors, a semiconductor shell encapsulating at least a fraction of a surface of the phosphors, and an insulator shell encapsulating at least a fraction of a surface of the phosphors, and phosphors of a distributed particle size. It is envisioned that an admixture of said energy modulation agents also may have enhanced effects and suitable biologic compatibility.

TABLE 11

| | | Emission | | | X-Ray | | | |
|---|---|---|---|---|---|---|---|---|
| | | Spectrum | Emiss | | Absorption | | | |
| Item | Phosphor | Peak Emission | Eff | Eff | K-edge | Specific | Crystal | |
| # | Color | (nm) | % | (Z) | (keV) | Gravity | Structure | Hygroscopic |
| 24 | Zn3(PO4)2:Tl+ | 310 | | | | | | N |
| 33 | BaF2 | 310 | | | | | | Slightly |
| 30 | CsI | 315 | | | | | | N |
| 28 | Ca3(PO4)2:Tl+ | 330 | | | | | | N |
| 4 | YTaO4 | 337 | | 59.8 | 67.42 | 7.5 | Monolithic | N |
| 38 | CsI:Na | 338 | | | | | | Y |
| 14 | BaSi2O5:Pb2+ | 350 | | | | | | N |
| 27 | Borosilicate | 350 | | | | | | N |
| 34 | LaCl3(Ce) | 350 | | | | | | Y |
| 16 | SrB4O7F:Eu2+ | 360 | | | | | | N |
| 20 | RbBr:Tl+ | 360 | | | | | | ? |
| 15 | (Ba,Sr,Mg,)3Si2O7:Pb2+ | 370 | | | | | | N |
| 17 | YAlO3:Ce3+ | 370 | | | | | | N |
| 37 | BC-422 | 370 | | | | | Organic | ? |
| 1 | BaFCl:Eu2+ | 380 | 13 | 49.3 | 37.38 | 4.7 | Tetragonal | N |
| 2 | BaSO4—:Eu2+ | 390 | 6 | [symbol] | 37.38 | 4.8 | Rhombic | N |
| 19 | BaFBr:Eu2+ | 390 | | | | | | ? |
| 30 | BC-420 | 391 | | | | | | ? |
| 35 | BC-414 | 392 | | | | | | ? |
| 25 | SrMgP207:Eu2+ | 394 | | | | | | N |
| 18 | BaBr2:Eu2+ | 400 | | | | | | N |
| 22 | (Sr,Ba)Al2Si2O8:Eu2+ | 400 | | | | | | |
| 5 | YTaO4:Nb(*) | 410 | 11 | 59.8 | 67.42 | 7.5 | Monolithic | N |
| 23 | Y2SiO5:Ce3+ | 410 | | | | | | N |
| 6 | CaWO4 | 420 | 5 | 61.8 | 69.48 | 6.1 | Tetragonal | N |
| 7 | LaOBr:Tb3+ | 420 | 20 | 49.3 | 38.92 | 6.3 | Tetragonal | N |
| 8 | Y2O2S:Tb3+ | 420 | 18 | 34.9 | 17.04 | 4.9 | Hexgonal | N |
| 13 | Lu2SiO5:Ce3+ | 420 | | | | | | N |
| 26 | Lu1.8Y0.2SiO5:Ce | 420 | | | | | | N |
| 9 | ZnS:Ag | 450 | 17 | 26.7 | 9.66 | 3.9 | Hexgonal | N |
| 29 | CdWO4 | 475 | | | | | | Slightly |
| 28 | Bi4Ge3O12(BGO) | 480 | | | | | | N |
| 10 | (Zn,Cd)S:Ag | 530 | 19 | 38.4 | 9.66/26.7 | 4.8 | Hexgonal | N |
| 11 | Gd2O2S:Tb3+ | 545 | 13 | 59.5 | 50.22 | 7.3 | Hexgonal | N |
| 12 | La2O2S:Tb3+ | 548 | 12.5 | 52.6 | 38.92 | 6.5 | Hexgonal | N |
| 31 | Y3Al5O12(Ce) | 550 | | | | | | N |
| 3 | LaOBr:Tm3+ | 360, 400 | 14 | 49.3 | 38.92 | 6.3 | Tetragonal | N |
| 32 | CaF2(Eu) | 435/500 | | | | | | N |

[symbol]

indicates data missing or illegible when filed

### X-Ray Delivery:

X-Ray energy can be delivered using orthovoltage sources, linear accelerators LINACs, hand held X-Ray imaging systems such as the one described in patent number U.S. Pat. No. 10,165,994 (the entire contents of which are incorporated herein by reference), dental Xray systems and X-Ray imaging systems including CT scanners and OBI in LINAC systems.

All of the flood beam X-Ray systems mentioned above can be used in conjunction with apertures to limit the spreading of X-Ray to undesirable areas around the target of interest. Such apertures exist and are widely available.

One additional modality described in the present invention for its precision entails the use of hollow metallic fibers to guide the X-Ray. Such fibers can be constructed by first coating a glass fiber using a metallic coating and building the metallic outer wall. The fibers can also be built by using a dip transfer technique of immersing the glass fiber inside a molten metal bath and pulling a fiber slowly enough to build a thick coating around its outer diameter. As the coated fiber leaves the molten metal vessel, the metal around the fiber cools and solidifies. A metallic jacketing can be formed in this manner.

Once the composited glass fiber and metallic jacketing is formed, the glass core can be etched away leaving a hollow metallic fiber. Once the X-Ray is coupled to the distal end of the fiber, the X-Ray energy remains guided in the metallic hollow core fiber by virtue of the grazing angles established in the hollow core.

Alternatively, a semiconductor crystal could be angularly rotated with one angular position passing a primary diffracted beam from the crystal through an x-ray aperture and an extreme angular position blocking the primary diffracted beam.

Regardless of how short x-ray pulses are generated, the incidence of these short x-ray pulses on a phosphor can promote generation of short-lived transient emissions from the phosphors in wavelength regions useful in the present invention for simulated biophoton emission.

### EC-PBM Effects on Biological Processes Common to Mammalian, Plant, Insects' Cells and Fish Cells.

One common denominator of all living cells is centered on energy production. For this reason, one embodiment of the present invention exposes Mitochondria to specific light emissions. In addition to Mitochondria, light exposure to angiogenesis and telomeres using experimental protocols specific to each is expected to yield information to either kill cells or foster growth, and thus may serve as another biomarker or sensor related to cell-to-cell communication. In other words, when cell-to-cell communication is expected to reduce a cell population, one may monitor for the “learned” light characteristics for less ATP production in the mitochondria indicating cell death. Conversely, when cell-to-cell communication is expected to increase a cell population, one may monitor for the “learned” light characteristics for more ATP production in the mitochondria indicating growth. Monitoring for light characteristics known to cause cell death can serve as a biomarker for cell-to-cell communication which induces cell death. Similarly, monitoring for light characteristics known to cause cell growth can serve as a biomarker for cell-to-cell communication which induces cell growth.

Five areas of specific application of the present invention include:
Mitochondria
Angiogenesis
Telomeres
Optical communication
Nerve growth/regeneration

### Constructive and Deconstructive Photonic Energy:

In one embodiment of the invention, photo-biomodulation (photo-stimulation) of various cells and tissues can identify which wavelengths of photonic energy are constructive and which are deconstructive to the mitochondria, angiogenesis, and telomeres. Viewed from this perspective, constructive photonic energy can be used to help grow healthy cells. These constructive wavelengths can be used for tissue regeneration or extending tissue heath to cite couple examples. On the other hand, de-constructive photonic energy can be used to prohibit the growth of cells which can be ultimately used for oncology treatments to reduce tumor burden or to arrest disease.

As above, monitoring for presence of constructive photonic energy at an untreated site may serve as a biomarker for cell-to-cell communication.

### Nerve Growth:

It has been established that neurons can emit photons. In one embodiment of the present invention, these biophotons/photonic communications may serve as signals between various neurons, in addition to the well-known electro-chemical signals. For such communication to be targeted, the low fluence of biophotons would likely travel in waveguides. It has been suggested, based on theoretical modeling, that myelinated axons could serve as photonic waveguides to facilitate/modulate such bio communication between nerve cells.

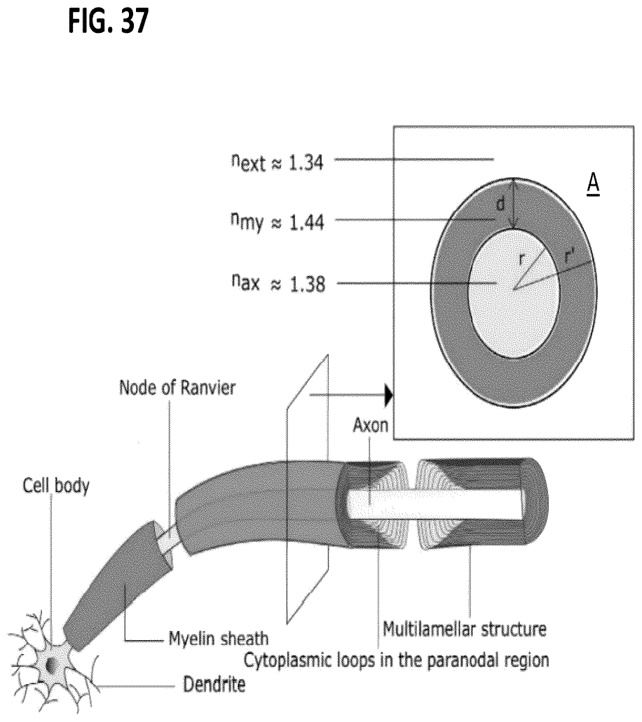

FIG. 37 is a schematic representation of a segment of a neuron, and an eigenmode of a cylindrical myelinated axon. Insert A shows different parts of a segment of a neuron whose myelinated axon is sliced longitudinally near the end of the segment. The inset depicts the cross section in the transverse plane. The compact myelin (shown in radial section) terminates in the paranodal region near the Node of Ranvier, with each closely apposed layer of myelin ending in a cytoplasm filled loop.

In one embodiment, the neurons are isolated with the necessary thickness of the myelin sheath, kept small in inhomogeneity, and suspended in a suitable solution to keep the cell alive for a time sufficient to couple one of the guided detectors into the axon, similar to the procedures adopted for verifying light guidance in other cells such as Muller cells. In one embodiment of the invention, by injecting light sensitive chemicals (e.g. AgNO3) either in the cytoplasmic loops in the paranodal region directly or in the oligodendrocytes, which would then circulate the chemical in the cytoplasmic loops, and possibly into some to the myelin too, an n-vivo detector light guidance (showing the presence of photons in the myelin sheath) is formed. Biophotonic signatures produced at the result of the photo oxidation of Ag+ (soluble ion) to Ag (insoluble metallic silver), would be visualized as dark granules or probed spectroscopically via SEM, or XPS.

In one embodiment of the present invention, the biophoton stimulation techniques described above applied inside the nerve would be useful in exploring and rendering therapeutic modalities for treating several neural based diseases including multiple sclerosis, Alzheimer's disease, Amyotrophic lateral sclerosis, Friedreich ataxia, Huntington's disease, Lewy body disease, Parkinson's disease and Spinal muscular atrophy, and provide for therapeutic application for nerve growth regeneration and making significant advances in the treatment of spinal cord injuries.

### The “Light Alphabet”

In one embodiment of the present invention the exogenous light signal consists of defined wavelength or wavelengths each having the desirable intensity and pulsation to induce a desirable biological change. The exogenous light input is hence encoded with the desirable information to induce the desirable change. Such encoded light is hereby referred to as the light alphabet, the photo-proteomic or the photonic signaling to drive a desirable biological change. This novel terminology is adopted to convey the signaling significance of the exogenous light input.

The most favorable exogenous light can be programmed into a computer using the appropriate software. The appropriate light alphabet input can therefore be delivered repeatably and reproducibly on demand using computer control.

Furthermore, when the exogenous light input induces a detectable endogenous light output leading to a favorable biological outcome, one optional course of action is to identify the waveform and intrinsic intensity of the endogenous light output and then program the exogenous light input to be the same as the endogenous light output. This process is called enrichment of endogenous signaling.

### Near Field Scanning Optical Microscopy

In one embodiment of the invention, the pulsed X-Ray sources described above can be used to stimulate down-converting particles on cell plates being monitored using a Near-field Scanning Optical Microscopy NSOM apparatus. A dedicated description is provided in the following paragraphs.

Near-Field Scanning Optics (NSOM) is conducted to obtain optical images with point-to-point resolution greater than that determined by the wavelength of light, approaching 10 nm for light of 350 to 650 nm. Light is transmitted down a fiber that has a tapered end that is metal coated. The taper can be selected to be far smaller than the wavelength of the light trapped in the fiber. As the super-constrained light beam emerges from the end of the fiber, its spatial expansion, which normally would decrease the light intensity as 1/r², is much more severe and the intensity decreases as nearly 1/r⁶. This makes every spot of the light have a very weak intensity except at the exact point where it exits the fiber. Thus, an imaging system would only pick up any illumination at exactly the position of the end of the fiber. As the fiber end is moved across a sample, spots are picked only from the positions of the fiber during a scan. Therefore, an image is formed using very small pixels, each coming from spots along the scan of the end of the fiber over the surface of interest. If the fiber is moved with an atomic force microscope, the positioning of the fiber and therefore the pixels have the resolution of the moving piezoelectric which can be as small as every 10 nm. Thus, the spatial resolution of the image is on the order of 10 nm and is capable of detecting morphological changes to cancer cells (for example) and provide another type of biomarker indicative of cell-to-cell communication to an untreated region.

In the present invention, there are provisions where the delivered light spot is in the order of an entire cell averaging 100 microns in diameter. In this manner, an entire cell can be flooded with a single wavelength or a broad spectrum light using controlled intensities of light having pulsations spanning from the nano seconds all the way to continuous waves.

In the present invention there are provisions for treating a group of cells all at once by utilizing fiber bundles to deliver single wavelengths or a broad-spectrum light using controlled intensities of light having pulsations spanning from the nano seconds all the way to continuous waves.

### RAMAN Spectroscopy on Individual Cells and Subcellular Components

Raman spectroscopy of individual cells can be performed using confocal microscopes, equipped with a CCD detector mounted on a high throughput holographic Raman spectrometer. However, in order to discriminate between Raman signals coming from different components of the cell, such as the nucleus or the mitochondria, it is preferable to use NSOM Raman spectroscopy. Here, as described above, laser light is precisely delivered to a spot that is on the order of 10 nm in size by using the NSOM microscope described above. The detection is done over the entire half sphere around the source in order to maximize the amount of light detected. By scanning the end of the fiber, Raman signals from different subcellular components can be differentiated and detected.

Raman signals are well known for various biological molecules and proteins, as well as the products of the mitochondria, TPA and DPA, which could serve as another example of biomarkers of the present invention indicative of cell-to-cell communication to an untreated region.

### Laser and X-Ray Excitation on Organ Tissue

In one embodiment of the present invention, simulation of biphoton emission and/or stimulation of a target site may use laser excitation of cells, and organ tissue in the UV. On such laser could be an off-the-shelf diode-pumped, pulsed Nd-YAG laser operating at 1,064 nm that is doubled twice to obtain 266 nm UV light.

Applications that require shorter wavelengths if needed will be obtained from the same diode-pumped Nd-YAG laser but using frequency multiplication of 5 to get pulses at 213 nm (as shown in FIG. 38). Pulse lengths of several tens of nanometers will be produced.

Typical conditions for producing stimulant/simulant light would be Nd:YAG source—1064, 532, 355, 266, 213 nm (EKSPLA NL202 or 204)—pulse duration 7-10 ns at a pulse rate of 1-1,000 Hz with 0.6 mJ at 266 nm and 0.3 mJ at 213 nm.

If necessary to reach shorter wavelengths for the stimulant/simulant light, excimer lasers may used. An excimer laser typically uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range.

ArF and KrF excimer lasers are widely used in high-resolution photolithography machines, a critical technology for microelectronic chip manufacturing. An excimer laser, which emits deep ultraviolet wavelengths consists of two elements, krypton or argon and fluorine, as the lasing medium. The principle behind the working of the laser is that krypton or argon absorbs energy from the power source and reacts with fluorine gas to produce krypton fluoride or argon fluoride as temporary complexes.

For example: An argon fluoride laser absorbs energy from a source, causing the argon gas to react with the fluorine gas producing argon monofluoride, a temporary complex, in an excited energy state:

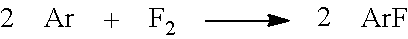

The complex can undergo spontaneous or stimulated emission, reducing its energy state to a metastable, but highly repulsive ground state. The ground state complex quickly dissociates into unbound atoms:

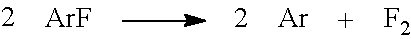

The result is an exciplex laser that radiates energy at 193 nm, which lies in the far ultraviolet portion of the spectrum, corresponding with the energy difference of 6.4 electron volts between the ground state and the excited state of the complex.

These lasers can be used to create electron-hole pairs in phosphors resulting in UV and optical emissions. The advantages of these lasers are their rapid pulsing capabilities. Typical conditions for producing stimulant light would be: ArF Excimer laser 193 nm (Xiton photonics IXIO 193 XLM) diode-pumped laser with Q-switch, <10 ns pulse duration at 6 kHz rate with 0.0016 mJ per pulse.

### Photo-Spectrometers:

For UV studies. a monochromator and spectrometer may be used to detect bio-photon emissions. Deep UV spectrometers exist off the shelf from J-Y Horiba that use CCD and can detect wavelengths as short as 150 nm. (Horiba VU90 spectrometer) CCD detectors with 115-320 nm range. These spectrometers operate either in vacuum or in pure nitrogen gas flush. Sample temperature control is critical and will be accomplished with a Peltier controller.

Fluorescence spectroscopy can be performed. Continuous N2 flush will be necessary. Temperature control via Peltier will be advantageous. Conventional spectroscopy with orthogonal detection may be useful—as in Fluorescence.

### Transient Wavelengths and Time Resolved Measurements:

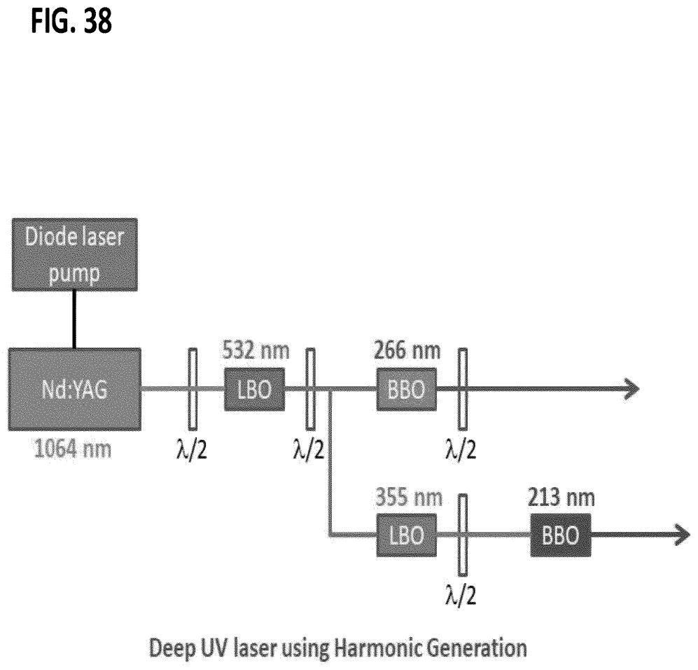

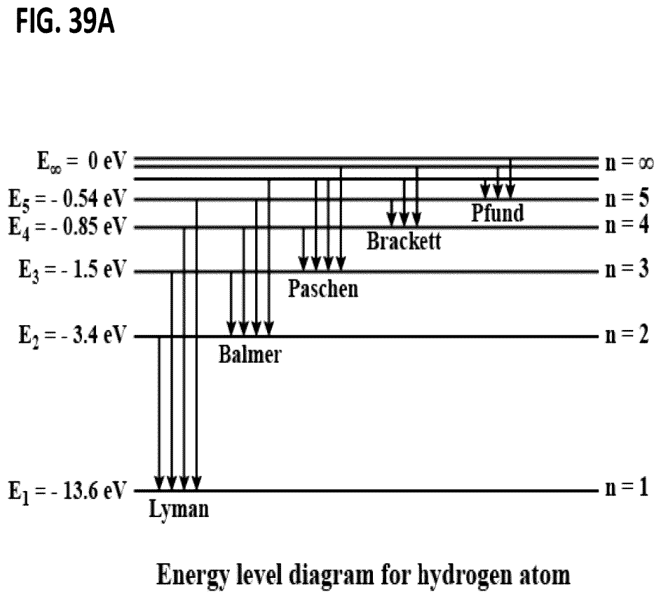

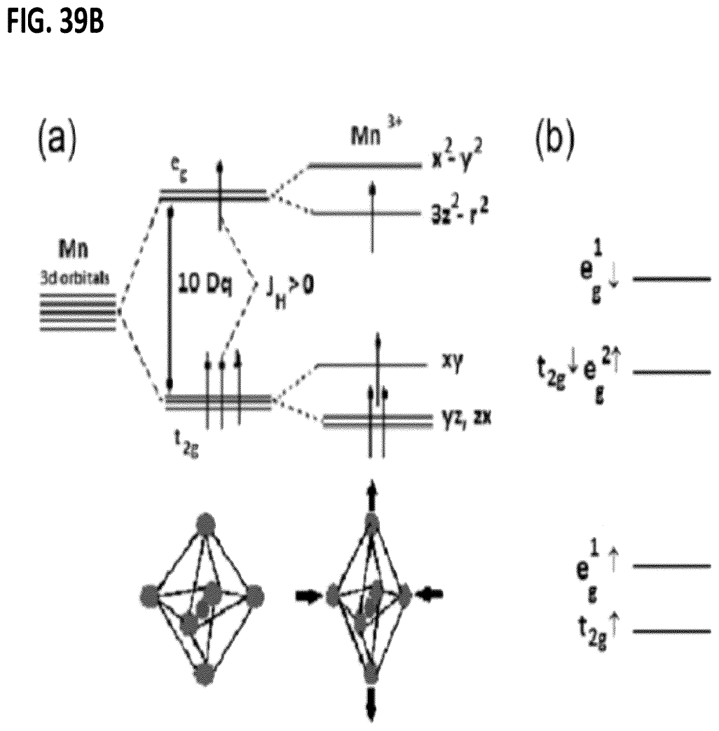

In one embodiment of the invention, low intensity transient emissions are utilized as a source stimulating a biological change or as an artificial source of biophoton emissions (that is a biophoton simulant). Some of transient emissions can be caused when an X-Ray photon overcomes the binding energy of an inner core electron which gets ejected from the atom and is immediately replaced by an electron from an outer shell electron (of lesser energy). The transitions within the atom from the possible excited states to the ground states give rise to various transient emission. In fact, a series of emissions can take place all of which are short lived. The energy diagram of the simplest atom (i.e. Hydrogen) is illustrated in FIG. 39A:

An electron cloud may be considered a spherical distribution of charge centered around the atomic nucleus. As such the electrons are bound to the positively charged nucleus by electrostatic forces. These electrostatic forces act as springs to restore the electrons to their shell if they shift due to thermal randomization for example or due mechanical impacts that impart vibrational motions (phonons).

Atoms however are found in gases, liquids or solids. Solids are typically either amorphous, crystalline or polycrystalline. In a crystalline and polycrystalline lattice structures an atom is retained in place with various biding forces. These forces retain the atom in place as part of the large atomic grouping forming the lattice. Phonon vibrations across a lattice structure induce cooperative movements amongst large atomic groupings. There is also the establishment of harmonics or subharmonics above and below the natural frequency of vibration of a harmonic oscillator; if, the conditions allow for their establishment. The emissions of the electrons inside materials can measured which confirm their transitions across various energy levels.

In one embodiment of the invention, transient biophotons are measured and monitored (e.g. at low frequency and fluence.) In turn, artificially produced photons can mimic the observed characteristics. Due to biological constraints of photonic transduction in biological waveguides (i.e. thickness, composition, bends, refractive index changes due to lipids, etc . . . ) but also related to intrinsic energy states resident in the color centers, it is expected that many simulated biphotons will have a short transient characteristic. In a specific example, a molecular energy diagram for the transition metal manganese is shown in FIG. 39B. While these ionic species may reside in multiple oxidation states (i.e. Mn+2, Mn+3, Mn+4, Mn+5. Mn+7, electronic transitions in each oxidation state for these ionic species can be described by a specific energy/molecular orbital diagram as indicated in FIG. 39.

These diagrams can be perturbed via environmental crystal field splitting. In this example, crystal field splitting of the Mn ion d-levels and electronic occupation of the mixed-valence Mn ions lifts the degeneracy of the e g and t 2 g levels by an in-plane contraction and out-of-plane elongation of the oxygen octahedron. This now results in potentially new color centers that may yield unexpected photonic emissions depending upon the extent of the crystal field splitting of the environment. To explain transient and low fluence emissions, it is well established, spectroscopically, that color centers with different electronic energy levels, and transitions between such levels often involve the emission or absorption of light (photons). An absorbed photon can deliver the energy for an atom or ion to get into a higher-lying energy level, whereas spontaneous or stimulated emission releases energy which was previously stored in the atom or ion, as might occur in laser transitions.

The likelihood of such transitions depends on the electronic levels involved. Strong transitions are those where certain molecular/electronic “selection rules” are satisfied. In one specific example, dipole transitions can occur only between energy levels with the angular momentum parameter 1 differing by one. Therefore, dipole transitions between energy levels with same parity are not allowed, i.e. they are forbidden. The mechanism is not binary—there are “weakly allowed” transitions.

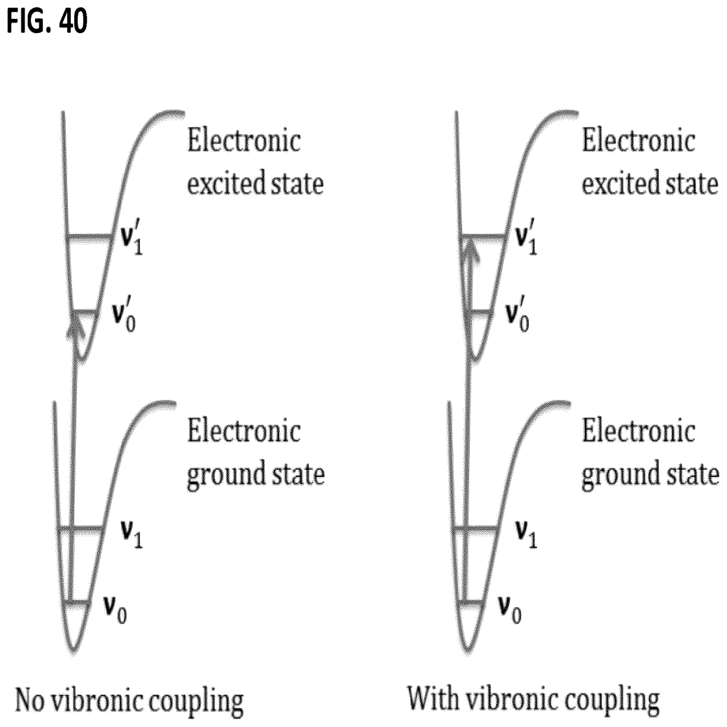

Dipole-forbidden transitions between energy levels may nevertheless occur based on other mechanisms such as quadrupole transitions. Environmental factors can and do influence photonic emissions. For example ions embedded in a crystal lattice or in a glass, internal electric and magnetic fields can break certain symmetries, so that e.g. originally dipole-forbidden transitions become possible by mixing of states with different parity. Such processes, however, are usually much less likely, i.e., they exhibit a small oscillator strength (and subsequent low intensity and transient emission). These resulting transitions are sometimes called weakly allowed transitions rather than forbidden transitions, because there are mechanisms for such transitions, although not strong ones. While such transitions may be of the order of a few nanoseconds, some color centers have lifetimes of milliseconds or even many seconds, and ions in crystals or glasses typically between microseconds and milliseconds. Such long-lived levels are called metastable states resulting in low frequency, transient and low intensity photonic emissions. Additionally, productive interactions between electronic states and vibrational states can produce new emissions—so called vibronic coupling—and can lead to new, unexpected emissions with variable photonic lifetimes and intensities. FIG. 40 is a representative set of such energy band diagrams.

### Time Resolved Spectroscopy:

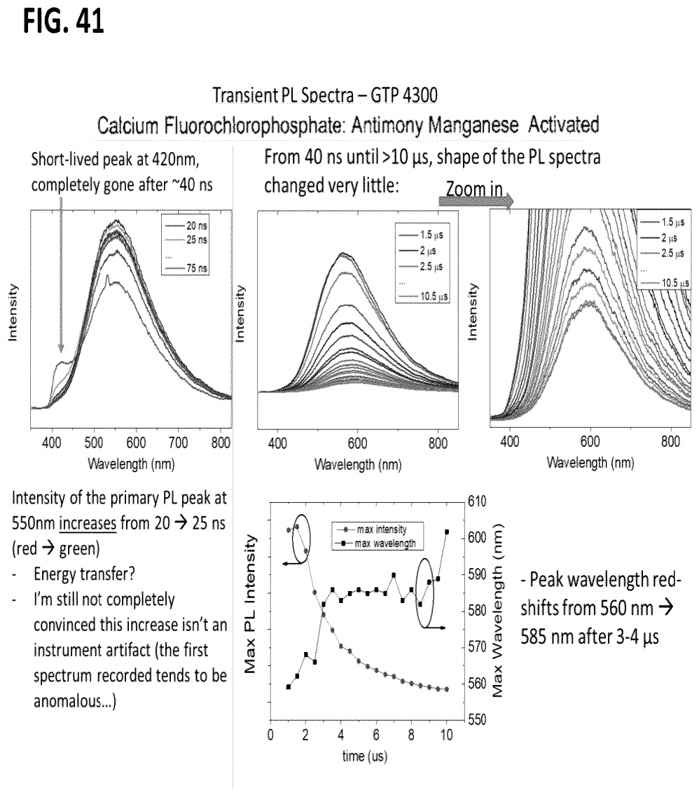

FIG. 41 describes cascade of events and which energy states feed the other.

When exposed to high energy EM waves, such as UV and x-ray sources, biological specimens are expected to exhibit very low level transient light coming from the specimens. These emissions can be measured with time resolved spectroscopy. Here, a photomultiplier tube with rapid electronics is used to replace the CCD detectors in the spectrometers for nanosecond and longer processes. The laser sources are Q-switched and therefore produce 10 ns pulses.

Pulse the X-Ray Source: (Anton Paar—PRIMAX 100 50W with a Spot Diameter of 45 Microns)

Pulsing an x-ray beam can be used in one embodiment of the invention. The pulsed x-ray beam can produce pulses by rapid changes in the reflectivity of x-ray mirrors to thereby produce pulses on the order of 100 ns.

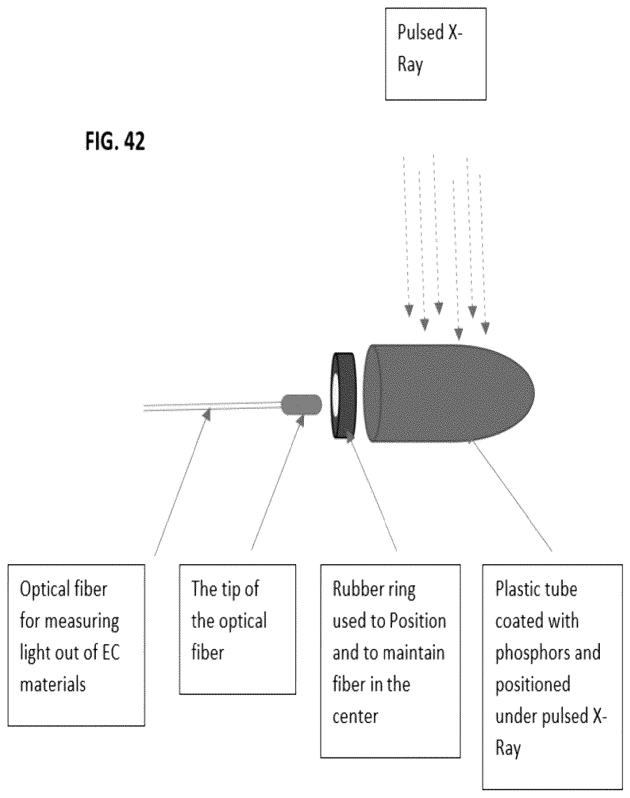

FIG. 42 is a diagram of an apparatus set up to best measure light emission of EC under pulsed x-ray beam production. A plastic tube is coated with slurry of phosphors. The slurry is dried and the solvents are evaporated leaving behind a 10 micron coating on the inner walls of a test tube. The optical probe is inserted using a positioning ring. This set up maximizes the measurement of optical emissions under Xray energy.

### Oxidative Stress:

One aspect of this invention deals with a direct counter action of oxidation using specific materials capable of acting as antioxidants. Severe reduction in telomere length has been linked to oxidative stress of the mitochondria.

Reduction of ROS (reactive oxygen species) mediated by H₂O₂ bio-photo—modulation of telomere length:

The presence of H₂O₂ in many biological environments can lead to the formation of ROS via a variety of mechanisms. In one specific reaction Ferrous iron (Fe⁺²) can catalyze the decomposition of H₂O₂ via the formation of highly reactive hydroxide radical and concomitant production of hydroxide ion.

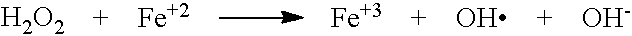

A similar decomposition reaction might be expected in the presence of light leading to the formation of the reactive superoxide anion O₂⁻:

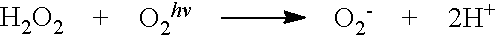

In either case, the generation of ROS species is dependent upon the presence of hydrogen peroxide—thus a reduction in the level of H₂O₂ could mitigate the formation of these undesirable species.

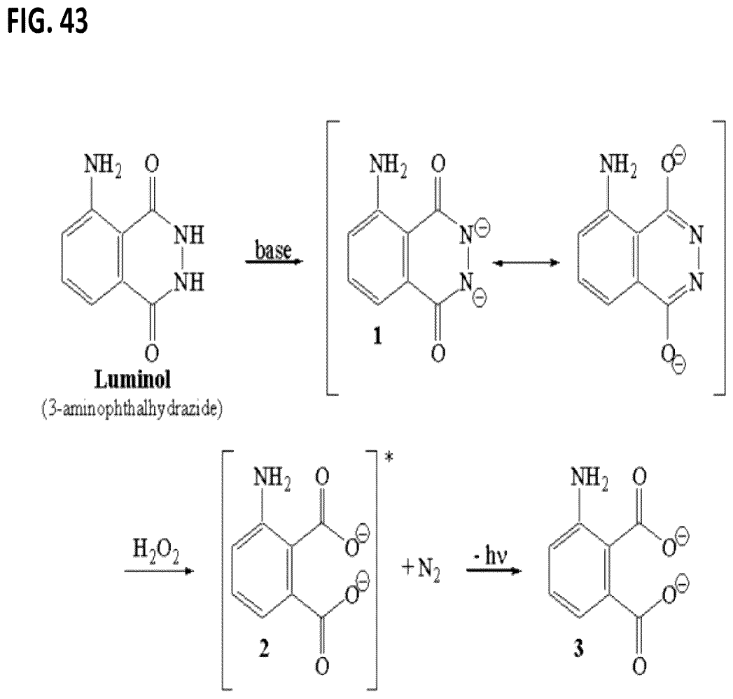

In one embodiment of the invention, ultra sensitive detection of photons emitted upon the chemical conversion of luminol to it corresponding di-carboxylic acid with the production of a photon of light. This chemical conversion is shown in FIG. 43

Luminol is converted by the basic solution into the resonance-stabilized dianion 1, which is oxidized by the hydrogen peroxide into the dicarboxylate ion 2, accompanied by the loss of molecular nitrogen, N₂. When the molecule 2 is formed, it is in an excited (higher energy) electronic state, and sheds its “extra” energy by emitting a photon of light (hn), allowing the molecule to go to its ground state form (3). The use of a photometer can be used to detect and quantify the amount of light produced and subsequently estimate the reduction in the amount of H₂O₂ since the reaction is stoichiometric with respect to peroxide concentration. Depending on the extent of solution buffering, a blue glow produced by this reaction can persist for a couple of minutes.

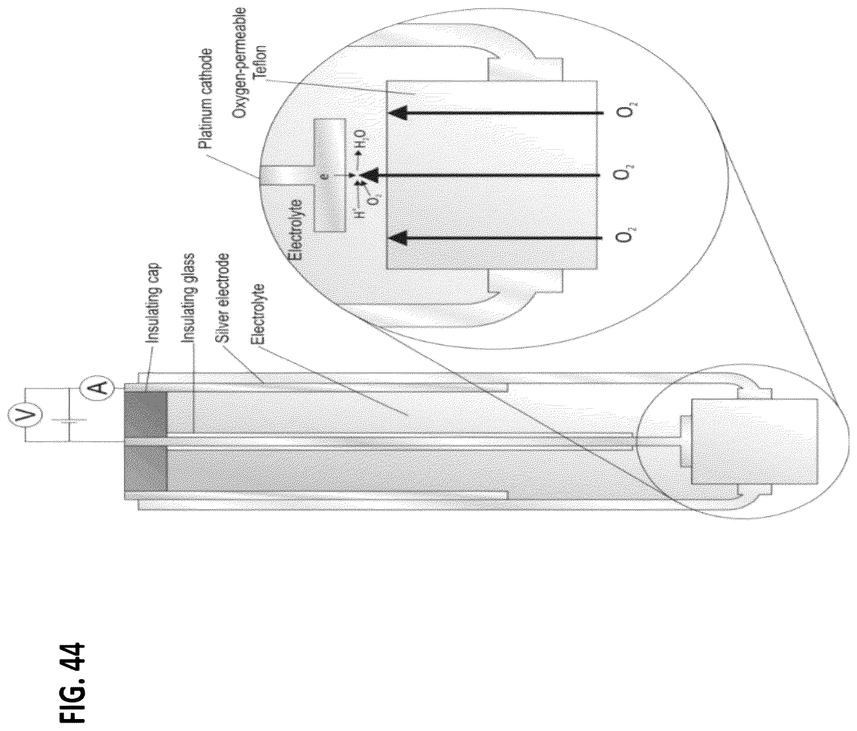

In another example, the decomposition of H₂O₂ can be monitored via the use of a commercially available electrochemical oxygen electrode as shown in FIG. 44 and often referred to as a Clark electrode. The Clark electrode is an electrode that measures ambient oxygen partial pressure in a liquid using a catalytic platinum surface according to the net reaction:

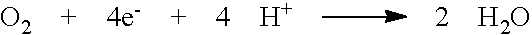

In the peroxide, in the presence of phosphors and X-Ray irradiation, molecular oxygen should be generated:

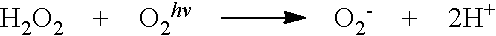

The amount of molecular oxygen produced is directly related the molar concentration of peroxide, and thus a reduction of peroxide in the aqueous solution can be quantified.

These types of reactions and the monitoring of hydrogen peroxide levels could be used in the cuvettes described above or in a in vivo biphoton probe, where a living cell source of cell-to-cell communication (e.g., an isolated container of cancer biopsy materials from the patient being treated) would be “dosed” with hydrogen peroxide to generate cell death in the isolated container), thereby inducing cell death in an untreated tumor nearby. In one embodiment of the present invention, the monitored hydrogen peroxide levels in the isolated container would be an indirect measure of the expected cell death in the tumor.

### Restoring a Healthy Equilibrium in Living Tissue:

In a review titled “The Give-and-Take Interaction Between the Tumor Microenvironment and Immune Cells Regulating Tumor Progression and Repression” authored by Simon Pernot, Serge Evrard and Abdel-Majid Khatib* a summary of how deregulations at the single cell level may contribute to global cancer initiation, progression and dissemination is provided. Citation: Frontiers in Immunology|www.frontiersin.org 1 Apr. 2022|Volume 13|Article 850856.

The authors indicate that it is now evident that cancer cells disrupt the rules and function of normal cells. Indeed, cancer cells divide and proliferate when is not necessary, do not die when is required, take advantage of the resources of other normal cells and perturb the harmony of the normal tissue environment. Furthermore, while the collaborating “normal” cells have limited proliferative capacity, tumor cells can resist to cell death and escape from the immune system.

In this review the authors address the importance of the major cellular interactions in the tumor microenvironment that control tumor cells and how these interactions influence the causality of cancer and neoplastic evolution. The authors considered the following: 1—Dynamic Reciprocity Denotes bidirectional communication in all kinds of cells, involving specifically the nucleus and cellular extracellular matrix (ECM) elements & 2—Reciprocal Cellular Interaction Bidirectional interaction between cells and their microenvironment.

During this interaction, cells within a specific tissue express and produce a panel of signals/mediators to which other tissues and cells can react and reply. In turn, the responding cells produce distinct signals to which the signaling cells also respond. In this interaction, all the involved cells are signaling and responding elements. WHAT KIND OF SIGNALS?

Tumors are considered as new developing organs that contain both malignant cells and a wide range of non-malignant cells that lose their ability to continuously preserve tissue homeostasis and architecture. The main non-malignant cells that participate in the constitution of the tumor tissue are immune cells (e.g. macrophages/TAMs, NK, and T cells), fibroblasts/CAFs, endothelial cells and adipocytes/CAAs (referenced publication by Hanahan D, Coussens L M. Accessories to the Crime: Functions of Cells Recruited to the Tumor Microenvironment. Cancer Cell (2012) 21:309-22. doi: 10.1016/j.ccr.2012.02.022).

Communication between cancer cells and these non-malignant cells seem to control the outcome and the evolution of the developed tumors. In normal tissues the cellular interactions can be mediated through physical communication involving receptors and/or various ECM constituents and ECM-imbedded enzymes and through diverse range of mediators produced by the involved interacting cells (referenced publication by Burnet F M. The Concept of Immunological Surveillance. Prog Exp Tumor Res (1970) 13:1-27. doi: 10.1159/000386035). These include growth factors, chemokines and cytokines and other mediators of which the expression and production are strictly regulated (referenced publication by Ungefroren H, Sebens S, Seidl D, Lehnert H, Hass R. Interaction of Tumor Cells With the Microenvironment. Cell Commun Signal (2011) 9:18. doi: 10.1186/1478-811X-9-18). The proteolytic activation of these molecules or expression was reported to be also required for cancer cell interaction with the TME. Thereby, the disturbance of the tissue homeostasis generates changes in the activity and the function of the communicating cells. In turn these changes affect cell proliferation, migration and survival leading to tumor progression or repression.

By infiltrating tumors, immune cells mediate cytotoxic effect on cancer cells. The immunological surveillance is in play. Neo-antigens expression on tumor cells are able to induce immunological events against cancer cells and if successful, regression of the tumor takes place, and no clinical hint of its existence remains. However, the efficacy of the tumor-infiltrating immune cells can be evaded by cancer cells using complex mechanisms where various immune cells are driven to participate in tumor initiation and progression. The interaction of immune cells and cancer cells are mainly summarized in three major phases (reference publication by Anderson K G, Stromnes I M, Greenberg P D. Obstacles Posed by the Tumor Microenvironment to T Cell Activity: A Case for Synergistic Therapies.) These include: 1—the cancer cells clearance phase or elimination, where the immune cells are able to eradicate the newly transformed cells, 2—the equilibrium phase, that corresponds to a balance between the eradicated and the newly formed cancer cells and 3—the escape phase characterized by the accumulation of tumor-cell variant subpopulations (or clones) occurred during the equilibrium phase. This process is the direct consequence of the heterogeneity of the transformed cells that subsequently results in the development of cellular mechanisms allowing immune cells escape or suppression. The generated cancer cell clones increase their ability to grow and proliferate in an immunocompetent environment.

In the reference publication by Dunn G P, Old U, Schreiber R D. The Three Es of Cancer Immunoediting. Annu Rev Immunol (2004) 22:329-60. doi: 10.1146/annurev. immunol. 22.012703. 104803, the authors discuss how, after a century of controversy, the notion that the immune system regulates cancer development is experiencing a new resurgence. An overwhelming amount of data from animal models—together with compelling data from human patients—indicate that a functional cancer immunosurveillance process indeed exists that acts as an extrinsic tumor suppressor. However, it has also become clear that the immune system can facilitate tumor progression, at least in part, by sculpting the immunogenic phenotype of tumors as they develop. The recognition that immunity plays a dual role in the complex interactions between tumors and the host prompted a refinement of the cancer immunosurveillance hypothesis into one termed “cancer immunoediting.” In the referenced review, the authors summarize the history of the cancer immunosurveillance controversy and discuss its resolution and evolution into the three Es of cancer immunoediting—elimination, equilibrium, and escape.

### The Elimination Phase:

In the elimination phase, immune cells such as T cells and macrophages are able to distinguish tumor cells from normal cells through expression of specific molecules such as the ligands for NKG2D on tumor cells. Throughout cell transformation, the release of various proinflammatory molecules and chemokines by tumor cells is enough to activate the innate immune system. Following cancer cells recognition, the immune cells secrete various molecules such as IFN-g and perform that eliminate the emerging tumor cells. These processes are then gradually increased leading to high production of cytokines and chemokines that permit the recruitment of more immune cells. In turn, the activated immune cells participate in the accumulation of cytotoxic products such as perform, reactive oxygen or TRAIL. Thereby, this positive loop of immune cells recruitment and activation enhances the generation of tumor antigens of dead tumor cells. Subsequently, the generated antigens induce the activation of the adaptive immune system that in turn participate in the ongoing cancer cells elimination process. Thus, this phase of cancer cells and immune cells interaction is an uninterrupted mechanism where the newly formed cancer cells expressing specific markers and cytokines are identified by immune cells and eliminated.

One aspect of this invention is to utilize at least one wavelength of light and the appropriate treatment regimen to promote the secretion of molecules such as IFN-g and perform that eliminate the emerging tumor cells. Tracking the presence of these or similar molecules at an untreated site may serve as a biomarker of cell-to-cell communication in the present invention.

### The Equilibrium Phase:

The T lymphocytes are the main immune cells involved in the equilibrium phase where they secure the preservation of this cancer cells and immune cells communication. Cytokines like IFN and TNF were also found to be involved during these interactions (Müller-Hermelink N, Braumüller H, Pichler B, Wieder T, Mailhammer R, Schaak K, et al. TNFR1 Signaling and IFN-Gamma Signaling Determine Whether T Cells Induce Tumor Dormancy or Promote Multistage). In addition, IFN was found to be a key player in the progression from the elimination phase to equilibrium phase through its selective immunoediting pressure. Despite the elimination of abundant emerging tumor cells, several tumor cell variants with different mutations may resist and survive to the activated immune cells. During this period, the heterogeneity and genetic instability of cancer cells that subsist the elimination phase are the main causes. Of these processes the nucleotide-excision repair instability (NIN), microsatellite instability (MIN), and the chromosomal instability (CIN) are the major genetic instability that were found to provide tumor cells variants with reduced immunogenicity and/or ability to grow in a highly immunocompetent environment. The altered environment with accumulated cytokines and hypoxia also favors immunosuppression leading to the escape phase.

As summarized by Muller-Hermelink N et. al. Immune responses may arrest tumor growth by inducing tumor dormancy. The mechanisms leading to either tumor dormancy or promotion of multistage carcinogenesis by adaptive immunity are poorly characterized. Analyzing T antigen (Tag)-induced multistage carcinogenesis in pancreatic islets, we show that Tag-specific CD4+ T cells home selectively into the tumor microenvironment around the islets, where they either arrest or promote transition of dysplastic islets into islet carcinomas. Through combined TNFR1 signaling and IFN-γ signaling, Tag-specific CD4+ T cells induce antiangiogenic chemokines and prevent αvβ3 integrin expression, tumor angiogenesis, tumor cell proliferation, and multistage carcinogenesis, without destroying Tag-expressing islet cells. In the absence of either TNFR1 signaling or IFN-γ signaling, the same T cells paradoxically promote angiogenesis and multistage carcinogenesis. Thus, tumor-specific T cells can directly survey multistage carcinogenesis through cytokine signaling. Tracking tumor-specific T cells at an untreated site may serve as a biomarker of cell-to-cell communication in the present invention.

### The Escape Phase:

Tumor cell variants derived from the equilibrium phase carry various genetic changes that confer cancer cells resistance to immune detection and elimination, allowing the tumors to expand and grow. At this phase, cancer cells use various strategies to avoid the immunosurveillance of the innate and/or adaptive immune system. The cancer cells can repress the anti-tumoral immune responses through the production of immunosuppressive cytokines such as TGF-b and IL-10 or through the recruitment of T cells with immunosuppressive activities such as the regulatory T cells, MSCs and MDSCs (Khong H T, Restifo N P. Natural Selection of Tumor Variants in the Generation of “Tumor Escape” Phenotypes. Nat Immunol (2002) 3:999-1005. doi: 10.1038/ni1102-999). The alterations that occur on tumor cells can also affect tumor recognition by immune cells. These include altered cell surface antigen expression, loss of MHC elements, liberation of NKG2D ligands, and resistance to IFN-g effect. The expression of aberrant Cancer cells and immune cells communication. From immune surveillance to immune escape, exposure to internal and/or external risk factors induced cell transformation (I) of which the development is repressed by intrinsic tumor suppressing mechanisms (e. g. tumor suppressor genes and apoptosis) (II). During the elimination phase, immune cells such as natural killer cells (NK), and T cells are able to recognize (e.g. through presence of NKG2D ligand) and eliminate tumor cells. The equilibrium phase involves the continuous elimination of tumor cells by immune cells secreting cytotoxic agents (INF, perforin and others) and the accumulation of resistant cancer cell variants (III). At the escape phase, as a result of heterogeneity, tumor cells that are less immunogenic are able to escape immunosurveillance and secrete cytokines and chemokines that recruit immunosuppressive cells (Tregs, MSCs and MDSCs), which suppress the antitumor immune responses through different pathways including T cell and NK cell activity repression (IV). Pernot et al. Reciprocal Interaction in Tumor Microenvironment Frontiers in Immunology|www.frontiersin.org 4 Apr. 2022|Volume 13|Article 850856 antigens on tumor cells also affects the anti-tumoral response by inhibiting the proliferative response of immune cells. Production of MSCs with potent immunosuppressive function may also contribute to inactivation of T cells through various mediators such as nitric oxide (NO) that limit the proliferation and mediate apoptosis of T cells. Tracking cytokines and chemokines at an a treated site and at an untreated site. serves as a biomarker of cell-to-cell communication in the present invention.

As summarized by Khong H T et. al., the idea that tumors must “escape” from immune recognition contains the implicit assumption that tumors can be destroyed by immune responses either spontaneously or as the result of immunotherapeutic intervention. Simply put, there is no need for tumor escape without immunological pressure. “Natural selection” of heterogeneous tumor cells results in the survival and proliferation of variants that happen to possess genetic and epigenetic traits that facilitate their growth and immune evasion. Tumor escape variants are likely to emerge after treatment with increasingly effective immunotherapies. Tracking the tumor escape variants at a treated site and at an untreated site. serves as a biomarker of cell-to-cell communication in the present invention.

### Cancer Associated Fibroblasts:

In normal tissues, fibroblasts are responsible for tissue integrity. These cells can reply to tissue damage by their differentiation to myofibroblasts that in turn coordinate the wound healing process and the repair of the damaged tissue. These biological functions are mediated by ECM synthesis and remodeling and through the permanent fibroblasts interaction with immune cells. Fibroblasts within the tumor microenvironment that exhibit a cancer-associated phenotype are denoted as “cancer-associated fibroblasts” or CAFs. Tracking fibroblasts at an untreated site. serves as a biomarker of cell-to-cell communication in the present invention.

### Pro-Tumorigenic Function of CAFs:

The secreted molecules by the CAFs are the main driver of their pro-tumorigenic function. Of the mediators involved in the cross talk between CAFs and cancer cells are various chemokines (e.g. CXCL12, CCL7), growth factors (TGFbs, FGFs and HGF) and several ECM proteins (collagens and proteoglycans). These molecules induce tumor progression by directly increasing cancer cell survival, proliferation, stemness, and the acquisition of the metastatic phenotype. On the other hand, cancer cells also secrete similar molecules that stimulate CAFs activity and thereby leading to the generation of a set of signaling activities converging to the promotion of tumor progression. The cancer cells and CAF-derived molecules can also function in a positive feedback loop to increase and maintain CAFs activation. For example, both CAFs and cancer cells are able to secrete LIF to activate CAFs and their ECM remodeling that favors CAFs migration together with cancer cells in a combined manner. CAFs are also involved in the induction and enhancement of angiogenesis through their ability to produce and secrete various proangiogenic factors such as VEGF. Similarly, through their ability to secrete inflammatory molecules such as IL-1, IL-6 and TNFa, CAFs participate in inflammation-mediated tumor progression. Various of the CAFs secreted molecules participate as well in the generation of an immunosuppressive microenvironment that suppresses the immune system activity and favors tumor escape and progression. Furthermore, CAFs contribute to tumor progression through their role in the regulation of the metabolic activity within the tumor microenvironment. By direct contact with cancer cells CAFs can also facilitate metastasis. CAFs were found to mediate invasion of cancer cells through generation of migratory tracks in the ECM matrix where cancer cells follow CAFs to mediate cooperative invasion.

One aspect of this invention is to utilize at least one wavelength of light and the appropriate treatment regimen to inhibit the cross talk between CAFs and cancer cells which is mediated by various chemokines (e.g. CXCL12, CCL7), growth factors (TGFbs, FGFs and HGF) and several ECM proteins and the reduction or inhibition of pro inflammatory molecules such as IL-1. IL-6 and TNFa. Tracking these or similar species at an untreated site. serves as a biomarker of cell-to-cell communication in the present invention.

### Antitumor Function of CAFs:

Compared to the pro-tumorigenic function of CAFs, several secreted CAF mediators can exert antitumor functions such as IFNg. This factor promotes an anti-tumoral immune pressure against cancer cells through the recruitment of immune cells and their secreted molecules. Thus, the CAFs seemed to have a dual role probably linked to their heterogeneous populations and the nature of their secreted mediators.

### Angiogenesis: Endothelial Cells and Cancer Cells Interaction

Cancer cells need nutrients and oxygen derived from their close blood vessels in order to survive and proliferate. Tumors attain their vascularization by inducing the formation of new vascular vessels, a process known as tumor angiogenesis, or by using pre-existing vasculature, a phenomenon described as vascular co-option (Kuczynski E A, Vermeulen P B, Pezzella F, Kerbel R S, Reynolds A R. Vessel Co-Option in Cancer. Nat Rev Clin Oncol (2019) 16:469-93. doi: 10.1038/s41571-019-0181-9).

Kuczynski E A et. al, summarized that it was suggested that an attribute of tumor cells is their capacity to elicit continuously the growth of new capillary endothelium in vivo. Subsequently, Greene observed that tiny tumors implanted for more than a year in the anterior chamber of the guinea pig eye would not grow because they could not become vascularized. When these tumors were reimplanted in the muscle of a rabbit where they could become vascularized, they grew to a large size. The growth of tumors which have been implanted in any one of several different organs and maintained by a long-term perfusion stops when the tumor reaches a diameter of 3-4 mm. Further growth of tumor tissue in in vitro organ cultures cannot be sustained without neovascularization of the tumor. Neovascularization does not require direct contact by tumor cells since vessels have been elicited from the hamster cheek pottch by tumors contained in a Millipore filter. Similar outgrowth of new blood vessels was observed by Kuczynski when Millipore chambers containing cells of B-16 melanoma or Walker carcinoma were implanted into the dorsal air sac of rats. In their publication, the isolation of a soluble factor from human and animal neoplasms which is mitogenic for capillary endothelium is described. This factor induces growth of new capillaries, which may be responsible for tumor angiogenesis.

Tumor neovascularization can also affect the microenvironment of the growing tumor and cause tumor immunosuppression by recruiting immunosuppressive cells, and inhibiting cytotoxic T cell activity through angiogenic factors (Martin J D, Seano G, Jain R K. Normalizing Function of Tumor Vessels: Progress, Opportunities, and Challenges. Annu Rev Physiol (2019) 81:505-34. doi: 10.1146/annurev-physiol-020518-114700). In turn, the activated tumor microenvironment releases a large number of factors that promote tumor angiogenesis, establishing a tumor growth-promoting cycle. In addition, the formed tumor vessels are immature with high permeability and hypoxia that further facilitates tumor growth and metastasis.

Martin J D et. al. summarized that Abnormal blood and lymphatic vessels create a hostile tumor microenvironment characterized by hypoxia, low pH, and elevated interstitial fluid pressure. These abnormalities fuel tumor progression, immunosuppression, and treatment resistance. In 2001, the authors proposed a novel hypothesis that the judicious use of anti-angiogenesis agents—originally developed to starve tumors—could transiently normalize tumor vessels and improve the outcome of anticancer drugs administered during the window of normalization. In addition to providing preclinical and clinical evidence in support of this hypothesis, we also revealed the underlying molecular mechanisms. In parallel, we demonstrated that desmoplasia could also impair vascular function by compressing vessels, and that normalizing the extracellular matrix could improve vascular function and treatment outcome in both preclinical and clinical settings. Here, we summarize the progress made in understanding and applying the normalization concept to cancer and outline opportunities and challenges ahead to improve patient outcomes using various normalizing strategies.

One aspect of this invention is to utilize at least one wavelength of light and the appropriate treatment regimen to foster the right environment to improve the outcome of administered anticancer drug. Tracking the tumor regression at an untreated site. serves as a biomarker of cell-to-cell communication in the present invention.

While tumor cells mediate tumor angiogenesis by secreting soluble factors such as VEGF and PDGF that enhance endothelial cells proliferation, migration and vessels formation, endothelial cells secrete wide range of proinflammatory cytokines and chemokines (e.g. IL-1b, IL-6, TNFa, CXCL1 and CCL2) (Krishnaswamy G, Kelley J, Yerra L, Smith J K, Chi D S. Human Endothelium as a Source of Multifunctional Cytokines: Molecular Regulation and Possible Role in Human Disease. J Interferon Cytokine Res (1999) 19:91-104. doi: 10.1089/107999099314234) that stimulate cancer cells and nonmalignant cells within the tumor microenvironment, important process in potentiating inflammatory responses and immune cells activity regulation.

One aspect of this invention is to utilize at least one wavelength of light and the appropriate treatment regimen to inhibit the secretion of a wide range of proinflammatory cytokines and chemokines (such as IL-1b, IL-6, TNFa, CXCL1 and CCL2) Tracking the presence of these or similar species at an untreated site. serves as a biomarker of cell-to-cell communication in the present invention.

Krishnaswamy G et. al. summarized that Vascular endothelial growth factor (VEGF) is a key regulator of physiological angiogenesis during embryogenesis, skeletal growth and reproductive functions. VEGF has also been implicated in pathological angiogenesis associated with tumors, intraocular neovascular disorders and other conditions. The biological effects of VEGF are mediated by two receptor tyrosine kinases (RTKs), VEGFR-1 and VEGFR-2, which differ considerably in signaling properties. Non-signaling co-receptors also modulate VEGF RTK signaling. Currently, several VEGF inhibitors are undergoing clinical testing in several malignancies. VEGF inhibition is also being tested as a strategy for the prevention of angiogenesis, vascular leakage and visual loss in age-related macular degeneration.

One aspect of this invention is to utilize at least one wavelength of light and the appropriate treatment regimen to promote VEGG inhibition, angiogenesis, and vascular leakage. Tracking changes in these tumor characteristics at an untreated site. serves as a biomarker of cell-to-cell communication in the present invention.

To date, there is established agreement that the arrest of circulating cancer cells due to their physical interaction with endothelial cells is essential for their migration from the blood stream and successive growth into metastatic lesions. Specificity in the site of cancer cell arrest has been identified as one factor causative to the organ-specific metastatic patterns. This process involves specific interactions between the surface of circulating cancer cells and endothelial cells through various adhesion molecules such as selectins, integrins cadherins, and immunoglobulins. Some of these molecules are expressed constitutively and appear to have organ specificity in their distribution. Others are inducible and under the influence of environmental signals, such as cytokines. The induction of these molecules on endothelial cells was reported to be link the metastatic capacity of the interacting tumor cells. Subsequently, the growth of the colonizing metastatic cells is sustained by the overexpression and/or increased activity of other molecules such as growth factors and cytokines (. Khatib A-M, Auguste P, Fallavollita L, Wang N, Samani A, Kontogiannea M, et al. Characterization of the Host Proinflammatory Response to Tumor Cells During the Initial Stages of Liver Metastasis. Am J Pathol (2005) 167:749-59. doi: 10.1016/50002-9440(10)62048-2).

One aspect of this invention is to utilize at least one wavelength of light and the appropriate treatment regimen to inhibit the adhesion potency of molecules such as selectins, integrins cadherins, and immunoglobulin. Tracking the adhesion potency of those or similar molecules at an untreated site. serves as a biomarker of cell-to-cell communication in the present invention.

In the Hallmarks of Cancer publication (Hanahan D, Weinberg RA. Hallmarks of Cancer: The Next Generation. Cell (2011) 144:646-74. doi: 10.1016/j.cell.2011.02.013). The authors discuss the role of bone marrow in angiogenesis. A Variety of Bone Marrow-Derived Cells Contribute to Tumor Angiogenesis It is now clear that a repertoire of cell types originating in the bone marrow play crucial roles in pathological angiogenesis (Qian and Pollard, 2010; Zumsteg and Christofori, 2009; Murdoch et al., 2008; De Palma et al., 2007). These include cells of the innate immune system—notably macrophages, neutrophils, mast cells, and myeloid progenitors—that infiltrate premalignant lesions and progressed tumors and assemble at the margins of such lesions; the peri-tumoral inflammatory cells help to trip the angiogenic switch in previously quiescent tissue and to sustain ongoing angiogenesis associated with tumor growth, in addition to facilitating local invasion, as noted below. In addition, they can help protect the vasculature from the effects of drugs targeting endothelial cell signaling (Ferrara, 2010). Additionally, several types of bone marrow-derived “vascular progenitor cells” have been observed in certain cases to have migrated into neoplastic lesions and become intercalated into the neovasculature as pericytes or endothelial cells (Patenaude et al., 2010; Kovacic and Boehm, 2009; Lamagna and Bergers, 2006).

Tumor angiogenesis: causes, consequences, challenges and opportunities Roberta Lugano1·Mohanraj Ramachandran1·Anna Dimberg1 Received: 12 Jul. 2019/Revised: 10 Oct. 2019/Accepted: 21 Oct. 2019/Published online: 6 Nov. 2019© The Author(s) 2019 Abstract Tumor vascularization occurs through several distinct biological processes, which not only vary between tumor type and anatomic location, but also occur simultaneously within the same cancer tissue. These processes are orchestrated by a range of secreted factors and signaling pathways and can involve participation of non-endothelial cells, such as progenitors or cancer stem cells. Anti-angiogenic therapies using either antibodies or tyrosine kinase inhibitors have been approved to treat several types of cancer. However, the benefit of treatment has so far been modest, some patients not responding at all and others acquiring resistance. It is becoming increasingly clear that blocking tumors from accessing the circulation is not an easy task to accomplish. Tumor vessel functionality and gene expression often differ vastly when comparing different cancer subtypes, and vessel phenotype can be markedly heterogeneous within a single tumor. Here, below is a summary of the current understanding of cellular and molecular mechanisms involved in tumor angiogenesis associated with vascular targeting.

### Pericytes:

As noted earlier, pericytes represent a specialized mesenchymal cell type (related to smooth muscle cells) with finger-like projections that wrap around the endothelial tubing of blood vessels.

In normal tissues, pericytes are known to provide paracrine support signals to the normally quiescent endothelium. For example, Ang-1 secreted by pericytes conveys antiproliferative stabilizing signals that are received by the Tie2 receptors expressed on the surface of endothelial cells; some pericytes also produce low levels of VEGF that serve a trophic function in endothelial homeostasis (Gaengel et al., 2009; Bergers and Song, 2005). Pericytes also collaborate with the endothelial cells to synthesize the vascular basement membrane that anchors both pericytes and endothelial cells and helps vessel walls to withstand the hydrostatic pressure of blood flow. Genetic and pharmacological perturbation of the recruitment and association of pericytes has demonstrated the functional importance of these cells in supporting the tumor endothelium (Pietras and Ostman, 2010; Gaengel et al., 2009; Bergers and Song, 2005). For example, pharmacological inhibition of signaling through the PDGF receptor expressed by tumor pericytes and bone marrow-derived pericyte progenitors results in reduced pericyte coverage of tumor vessels, which in turn destabilizes vascular integrity and function (Pietras and Ostman, 2010; Raza et al., 2010; Gaengel et al., 2009); interestingly, and in contrast, the pericytes of normal vessels are not prone to such pharmacological disruption, providing another example of the differences in regulation of normal quiescent and tumor vasculature. An intriguing hypothesis, still to be fully substantiated, is that tumors with poor pericyte coverage of their vasculature may be more prone to permit cancer cell intravasation into the circulatory system, enabling subsequent hematogenous dissemination (Raza et al., 2010; Gerhardt and Semb, 2008).

Tracking changes in these pericytes can also serve as a biomarker of cell-to-cell communication in the present invention.

### Metastasis:

Abdel-Majid Khatib et. al. summarized that the influx of metastatic tumor cells into the liver triggers a rapid proinflammatory cytokine cascade. To further analyze this host response, we used intrasplenic/portal inoculation of green fluorescent protein-marked human and murine carcinoma cells and a combination of immunohistochemistry and confocal microscopy. The metastatic murine lung carcinoma H-59 or human colorectal carcinoma CX-1 cells triggered tumor necrosis factor (TNF)-a production by Kupffer cells located in sinusoidal vessels around the invading tumor cells. H-59 cells rapidly elicited a fourfold increase in the number of TNF-α+ Kupffer cells relative to basal levels within 2 hours and this response declined gradually after 6 hours. Increased cytokine production in these mice was confirmed by reverse transcriptase-polymerase chain reaction and enzyme-linked immunosorbent assay performed on isolated Kupffer cells. CX-1 cells elicited a more gradual response that peaked at 10 to 16 hours, remained high up to 48 hours, and involved CX-1-Kupffer cell attachment. Furthermore, the rapidly induced production of TNF-α was followed by increased expression of the vascular adhesion receptors E-selectin P-selectin, vascular cell adhesion molecule-1, and intercellular adhesion molecule-1 on sinusoidal endothelial cells. This proinflammatory response was tumor-specific and was not observed with nonmetastatic murine M-27 or human MIP-101 carcinoma cells. These results identify Kupffer cell-mediated TNF-α production as an early, tumor-selective host inflammatory response to liver-invading tumor cells that may influence the course of metastasis.

One aspect of this invention is to utilize at least one wavelength of light and the appropriate treatment regimen to inhibit Kupffer cell-mediated TNF-α production as an early, tumor-selective host inflammatory response to liver-invading tumor cells that may influence the course of metastasis. Tracking TNF-α production at an untreated site. serves as a biomarker of cell-to-cell communication in the present invention.

### Tumor-Associated Macrophages (TAMS) and Cancer Cells Communication:

In addition to cancer-associated fibroblasts (CAFs) and tumor vascular endothelial cells. the tumor-associated macrophages (TAMs) are also important constituents of the tumor microenvironment where they play a key role in the interaction of cancer cells with the immune components of the tumor microenvironment. TAMs were reported to promote tumor growth and only in the last decades, TAMs were divided into two broad categories: the pro-inflammatory M1 macrophages with antitumor properties and anti-inflammatory M2 macrophages with tumor-promoting functions.

By secreting molecules including EGF, FGF and TGFb the TAMs directly affect cancer cell proliferation.

Similarly, through the upregulated secretion of various pro-angiogenic factors, such VEGF-A, TNFa, FGF and others, TAMs promote vascular vessel formation and through production of molecules such as VEGF-C and VEGF-D the TAMs induce also the formation of lymphatic vessels. The release of several enzymes such as plasmin, and MMPs, TAMs also participate in tumor cells invasion and metastasis. TAMs can also promote metastasis through the release of exosomes containing various mRNA and oncogenic proteins.

One aspect of this invention is to utilize at least one wavelength of light and the appropriate treatment regimen to inhibit the secretion of EGF, FGF and TGFb and various pro-angiogenic factors such as VEGF-A, TNFa, FGF and other including VEGF-C and VEGF-D. Tracking the presence of these species at an untreated site. serves as a biomarker of cell-to-cell communication in the present invention.

By secreting wide range of cytokines and chemokines, such as CCL3, CCL5, CCL22 and TGFb, TAMs participate also in the recruitment of regulatory T cells (Treg) to the tumor microenvironment and suppress cytotoxic T cell functions.

In addition, TAMs can inhibit cytotoxic T-cell proliferation through several mechanism such as the secretion of IL-10, prostaglandins, TGF-b and reactive oxygen species (ROS) (Whiteside TL. The Tumor Microenvironment and its Role in Promoting Tumor Growth. Oncogene (2008) 27:5904-12. doi: 10.1038/onc.2008.271).

One aspect of this invention is to utilize at least one wavelength of light and the appropriate treatment regimen to inhibit the secretion of CCL3, CCL5, CCL22, IL-10, prostaglandins, TGF-b and reactive oxygen species (ROS). Tracking the secretion of these species and/or the amount of reactive oxygen at an untreated site. serves as a biomarker of cell-to-cell communication in the present invention.

Whiteside T L et. al further summarized that the tumor microenvironment is created by the tumor and dominated by tumor-induced interactions. Although various immune effector cells are recruited to the tumor site, their anti-tumor functions are downregulated, largely in response to tumor-derived signals. Infiltrates of inflammatory cells present in human tumors are chronic in nature and are enriched in regulatory T cells (Treg) as well as myeloid suppressor cells (MSC). Immune cells in the tumor microenvironment not only fail to exercise antitumor effector functions, but they are co-opted to promote tumor growth.

Sustained activation of the NF-κB pathway in the tumor milieu represents one mechanism that appears to favor tumor survival and drive abortive activation of immune cells. The result is tumor escape from the host immune system. Tumor escape is accomplished through the activation of one or several molecular mechanisms that lead to inhibition of immune cell functions or to apoptosis of anti-tumor effector cells. The ability to block tumor escape depends on a better understanding of cellular and molecular pathways operating in the tumor microenvironment. Novel therapeutic strategies that emerge are designed to change the pro-tumor microenvironment to one favoring acute responses and potent anti-tumor activity.

One aspect of this invention is to find at least one wavelength of light and the appropriate treatment regimen to deactivate the NF-κB pathway. Tracking deactivating of the NF-κB pathway at an untreated site. serves as a biomarker of cell-to-cell communication in the present invention.

TAMs express the programmed cell death ligand 1 (PD-L1), a ligand of the immune checkpoint receptor programmed cell death protein 1 (PD-1) that contributes to the generation of an immune-suppressive tumor microenvironment. This by repressing normal function of macrophages including cytokine release, antigen presentation and phagocytosis. Accordingly, PD-1 expression by TAMs increases with tumor progression and the blockade of PD-1 and PDL-1 interaction was found to reduce tumor growth in mice models.

One aspect of this invention is to utilize at least one wavelength of light and the appropriate treatment regimen to promote PD-1 and PDL-1 expression to reduce tumor growth. Tracking PD-1 and PDL-1 expressions at an untreated site can serve as a biomarker of cell-to-cell communication in the present invention.

Similarly, to escape immune system tumor cells express on their surface CD47 that functions as an inhibitor of phagocytosis following its interaction with the signal-regulatory protein alpha (SIRPa) expressed on macrophages cell surface. Indeed, SIRPa/CD47 pathway is referred to as the “do-not-eat-me” signal and tumor cells with CD47 expression can be recognized as self-normal cells and escape phagocytosis.

One aspect of this invention is to utilize at least one wavelength of light and the appropriate treatment regimen to prohibit the expression of CD47. Tracking CD47 at an untreated site can serve as a biomarker of cell-to-cell communication in the present invention.

M1-type macrophages use mainly two different mechanisms to directly eliminate tumor cells: 1—direct cytotoxic action that involves the release of multiple cytotoxic molecules such as ROS and NO and 2—through antibody-dependent cellular cytotoxicity (ADCC) that directly target tumor cells. Other indirect mechanisms were also reported to be involved in tumor cells elimination by M1-type macrophages such as the expression of cytokines (IFN-g, IL-1, and IL-6) that activate the cytotoxic Th1 cells leading to an antitumoral immune response activation.

One aspect of this invention is to utilize at least one wavelength of light and the appropriate treatment regimen to promote antibody-dependent cellular cytotoxicity (ADCC) that directly target tumor cells and the expression of cytokines (IFN-g, IL-1, and IL-6).

Tracking these or other cytokines at an untreated site can serve as a biomarker of cell-to-cell communication in the present invention.

### Adipocytes and Cancer Cells Interaction:

Increased use of lipids by cancer cells is a hallmark of cancer progression and dissemination (2) and in various cancers, the presence of adipocytes can be largely predominant in the tumor tissues. where they mediate various reciprocal interactions with cancer cells. This cross-talk can be either by means of physical interactions or through secreted factors. Thereby cancer cells mediate reprogramming of adipocyte metabolic activity that acquire a cancer-associated adipocyte (CAAs) phenotype.

Abdel-Majid Khatib et. al. concluded that in tumors, cells lose their normal behavior such as their ability to differentiate and communicate with each other in a way to maintain the homeostasis of a specific tissue or organ. However, cancer cells are also able to create a new and specific way to behave and communicate with each other and with the cells in their newly developed microenvironment. In this context, the microenvironment of normal tissue and the TME present various similarities and differences that result from the nature of their cellular interaction and the importance of the mediators and signaling pathways involved. Indeed, the tumor stroma provides exceptional organizational features where cancer cells respond to the constituents of the TME through modulation of the expression/activity of proteins involved in cell survival, proliferation and migration. On the other hand, tumor cell-derived signals activate and recruit various cells such as immune cells, fibroblasts, adipocytes and endothelial cells Tracking tumor-cell derived signals at an untreated site. serves as a biomarker of cell-to-cell communication in the present invention.
that acquired cancer-associated phenotype and influence the structure and the composition of the TME by releasing ECM enzymes and components, exosomes, cytokines, and growth factors, all influence cancer cell functions and metabolism. Although these activated associated-cancer cells often enhance tumor growth and invasion, their activation can also repress tumor growth and invasion using the same released mediators. Thus, in a reciprocal manner, tumor cells influence the stroma and vice versa, jointly driving cancer progression or repression. The identification and the understanding of all these cancer causal factors and the degree of the importance of each reciprocal interaction in a temporal and geographical point of view will strongly help for the development of new prognostic and therapeutic strategies. This by taking in account not only the characteristic of the tumors and their TME but also the nature and the levels of the interactions between the signaling and the responding cells in the developing TME. Tracking TME at an untreated site. serves as a biomarker of cell-to-cell communication in the present invention.

### Excessive Proliferative Signaling can Trigger Cell Senescence:

Hanahan et. al. state that the hallmarks of cancer comprise six biological capabilities acquired during the multistep development of human tumors. The hallmarks constitute an organizing principle for rationalizing the complexities of neoplastic disease. They include sustaining proliferative signaling, evading growth suppressors, resisting cell death, enabling replicative immortality, inducing angiogenesis, and activating invasion and metastasis. Underlying these hallmarks are genome instability, which generates the genetic diversity that expedites their acquisition, and inflammation, which fosters multiple hallmark functions. Conceptual progress in the last decade has added two emerging hallmarks of potential generality to this list—reprogramming of energy metabolism and evading immune destruction. In addition to cancer cells, tumors exhibit another dimension of complexity: they contain a repertoire of recruited, ostensibly normal cells that contribute to the acquisition of hallmark traits by creating the “tumor microenvironment.” Recognition of the widespread applicability of these concepts will increasingly affect the development of new means to treat human cancer.

Early studies of oncogene action encouraged the notion that ever-increasing expression of such genes and the signals manifested in their protein products would result in correspondingly increased cancer cell proliferation and thus tumor growth. More recent research has undermined this notion, in that excessively elevated signaling by oncoproteins such as RAS, MYC, and RAF can provoke counteracting responses from cells, specifically induction of cell senescence and/or apoptosis (Collado and Serrano, 2010; Evan and d'Adda di Fagagna, 2009; Lowe et al., 2004). For example, cultured cells expressing high levels of the Ras oncoprotein may enter into the non-proliferative but viable state called senescence; in contrast, cells expressing lower levels of this protein may avoid senescence and proliferate. Cells with morphological features of senescence, including enlarged cytoplasm, the absence of proliferation markers, and expression of the senescence-induced b-galactosidase enzyme, are abundant in the tissues of mice engineered to overexpress certain oncogenes (Collado and Serrano, 2010; Evan and d'Adda di Fagagna, 2009) and are prevalent in some cases of human melanoma (Mooi and Peeper, 2006). These ostensibly paradoxical responses seem to reflect intrinsic cellular defense mechanisms designed to eliminate cells experiencing excessive levels of certain types of signaling. Accordingly, the relative intensity of oncogenic signaling in cancer cells may represent compromises between maximal mitogenic stimulation and avoidance of these antiproliferative defenses.

One aspect of this invention is to utilize at least one wavelength of light and the appropriate treatment regimen that minimizes proinflammatory signaling and increase immunogenic properties without inducing cell senescence. Tracking the proinflammatory signaling or the immunogenic properties at an untreated site can serve as a biomarker of cell-to-cell communication in the present invention.

### Therapeutic Targeting:

The introduction of mechanism-based targeted therapies to treat human cancers has been heralded as one of the fruits of three decades of remarkable progress of research into the mechanisms of cancer pathogenesis. We do not attempt here to enumerate the myriad therapies that are under development or have been introduced of late into the clinic. Instead, we consider how the description of hallmark principles is beginning to inform therapeutic development at present and may increasingly do so in the future. The rapidly growing armamentarium of targeted therapeutics can be categorized according to their respective effects on one or more hallmark capabilities, as illustrated in the examples presented in the Figure below. Indeed, the observed efficacy of these drugs represents, in each case, a validation of a particular capability: if a capability is truly important for the biology of tumors, then its inhibition should impair tumor growth and progression. We note that most of the hallmark-targeting cancer drugs developed to date have been deliberately directed toward specific molecular targets that are involved in one way or another in enabling particular capabilities. Such specificity of action has been considered a virtue, as it presents inhibitory activity against a target while having, in principle, relatively fewer off-target effects and thus less nonspecific toxicity. In fact, resulting clinical responses have generally been transitory, being followed by almost-inevitable relapses. One interpretation of this history, supported by growing experimental evidence, is that each of the core hallmark capabilities is regulated by partially redundant signaling pathways. Consequently, a targeted therapeutic agent inhibiting one key pathway in a tumor may not completely shut off a hallmark capability, allowing some cancer cells to survive with residual function until they or their progeny eventually adapt to the selective pressure imposed by the therapy being applied. Such adaptation, which can be accomplished by mutation, epigenetic reprogramming, or remodeling of the stromal microenvironment, can reestablish the functional capability, permitting renewed tumor growth and clinical relapse. Given that the number of parallel signaling pathways supporting a given hallmark must be limited, it may become possible to target all of these supporting pathways therapeutically, thereby preventing the development of adaptive resistance. In response to therapy, cancer cells may also reduce their dependence on a particular hallmark capability, becoming more dependent on another; this represents a quite different form of acquired drug resistance. This concept is exemplified by recent discoveries of unexpected responses to antiangiogenic therapies.

Some have anticipated that effective inhibition of angiogenesis would render tumors dormant and might even lead to their dissolution (Folkman and Kalluri, 2004). Instead, the clinical responses to antiangiogenic therapies have been found to be transitory (Azam et al., 2010; Ebos et al., 2009; Bergers and Hanahan, 2008). In certain preclinical models, where potent angiogenesis inhibitors succeed in suppressing this hallmark capability, tumors adapt and shift from a dependence upon continuing angiogenesis to heightening the activity of another instead—invasiveness and metastasis (Azam et al., 2010: Ebos et al., 2009; Bergers and Hanahan, 2008). By invading nearby tissues, initially hypoxic cancer cells evidently gain access to normal, preexisting tissue vasculature.

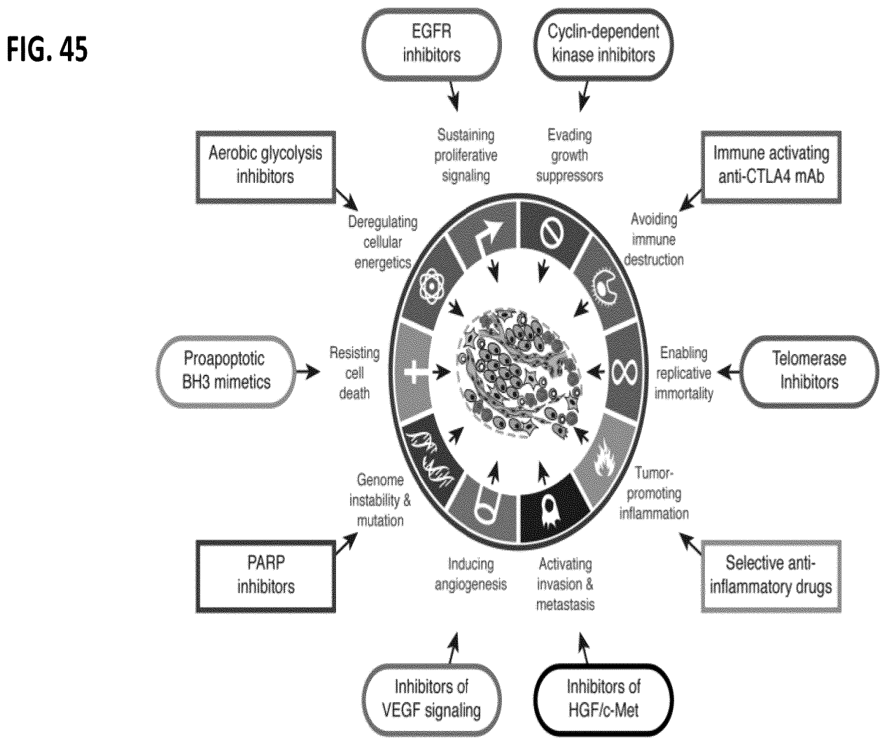

Initial clinical validation of this adaptive/evasive resistance is apparent in the increased invasion and local metastasis seen when human glioblastomas are treated with antiangiogenic therapies (Ellis and Reardon, 2009; Norden et al., 2009; Verhoeff et al., 2009). The applicability of this lesson to other human cancers has yet to be established. Analogous adaptive shifts in dependence on other hallmark traits may also limit efficacy of analogous hallmark-targeting therapies. For example, the deployment of apoptosis-inducing drugs may induce cancer cells to hyperactivate mitogenic signaling, enabling them to compensate for the initial attrition triggered by such treatments. Such considerations suggest that drug development and the design of treatment protocols will benefit from incorporating the concepts of functionally discrete hallmark capabilities and of the multiple biochemical pathways involved in supporting each of them. Thus, in one embodiment of the invention, selective co-targeting of multiple core and emerging hallmark capabilities and enabling characteristics (FIG. 45 below) in mechanism-guided combinations will result in more effective and durable therapies for human cancer.

Drugs that interfere with each of the acquired capabilities necessary for tumor growth and progression have been developed and are in clinical trials or in some cases approved for clinical use in treating certain forms of human cancer. Additionally, the investigational drugs are being developed to target each of the enabling characteristics and emerging hallmarks depicted in FIG. 3, which also hold promise as cancer therapeutics. The drugs listed are but illustrative examples; there is a deep pipeline of candidate drugs with different molecular targets and modes of action in development for most of these hallmarks.

Several aspects of the present invention pertain to enhancing the functionality of drugs designed to: Inhibit the VEGF signaling, inhibit HGF/c-Met, Selective inflammatory reactions, Telomerase inhibition, Immune activation anti CTLA4-mAb, cyclin dependent kinase inhibition, EGFR inhibition, Aeorobic glycolysis inhibition, proapoptotic BH3 mimetics, PARP inhibition.

By predisposing the biological environment to respond favorably to the effect of existing therapies, the biophoton emission and/or cell-to-cell communication occurring in and around treated cells using such therapies help lead the subject to a more favorable outcome.

### The Extracellular Matrix Directing Gene Expression:

The ECM plays a big role and a quick review is hereby provided. In a review published in J. Theor. Biol. (1982) 99, 31-68, Nina J. Bissell, H. Glenn Hall and Gordon Parry from the Laboratory of Cell Biology, Division of Biology & Medicine, Lawrence Berkeley Laboratory, University of California, Berkeley, California 94720, U.S.A. expanded on the concept of Dynamic reciprocity. Based on the existing literature, a model is presented that postulates a “dynamic reciprocity” between the extracellular matrix (ECM) on the one hand and the cytoskeleton and the nuclear matrix on the other hand. The ECM is postulated to exert physical and chemical influences on the geometry and the biochemistry of the cell via transmembrane receptors so as to alter the pattern of gene expression by changing the association of the cytoskeleton with the mRNA and the interaction of the chromatin with the nuclear matrix. This, in turn, would affect the ECM, which would affect the cell.

The concept that different but closely associated cell populations act on each other to generate new and diverse cell types during development was initially termed “embryonic induction” (Grobstein, 1955) and is now referred to as “tissue interaction”. Mesenchyme often plays a predominant role in directing the differentiation of epithelia. There are also examples of mesenchymal specificity in instructing tissue development in cross species combinations (Sengel, 1976).

The studies summarized were mostly descriptive and did not probe specifically the nature of the factors that comprise the instructive capabilities of the mesenchyme.

Numerous studies on induction of cartilage and chondrogenesis (Table 2), demonstrate that even after the chondrocytes or lens are formed, removal of ECM will lead to loss of differentiated functions, and its restoration will lead to re-differentiation.

Even after the tissues are formed, the ability to express tissue specific functions is still dependent on the nature and the integrity of the substrata and the extracellular environment. These studies, by necessity, all utilize cell culture. The very act of placing cells in culture leads to dramatic changes in the expression of the differentiated functions (for a review see Davidson, 1964; Bissell, 1981). However, considerable recent evidence indicates that certain functional losses can be reversed if the cells are plated on naturally occurring ECM or surfaces coated with ECM components, rather than on plastic or glass surfaces.

Investigators showed that if mammary epithelial cells are plated on a thick collagen gel, which subsequently is floated on the medium, the cells derived from pregnant or lactating mice regain their intracellular polarity and characteristic secretory morphology as well as some biochemical properties.

Furthermore, a striking feature of these experiments is the overriding influence of the substratum on the mammary phenotype, most probably a consequence of a change in cell shape. On a flat plastic substratum, cells from all stages of gland development assume a flattened morphology; and even in the presence of hormones, convert to a metabolic phenotype resembling fibroblasts. On floating collagen gels, the same cells assume a cuboidal shape. Here, the morphology becomes a biomarker of the present invention used to determine if an untreated site is responding in a predicted way to arrest cancer or disease progression.

The following tables provide information on characteristics of tissue formation which could also serve as biomarker(s) of cell-to-cell communication in the present invention at an untreated site.

TABLE 1
The influence of mesenchyme on tissue formation
EXTRACELLULAR MATRIX AND GENE EXPRESSION

| Instructive tissue | Affected tissue | Outcome | Reference |
|---|---|---|---|
| Leg mesoderm | Wing | Leg feathers | Saunders & |
| Feather-area mesoderm | Non feather ectoderm | Feathers | G [symbol] ling (1957) |
| Scale-area mesoderm | Feather area ectoderm | Scales | |
| Limb dermis mesenchyme | Epidermis from chick embryo back | Keratin formation | McLoughlin (1961) |
| Proventriculus (the anterior stomach) | Epidermis from chick embryo back | Mucus (as in gut) | |
| Gizzard (the posterior stomach) | Epidermis from chick embryo back | Mucus + cilia | |
| | | (secretion of mucus) | |
| Normal mesoderm (+/+) | Mutant ectoderm (PD) | +/+phenotype | Goetinck (1966) |
| | (polydactylous tissues) | | |
| PD mesoderm | Normal ectoderm (+/+) | PD phenotype | |
| Mammary mesenchyme | Mammary epithelium | Narrow, tubular structures | Kratochwil (1969) |
| | | (mammary) | |
| Salivary gland mesenchyme | Salivary epithelium | Flowerlike, bulbous structure | |
| | | (salivary) | |
| Salivary gland mesenchyme | Mammary epithelium | Flowerlike, bulbous structure | |
| | | (salivary) | |
| Bronchial mesoderm | Bronchial epithelium | Bronchial morphology | Spooner & |
| | | (secondary bulbous structures) | Wessells (1970) |
| Salivary mesoderm | Salivary epithelium | Salivary morphology | |
| Salivary mesoderm | Bronchial epithelium | Salivary morphology | |
| Gizzard mesenchyme | Rabbit stomach epithelium | One cell thick layer of epithelium | David (1972) |
| Vitelline membrane from chick egg | Rabbit stomach epithelium | Dividing epithelium with mounds | |
| Embryonic rabbit mammary gland | Avian flank epithelium | Mammary-like epithelium with | Propper (1975) |
| mesenchyme | | tubule formation | |
| Urogenital sinus mesenchyme | Urinary bladder epithelium | Induction of prostatic acini & | Cunha et al. (1980) |
| | | androgen binding sites | |

[symbol]

indicates data missing or illegible when filed

TABLE 2
The influence of ECM or its components on Tissue development

| Inducer | Cell system | Outcome | Reference |
|---|---|---|---|
| Collagen coated dishes | Myoblasts | Fused myotubes | Hauschka & Konigsberg (1966) |
| [symbol] ( [symbol] )-CB, peptide | Myoblasts | Fused myotubes | Hauschka & White (1972) |
| Collagen [symbol] (inferred from | Lung and salivary gland | Branching morphogenesis | Wessels & Cohen (1968) |
| collagenase treatment) | | | |
| GAGs [symbol] | Lung and salivary gland | Branching morphogenesis | Bernfield et al. (1972) |
| Collagen [symbol] (inferred from | Lung & salivary gland | Branching morphogenesis | Spooner & Faubian (1980) |
| metabolic inhibitors) | | | |
| Lens ECM (capsule) | Lens epithelium | Formation of lens stroma | Dodson & Hay (1971, 1974) |
| | | | Hay & Dodson, (1973) |
| Purified collagen | Lens epithelium | Formation of lens stroma | Hay & Meier (1976) |
| Collagen (inferred from | Chick embryonic | Feather morphogenesis | S [symbol] art, Garber & |
| collagenase treatment) | skin tracts | | Moscona (1972) |
| ECM and GAG of notochord | Embryonic somites | Somite chondrogenesis | Strudel (1971) |
| | | | Lash & Kosher (1975) |
| Chondromuco protein | Chick somitic | Chondrogenesis | Kosher et al. (1973) |
| | scleratomal cells | | |
| Collagen substratum, types I & | Chick somitic | Chondrogenesis | Kosher & Church (1975) |
| II collagen or procollage [symbol] | sclerotomal cells | | Lach & Vasan (1977) |
| | | | Lash & Vasan (1978) |
| ECM from pigmented | Clonal neural crest or | Chondrogenesis | Newsome (1976) |
| epithelium | periocular mesenchymal cells | | |
| Bone matrix | Pro-natal muscle | Chondrogenesis | Urist et al. (1975) |
| | connective tissue | | |
| Hyaluronate or its | Chick embryo somitcs | Inhibition of chondrogenesis | Toole et al. (1972) |
| tetrasacharide product | | | Toole (1973) |
| Demineralized diaphyseal | Fibroblasts | Chondroblasts and osteoblasts | Reddi (1975) |
| bone matrix | | | |
| Demineralized diaphyseal | Fibroblasts | Inhibition of chondrogenesis | Reddi (1975) |
| bone matrix + polyanions | | and osteogenesis | |
| ECM (fibronectin) | Mesenchymal precursor | Chondroblasts and | Weiss & Reddi (1980) |
| | cells from rat | osteoblasts | |
| GAGs (hyaluronic acid) | Neural crest trunk | Migration, differentiation | Derby (1978) |

[symbol]

See text [symbol]
indicates data missing or illegible when filed

TABLE 3
The influence of ECM or its components on the expression of differentiated functions

| ECM component | Cell type | Outcome | References |
|---|---|---|---|
| Sulfate GAGs | Chondrocytes (embryonic chick) | Increased sulfated GAG | Nevo & [symbol] (1972) |
| Hyaluronic acid | Chondrocytes (adult [symbol] ) | [symbol] of [symbol] GAG | [symbol] , Hardingham & |
| | | synthesis | Muir (197 [symbol] ) |
| Enamel organ ECM | Mouse enamel organ | Inhibition of enamel protein | [symbol] et al. (197 [symbol] ) |
| | epithelium | [symbol] ; stimulation of | |
| | | collagen | |
| Floating collagen gels | Rat hepatocytes | Maintenance of morphology, | Michalop [symbol] & P [symbol] |
| ( [symbol] ) | | increased [symbol] induction | (197 [symbol] ) |
| Floating collagen gels | Mammary epithelial cells | Morphological and biochemical | [symbol] & [symbol] (1977) |
| ( [symbol] ) | | differentiation | [symbol] et al. (1977, |
| | | | 1981) |
| ECM from corneal | Tumor of normal cells | Proliferation | [symbol] etal. (1980) |
| endothelial cells | incapable of growth on plastic | | [symbol] et al. (1980 [symbol] |
| Fetal calf serum induced | Adrenal cortical cells | Inhibition of [symbol] | Turley (1980) |
| ECM or hyaluronic acid | | [symbol] , onset of [symbol] , | |
| | | “Fibroblastic morphology” | |
| Type IV collagen | Primary [symbol] | Growth in absence of [symbol] GF | [symbol] et al. (1981) |
| | cells | | |
| Biomatrix [symbol] | Rat hepatocytes | [symbol] stabilization of | Rojkind et al. (1980) |
| rat liner | | [symbol] tissue-specific functions | |
| | | and increased survival | |
| ECM from normal primary | Primary normal tendon cells | Increased proliferation in | Bissell et al. (1981, |
| avian tendon | | [symbol] serum, increased | and unpublished results) |
| | | collagen synthesis | |
| Lens ECM, collagen gel, | Corneal epithelial cells | Inhibition of [symbol] , | Sugrue & Hay (1981) |
| soluble collagen, [symbol] , | | organization of [symbol] | |
| fibronectin | | [symbol] | |
| Collagen gels and [symbol] | Thyroid cells on preformed | Follicle formation, | Chamband et al. (1981) |
| collagen layers | monolayers | establishment of polarity | |
| Two collagen layers | Mammary and kidney epithelial | Lumen formation, establishment | Hall et al. (1982) |
| | cell lines | of polarity, changes in | |
| | | biosynthetic patterns | |
| Type I collagens | Mouse mammary cell lines | Reduction in GAG degradation | David & Bernfield (1981) |
| ECM of [symbol] | Fatal bovine [symbol] | Formation at insoluble crossed | Mecham et al. (1981) |
| | [symbol] minces | linked elastin | |
| Fibronectin (CSP) | 1,929, SV-1, RSV-transformed | Decreased [symbol] , restoration | Yamada et al. (1976) |
| | chick embryo fibroblasts | of “normal” morphology | |
| Fibronectin (LETS) | HSV-transformed NIL | “Normal morphology”, increased | A [symbol] et al. (1977) |
| | hamster fibroblasts | adhesion, reorganization of | |
| | | actin cables | |
| ECM from normal primary | RSV-transformed tendon | “Normal morphology”, changes | Bissell et al. (1981, |
| avian lenders | cells or chick embryo | in polypeptide synthesis, | and unpublished results) |
| | fibroblasts | increased fibronectin | |
| Rat tail collagen | Primary mouse mammary | Synthesis and secretion of | Parry et al. (1982); |
| (Roatation) | epithelial cells | the entire pattern of | and Lee, Parry & Bissell, |
| | | milk-specific proteins | unpublished results) |

[symbol]

See text [symbol]
indicates data missing or illegible when filed

The proliferative effect of ECM in vivo is exemplified by the maintenance of growth of stratified epidermis. The cells in contact with the basal lamina actively undergo division, whereas those cells that are released from this natural substratum cease dividing, move upward, flatten and differentiate into upper stratified layers.

However, in real tissue burdened with disease, the situation may be different. One aspect of the present invention is to utilize the regimen of wavelengths (from monochromatic to polychromatic) that can restore the expression of healthy tissue specific functions. Recognizing that the equilibrium that exits between the various tissues in an organ is disrupted, in one embodiment of the invention, a new equilibrium is established, and tumor suppression is achieved. Regardless, tracking tumor regression in an untested site serves as a biomarker of cell-to-cell communication of the present invention.

### XPACT Close Loon Feedback:

XPACT provides a means by which some chemokines can be modulated in-vivo in real diseased patients. The XPACT therapy consists of 4 treatments given every other day (8 treatments over 8 days), followed by a fifth treatment administered about 30 days past the 4^{th} treatment (there is no exact schedule for each individual patients). As such all patient are treated in the same manner.

However, there is a way to treat each patient differently based on the modulation of important biomarkers. If pro-inflammatory cytokines are on the rise, then the patient's treatment could be postponed or delayed by one or a few days. If on the other hand pro-inflammatory cytokines are decreasing, then the patient is better served by prompt treatment.

The closed loop feedback can be based on one or preferably more than one cytokine biomarker. If all of the interrogated biomarkers are trending the same way, then the signal on treat now vs. delay treatment becomes stronger. Therefore, the treatment schedule is based on the patient's need rather than based on an average that could not account for the particulars of the patient's immune system. This is because every patient has a distinct immune system leading to wide ranging responses to treatments.

Furthermore, certain cancer types are more conducive to secreting select chemokines more than others. Therefore, a select list of cytokines for each cancer type is possible and the closed loop feedback on treatment can be based on cancer specific secreted biomarkers.

In one example, several cytokines and growth factors have monitored during the treatment of canine patients including IL1, IL2, IL6, TGF-Beta, MCP1, Interferon gamma (IFN-γ), TNF-A, and identified as biomarkers for the present invention and at least detectable in blood tests and/or other assays.

### Interleukins:

Interleukins (ILs) are a group of cytokines (secreted proteins and signal molecules) that are expressed and secreted by white blood cells (leukocytes) as well as some other body cells. The human genome encodes more than 50 interleukins and related protein.

Interleukin 1 alpha and interleukin 1 beta (IL1 alpha and IL1 beta) are cytokines that participate in the regulation of immune responses, inflammatory reactions, and hematopoiesis.

Interleukin-2 (IL-2) is an interleukin, a type of cytokine signaling molecule in the immune system. It is a 15.5-16 kDa protein[5] that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells.

Interleukin 6 (IL6), also referred to as B-cell stimulatory factor-2 (BSF-2) and interferon beta-2, is a cytokine involved in a wide variety of biological functions. It plays an essential role in the final differentiation of B cells into immunoglobulin-secreting cells, as well as inducing myeloma/plasmacytoma growth, nerve cell differentiation, and, in hepatocytes, acute-phase reactants.

A number of other cytokines may be grouped with IL6 on the basis of sequence similarity. These include granulocyte colony-stimulating factor (GCSF) and myelomonocytic growth factor (MGF). GCSF acts in hematopoiesis by affecting the production, differentiation, and function of 2 related white cell groups in the blood. MGF also acts in hematopoiesis, stimulating proliferation and colony formation of normal and transformed avian cells of the myeloid lineage.

Cytokines of the IL6/GCSF/MGF family are glycoproteins of about 170 to 180 amino acid residues that contain four conserved cysteine residues involved in two disulphide bonds. They have a compact, globular fold (similar to other interleukins), stabilised by the two disulphide bonds. One half of the structure is dominated by a 4-alpha-helix bundle with a left-handed twist; [23] the helices are anti-parallel, with two overhand connections, which fall into a double-stranded anti-parallel beta-sheet. The fourth alpha-helix is important to the biological activity of the molecule.

### IFN-γ:

Interferon gamma (IFN-γ) is a dimerized soluble cytokine that is the only member of the type II class of interferons. The existence of this interferon, which early in its history was known as immune interferon, was described by E. F. Wheelock as a product of human leukocytes stimulated with phytohemagglutinin, and by others as a product of antigen-stimulated lymphocytes. It was also shown to be produced in human lymphocytes. or tuberculin-sensitized mouse peritoneal lymphocytes challenged with Mantoux test (PPD); the resulting supernatants were shown to inhibit growth of vesicular stomatitis virus. Those reports also contained the basic observation underlying the now widely employed IFN-γ release assay used to test for tuberculosis. In humans, the IFN-γ protein is encoded by the IFNG gene.

IFN-γ, or type II interferon, is a cytokine that is critical for innate and adaptive immunity against viral, some bacterial and protozoan infections. IFN-γ is an important activator of macrophages and inducer of major histocompatibility complex class II molecule expression. Aberrant IFN-γ expression is associated with a number of autoinflammatory and autoimmune diseases. The importance of IFN-γ in the immune system stems in part from its ability to inhibit viral replication directly, and most importantly from its immunostimulatory and immunomodulatory effects. IFN-γ is produced predominantly by natural killer cells (NK) and natural killer T cells (NKT) as part of the innate immune response, and by CD4 Th1 and CD8 cytotoxic T lymphocyte (CTL) effector T cells once antigen-specific immunity develops as part of the adaptive immune response. IFN-γ is also produced by non-cytotoxic innate lymphoid cells (ILC), a family of immune cells first discovered in the early 2010s.

IFN-γ is secreted by T helper cells (specifically, Th1 cells), cytotoxic T cells (TC cells), macrophages, mucosal epithelial cells and NK cells. IFN-γ is both an important autocrine signal for professional APCs in early innate immune response, and an important paracrine signal in adaptive immune response. The expression of IFN-γ is induced by the cytokines IL-12, IL-15, IL-18, and type I IFN. IFN-γ is the only Type II interferon and it is serologically distinct from Type I interferons; it is acid-labile, while the type I variants are acid-stable.

IFN-γ has antiviral, immunoregulatory, and anti-tumor properties. It alters transcription in up to 30 genes producing a variety of physiological and cellular responses. Among the effects are:
Promotes NK cell activity
Increases antigen presentation and lysosome activity of macrophages.
Activates inducible nitric oxide synthase (iNOS)
Induces the production of IgG2a and IgG3 from activated plasma B cells
Causes normal cells to increase expression of class I MHC molecules as well as class II MHC on antigen-presenting cells—to be specific, through induction of antigen processing genes, including subunits of the immunoproteasome (MECL1, LMP2, LMP7), as well as TAP and ERAAP in addition possibly to the direct upregulation of MHC heavy chains and B2-microglobulin itself
Promotes adhesion and binding required for leukocyte migration
Induces the expression of intrinsic defense factors—for example, with respect to retroviruses, relevant genes include TRIM5alpha, APOBEC, and Tetherin, representing directly antiviral effects
Primes alveolar macrophages against secondary bacterial infections.

IFN-γ is the primary cytokine that defines Th1 cells: Th1 cells secrete IFN-γ, which in turn causes more undifferentiated CD4+ cells (Th0 cells) to differentiate into Th1 cells, representing a positive feedback loop—while suppressing Th2 cell differentiation. (Equivalent defining cytokines for other cells include IL-4 for Th2 cells and IL-17 for Th17 cells.)

NK cells and CD8+ cytotoxic T cells also produce IFN-γ. IFN-γ suppresses osteoclast formation by rapidly degrading the RANK adaptor protein TRAF6 in the RANK-RANKL signaling pathway, which otherwise stimulates the production of NF-κB.

### TNF-α:

Tumor necrosis factor (TNF, cachexin, or cachectin; often called tumor necrosis factor alpha or TNF-α) is an adipokine and a cytokine. TNF is a member of the TNF superfamily, which consists of various transmembrane proteins with a homologous TNF domain.

As an adipokine, TNF promotes insulin resistance, and is associated with obesity-induced type 2 diabetes. As a cytokine, TNF is used by the immune system for cell signaling. If macrophages (certain white blood cells) detect an infection, they release TNF to alert other immune system cells as part of an inflammatory response.

TNF signaling occurs through two receptors: TNFR1 and TNFR2. TNFR1 is constitutively expressed on most cell types, whereas TNFR2 is restricted primarily to endothelial, epithelial, and subsets of immune cells. TNFR1 signaling tends to be pro-inflammatory and apoptotic, whereas TNFR2 signaling is anti-inflammatory and promotes cell proliferation. Suppression of TNFR1 signaling has been important for treatment of autoimmune disease, whereas TNFR2 signaling promotes wound healing.

TNF-α exists as a transmembrane form (mTNF-α) and as a soluble form (sTNF-α). sTNF-α results from enzymatic cleavage of mTNF-α, by a process called substrate presentation. mTNF-α is mainly found on monocytes/macrophages where it interacts with tissue receptors by cell-to-cell contact. sTNF-α selectively binds to TNFR1, whereas mTNF-a binds to both TNFR1 and TNFR2. TNF-α binding to TNFR1 is irreversible, whereas binding to TNFR2 is reversible.

The primary role of TNF is in the regulation of immune cells. TNF, as an endogenous pyrogen, is able to induce fever, apoptotic cell death, cachexia, and inflammation, inhibit tumorigenesis and viral replication, and respond to sepsis via IL-1 and IL-6-producing cells. Dysregulation of TNF production has been implicated in a variety of human diseases including Alzheimer's disease, cancer, major depression, psoriasis and inflammatory bowel disease (IBD). Though controversial, some studies have linked depression and IBD to increased levels of TNF.

Under the name tasonermin, TNF is used as an immunostimulant drug in the treatment of certain cancers. Drugs that counter the action of TNF are used in the treatment of various inflammatory diseases, for instance rheumatoid arthritis.

### MCP1:

The chemokine (C-C motif) ligand 2 (CCL2) is also referred to as monocyte chemoattractant protein 1 (MCPT) and small inducible cytokine A2. CCL2 is a small cytokine that belongs to the CC chemokine family. CCL2 tightly regulates cellular mechanics[5] and thereby recruits monocytes, memory T cells, and dendritic cells to the sites of inflammation produced by either tissue injury or infection.[6][7]

CCL2 is a monomeric polypeptide, with a molecular weight of approximately 13-15 kDa depending on levels of glycosylation. CCL2 is anchored in the plasma membrane of endothelial cells by glycosaminoglycan side chains of proteoglycans. CCL2 is primarily secreted by monocytes, macrophages and dendritic cells. Platelet derived growth factor is a major inducer of CCL2 gene. CCL2 exhibits a chemotactic activity for monocytes and basophils. However, it does not attract neutrophils or eosinophils. After deletion of the N-terminal residue, CCL2 loses its attractivity for basophils and becomes a chemoattractant of eosinophils. Basophils and mast cells that are treated with CCL2 release their granules to the intercellular space. This effect can be also potentiated by a pre-treatment with IL-3 or even by other cytokines. CCL2 augments monocyte anti-tumor activity and it is essential for formation of granulomas. CCL2 protein become a CCR2 antagonist when it is cleaved by metalloproteinase MMP-12

CCL2 can be found at the sites of tooth eruption and bone degradation. In the bone, CCL2 is expressed by mature osteoclasts and osteoblasts and it is under control of nuclear factor κB (NFκB). In the human osteoclasts, CCL2 and RANTES (regulated on activation normal T cell expressed and secreted). Both MCP-1 and RANTES induce formation of TRAP-positive, multinuclear cells from M-CSF-treated monocytes in the absence of RANKL, but produced osteoclasts that lacked cathepsin K expression and resorptive capacity. CCL2 and RANTES may act as autocrine loop in human osteoclast differentiation.

The CCL2 chemokine is also expressed by neurons, astrocytes and microglia. The expression of CCL2 in neurons is mainly found in the cerebral cortex, globus pallidus, hippocampus, paraventricular and supraoptic hypothalamic nuclei, lateral hypothalamus, substantia nigra, facial nuclei, motor and spinal trigeminal nuclei, gigantocellular reticular nucleus and in Purkinje cells in the cerebellum.

### TGF-β:

Transforming growth factor beta (TGF-β) is a multifunctional cytokine belonging to the transforming growth factor superfamily that includes three different mammalian isoforms (TGF-β 1 to 3, HGNC symbols TGFB1, TGFB2, TGFB3) and many other signaling proteins. TGFB proteins are produced by all white blood cell lineages.

Activated TGF-β complexes with other factors to form a serine/threonine kinase complex that binds to TGF-β receptors. TGF-β receptors are composed of both type 1 and type 2 receptor subunits. After the binding of TGF-β, the type 2 receptor kinase phosphorylates and activates the type 1 receptor kinase that activates a signaling cascade. This leads to the activation of different downstream substrates and regulatory proteins, inducing transcription of different target genes that function in differentiation, chemotaxis, proliferation, and activation of many immune cells.

TGF-β is secreted by many cell types, including macrophages, in a latent form in which it is complexed with two other polypeptides, latent TGF-beta binding protein (LTBP) and latency-associated peptide (LAP). Serum proteinases such as plasmin catalyze the release of active TGF-β from the complex. This often occurs on the surface of macrophages where the latent TGF-β complex is bound to CD36 via its ligand, thrombospondin-1 (TSP-1). Inflammatory stimuli that activate macrophages enhance the release of active TGF-β by promoting the activation of plasmin. Macrophages can also endocytose IgG-bound latent TGF-β complexes that are secreted by plasma cells and then release active TGF-β into the extracellular fluid. Among its key functions is regulation of inflammatory processes, particularly in the gut. TGF-β also plays a crucial role in stem cell differentiation as well as T-cell regulation and differentiation.

Because of its role in immune and stem cell regulation and differentiation, it is a highly researched cytokine in the fields of cancer, auto-immune diseases, and infectious disease.

The TGF-β superfamily includes endogenous growth inhibiting proteins; an increase in expression of TGF-β often correlates with the malignancy of many cancers and a defect in the cellular growth inhibition response to TGF-β. Its immunosuppressive functions then come to dominate, contributing to oncogenesis. The dysregulation of its immunosuppressive functions is also implicated in the pathogenesis of autoimmune diseases, although their effect is mediated by the environment of other cytokines present.

### Close Loon Feedback Reduction to Practice:

Especially the following biomarkers may be used in the invention for close loop feedback: IL1, IL2, IL6, TGF-Beta, MCP1, Interferon gamma (IFN-γ), TNF-A.

These biomarkers are just a small representation of what is possible to monitor during the evolution of a treatment. This list is therefore an example through which a close loop process can treat tumors effectively and drive a partial remission, a complete remission or to halt (arrest) tumor growth. As discussed previously growth factors such as TGF-beta can be secreted to promote growth; therefore, the careful monitoring of TGF-beta can be used as an indication of tumor behavior.

Ideally two groups should be identified: A- biomarkers that should decrease with a successful treatment and these encompass (TGF-beta, IL1 and IL6) and B-biomarkers that should increase with a successful treatment and these encompass (IFG, IL2, MCP1 and TNF-A).

The following results were obtained canine patients undergoing XPACT. The tumor was measured using calipers and the tumor volume was calculated. The tumor from Each of the patients in this example were plotted over time to check the tumor size modulation. The biomarkers were also plotted over the time frame. The biomarkers that were supposed to decrease with treatment were scored. The biomarkers that were supposed to increase with treatment were also scored. A scoring system was developed to rank by order of importance.

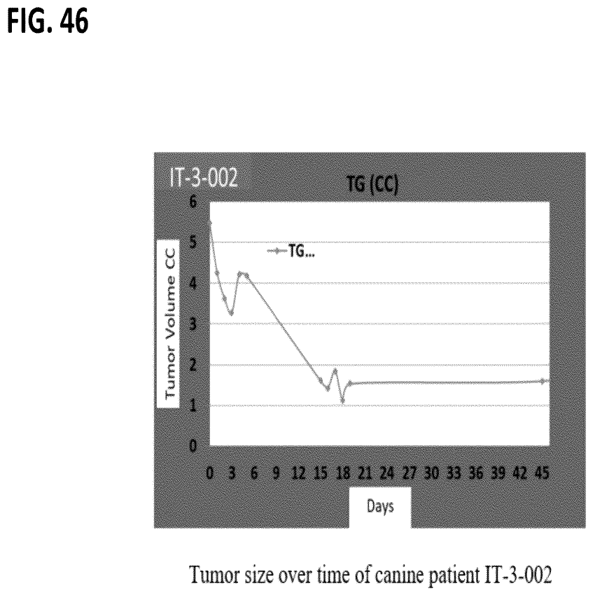

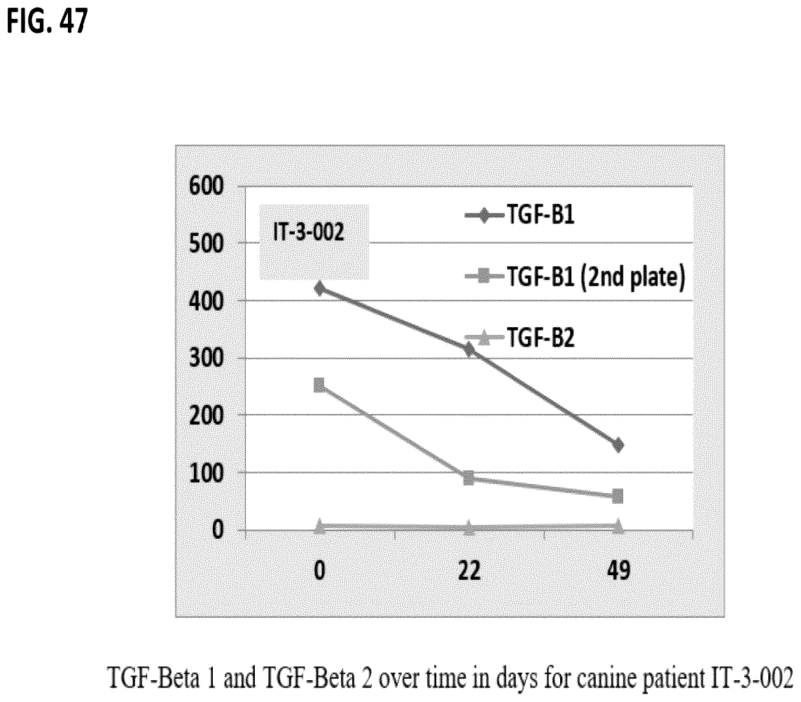

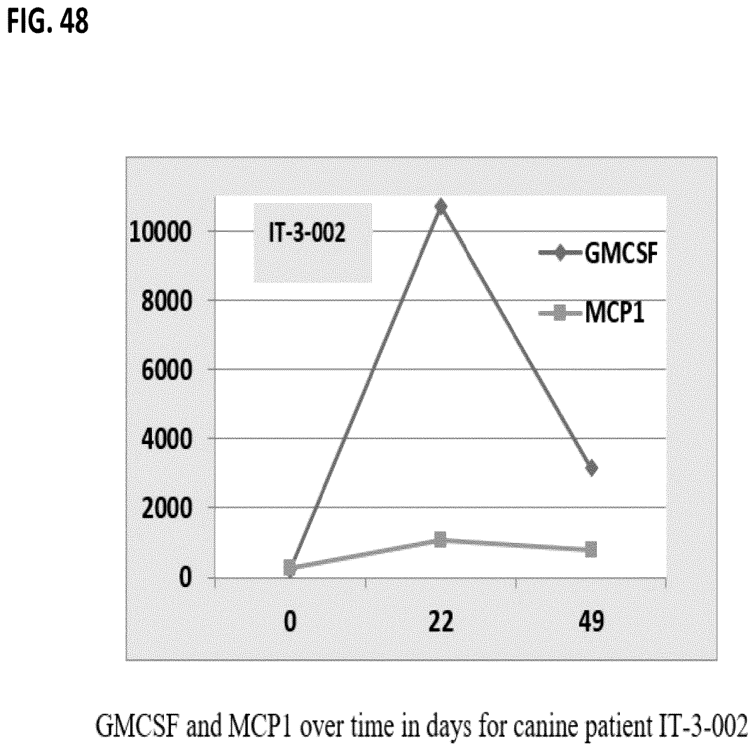

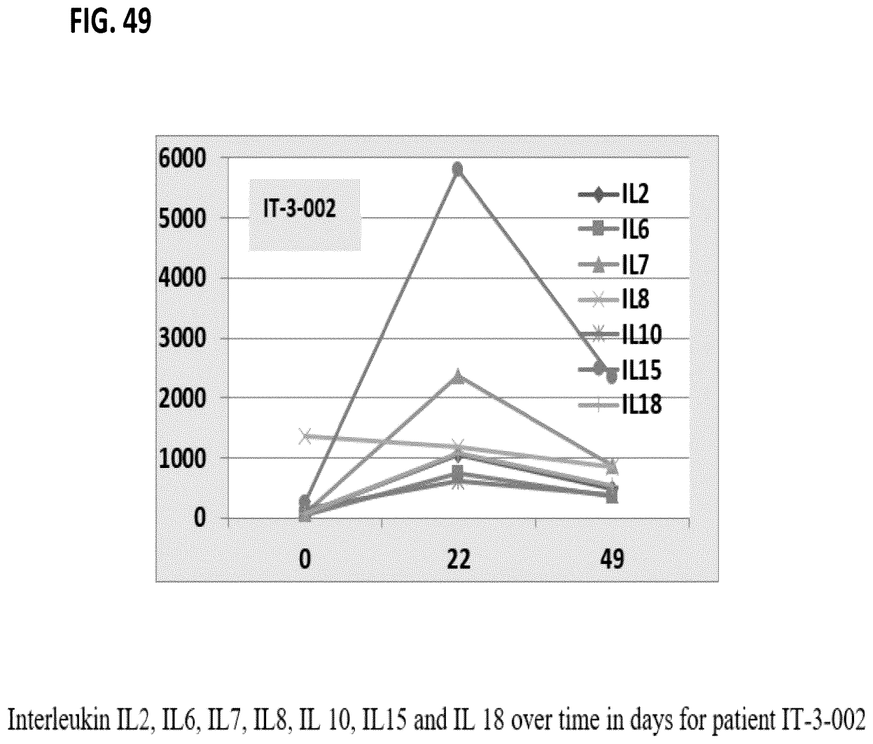

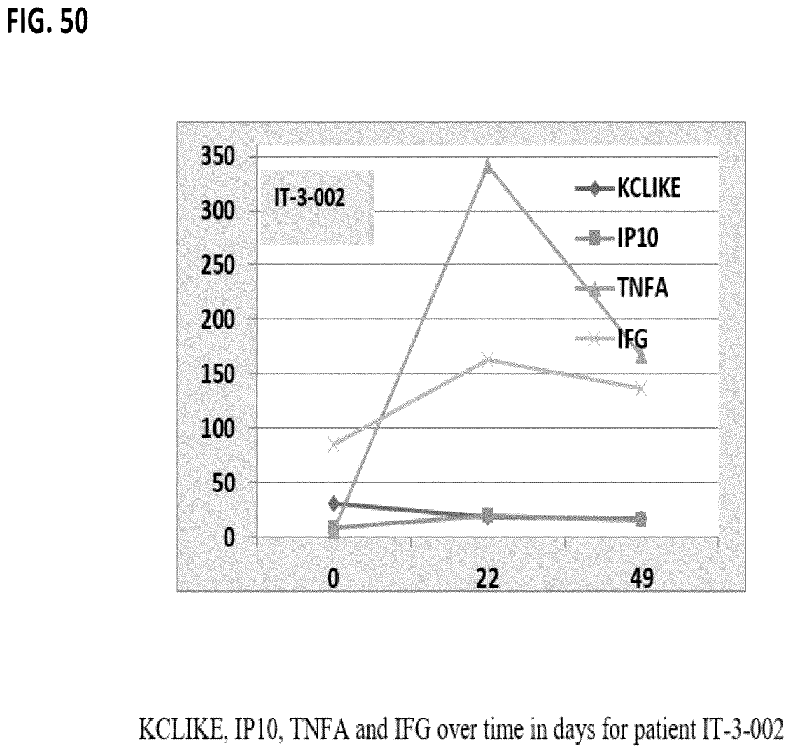

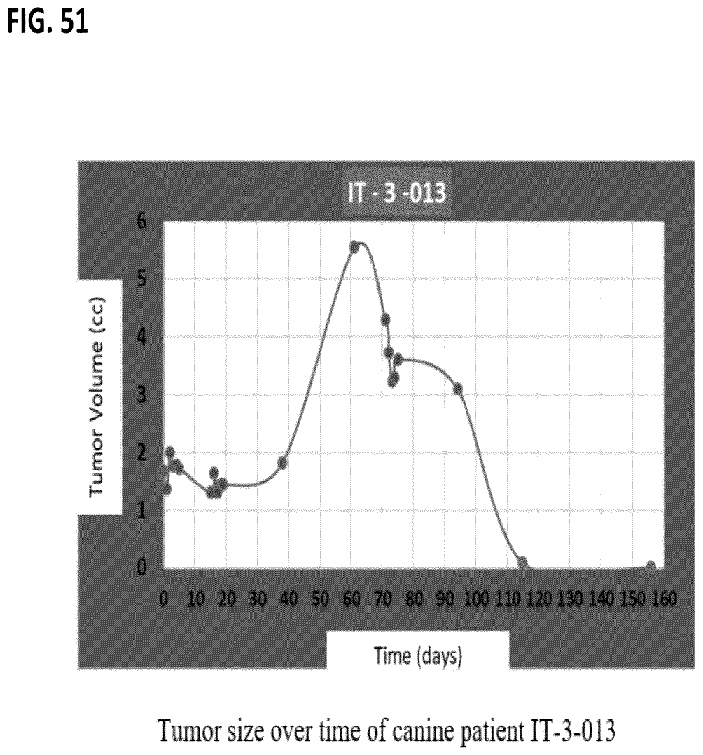

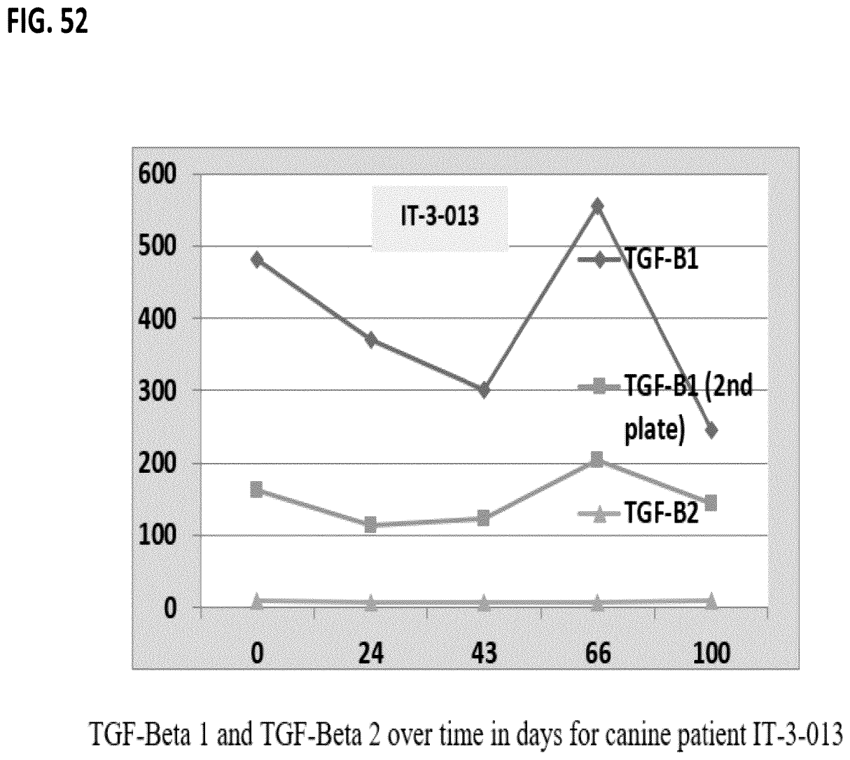

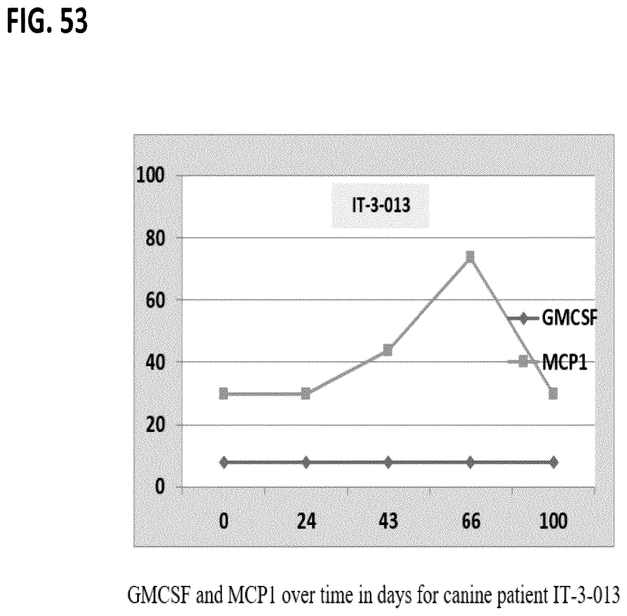

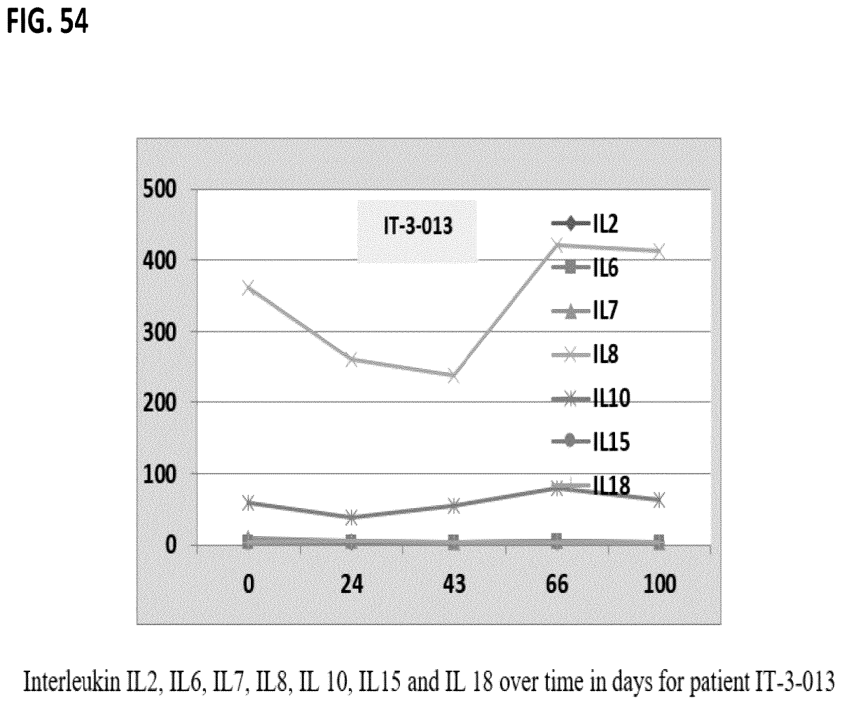

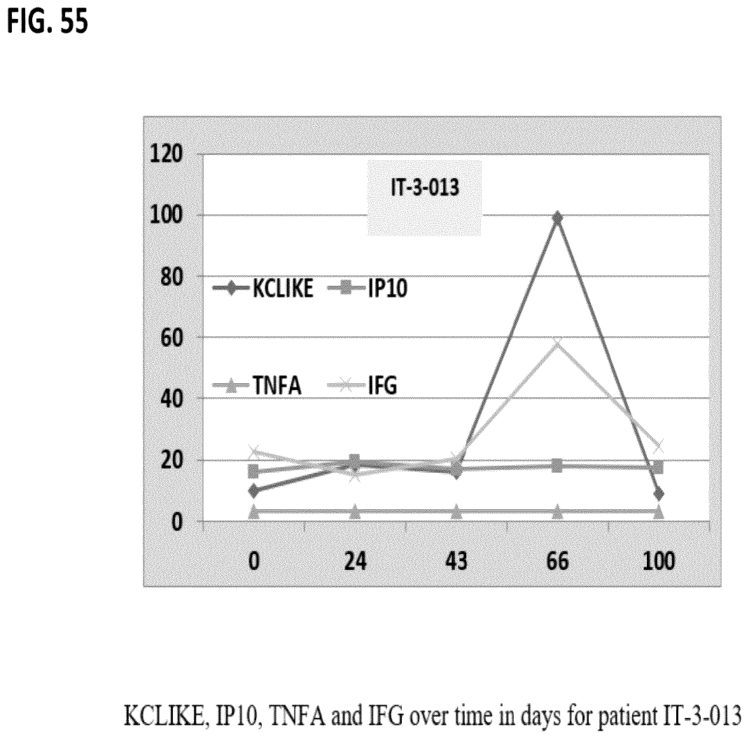

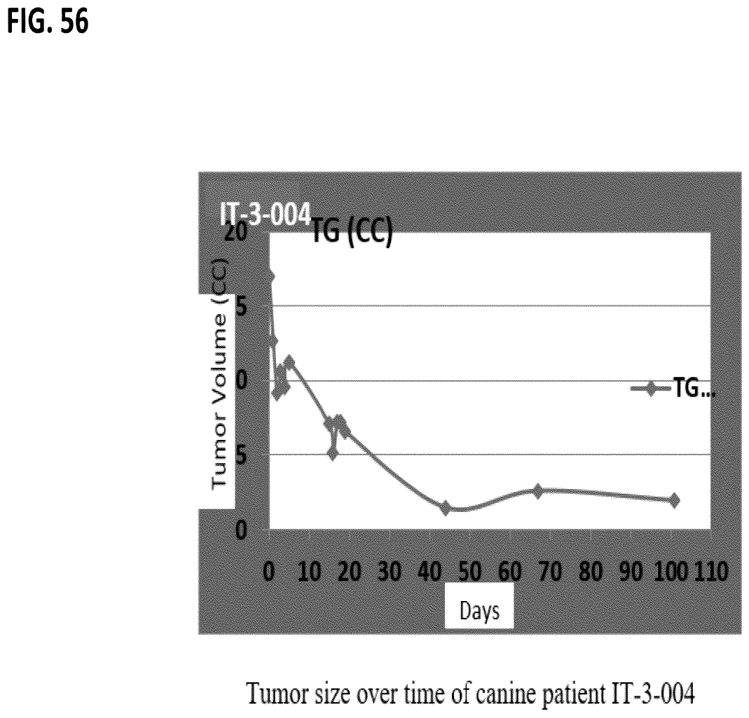

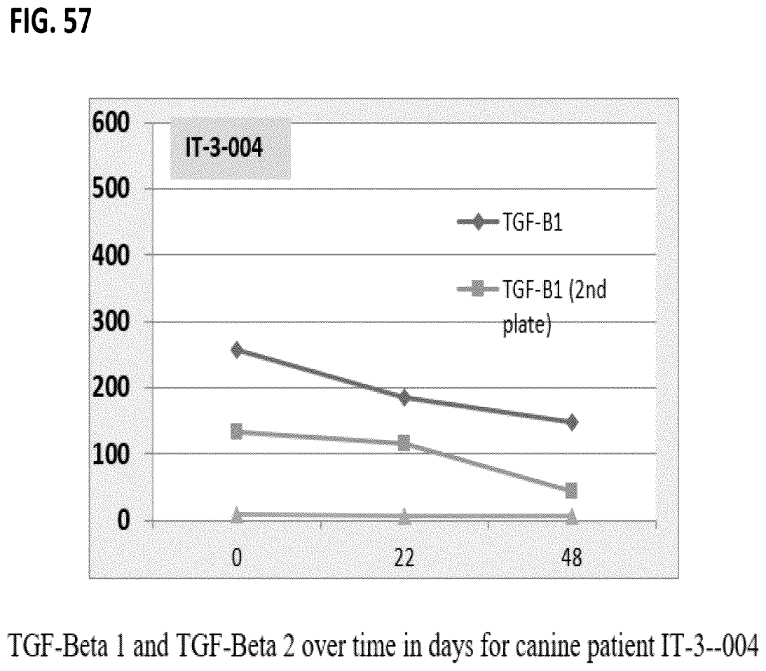

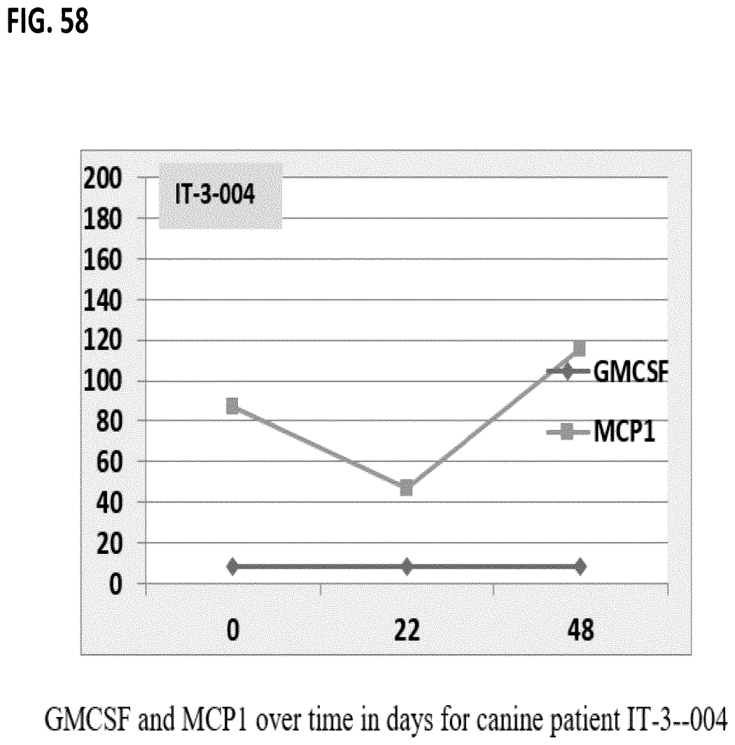

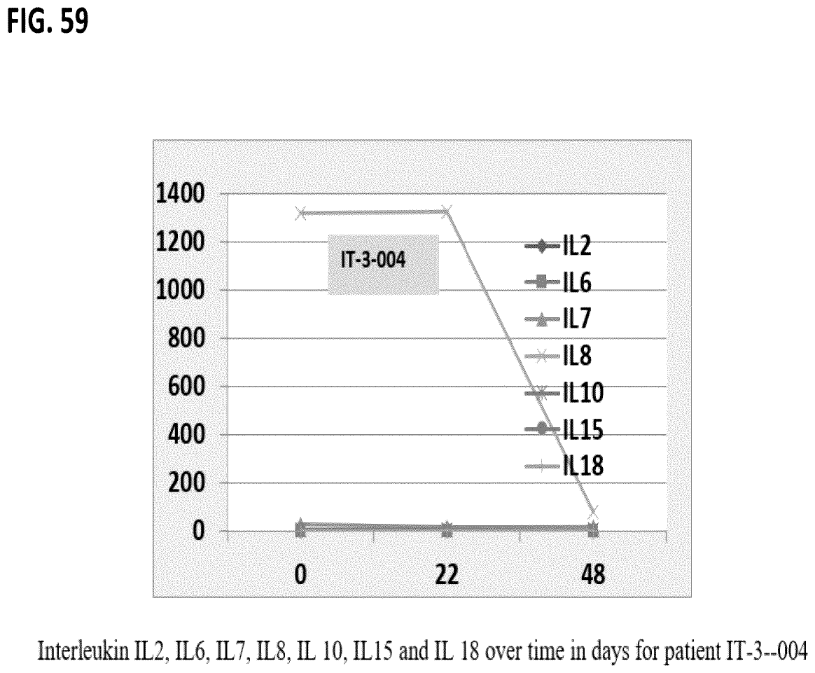

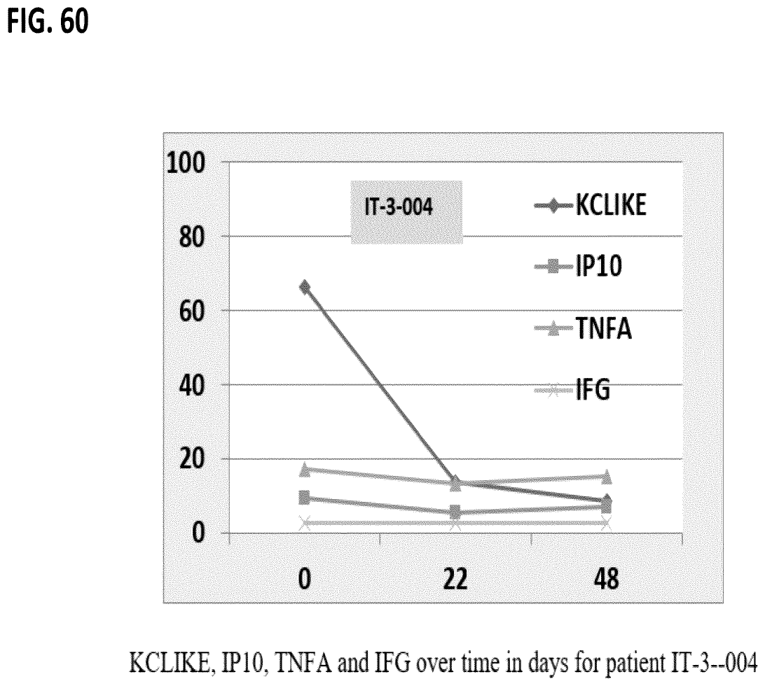

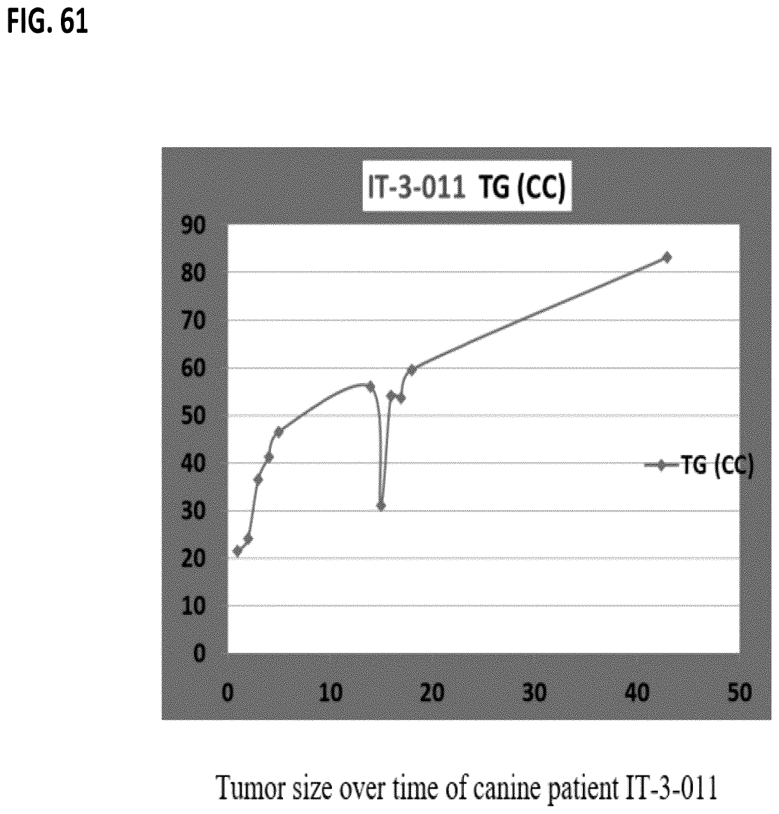

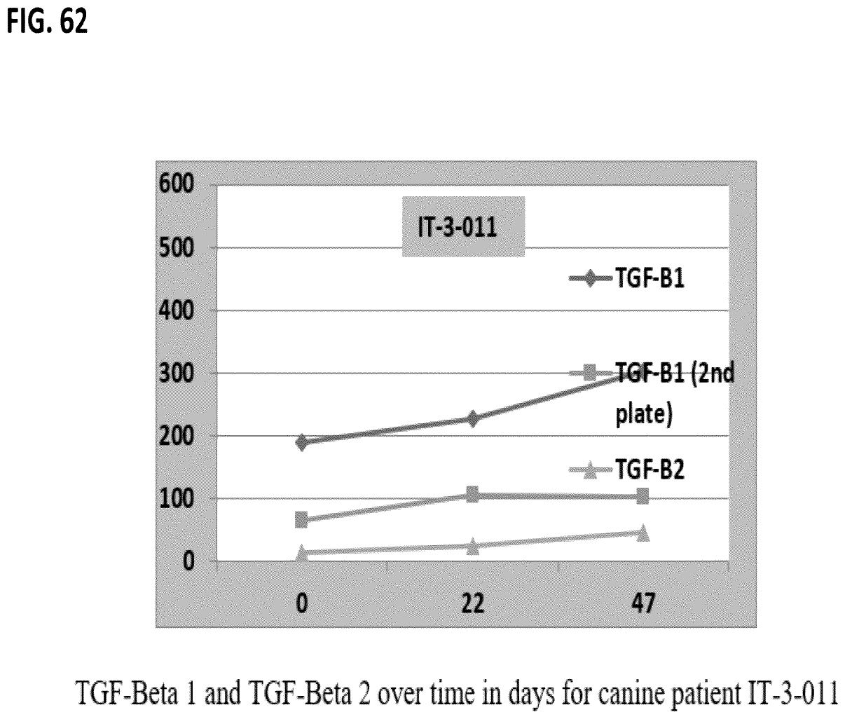

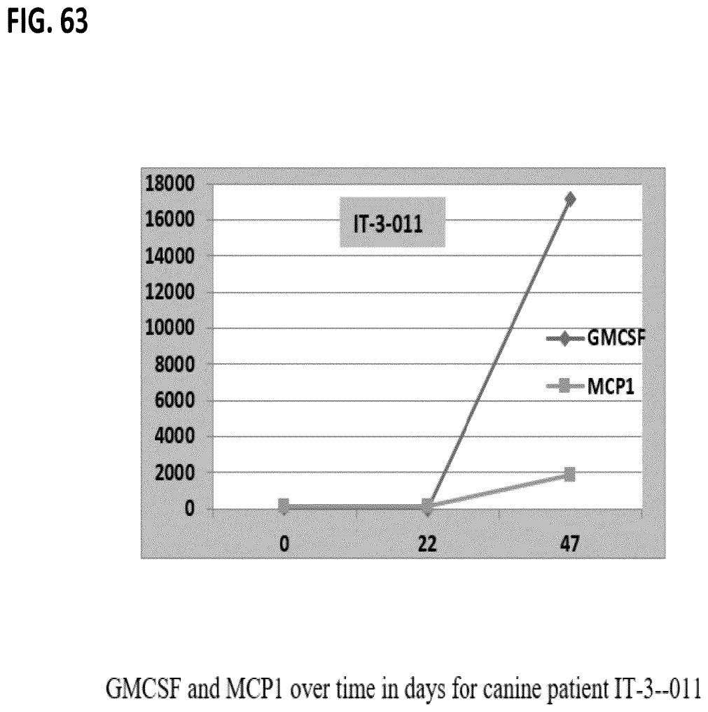

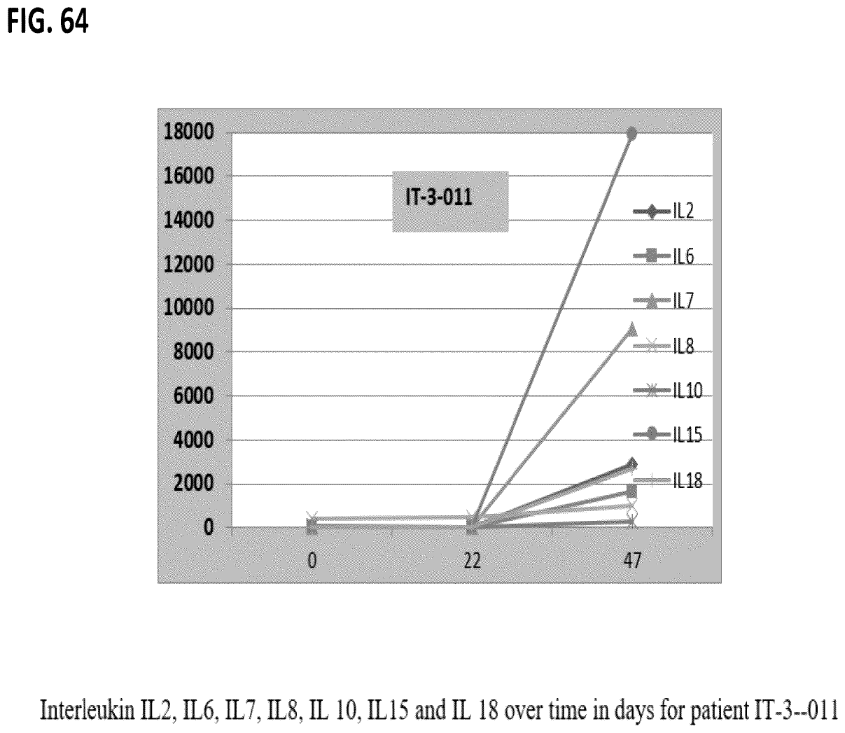

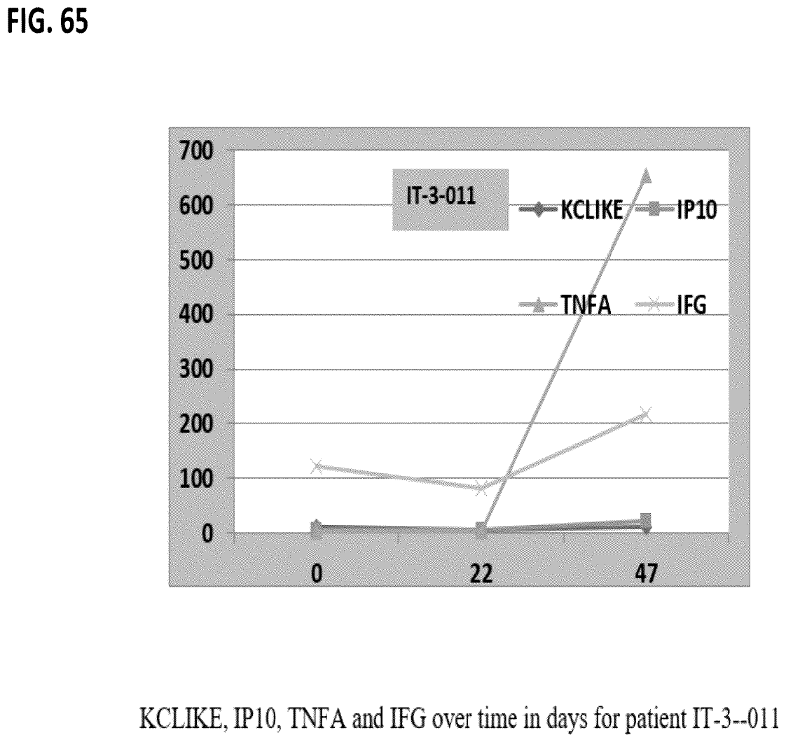

FIGS. 46-65 show the experimental data from canine trials where bioimarkers were measured and tracked with cancer progression/regression.

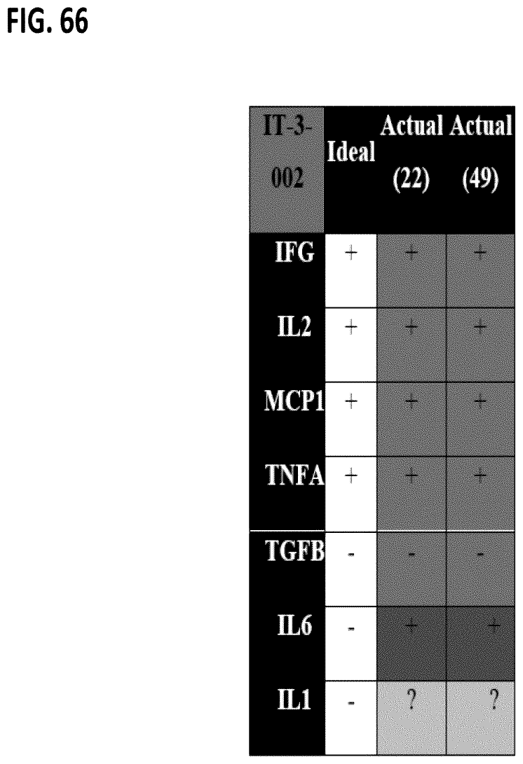

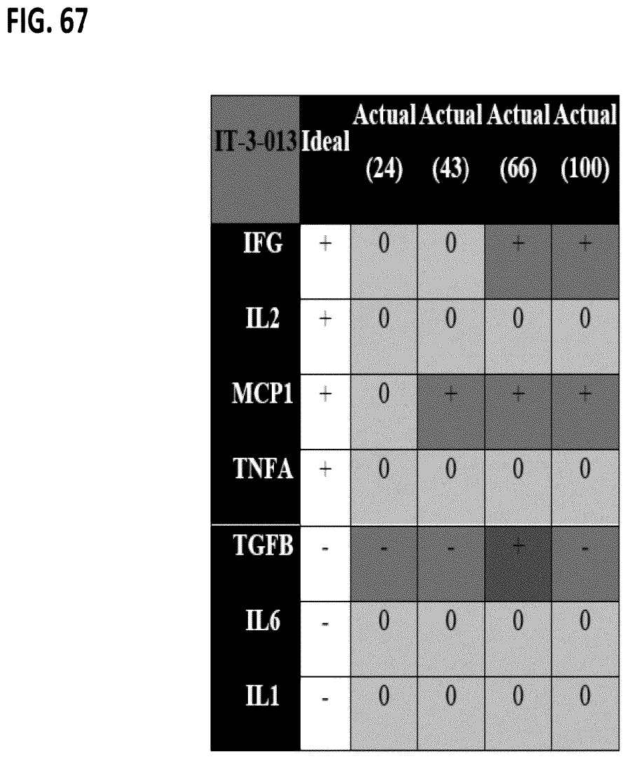

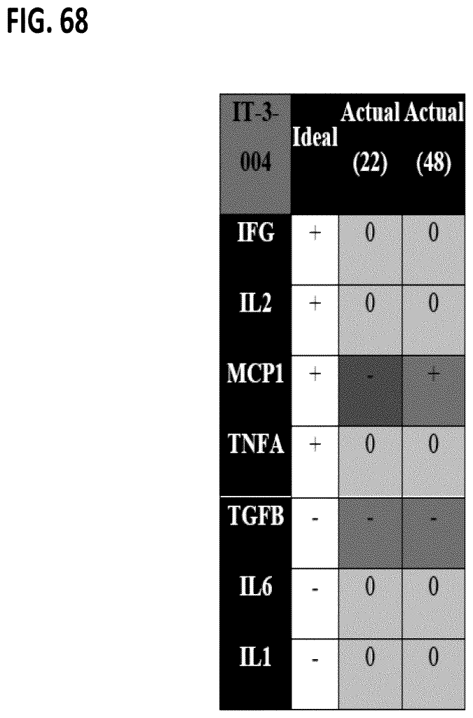

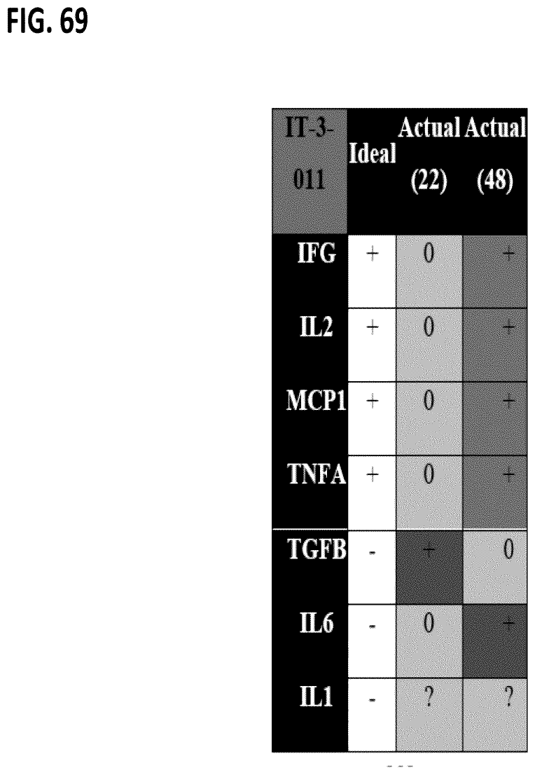

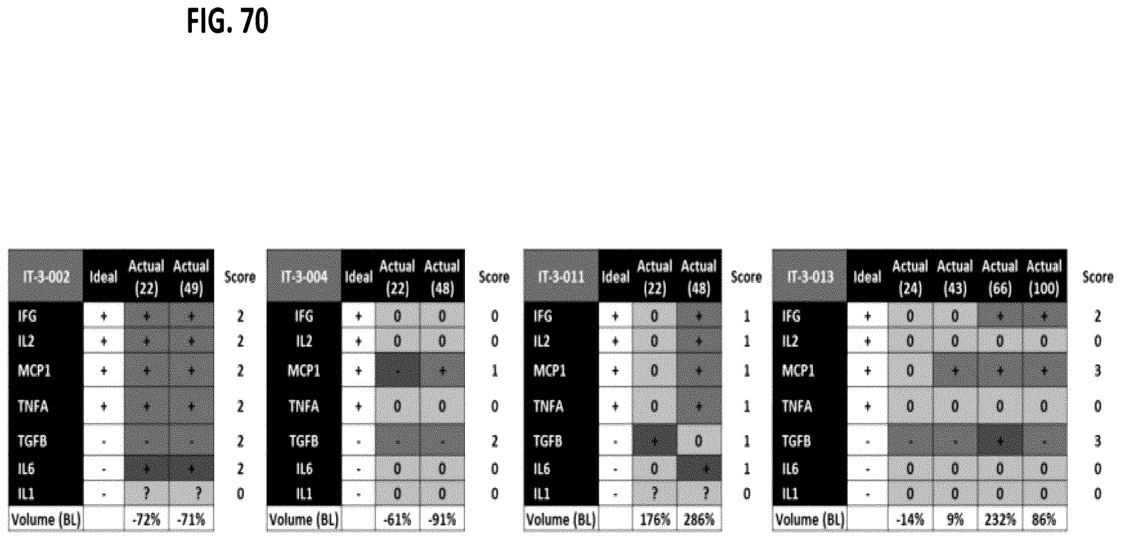

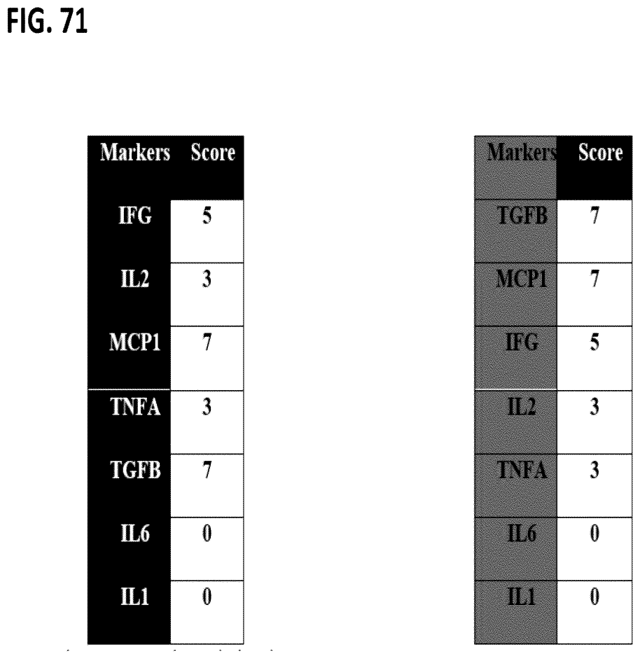

FIGS. 66-71 show the statistical analysis on the data. A plus (+) sign is assigned when a biomarker is behaving as It should be. A negative sign (−) is assigned when a biomarker is behaving opposite to how it should be. The number of positive signs is then tabulated for the patient.

To further resolve the data, each behavior aligned with the ideal response is assigned one point. The biomarkers are then ranked by order of importance. In one embodiment of the invention, the most important (ones with the highest correlation) biomarkers (TGF beta and MCP1) can be used to treat a patient using a closed loop feedback using intrinsic biomarkers behavior.

In one aspect of the invention, XPACT onco-therapy is delivered with specificity to the patient without resorting to averages from databases that do not reflect the patient's immune system characteristics.

The above noted biomarkers can be detected and assayed using various available technologies, including, but not limited to, standard available blood tests, as well as assays available from companies such as Ultivue, Inc. of Cambridge, Massachusetts or NanoString of Seattle, Washington.

The present invention includes, but is not limited to, the following embodiments:
Embodiment 1. A method for detecting and/or monitoring communication within a cell or between cells, comprising:
placing, in a region of interest within a living organism, a detector configured to monitor one or more signals emitted within a cell or between cells in a plurality of cells, wherein the region of interest is adjacent to the cell or plurality of cells undergoing a biological change;
collecting the one or more signals emitted from said cell or plurality of cells; and
identifying one or more characteristics associated with the one or more signals and correlating the one or more characteristics to the biological change.
Embodiment 2. The method of Embodiment 1, wherein the detector is a biophoton detector and the one or more signals are electromagnetic signals.
Embodiment 3. The method of Embodiment 2, wherein the electromagnetic signals are a wavelength or wavelength range.
Embodiment 4. The method of any one of Embodiments 1 to 3, wherein the biophoton detector is a fractal antenna comprising:
a repeating pattern of electrical conductors interconnected together whereby the fractal antenna has a plurality of different resonant frequencies; and
a substrate supporting the electrical conductors, wherein the substrate comprises a tube or cannula,
wherein the substrate and the electrical conductors are biocompatible, or are encased in a biocompatible material.
Embodiment 5. The method of any one of Embodiments 1 to 3, wherein the biophoton detector is a fractal antenna comprising:
a repeating pattern of electrical conductors interconnected together whereby the fractal antenna has a plurality of different resonant frequencies; and
a substrate supporting the electrical conductors, wherein the substrate comprises a wafer having a primary surface supporting the electrical conductors
wherein the substrate and the electrical conductors are biocompatible, or are encased in a biocompatible material.
Embodiment 6. A method for stimulating communication within a cell or between cells, comprising:
inserting into a region of interest within a living organism a signal emitter emitting a signal having a predetermined characteristic or combination of characteristics; and
causing the signal emitter to emit the signal having a predetermined characteristic or combination of characteristics;
wherein the predetermined characteristic or combination of characteristics are correlated to trigger a desired biological change within the cell or cells, said desired biological change being communicated within a cell or between cells to propagate the desired biological change.
Embodiment 7. The method of Embodiment 6, wherein the signal emitter is a biophoton emitter and the signal is emission at a predetermined wavelength or combination of wavelengths.
Embodiment 8. The method of Embodiment 7, wherein the biophoton emitter is one or more optical fibers configured to emit the predetermined wavelength or combination of wavelengths.
Embodiment 9. The method of one of Embodiments 7 or 8, wherein the biophoton emitter is a single optical fiber and the predetermined wavelength or wavelength range is a specified wavelength.
Embodiment 10. The method of one of Embodiments 7 or 8, wherein the biophoton emitter is a plurality of optical fibers and the predetermined wavelength or combination of wavelengths the combination of wavelengths.
Embodiment 11. The method of Embodiment 10, wherein the plurality of optical fibers are configured as an optical fiber bundle having dimensions permitting insertion into a living organism with minimal disruption of surrounding cells or tissues of the living organism.
Embodiment 12. The method of one of Embodiments 10 or 11, wherein the plurality of optical fibers are configured to each deliver a different wavelength of emission from one another.
Embodiment 13. The method of any one of Embodiments 10 to 12, wherein the plurality of optical fibers are configured to emit the combination of wavelengths simultaneously.
Embodiment 14. The method of one of Embodiments 10 or 11, wherein the plurality of optical fibers are configured such that each individual optical fiber in the plurality of optical fibers has its specified wavelength of emission.
Embodiment 15. The method of any one of Embodiments 10 to 14, wherein the plurality of optical fibers are configured such that the plurality of optical fibers will emit their specified wavelength of emission in a predetermined sequence.
Embodiment 16. The method of one of Embodiments 6 or 7, wherein the biophoton emitter is one or more energy modulation agents that can convert an applied penetrating energy into an emitted biophotonic energy of the predetermined wavelength or combination of wavelengths, wherein the method further comprises applying the applied penetrating energy from an applied energy source.
Embodiment 17. A method of treating a subject having a disease, disorder, or condition, comprising:
testing affected cells of a subject for the presence of one or more biomarkers correlated to treatment of the disease, disorder, or condition;
determining a wavelength or wavelength range of photon that triggers the affected cells to communicate and alter a level of the one or more biomarkers in a desirable manner associated with treatment of the disease, disorder, or condition; and
treating the affected cells of the subject in vivo with radiation at the determined wavelength or wavelength range to cause a desirable change in the level of the one or more biomarkers in vivo, thus treating the disease, disorder, or condition.
Embodiment 18. A method of treating a subject comprising:
initiating a change in a cellular environment of cells in a first region of a biological material inside the subject; and
due to a change in biological or chemical activity of the cells in the first region, inducing a biological change in a second region inside the subject; and
monitoring biophoton emission from the first region, into the second region, or from the second region.
Embodiment 19. The method of Embodiment 18, wherein the change in the cellular environment of the cells in the first region is stimulated by light stimulation of the cells in the first region.
Embodiment 20. The method of one of Embodiments 18 or 19, wherein the change in the cellular environment of the cells in the first region is tracked by the monitoring of the biomarkers in the cells in the first region.
Embodiment 21. The method of any one of Embodiments 18 to 20, wherein the induced biological change in the second region inside the subject is monitored by detecting the biomarkers in cells in the second region and correlating the biomarkers with the subject treatment.
Embodiment 22. The method of any one of Embodiments 18 to 21, wherein the induced biological change in the second region are local changes in proximity to the first site and/or global changes throughout the patient.
Embodiment 23. The method of any one of Embodiments 18 to 22, wherein the induced biological change in the second region occurs by cell-to-cell communication from the first site treated with stimulant light to a remote site not stimulated.
Embodiment 24. The method of any one of Embodiments 18 to 23, further comprising defining for the first region a region inside the subject proximate the second region.
Embodiment 25. The method of Embodiment 24, wherein the region inside the subject is formed of the subject's own tissue.
Embodiment 26. The method of Embodiment 24, wherein the region inside the subject is biological material implanted inside the subject.
Embodiment 27. The method of any one of Embodiments 18 to 26, further comprising defining for the first region a region inside the subject remote from the second region.
Embodiment 28. The method of Embodiment 27, wherein the region inside the subject is formed of the subject's own tissue.
Embodiment 29. The method of Embodiment 27, wherein the region inside the subject is biological material implanted inside the subject.
Embodiment 30. The method of any one of Embodiments 18 to 29, further comprising defining for the first region a region outside the subject coupled physically to the second region.
Embodiment 31. The method of any one of Embodiments 18 to 29, further comprising defining for the first region a region inside the subject overlapping the second region.
Embodiment 32. The method of any one of Embodiments 18 to 31, wherein providing comprises segregating the biological material of the first region from the second region by an artificial material.
Embodiment 33. The method of Embodiment 32, wherein the artificial material comprises a permeable material capable of transmission of chemical agents produced by the biological material from the first region into the second region.
Embodiment 34. The method of Embodiment 32, wherein the artificial material comprises a material capable of transmission of biophotons therethrough.
Embodiment 35. The method of Embodiment 32, wherein the artificial material comprises a material capable of transmission of sonic waves therethrough.
Embodiment 36. The method of Embodiment 32, wherein the artificial material comprises a material capable of transmission of ultraviolet light therethrough.
Embodiment 37. The method of Embodiment 32, wherein the artificial material comprises a material capable of transmission of infrared light therethrough.
Embodiment 38. The method of Embodiment 32, wherein the artificial material comprises a material capable of transmission of electrical signals therethrough.
Embodiment 39. The method of any one of Embodiments 18 to 38, wherein the first region and the second region are quantum entangled regions.
Embodiment 40. The method of any one of Embodiments 18 to 39, wherein the initiating a change in the first region comprises causing cell death of the biological material of the first region.
Embodiment 41. The method of any one of Embodiments 18 to 39, wherein the initiating a change in the first region comprises causing cell growth of the biological material of the first region.
Embodiment 42. The method of any one of Embodiments 18 to 39, wherein the initiating a change in the first region comprises imposing an electric field in the first region to promote ion pumping through cells in the biological material of the first region.
Embodiment 43. The method of any one of Embodiments 18 to 39, wherein the initiating a change in the first region comprises imposing an electric field in the first region to retard ion pumping through cells in the biological material of the first region.
Embodiment 44. The method of any one of Embodiments 18 to 39, wherein the initiating a change in the first region comprises changing a rate of transport of reagents through cell membranes cells in the biological material of the first region.
Embodiment 45. The method of Embodiment 44, wherein changing a rate of transport comprises changing a probability of tunneling of the reagents through cell membranes.
Embodiment 46. The method of Embodiment 45, wherein the changing a probability of tunneling comprises applying an electric field to promote or retard transmission of the reagents through the cell membranes in the biological material of the first region.
Embodiment 47. The method of one of Embodiments 45 or 46, wherein the changing a probability of tunneling comprises applying a photon flux to the reagents to increase an energy of the reagents.
Embodiment 48. The method of any one of Embodiments 45 to 47, wherein the changing a probability of tunneling comprises applying a drug which thickens the cell membranes.
Embodiment 49. The method of any one of Embodiments 45 to 48, wherein changing a probability of tunneling comprises applying a drug which dilates or constricts pores in the cell membranes.
Embodiment 50. The method of Embodiment 48, wherein the drug is isolated only to the first region so that toxicity of the drug does not affect the subject.
Embodiment 51. The method of Embodiment 49, wherein the drug is isolated only to the first region so that toxicity of the drug does not affect the subject.
Embodiment 52. The method of any one of Embodiments 18 to 39, wherein the initiating a change in the first region comprises changing a rate of enzymatic reactions occurring in the biological material.
Embodiment 53. The method of any one of Embodiments 18 to 39, wherein the initiating a change in the first region comprises changing a rate of catalysis reactions occurring in the biological material.
Embodiment 54. The method of any one of Embodiments 18 to 39, wherein the initiating a change in the first region comprises changing a rate of photosynthesis occurring in the biological material.
Embodiment 55. The method of any one of Embodiments 18 to 39, wherein the initiating a change in the first region comprises changing genomics of the biological material in the first region.
Embodiment 56. The method of Embodiment 55, wherein the changing genomics in the first region induces the therapeutic change in the second region.
Embodiment 57. The method of any one of Embodiments 18 to 56, further comprising coupling to the second region via interactions of DNA molecules along a pathway from the first region to the second region.
Embodiment 58. The method of Embodiment 57, wherein the coupling comprises having the pathway comprise signaling DNA.
Embodiment 59. The method of one of Embodiments 57 or 58, wherein the coupling comprises transporting charge along the signaling DNA.
Embodiment 60. The method of any one of Embodiments 18 to 39, wherein the initiating a change in the first region comprises removing a protein that normally binds to signaling DNA in the biological material of the first region.
Embodiment 61. The method of any one of Embodiments 18 to 60, further comprising:
surgically defining the first region from a diseased organ in the subject;
applying a treatment to the first region to promote cell death; and
thereby inducing cell death as the biological change in the second region of the subject.
Embodiment 62. The method of Embodiment 61, wherein applying a treatment comprises:
selectively treating the surgically defined first region to induce cell death.
Embodiment 63. The method of Embodiment 62, wherein the selectively treating comprises chemically inducing cell death in the surgically defined first region.
Embodiment 64. The method of Embodiment 62, wherein the selectively treating comprises inducing cell death in the surgically defined first region by radiation.
Embodiment 65. The method of Embodiment 64, wherein the radiation is ultraviolet light.
Embodiment 66. The method of Embodiment 64, wherein the radiation is x-rays, gamma rays, protons, or other high energy photon or particle sources.
Embodiment 67. The method of any one of Embodiments 18 to 66, wherein the biological change in the second region comprises a change in neuron activity.
Embodiment 68. The method of Embodiment 67, wherein the change in neuron activity comprises stimulation and/or control of neural communication.
Embodiment 69. The method of any one of Embodiments 18 to 68, further comprising monitoring in the first region or the second region biomarkers indicative a change in cell function inside a living cell.
Embodiment 70. The method of any one of Embodiments 18 to 69, further comprising monitoring in the first region or the second region biomarkers indicative a change in cell function inside a living cell exposed to a stimulant light.
Embodiment 71. The method of any one of Embodiments 18 to 70, further comprising monitoring in the first region or the second region biomarkers indicative a change in cell function inside a living cell not exposed to a stimulant light but otherwise coupled to a living cell exposed to the stimulant light.
Embodiment 72. The method of any one of Embodiments 18 to 71, further comprising correlating cell function with biomarker production.
Embodiment 73. The method of any one of Embodiments 18 to 72, further comprising correlating cell function with both biomarker production and stimulant light exposure.
Embodiment 74. The method of any one of Embodiments 18 to 73, further comprising providing a stimulant light treatment based on measured biomarker production occurring inside a living cell exposed to a stimulant light.
Embodiment 75. The method of any one of Embodiments 18 to 74, further comprising providing a subject treatment based on measured biomarker production occurring inside a living cell.
Embodiment 76. The method of any one of Embodiments 18 to 75, further comprising providing a subject treatment based on measured biomarker production occurring inside a living cell exposed to a stimulant light.
Embodiment 77. The method of any one of Embodiments 18 to 76, further comprising generating and correlating a response at a non-treatment site comprising the second sited in a patient in response to a stimulant light treatment at another site.
Embodiment 78. A method of treating a subject comprising:
initiating a change in a cellular environment of cells in a first region of a biological material inside the subject; and
due to a change in biological or chemical activity of the cells in the first region, inducing a biological change in a second region inside the subject by enhancing coupling of the first region to the second region.
Embodiment 79. The method of Embodiment 78, wherein the coupling is enhanced by an applied magnetic field extending from the first region to the second region.
Embodiment 80. The method of one of Embodiments 78 or 79, wherein the coupling is enhanced by an applied electric field extending from the first region to the second region.
Embodiment 81. The method of any one of Embodiments 78 to 80, wherein the coupling is enhanced by a light pipe extending from the first region to the second region.
Embodiment 82. The method of any one of Embodiments 78 to 81, wherein the coupling is enhanced by an acoustic waveguide extending from the first region to the second region.
Embodiment 83. The method of any one of Embodiments 78 to 82, wherein the coupling is enhanced by growth of nanotubes connecting cells from the first region to the second region.
Embodiment 84. The method of any one of Embodiments 78 to 83, wherein the coupling is enhanced by chemical transport of biomarkers from the first region to the second region.
Embodiment 85. A fractal antenna comprising:
a repeating pattern of electrical conductors interconnected together whereby the fractal antenna has a plurality of different resonant frequencies; and
a substrate supporting the electrical conductors,
wherein the substrate and the electrical conductors are biocompatible, or are encased in a biocompatible material.
Embodiment 86. The antenna of Embodiment 85, wherein
the substrate comprises a tube or a cannula, and
the electrical conductors are disposed against the tube or the cannula.
Embodiment 87. The antenna of Embodiment 86, wherein the tube or the cannula is flexible.
Embodiment 88. The antenna of Embodiment 85, wherein the substrate comprises a wafer having a primary surface supporting the electrical conductors.
Embodiment 89. The antenna of Embodiment 88, wherein the wafer is flexible.
Embodiment 90. The antenna of any one of Embodiments 85 to 89, wherein the electrical conductors are connected to a detector for measuring voltages induced on the electrical conductors.
Embodiment 91. The antenna of any one of Embodiments 85 to 90, wherein the substrate comprises a polymer or plastic.
Embodiment 92. The antenna of any one of Embodiments 85 to 90, wherein the substrate comprises a biological material.
Embodiment 93. The antenna of Embodiment 92, wherein the biological material is taken from a biopsy of a subject and disposed in vicinity of the electrical conductors.
Embodiment 94. The antenna of Embodiment 93, wherein, in response to a change in biological activity of the biological material, the electrical conductors sense a voltage indicative of biophoton emission from the biological material.
Embodiment 95. The antenna of Embodiment 92, wherein the biological material is of a subject.
Embodiment 96. The antenna of Embodiment 95, wherein, in response to a clinical treatment of the biological material, a change in biological activity of the biological material produces biophoton emission from the biological material, which is detected by the electrical conductors.
Embodiment 97. The antenna of Embodiment 96, wherein the clinical treatment comprises in vivo photon stimulation of the biological material.
Embodiment 98. The antenna of one of Embodiments 96 or 97, wherein the clinical treatment comprises in vivo photon stimulation of a photoactive drug in the biological material.
Embodiment 99. The antenna of any one of Embodiments 96 to 98, wherein the clinical treatment comprises cell to cell communication to the biological material.
Embodiment 100. The antenna of any one of Embodiments 96 to 99, wherein the biophoton emission detected from the biological material is used as feedback for the clinical treatment.
Embodiment 101. The antenna of any one of Embodiments 96 to 100, wherein the substrate comprises an artificially grown organism.
Embodiment 102. The antenna of Embodiment 101, wherein biophoton emission from the artificially grown organism is coupled to a treatment site in a subject.
Embodiment 103. The antenna of Embodiment 101, wherein the biophoton emission is detected by the electrical conductors and used as feedback for treating the treatment site.
Embodiment 104. The antenna of any one of Embodiments 101 to 103, wherein the electrical conductors are disposed in vivo nearby a treatment site to measure biophoton emission from the treatment site.
Embodiment 105. The antenna of any one of Embodiments 101 to 104, wherein the electrical conductors are disposed in vivo nearby an untreated site to monitor biophoton emission from a treated site to the untreated site.
Embodiment 106. A method for converting mitochondrial energy production in a patient in need thereof from a fermentation based process to an oxidative phosphorylation (Ox-Phos) based process, comprising:
monitoring mitochondria in cancer cells for light emissions and/or chemical signals produced during the fermentation process;
monitoring mitochondria in healthy cells for light emissions and/or chemical signals during the Ox-Phos process; in order to distinguish the cell-to-cell communication signals emitted by cancer cells and healthy cells;
imparting an external energy at the cellular level either directly using a fiber optic or indirectly using an energy modulation agent having an emission to trigger return of the cancer cells to an Ox-Phos process and monitoring the mitochondrial energy production signals at different time points; and
changing the frequency, wavelength, or both of the imparted energy until the signals produced during the mitochondrial energy production are representative of a healthy Ox-Phos energy production process.
Embodiment 107. The method of Embodiment 106, wherein the external energy is applied indirectly using an energy modulation agent.
Embodiment 108. The method of Embodiment 107, wherein the energy modulation agent is a downconverter.
Embodiment 109. The method of Embodiment 107, wherein the energy modulation agent is an upconverter.
Embodiment 110. The method of Embodiment 106, wherein the external energy is applied directly using a fiber optic.

Numerous modifications and variations of the invention are possible in light of the above teachings. It is therefore to be understood that within the scope of the appended claims, the invention may be practiced otherwise than as specifically described herein.

1. A method for detecting and/or monitoring communication within a cell or between cells, comprising:
placing, in a region of interest within a living organism, a detector configured to monitor one or more signals emitted within a cell or between cells in a plurality of cells, wherein the region of interest is adjacent to the cell or plurality of cells undergoing a biological change;
collecting the one or more signals emitted from said cell or plurality of cells; and
identifying one or more characteristics associated with the one or more signals and correlating the one or more characteristics to the biological change.

2. The method of claim 1, wherein the detector is a biophoton detector and the one or more signals are electromagnetic signals.

3. The method of claim 2, wherein the electromagnetic signals are a wavelength or wavelength range.

4. The method of claim 2, wherein the biophoton detector is a fractal antenna comprising:
a repeating pattern of electrical conductors interconnected together whereby the fractal antenna has a plurality of different resonant frequencies; and
a substrate supporting the electrical conductors, wherein the substrate comprises a tube or cannula,
wherein the substrate and the electrical conductors are biocompatible, or are encased in a biocompatible material.

5. The method of claim 2, wherein the biophoton detector is a fractal antenna comprising:
a repeating pattern of electrical conductors interconnected together whereby the fractal antenna has a plurality of different resonant frequencies; and
a substrate supporting the electrical conductors, wherein the substrate comprises a wafer having a primary surface supporting the electrical conductors
wherein the substrate and the electrical conductors are biocompatible, or are encased in a biocompatible material.

6. A method for stimulating communication within a cell or between cells, comprising:
inserting into a region of interest within a living organism a signal emitter emitting a signal having a predetermined characteristic or combination of characteristics; and
causing the signal emitter to emit the signal having a predetermined characteristic or combination of characteristics;
wherein the predetermined characteristic or combination of characteristics are correlated to trigger a desired biological change within the cell or cells, said desired biological change being communicated within a cell or between cells to propagate the desired biological change.

7. The method of claim 6, wherein the signal emitter is a biophoton emitter and the signal is emission at a predetermined wavelength or combination of wavelengths.

8. The method of claim 7, wherein the biophoton emitter is one or more optical fibers configured to emit the predetermined wavelength or combination of wavelengths.

9. The method of claim 8, wherein the biophoton emitter is a single optical fiber and the predetermined wavelength or wavelength range is a specified wavelength.

10. The method of claim 8, wherein the biophoton emitter is a plurality of optical fibers and the predetermined wavelength or combination of wavelengths the combination of wavelengths.

11. The method of claim 10, wherein the plurality of optical fibers are configured as an optical fiber bundle having dimensions permitting insertion into a living organism with minimal disruption of surrounding cells or tissues of the living organism.

12. The method of claim 10, wherein the plurality of optical fibers are configured to each deliver a different wavelength of emission from one another.

13. The method of claim 10, wherein the plurality of optical fibers are configured to emit the combination of wavelengths simultaneously.

14. The method of claim 10, wherein the plurality of optical fibers are configured such that each individual optical fiber in the plurality of optical fibers has its specified wavelength of emission.

15. The method of claim 14, wherein the plurality of optical fibers are configured such that the plurality of optical fibers will emit their specified wavelength of emission in a predetermined sequence.

16. The method of claim 7, wherein the biophoton emitter is one or more energy modulation agents that can convert an applied penetrating energy into an emitted biophotonic energy of the predetermined wavelength or combination of wavelengths, wherein the method further comprises applying the applied penetrating energy from an applied energy source.

17. A method of treating a subject having a disease, disorder, or condition, comprising:
testing affected cells of a subject for the presence of one or more biomarkers correlated to treatment of the disease, disorder, or condition;
determining a wavelength or wavelength range of photon that triggers the affected cells to communicate and alter a level of the one or more biomarkers in a desirable manner associated with treatment of the disease, disorder, or condition; and
treating the affected cells of the subject in vivo with radiation at the determined wavelength or wavelength range to cause a desirable change in the level of the one or more biomarkers in vivo, thus treating the disease, disorder, or condition.

18. A method of treating a subject comprising:
initiating a change in a cellular environment of cells in a first region of a biological material inside the subject; and
due to a change in biological or chemical activity of the cells in the first region, inducing a biological change in a second region inside the subject; and
monitoring biophoton emission from the first region, into the second region, or from the second region.

19. The method of claim 18, wherein the change in the cellular environment of the cells in the first region is stimulated by light stimulation of the cells in the first region.

20. The method of claim 18, wherein the change in the cellular environment of the cells in the first region is tracked by the monitoring of the biomarkers in the cells in the first region.

21. The method of claim 18, wherein the induced biological change in the second region inside the subject is monitored by detecting the biomarkers in cells in the second region and correlating the biomarkers with the subject treatment.

22. The method of claim 18, wherein the induced biological change in the second region are local changes in proximity to the first site and/or global changes throughout the patient.

23. The method of claim 18, wherein the induced biological change in the second region occurs by cell-to-cell communication from the first site treated with stimulant light to a remote site not stimulated.

24. The method of claim 18, further comprising defining for the first region a region inside the subject proximate the second region.

25. The method of claim 24, wherein the region inside the subject is formed of the subject's own tissue.

26. The method of claim 24, wherein the region inside the subject is biological material implanted inside the subject.

27. The method of claim 18, further comprising defining for the first region a region inside the subject remote from the second region.

28. The method of claim 27, wherein the region inside the subject is formed of the subject's own tissue.

29. The method of claim 27, wherein the region inside the subject is biological material implanted inside the subject.

30. The method of claim 18, further comprising defining for the first region a region outside the subject coupled physically to the second region.

31. The method of claim 18, further comprising defining for the first region a region inside the subject overlapping the second region.

32. The method of claim 18, wherein providing comprises segregating the biological material of the first region from the second region by an artificial material.

33. The method of claim 32, wherein the artificial material comprises a permeable material capable of transmission of chemical agents produced by the biological material from the first region into the second region.

34. The method of claim 32, wherein the artificial material comprises a material capable of transmission of biophotons therethrough.

35. The method of claim 32, wherein the artificial material comprises a material capable of transmission of sonic waves therethrough.

36. The method of claim 32, wherein the artificial material comprises a material capable of transmission of ultraviolet light therethrough.

37. The method of claim 32, wherein the artificial material comprises a material capable of transmission of infrared light therethrough.

38. The method of claim 32, wherein the artificial material comprises a material capable of transmission of electrical signals therethrough.

39. The method of claim 18, wherein the first region and the second region are quantum entangled regions.

40. The method of claim 18, wherein the initiating a change in the first region comprises causing cell death of the biological material of the first region.

41. The method of claim 18, wherein the initiating a change in the first region comprises causing cell growth of the biological material of the first region.

42. The method of claim 18, wherein the initiating a change in the first region comprises imposing an electric field in the first region to promote ion pumping through cells in the biological material of the first region.

43. The method of claim 18, wherein the initiating a change in the first region comprises imposing an electric field in the first region to retard ion pumping through cells in the biological material of the first region.

44. The method of claim 18, wherein the initiating a change in the first region comprises changing a rate of transport of reagents through cell membranes cells in the biological material of the first region.

45. The method of claim 44, wherein changing a rate of transport comprises changing a probability of tunneling of the reagents through cell membranes.

46. The method of claim 45, wherein the changing a probability of tunneling comprises applying an electric field to promote or retard transmission of the reagents through the cell membranes in the biological material of the first region.

47. The method of claim 45, wherein the changing a probability of tunneling comprises applying a photon flux to the reagents to increase an energy of the reagents.

48. The method of claim 45, wherein the changing a probability of tunneling comprises applying a drug which thickens the cell membranes.

49. The method of claim 45, wherein changing a probability of tunneling comprises applying a drug which dilates or constricts pores in the cell membranes.

50. The method of claim 49, wherein the drug is isolated only to the first region so that toxicity of the drug does not affect the subject.

51. The method of claim 49, wherein the drug is isolated only to the first region so that toxicity of the drug does not affect the subject.

52. The method of claim 18, wherein the initiating a change in the first region comprises changing a rate of enzymatic reactions occurring in the biological material.

53. The method of claim 18, wherein the initiating a change in the first region comprises changing a rate of catalysis reactions occurring in the biological material.

54. The method of claim 18, wherein the initiating a change in the first region comprises changing a rate of photosynthesis occurring in the biological material.

55. The method of claim 18, wherein the initiating a change in the first region comprises changing genomics of the biological material in the first region.

56. The method of claim 55, wherein the changing genomics in the first region induces the therapeutic change in the second region.

57. The method of claim 18, further comprising coupling to the second region via interactions of DNA molecules along a pathway from the first region to the second region.

58. The method of claim 57, wherein the coupling comprises having the pathway comprise signaling DNA.

59. The method of claim 57, wherein the coupling comprises transporting charge along the signaling DNA.

60. The method of claim 18, wherein the initiating a change in the first region comprises removing a protein that normally binds to signaling DNA in the biological material of the first region.

61. The method of claim 18, further comprising:
surgically defining the first region from a diseased organ in the subject;
applying a treatment to the first region to promote cell death; and
thereby inducing cell death as the biological change in the second region of the subject.

62. The method of claim 61, wherein applying a treatment comprises:
selectively treating the surgically defined first region to induce cell death.

63. The method of claim 61, wherein the selectively treating comprises chemically inducing cell death in the surgically defined first region.

64. The method of claim 61, wherein the selectively treating comprises inducing cell death in the surgically defined first region by radiation.

65. The method of claim 64, wherein the radiation is ultraviolet light.

66. The method of claim 64, wherein the radiation is x-rays, gamma rays, protons, or other high energy photon or particle sources.

67. The method of claim 18, wherein the biological change in the second region comprises a change in neuron activity.

68. The method of claim 67, wherein the change in neuron activity comprises stimulation and/or control of neural communication.

69. The method of claim 18, further comprising monitoring in the first region or the second region biomarkers indicative a change in cell function inside a living cell.

70. The method of claim 18, further comprising monitoring in the first region or the second region biomarkers indicative a change in cell function inside a living cell exposed to a stimulant light.

71. The method of claim 18, further comprising monitoring in the first region or the second region biomarkers indicative a change in cell function inside a living cell not exposed to a stimulant light but otherwise coupled to a living cell exposed to the stimulant light.

72. The method of claim 18, further comprising correlating cell function with biomarker production.

73. The method of claim 18, further comprising correlate cell function with both biomarker production and stimulant light exposure.

74. The method of claim 18, further comprising providing a stimulant light treatment based on measured biomarker production occurring inside a living cell exposed to a stimulant light.

75. The method of claim 18, further comprising providing a subject treatment based on measured biomarker production occurring inside a living cell.

76. The method of claim 18, further comprising providing a subject treatment based on measured biomarker production occurring inside a living cell exposed to a stimulant light.

77. The method of claim 18, further comprising generating and correlating a response at a non-treatment site comprising the second sited in a patient in response to a stimulant light treatment at another site.

78. A method of treating a subject comprising:
initiating a change in a cellular environment of cells in a first region of a biological material inside the subject; and
due to a change in biological or chemical activity of the cells in the first region, inducing a biological change in a second region inside the subject by enhancing coupling of the first region to the second region.

79. The method of claim 78, wherein the coupling is enhanced by an applied magnetic field extending from the first region to the second region.

80. The method of claim 78, wherein the coupling is enhanced by an applied electric field extending from the first region to the second region.

81. The method of claim 78, wherein the coupling is enhanced by a light pipe extending from the first region to the second region.

82. The method of claim 78, wherein the coupling is enhanced by an acoustic waveguide extending from the first region to the second region.

83. The method of claim 78, wherein the coupling is enhanced by growth of nanotubes connecting cells from the first region to the second region.

84. The method of claim 78, wherein the coupling is enhanced by chemical transport of biomarkers from the first region to the second region.

85. A fractal antenna comprising:
a repeating pattern of electrical conductors interconnected together whereby the fractal antenna has a plurality of different resonant frequencies; and
a substrate supporting the electrical conductors,
wherein the substrate and the electrical conductors are biocompatible, or are encased in a biocompatible material.

86. The antenna of claim 85, wherein
the substrate comprises a tube or a cannula, and
the electrical conductors are disposed against the tube or the cannula.

87. The antenna of claim 86, wherein the tube or the cannula is flexible.

88. The antenna of claim 85, wherein the substrate comprises a wafer having a primary surface supporting the electrical conductors.

89. The antenna of claim 88, wherein the wafer is flexible.

90. The antenna of claim 85, wherein the electrical conductors are connected to a detector for measuring voltages induced on the electrical conductors.

91. The antenna of claim 85, wherein the substrate comprises a polymer or plastic.

92. The antenna of claim 85, wherein the substrate comprises a biological material.

93. The antenna of claim 92, wherein the biological material is taken from a biopsy of a subject and disposed in vicinity of the electrical conductors.

94. The antenna of claim 93, wherein, in response to a change in biological activity of the biological material, the electrical conductors sense a voltage indicative of biophoton emission from the biological material.

95. The antenna of claim 92, wherein the biological material is of a subject.

96. The antenna of claim 95, wherein, in response to a clinical treatment of the biological material, a change in biological activity of the biological material produces biophoton emission from the biological material, which is detected by the electrical conductors.

97. The antenna of claim 96, wherein the clinical treatment comprises in vivo photon stimulation of the biological material.

98. The antenna of claim 96, wherein the clinical treatment comprises in vivo photon stimulation of a photoactive drug in the biological material.

99. The antenna of claim 96, wherein the clinical treatment comprises cell to cell communication to the biological material.

100. The antenna of claim 96, wherein the biophoton emission detected from the biological material is used as feedback for the clinical treatment.

101. The antenna of claim 96, wherein the substrate comprises an artificially grown organism.

102. The antenna of claim 101, wherein biophoton emission from the artificially grown organism is coupled to a treatment site in a subject.

103. The antenna of claim 101, wherein the biophoton emission is detected by the electrical conductors and used as feedback for treating the treatment site.

104. The antenna of claim 101, wherein the electrical conductors are disposed in vivo nearby a treatment site to measure biophoton emission from the treatment site.

105. The antenna of claim 101, wherein the electrical conductors are disposed in vivo nearby an untreated site to monitor biophoton emission from a treated site to the untreated site.

106. A method for converting mitochondrial energy production in a patient in need thereof from a fermentation based process to an oxidative phosphorylation (Ox-Phos) based process, comprising:
monitoring mitochondria in cancer cells for light emissions and/or chemical signals produced during the fermentation process;
monitoring mitochondria in healthy cells for light emissions and/or chemical signals during the Ox-Phos process; in order to distinguish the cell-to-cell communication signals emitted by cancer cells and healthy cells;
imparting an external energy at the cellular level either directly using a fiber optic or indirectly using an energy modulation agent having an emission to trigger return of the cancer cells to an Ox-Phos process and monitoring the mitochondrial energy production signals at different time points; and
changing the frequency, wavelength, or both of the imparted energy until the signals produced during the mitochondrial energy production are representative of a healthy Ox-Phos energy production process.

107. The method of claim 106, wherein the external energy is applied indirectly using an energy modulation agent.

108. The method of claim 107, wherein the energy modulation agent is a downconverter.

109. The method of claim 107, wherein the energy modulation agent is an upconverter.

110. The method of claim 106, wherein the external energy is applied directly using a fiber optic.
